(19) United States
(12) Patent Application Publication
(10) Pub. No.: US 2021/0380691 A1
(43) Pub. Date: Dec. 9, 2021

(54) MULTIFUNCTIONAL MOLECULES THAT BIND TO T CELLS AND USES THEREOF TO TREAT AUTOIMMUNE DISORDERS

(71) Applicant: Marengo Therapeutics, Inc., Cambridge, MA (US)

(72) Inventors: Andreas Loew, Boston, MA (US); Seng-Lai Tan, Sudbury, MA (US); Jonathan Hsu, Waltham, MA (US); Brian Edward Vash, Cambridge, MA (US); Stephanie J. Maiocco, Arlington, MA (US); Nidhi Malhotra, Boston, MA (US); Madan Katragadda, Acton, MA (US)

(21) Appl. No.: 17/402,318

(22) Filed: Aug. 13, 2021

Related U.S. Application Data
(63) Continuation of application No. PCT/US2020/019321, filed on Feb. 21, 2020.
(60) Provisional application No. 62/808,713, filed on Feb. 21, 2019.
(60) Provisional application No. 62/957,045, filed on Jan. 3, 2020.

(51) Int. Cl. C07K 16/28; G01N 33/574

(52) U.S. Cl. CPC C07K 16/2809; C07K 16/2803; G01N 33/574; C07K 2317/24; C07K 2317/92; C07K 2317/732; C07K 2317/734; C07K 2317/31

(57) ABSTRACT

Multifunctional molecules that include i) an antigen binding domain that binds to a TCR variable beta chain (TCRBV) antigen; and one, two or all of: (ii) an immune cell engager (e.g., chosen from an NK cell engager, a T cell engager, a B cell engager, a dendritic cell engager, or a macrophage cell engager); (iii) a cytokine molecule or cytokine inhibitor molecule; and/or (iv) a death receptor signal enhancer. Additionally, disclosed are nucleic acids encoding the same, methods of producing the aforesaid molecules, and methods of treating autoimmune diseases using the aforesaid molecules.

### RELATED APPLICATIONS

This application is a continuation of International Application No. PCT/US2020/019321, filed on Feb. 21, 2020, which claims the benefit of U.S. Provisional Application 62/808,713 filed on Feb. 21, 2019 and U.S. Provisional Application 62/957,045 filed on Jan. 3, 2020, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Feb. 19, 2020, is named 53676-734.301_SL.txt and is 1,519,578 bytes in size.

### BACKGROUND

T cell mediated antigen recognition depends on the interaction of the T cell receptor (TCR) with the antigen-major histocompatibility complex (MHC). The heterodimeric TCRs consist of a combination of α and β chains (αβ TCR) expressed by the majority of T cells, or γδ chains (γδ TCR) present only in about 1-5% of the T cells. A highly diverse TCR repertoire is a fundamental property of an effective immune system. However, the immune repertoire can change greatly with the onset and progression of diseases, such as cancer, autoimmune, inflammatory, and infectious diseases.

Autoimmunity may result from abnormal regulation of the immune system. This may be manifested by autoreactive TCR clones that attack a patient's own cells. There is a need for improved therapies for autoimmune diseases.

### SUMMARY OF THE INVENTION

The disclosure relates, inter alia, to novel multispecific or multifunctional molecules that include (i) an antigen binding domain that binds to a TCR variable beta chain (TCRBV) antigen on a T cell (e.g., a TCRBV antigen corresponding to a biased TCRBV clonotype); and one, two or all of: (ii) an immune cell engager (e.g., chosen from an NK cell engager, a T cell engager, a B cell engager, a dendritic cell engager, or a macrophage cell engager); (iii) a cytokine molecule; and/or (iv) a stromal modifying moiety. The terms “multispecific” or “multifunctional” are used interchangeably herein.

Without wishing to be bound by theory, a TCR bias may exist in autoimmune diseases. This bias may be associated with dominant autoreactive TCR clones responsible for disease or associated with symptoms. Re-balancing the TCR repertoire, e.g., by eliminating or depleting T cells comprising an autoreactive clonotype, may treat the associated autoimmune disease and/or reduce symptoms of the autoimmune disease. Accordingly, the multispecific or multifunctional molecules disclosed herein are expected to target (e.g., localize, bridge and/or activate) an immune cell (e.g., an immune effector cell chosen from an NK cell, a T cell, a B cell, a dendritic cell or a macrophage), at a target cell (e.g., a T cell comprising a biased TCRBV clonotype or comprising a TCRBV antigen corresponding to a biased TCRBV clonotype). Increasing the proximity and/or activity of the immune cell using the multispecific molecules described herein is expected to enhance an immune response against the target cell (e.g., the T cell comprising a TCRBV, e.g., TCRBV antigen (e.g., a TCRBV antigen corresponding to a biased TCRBV clonotype), thereby providing a more effective therapy (e.g., a more effective autoimmune disease therapy). Without being bound by theory, a targeted, localized immune response against the target cell (e.g., a T cell comprising a biased TCRBV clonotype, e.g., and not T cells not comprising the biased TCRBV clonotype) is believed to reduce the effects of systemic toxicity of the multispecific molecules described herein. A targeted immune response against the autoreactive T cell population that targets non-autoreactive T cells to a lesser degree (e.g., does not target non-autoreactive T cells) is believed to have fewer deleterious effects than systemic ablation of all T cells.

Accordingly, provided herein are, inter alia, multispecific molecules (e.g., multispecific or multifunctional antibody molecules) that include the aforesaid moieties, nucleic acids encoding the same, methods of producing the aforesaid molecules, and methods of treating autoimmune disease using the aforesaid molecules. Also provided herein are anti-TCRβV antibody molecules, nucleic acids encoding the same, methods of producing the aforesaid molecules, and methods of treating autoimmune disease using the anti-TCRβV antibody molecules.

Further provided are methods for depletion (e.g., in vivo depletion) of biased TCRBV clonotypes, e.g., in the context of autoimmune disease with a multispecific molecule or an anti-TCRβV antibody molecule. In some embodiments, the method involves identifying in a patient a clonal bias in TCRBV usage, e.g., associated with the autoreactive subpopulation, and responsive to this analysis administering a multifunctional molecule targeted to the TCRBV antigen corresponding to the biased TCRBV clonotype to decrease, e.g., eliminate, the clonal bias and promote, e.g., establish, a normal TCRBV distribution.

Accordingly, in one aspect, the disclosure features a multifunctional molecule, comprising:

(i) a first antigen binding domain that binds to, e.g., selectively binds to, T cell receptor variable beta (TCRBV), e.g., a TCRBV antigen, and
(ii) one, two, or all of:
(a) an immune cell engager chosen from an NK cell engager, a T cell engager, a B cell engager, a dendritic cell engager, or a macrophage cell engager;
(b) a cytokine molecule or cytokine inhibitor molecule; and
(c) a death receptor signal engager.

In some embodiments, first antigen binding domain comprises an anti-TCRβV antibody molecule, e.g., as described herein.

In another aspect, the disclosure features a nucleic acid molecule encoding a multifunctional molecule disclosed herein.

In another aspect, the disclosure features a vector, e.g., an expression vector, comprising the nucleic acid molecules disclosed herein.

In another aspect, the disclosure features a host cell comprising a nucleic acid molecule or vector disclosed herein.

In another aspect, the disclosure features a method of making, e.g., producing, a multifunctional molecule disclosed herein, comprising culturing a host cell disclosed herein under suitable conditions, e.g., conditions suitable for gene expression and/or homo- or heterodimerization.

In another aspect, the disclosure features a pharmaceutical composition comprising a multifunctional molecule disclosed herein.

In another aspect, the disclosure features a method of treating a TCR bias, comprising administering to a subject in need thereof a multifunctional molecule disclosed herein, wherein the multifunctional molecule is administered in an amount effective to treat the TCR bias.

In another aspect, the disclosure features a method of treating an autoimmune disease (e.g., an autoimmune disease associated with a TCR bias), comprising administering to a subject in need thereof a multifunctional molecule disclosed herein, wherein the multifunctional molecule is administered in an amount effective to treat the autoimmune disease.

In another aspect, the disclosure features a method of identifying a subject in need of treatment for TCR bias or an autoimmune disease (e.g., associated with a TCR bias) using a multifunctional molecule disclosed herein, comprising determining (e.g., directly determining or indirectly determining, e.g., obtaining information regarding) whether a subject has a TCR bias (e.g., a biased TCRBV clonotype) and/or an autoimmune disease associated with said bias, wherein:

responsive to determining that the subject has a TCR bias (e.g., a biased TCRBV clonotype) and/or an autoimmune disease associated with said bias, identifying the subject as a candidate for treatment using a multifunctional molecule comprising an antigen binding domain that binds to the TCRBV antigen.

In another aspect, the disclosure features a method of evaluating a subject in need of treatment for a TCR bias (e.g., a biased TCRBV clonotype) and/or an autoimmune disease associated with said bias, comprising determining (e.g., directly determining or indirectly determining, e.g., obtaining information regarding) whether a subject has a TCR bias (e.g., a biased TCRBV clonotype).

In yet another aspect, disclosed herein is a method of treating an autoimmune disease (e.g., an autoimmune disease associated with a TCR bias), in a subject in need thereof, comprising administering to said subject an effective amount, e.g., a therapeutically effective amount, of an antibody molecule which binds (e.g., specifically binds) to a T cell receptor beta variable region (TCRβV) (“anti-TCRβV antibody molecule”), thereby treating the disorder.

In another aspect, the disclosure provides a method of depleting a population of T cells in a subject having an autoimmune disorder (e.g., an autoimmune disease associated with a TCR bias), comprising, contacting the T cell population with an effective amount of an antibody molecule which binds (e.g., specifically binds) to a T cell receptor beta variable region (TCRβV) (“anti-TCRβV antibody molecule”).

In some embodiments, the contacting occurs in vivo or in vitro.

In some embodiments, the anti-TCRβV antibody molecule is not an antibody molecule disclosed in U.S. Pat. No. 5,861,155.

In some embodiments, the anti-TCRβV antibody molecule binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule comprises an Fc region, e.g., an Fc region having effector function, e.g., antibody dependent cell-mediated cytotoxicity (ADCC), Antibody-dependent cellular phagocytosis (ADCP) and/or complement dependent cytotoxicity (CDC).

In some embodiments, the anti-TCRβV antibody molecule, the anti-TCRβV antibody molecule comprises an Fc region with enhanced effector function, e.g., as compared to a wildtype Fc region.

In some embodiments, the anti-TCRβV antibody molecule, the anti-TCRβV antibody molecule comprises a human IgG1 region or a human IgG4 region.

In another aspect, the disclosure features a nucleic acid molecule encoding an anti-TCRβV antibody molecule disclosed herein.

In another aspect, the disclosure features a vector, e.g., an expression vector, comprising the nucleic acid molecules disclosed herein.

In another aspect, the disclosure features a host cell comprising a nucleic acid molecule or vector disclosed herein.

In another aspect, the disclosure features a method of making, e.g., producing, an anti-TCRβV antibody molecule disclosed herein, comprising culturing a host cell disclosed herein under suitable conditions, e.g., conditions suitable for gene expression and/or homo- or heterodimerization.

In another aspect, the disclosure features a pharmaceutical composition comprising an anti-TCRβV antibody molecule disclosed herein.

Additional features of any of the aforesaid multifunctional molecules, nucleic acids, vectors, host cells, or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### ENUMERATED EMBODIMENTS

1. A multifunctional molecule, comprising:

(i) a first antigen binding domain that binds to, e.g., selectively binds to, T cell receptor variable beta (TCRBV), e.g., a TCRBV antigen,
and
(ii) one, two, or all of:

(a) an immune cell engager chosen from an NK cell engager, a T cell engager, a B cell engager, a dendritic cell engager, or a macrophage cell engager;

(b) a cytokine molecule or cytokine inhibitor molecule; and

(c) a death receptor signal engager.

2. The multifunctional molecule of embodiment 1, wherein the TCRBV antigen corresponds to a biased TCRBV clonotype, e.g., present in a subject, e.g., a patient, e.g., a subject or a patient with an autoimmune disease.

3. The multifunctional molecule of any preceding embodiment, wherein the multifunctional molecule:

(i) binds specifically to a TCRBV antigen, e.g., the same or similar epitope as the epitope recognized by an anti-TCRBV antibody molecule as described herein;
(ii) shows the same or similar binding affinity or specificity, or both, as an anti-TCRBV antibody molecule as described herein;
(iii) inhibits, e.g., competitively inhibits, the binding of an anti-TCRBV antibody molecule as described herein;
(iv) binds the same or an overlapping epitope with an anti-TCRBV antibody molecule as described herein; or
(v) competes for binding, and/or binds the same epitope, with an anti-TCRBV antibody molecule as described herein.

4. The multifunctional molecule of embodiment 3, wherein the antigen binding domain comprises one or more CDRs, framework regions, variable domains, heavy or light chains, or an antigen binding domain chosen from Tables 13 or 14, or a sequence substantially identical thereto.

5. The multifunctional molecule of any of embodiments 1-4, wherein the antigen binding domain specifically binds to TCRβ V6 (e.g., TCRβ V6-5*01).

6. The multifunctional molecule of embodiment 5, wherein the antigen binding domain comprises at least one (e.g., one, two, three, or four) variable region or an antigen-binding fragment thereof, from Antibody A-H.1 or Antibody A-H.2, or as described in Table 1A, or encoded by the nucleotide sequence in Table 1A, or a sequence substantially identical (e.g., at least 80%, 85%, 90%, 92%, 95%, 97%, 98%, 99% or higher identical) to any of the aforesaid sequences.

7. The multifunctional molecule of either of embodiments 5 or 6, wherein the antigen binding domain comprises at least one, two, or three CDRs (or collectively all of the CDRs) from a heavy chain variable region comprising an amino acid sequence shown in Table 1A, or encoded by a nucleotide sequence shown in Table 1A (or a sequence with one, two, three, four, five, six or more changes, e.g., amino acid substitutions or deletions, relative to the amino acid sequence shown in Table 1A, or encoded by a nucleotide sequence shown in Table 1A).

8. The multifunctional molecule of any of embodiments 5-7, wherein the antigen binding domain comprises at least one, two, or three CDRs (or collectively all of the CDRs) from a light chain variable region comprising an amino acid sequence shown in Table 1A, or encoded by a nucleotide sequence shown in Table 1A (or a sequence with one, two, three, four, five, six or more changes, e.g., amino acid substitutions or deletions, relative to the amino acid sequence shown in Table 1A, or encoded by a nucleotide sequence shown in Table 1A).

9. The multifunctional molecule of any of embodiments 5-8, wherein the antigen binding domain comprises:

(i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

10. The multifunctional molecule of any of embodiments 5-8, wherein the antigen binding domain comprises a LC CDR1, LC CDR2, and LC CDR3 of SEQ ID NO: 2, and a HC CDR1, HC CDR2, and HC CDR3 of SEQ ID NO: 1.

11. The multifunctional molecule of any of embodiments 5-8, wherein the antigen binding domain comprises a LC CDR1, LC CDR2, and LC CDR3 of SEQ ID NO: 10, and a HC CDR1, HC CDR2, and HC CDR3 of SEQ ID NO: 9.

12. The multifunctional molecule of any of embodiments 5-8, wherein the antigen binding domain comprises a LC CDR1, LC CDR2, and LC CDR3 of SEQ ID NO: 11, and a HC CDR1, HC CDR2, and HC CDR3 of SEQ ID NO: 9.

13. The multifunctional molecule of any of embodiments 5-8, wherein the antigen binding domain comprises:

(i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a HC CDR1 amino acid sequence of SEQ ID NO: 3, a HC CDR2 amino acid sequence of SEQ ID NO: 4, or a HC CDR3 amino acid sequence of SEQ ID NO: 5.

14. The multifunctional molecule of any of embodiments 5-8, wherein the antigen binding domain comprises:

(i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

15. The multifunctional molecule of any of embodiments 5-8, wherein the antigen binding domain comprises:

(i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a HC CDR1 amino acid sequence of SEQ ID NO: 45, a HC CDR2 amino acid sequence of SEQ ID NO: 46, or a HC CDR3 amino acid sequence of SEQ ID NO: 47.

16. The multifunctional molecule of any of embodiments 5-8, wherein the antigen binding domain comprises:

(i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

17. The multifunctional molecule of any of embodiments 5-8, wherein the antigen binding domain comprises:

(i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a HC CDR1 amino acid sequence of SEQ ID NO: 48, a HC CDR2 amino acid sequence of SEQ ID NO: 49, or a HC CDR3 amino acid sequence of SEQ ID NO: 50.

18. The multifunctional molecule of any of embodiments 5-8, wherein the antigen binding domain comprises:

(i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

19. The multifunctional molecule of any of embodiments 5-18, wherein the antigen binding domain comprises a light chain variable region (VL) comprising a light chain framework region 1 (VLFWR1) of Antibody A-H.1 or Antibody A-H.2, e.g., as shown in FIG. 1B, e.g., of SEQ ID NOs: 2, 10, or 11.

20. The multifunctional molecule of any of embodiments 5-19, wherein the antigen binding domain comprises a light chain variable region (VL) comprising a light chain framework region 2 (VLFWR2) of Antibody A-H.1 or Antibody A-H.2, e.g., as shown in FIG. 1B, e.g., of SEQ ID NOs: 2, 10, or 11.

21. The multifunctional molecule of any of embodiments 5-20, wherein the antigen binding domain comprises a light chain variable region (VL) comprising a light chain framework region 3 (VLFWR3) of Antibody A-H.1 or Antibody A-H.2, e.g., as shown in FIG. 1B, e.g., of SEQ ID NOs: 2, 10, or 11.

22. The multifunctional molecule of any of embodiments 5-21, wherein the antigen binding domain comprises a light chain variable region (VL) comprising a light chain framework region 4 (VLFWR4) of Antibody A-H.1 or Antibody A-H.2, e.g., as shown in FIG. 1B, e.g., of SEQ ID NOs: 2, 10, or 11.

23. The multifunctional molecule of any of embodiments 5-22, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 2.

24. The multifunctional molecule of any of embodiments 5-22, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 10.

25. The multifunctional molecule of any of embodiments 5-22, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 11.

26. The multifunctional molecule of any of embodiments 5-25, wherein the antigen binding domain comprises a light chain variable domain comprising a framework region, e.g., framework region 1 (VLFWR1), comprising a change, e.g., a substitution (e.g., a conservative substitution) at position 10 according to Kabat numbering, wherein the change at position 10 is to Phenylalanine, e.g., a Serine to Phenylalanine substitution.

27. The multifunctional molecule of any of embodiments 5-26, wherein the antigen binding domain comprises a light chain variable domain comprising a framework region, e.g., framework region 2 (VLFWR2), comprising one or more (e.g., one or two) changes, e.g., substitutions (e.g., a conservative substitution), at positions selected from 36 and 46 according to Kabat numbering, wherein the change at position 36 is to Histidine, e.g., a Tyrosine to Histidine substitution, and the change at position 46 is to Alanine, e.g., an Arginine to Alanine substitution.

28. The multifunctional molecule of any of embodiments 5-27, wherein the antigen binding domain comprises a light chain variable domain comprising a framework region, e.g., framework region 3 (VLFWR3), comprising a change, e.g., substitution (e.g., a conservative substitution), at position 87 according to Kabat numbering, wherein the change at position 87 is to Phenylalanine, e.g., a Tyrosine to Phenylalanine substitution.

29. The multifunctional molecule of any of embodiments 5-28, wherein the antigen binding domain comprises a light chain variable domain comprising (a) a framework region 1 (VLFWR1) comprising a Phenylalanine at position 10, e.g., a substitution at position 10 according to Kabat numbering, e.g., a Serine to Phenyalanine substitution; (b) a framework region 2 (VLFWR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (VLFWR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 10.

30. The multifunctional molecule of any of embodiments 5-28, wherein the antigen binding domain comprises a light chain variable domain comprising (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 11.

31. The multifunctional molecule of any of embodiments 5-30, wherein the antigen binding domain comprises a heavy chain variable region (VH) comprising a heavy chain framework region 1 (VHFWR1) of Antibody A-H.1 or Antibody A-H.2, e.g., as shown in FIG. 1A, e.g., of SEQ ID NOs: 1 or 9.

32. The multifunctional molecule of any of embodiments 5-31, wherein the antigen binding domain comprises a heavy chain variable region (VH) comprising a heavy chain framework region 2 (VHFWR2) of Antibody A-H.1 or Antibody A-H.2, e.g., as shown in FIG. 1A, e.g., of SEQ ID NOs: 1 or 9.

33. The multifunctional molecule of any of embodiments 5-32, wherein the antigen binding domain comprises a heavy chain variable region (VH) comprising a heavy chain framework region 3 (VHFWR3) of Antibody A-H.1 or Antibody A-H.2, e.g., as shown in FIG. 1A, e.g., of SEQ ID NOs: 1 or 9.

34. The multifunctional molecule of any of embodiments 5-33, wherein the antigen binding domain comprises a heavy chain variable region (VH) comprising a heavy chain framework region 4 (VHFWR4) of Antibody A-H.1 or Antibody A-H.2, e.g., as shown in FIG. 1A, e.g., of SEQ ID NOs: 1 or 9.

35. The multifunctional molecule of any of embodiments 5-34, wherein the antigen binding domain comprises a heavy chain variable region (VH) comprising a heavy chain framework region 1 (VHFWR1), a heavy chain framework region 2 (VHFWR2), a heavy chain framework region 3 (VHFWR3), and a heavy chain framework region 4 (VHFWR4) of SEQ ID NO: 1.

36. The multifunctional molecule of any of embodiments 5-34, wherein the antigen binding domain comprises a heavy chain variable region (VH) comprising a heavy chain framework region 1 (VHFWR1), a heavy chain framework region 2 (VHFWR2), a heavy chain framework region 3 (VHFWR3), and a heavy chain framework region 4 (VHFWR4) of SEQ ID NO: 9.

37. The multifunctional molecule of any of embodiments 5-36, wherein the antigen binding domain comprises a heavy chain variable domain comprising a framework region, e.g., framework region 3 (VHFWR3), comprising one or more (e.g., one or two) changes, e.g., substitutions (e.g., a conservative substitution), at positions selected from 73 and 94 according to Kabat numbering, wherein the change at position 73 is to Threonine, e.g., a Glutamic Acid to Threonine substitution, and the change at position 94 is to Glycine, e.g., an Arginine to Glycine substitution.

38. The multifunctional molecule of any of embodiments 5-37, wherein the antigen binding domain comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

39. The multifunctional molecule of any of embodiments 5-18, wherein the antigen binding domain comprises the heavy chain framework regions 1-4 of Antibody A-H.1, e.g., SEQ ID NO: 9; and the light chain framework regions 1-4 of Antibody A-H.1, e.g., SEQ ID NO: 10, or as shown in FIGS. 1A and 1B.

40. The multifunctional molecule of any of embodiments 5-18, wherein the antigen binding domain comprises the heavy chain framework regions 1-4 of Antibody A-H.2, e.g., SEQ ID NO: 9; and the light chain framework regions 1-4 of Antibody A-H.2, e.g., SEQ ID NO: 11, or as shown in FIGS. 1A and 1B.

41. The multifunctional molecule of any of embodiments 5-18, wherein the antigen binding domain comprises:

a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or
a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

42. The multifunctional molecule of any of embodiments 5-18, wherein the antigen binding domain comprises:

a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or
a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

43. The multifunctional molecule of any of embodiments 1-4, wherein the antigen binding domain specifically binds to TCRβ V12 (e.g., TCRβ V12-3*01).

44. The multifunctional molecule of embodiment 43, wherein the antigen binding domain comprises at least one (e.g., one, two, three, or four) variable region or an antigen-binding fragment thereof, as described in Table 2A, or encoded by the nucleotide sequence in Table 2A, or a sequence substantially identical (e.g., at least 80%, 85%, 90%, 92%, 95%, 97%, 98%, 99% or higher identical) to any of the aforesaid sequences.

45. The multifunctional molecule of either of embodiments 43 or 44, wherein the antigen binding domain comprises at least one, two, or three CDRs (or collectively all of the CDRs) from a heavy chain variable region comprising an amino acid sequence shown in Table 2A, or encoded by a nucleotide sequence shown in Table 2A (or a sequence with one, two, three, four, five, six or more changes, e.g., amino acid substitutions or deletions, relative to the amino acid sequence shown in Table 2A, or encoded by a nucleotide sequence shown in Table 2A).

46. The multifunctional molecule of any of embodiments 43-45, wherein the antigen binding domain comprises at least one, two, or three CDRs (or collectively all of the CDRs) from a light chain variable region comprising an amino acid sequence shown in Table 2A, or encoded by a nucleotide sequence shown in Table 2A (or a sequence with one, two, three, four, five, six or more changes, e.g., amino acid substitutions or deletions, relative to the amino acid sequence shown in Table 2A, or encoded by a nucleotide sequence shown in Table 2A).

47. The multifunctional molecule of any of embodiments 43-46, wherein the antigen binding domain comprises:

(i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29, or SEQ ID NO: 30, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24, or SEQ ID NO: 25.

48. The multifunctional molecule of any of embodiments 43-47, wherein the antigen binding domain comprises:

(i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or
(ii) a HC CDR1 amino acid sequence of SEQ ID NO: 17, a HC CDR2 amino acid sequence of SEQ ID NO: 18, or a HC CDR3 amino acid sequence of SEQ ID NO: 19.

49. The multifunctional molecule of any of embodiments 43-47, wherein the antigen binding domain comprises:

(i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or
(ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

50. The multifunctional molecule of any of embodiments 43-47, wherein the antigen binding domain comprises:

(i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a HC CDR1 amino acid sequence of SEQ ID NO: 57, a HC CDR2 amino acid sequence of SEQ ID NO: 58, or a HC CDR3 amino acid sequence of SEQ ID NO: 59.

51. The multifunctional molecule of any of embodiments 43-47, wherein the antigen binding domain comprises:

(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

52. The multifunctional molecule of any of embodiments 43-47, wherein the antigen binding domain comprises:

(i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or
(ii) a HC CDR1 amino acid sequence of SEQ ID NO: 60, a HC CDR2 amino acid sequence of SEQ ID NO: 61, or a HC CDR3 amino acid sequence of SEQ ID NO: 62.

53. The multifunctional molecule of any of embodiments 43-47, wherein the antigen binding domain comprises:

(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

54. The multifunctional molecule of any of embodiments 43-53, wherein the antigen binding domain comprises a light chain variable region (VL) comprising a light chain framework region 1 (VLFWR1) e.g., as shown in FIG. 2B, e.g., of SEQ ID NOs: 16 or 26-30.

55. The multifunctional molecule of any of embodiments 43-54, wherein the antigen binding domain comprises a light chain variable region (VL) comprising a light chain framework region 2 (VLFWR2) e.g., as shown in FIG. 2B, e.g., of SEQ ID NOs: 16 or 26-30.

56. The multifunctional molecule of any of embodiments 43-55, wherein the antigen binding domain comprises a light chain variable region (VL) comprising a light chain framework region 3 (VLFWR3) e.g., as shown in FIG. 2B, e.g., of SEQ ID NOs: 16 or 26-30.

57. The multifunctional molecule of any of embodiments 43-56, wherein the antigen binding domain comprises a light chain variable region (VL) comprising a light chain framework region 4 (VLFWR4) e.g., as shown in FIG. 2B, e.g., of SEQ ID NOs: 16 or 26-30.

58. The multifunctional molecule of any of embodiments 43-57, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 16.

59. The multifunctional molecule of any of embodiments 43-57, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 26.

60. The multifunctional molecule of any of embodiments 43-57, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 27.

61. The multifunctional molecule of any of embodiments 43-57, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 28.

62. The multifunctional molecule of any of embodiments 43-57, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 29.

63. The multifunctional molecule of any of embodiments 43-57, wherein the antigen binding domain comprises a light chain variable region (VL) comprising a light chain framework region 1 (VLFWR1), a light chain framework region 2 (VLFWR2), a light chain framework region 3 (VLFWR3), and a light chain framework region 4 (VLFWR4) of SEQ ID NO: 30.

64. The multifunctional molecule of any of embodiments 43-57, wherein the antigen binding domain comprises a light chain variable domain comprising a framework region, e.g., framework region 1 (VLFWR1), comprising one or more (e.g., one, two, or three) changes, e.g., substitutions (e.g., a conservative substitution), at positions selected from 1, 2, and 4 according to Kabat numbering, wherein the change at position 1 is to Aspartic Acid, e.g., a Alanine to Aspartic Acid substitution, the change at position 2 is to Asparagine, e.g., an Isoleucine to Asparagine substitution, and the change at position 4 is to Leucine, e.g., a Methionine to Leucine substitution.

65. The multifunctional molecule of any of embodiments 43-57 or 64, wherein the antigen binding domain comprises a light chain variable domain comprising a framework region, e.g., framework region 3 (VLFWR3), comprising one or more (e.g., one, two, or three) changes, e.g., substitutions (e.g., a conservative substitution), at positions selected from 66, 69, and 71 according to Kabat numbering, wherein the change at position 66 is to Glycine, e.g., a Lysine to Glycine substitution, the change at position 69 is to Asparagine, e.g., an Tyrosine to Asparagine substitution, and the change at position 71 is to Tyrosine, e.g., a Phenylalanine to Tyrosine substitution.

66. The multifunctional molecule of any of embodiments 43-57, 64, or 65, wherein the antigen binding domain comprises a light chain comprising a framework region 1 (FR1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26.

67. The multifunctional molecule of any of embodiments 43-57, 64, or 65, wherein the antigen binding domain comprises a light chain comprising (a) a framework region 1 (FR1) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27.

68. The multifunctional molecule of any of embodiments 43-57, 64, or 65, wherein the antigen binding domain comprises a light chain comprising (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28.

69. The multifunctional molecule of any of embodiments 43-57, 64, or 65, wherein the antigen binding domain comprises a light chain comprising (a) a framework region 1 (FR1) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29.

70. The multifunctional molecule of any of embodiments 43-69, wherein the antigen binding domain comprises a heavy chain variable region (VH) comprising a heavy chain framework region 1 (VHFWR1) e.g., as shown in FIG. 2A, e.g., of SEQ ID NOs: 15 or 23-25.

71. The multifunctional molecule of any of embodiments 43-70, wherein the antigen binding domain comprises a heavy chain variable region (VH) comprising a heavy chain framework region 2 (VHFWR2) e.g., as shown in FIG. 2A, e.g., of SEQ ID NOs: 15 or 23-25.

72. The multifunctional molecule of any of embodiments 43-71, wherein the antigen binding domain comprises a heavy chain variable region (VH) comprising a heavy chain framework region 3 (VHFWR3) e.g., as shown in FIG. 2A, e.g., of SEQ ID NOs: 15 or 23-25.

73. The multifunctional molecule of any of embodiments 43-72, wherein the antigen binding domain comprises a heavy chain variable region (VH) comprising a heavy chain framework region 4 (VHFWR4) e.g., as shown in FIG. 2A, e.g., of SEQ ID NOs: 15 or 23-25.

74. The multifunctional molecule of any of embodiments 43-73, wherein the antigen binding domain comprises a heavy chain variable region (VH) comprising a heavy chain framework region 1 (VHFWR1), a heavy chain framework region 2 (VHFWR2), a heavy chain framework region 3 (VHFWR3), and a heavy chain framework region 4 (VHFWR4) of SEQ ID NO: 23.

75. The multifunctional molecule of any of embodiments 43-73, wherein the antigen binding domain comprises a heavy chain variable region (VH) comprising a heavy chain framework region 1 (VHFWR1), a heavy chain framework region 2 (VHFWR2), a heavy chain framework region 3 (VHFWR3), and a heavy chain framework region 4 (VHFWR4) of SEQ ID NO: 24.

76. The multifunctional molecule of any of embodiments 43-73, wherein the antigen binding domain comprises a heavy chain variable region (VH) comprising a heavy chain framework region 1 (VHFWR1), a heavy chain framework region 2 (VHFWR2), a heavy chain framework region 3 (VHFWR3), and a heavy chain framework region 4 (VHFWR4) of SEQ ID NO: 25.

77. The multifunctional molecule of any of embodiments 43-73, wherein the antigen binding domain comprises a heavy chain comprising the heavy chain framework regions 1-4 of SEQ ID NOs: 23, 24, or 25; and a light chain comprising the light chain framework regions 1-4 of SEQ ID NOs: 26, 27, 28, 29, or 30.

78. The multifunctional molecule of any of embodiments 43-73, wherein the antigen binding domain comprises:

a VH domain comprising an amino acid sequence chosen from the amino acid sequence of SEQ ID NO: 23, SEQ ID NO:24, or SEQ ID NO: 25, an amino acid sequence at least about 85%, 90%, 95%, 99% or more identical to the amino acid sequence SEQ ID NO: 23, SEQ ID NO:24, or SEQ ID NO:25, or an amino acid sequence which differs by no more than 1, 2, 5, 10, or 15 amino acid residues from the amino acid sequence of SEQ ID NO: 23, SEQ ID NO:24, or SEQ ID NO:25; and/or
a VL domain comprising an amino acid sequence chosen from the amino acid sequence of SEQ ID NO: 26, SEQ ID NO: 27, SEQ ID NO: 28, SEQ ID NO: 29, or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29, or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29, or SEQ ID NO: 30.

79. The multifunctional molecule of any of embodiments 43-73, wherein the antigen binding domain comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

80. The multifunctional molecule of any of embodiments 43-73, wherein the antigen binding domain comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

81. The multifunctional molecule of any of embodiments 43-73, wherein the antigen binding domain comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

82. The multifunctional molecule of any of embodiments 43-73, wherein the antigen binding domain comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

83. The multifunctional molecule of any of embodiments 43-73, wherein the antigen binding domain comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

84. The multifunctional molecule of any of embodiments 43-73, wherein the antigen binding domain comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

85. The multifunctional molecule of any of embodiments 43-73, wherein the antigen binding domain comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

86. The multifunctional molecule of any of embodiments 43-73, wherein the antigen binding domain comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

87. The multifunctional molecule of any of embodiments 43-73, wherein the antigen binding domain comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

88. The multifunctional molecule of any of embodiments 43-73, wherein the antigen binding domain comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

89. The multifunctional molecule of any of embodiments 43-73, wherein the antigen binding domain comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

90. The multifunctional molecule of any of embodiments 43-73, wherein the antigen binding domain comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

91. The multifunctional molecule of any of embodiments 43-73, wherein the antigen binding domain comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

92. The multifunctional molecule of any of embodiments 43-73, wherein the antigen binding domain comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

93. The multifunctional molecule of any of embodiments 43-73, wherein the antigen binding domain comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

94. The multifunctional molecule of any one of embodiments 1-93, wherein the first antigen binding domain has a higher affinity for a T cell receptor comprising the TCRBV antigen, optionally wherein the K_{D} for the binding between the first antigen binding domain and the T cell receptor comprising the TCRBV antigen is no more than 40%, 30%, 20%, 10%, 1%, 0.1%, or 0.01% of the K_{D} for the binding between the first antigen binding domain and a T cell receptor not comprising the TCRBV antigen.

95. The multifunctional molecule of any preceding embodiment, wherein binding of the first antigen binding domain to the TCRBV antigen, e.g., on a lymphocyte (e.g., T cell), does not activate the lymphocyte, e.g., T cell.

96. The multifunctional molecule of any preceding embodiment, wherein binding of the first antigen binding domain to the TCRBV antigen, e.g., on a lymphocyte (e.g., T cell), does not appreciably activate lymphocyte, e.g., T cell, (e.g., as measured by T cell proliferation, expression of a T cell activation marker (e.g., CD69 or CD25), and/or expression of a cytokine (e.g., TNFα and IFNγ).

97. The multifunctional molecule of any preceding embodiment, wherein the multifunctional molecule preferentially binds to a lymphocyte comprising the TCRBV antigen over a lymphocyte not comprising the TCRBV antigen, optionally wherein the binding between the multifunctional molecule and the lymphocyte comprising the TCRBV antigen is more than 10, 20, 30, 40, or 50-fold greater than the binding between the multifunctional molecule and a lymphocyte not comprising the TCRBV antigen.

98. The multifunctional molecule of any one of embodiments 1-97, wherein the multifunctional molecule comprises an immune cell engager chosen from an NK cell engager, a T cell engager, a B cell engager, a dendritic cell engager, or a macrophage cell engager.

99. The multifunctional molecule of embodiment 98, wherein the immune cell engager binds to and activates an immune cell, e.g., an effector cell.

100. The multifunctional molecule of embodiment 98, wherein the immune cell engager binds to, but does not activate, an immune cell, e.g., an effector cell.

101. The multifunctional molecule of any one of embodiments 98-100, wherein the immune cell engager is a T cell engager, e.g., a T cell engager that mediates binding to and activation of a T cell, or a T cell engager that mediates binding to but not activation of a T cell.

102. The multifunctional molecule of embodiment 101, wherein the T cell engager binds to TCRα, TCRγ, TCRζ, ICOS, CD28, CD27, HVEM, LIGHT, CD40, 4-1BB, OX40, DR3, GITR, CD30, TIMI, SLAM, CD2, CD3, or CD226, e.g., the T cell engager is an anti-CD3 antibody molecule.

103. The multifunctional molecule of any one of embodiments 98-100, wherein the immune cell engager is an NK cell engager, e.g., an NK cell engager that mediates binding to and activation of an NK cell, or an NK cell engager that mediates binding to but not activation of an NK cell.

104. The multifunctional molecule of embodiment 103, wherein the NK cell engager is chosen from an antibody molecule, e.g., an antigen binding domain, or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160, e.g., the NK cell engager is an antibody molecule or ligand that binds to (e.g., activates) NKp30.

105. The multifunctional molecule of embodiment 103, wherein the NK cell engager is an antibody molecule, e.g., an antigen binding domain.

106. The multifunctional molecule of either of embodiments 104 or 105, wherein the NK cell engager is capable of engaging an NK cell.

107. The multifunctional molecule of any one of embodiments 103-106, wherein the NK cell engager is an antibody molecule, e.g., an antigen binding domain, that binds to NKp30, NKp46, NKG2D, or CD16.

108. The multifunctional molecule of any preceding embodiment, wherein the multifunctional molecule:

(i) binds specifically to an epitope of NKp30, NKp46, NKG2D, or CD16, e.g., the same or similar epitope as the epitope recognized by an anti-NKp30, anti-NKp46, anti-NKG2D, or anti-CD16 antibody molecule as described herein;
(ii) shows the same or similar binding affinity or specificity, or both, as an anti-NKp30, anti-NKp46, anti-NKG2D, or anti-CD16 antibody molecule as described herein;
(iii) inhibits, e.g., competitively inhibits, the binding of an anti-NKp30, anti-NKp46, anti-NKG2D, or anti-CD16 antibody molecule as described herein;
(iv) binds the same or an overlapping epitope with an anti-NKp30, anti-NKp46, anti-NKG2D, or anti-CD16 antibody molecule as described herein; or
(v) competes for binding, and/or binds the same epitope, with an anti-NKp30, anti-NKp46, anti-NKG2D, or anti-CD16 antibody molecule as described herein.

109. The multifunctional molecule of any of embodiments 103-108, wherein the anti-NKp30 or anti-NKp46 antibody molecule comprises one or more CDRs, framework regions, variable domains, heavy or light chains, or an antigen binding domain chosen from Tables 7-10, or a sequence substantially identical thereto.

110. The multifunctional molecule of any of embodiments 103-109, wherein the NK cell engager is an antibody molecule, e.g., an antigen binding domain, that binds to NKp30.

111. The multifunctional molecule of any of embodiments 103-110, wherein lysis of the lymphocyte, e.g., lymphocyte comprising a TCRBV antigen corresponding to a biased TCRBV clonotype, is mediated by NKp30.

112. The multifunctional molecule of any of embodiments 103-111, wherein the multifunctional molecule does not activate the NK cell when incubated with the NK cell in the absence of the TCRBV antigen.

113. The multifunctional molecule of any of embodiments 103-112, wherein the multifunctional molecule activates the NK cell when the NK cell is a NKp30 expressing NK cell and when the TCRBV antigen is also present.

114. The multifunctional molecule of any of embodiments 103-113, wherein the multifunctional molecule does not activate the NK cell when the NK cell is not a NKp30 expressing NK cell and the TCRBV antigen is also present.

115. The multifunctional molecule of any of embodiments 103-113, wherein the NK cell engager comprises:

(i) a heavy chain variable region (VH) comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 6065 (or a sequence with no more than 1, 2, 3, or 4 mutations, e.g., substitutions, additions, or deletions).

116. The multifunctional molecule of embodiment 115, wherein the NK cell engager comprises:

(i) a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and
(ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 6065.

117. The multifunctional molecule of any of embodiments 103-116, wherein the NK cell engager comprises:

(1) a heavy chain variable region (VH) comprising a heavy chain framework region 1 (VHFWR1) amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, or 6 mutations, e.g., substitutions, additions, or deletions, therefrom), or a VHFWR4 amino acid sequence of SEQ ID NO: 6006 (or a sequence with no more than 1, 2, 3, 4, 5, or 6 mutations, e.g., substitutions, additions, or deletions, therefrom), and/or
(2) a light chain variable region (VL) comprising a light chain framework region 1 (VLFWR1) amino acid sequence of SEQ ID NO: 6066 (or a sequence with no more than 1, 2, 3, 4, 5, or 6 mutations, e.g., substitutions, additions, or deletions, therefrom), a VLFWR2 amino acid sequence of SEQ ID NO: 6067 (or a sequence with no more than 1, 2, 3, 4, 5, or 6 mutations, e.g., substitutions, additions, or deletions, therefrom), a VLFWR3 amino acid sequence of SEQ ID NO: 6068 (or a sequence with no more than 1, 2, 3, 4, 5, or 6 mutations, e.g., substitutions, additions, or deletions, therefrom), or a VLFWR4 amino acid sequence of SEQ ID NO: 6069 (or a sequence with no more than 1, 2, 3, 4, 5, or 6 mutations, e.g., substitutions, additions, or deletions, therefrom).

118. The multifunctional molecule of embodiment 117, wherein the NK cell engager comprises:

(1) a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and
(2) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 6068, or a VLFWR4 amino acid sequence of SEQ ID NO: 6069.

119. The multifunctional molecule of any one of embodiments 103-118, wherein the NK cell engager comprises:

(i) a VH comprising the amino acid sequence of SEQ ID NO: 6121 (or an amino acid sequence having at least about 75%, 80%, 85%, 90%, 95%, or 99% sequence identity to SEQ ID NO: 6121), and/or
(ii) a VL comprising the amino acid sequence of SEQ ID NO: 6135 (or an amino acid sequence having at least about 93%, 95%, or 99% sequence identity to SEQ ID NO: 6135).

120. The multifunctional molecule of either of embodiments 103-119, wherein the NK cell engager comprises a heavy chain comprising the amino acid sequence of SEQ ID NOs: 6148 or 6149 (or an amino acid sequence having at least about 75%, 80%, 85%, 90%, 95%, or 99% sequence identity to SEQ ID NOs: 6148 or 6149).

121. The multifunctional molecule of either of embodiments 103-120, wherein the NK cell engager comprises a light chain comprising the amino acid sequence of SEQ ID NO: 6150 (or an amino acid sequence having at least about 75%, 80%, 85%, 90%, 95%, or 99% sequence identity to SEQ ID NO: 6150).

122. The multifunctional molecule of either of embodiments 103-121, wherein the NK cell engager comprises a heavy chain comprising the amino acid sequence of SEQ ID NOs: 6148 or 6149 (or an amino acid sequence having at least about 75%, 80%, 85%, 90%, 95%, or 99% sequence identity to SEQ ID NOs: 6148 or 6149), and a light chain comprising the amino acid sequence of SEQ ID NO: 6150 (or an amino acid sequence having at least about 75%, 80%, 85%, 90%, 95%, or 99% sequence identity to SEQ ID NO: 6150).

123. The multifunctional molecule of any of embodiments 103-116, wherein the NK cell engager comprises a heavy chain variable region (VH) comprising a heavy chain framework region 1 (VHFWR1) amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, or 6 mutations, e.g., substitutions, additions, or deletions, therefrom), or a VHFWR4 amino acid sequence of SEQ ID NO: 6017 (or a sequence with no more than 1, 2, 3, 4, 5, or 6 mutations, e.g., substitutions, additions, or deletions, therefrom).

124. The multifunctional molecule of embodiment 123, wherein the NK cell engager comprises a heavy chain variable region (VH) comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, or a VHFWR4 amino acid sequence of SEQ ID NO: 6017.

125. The multifunctional molecule of embodiment 124, wherein the NK cell engager comprises a VH comprising the amino acid sequence of SEQ ID NO: 6123 (or an amino acid sequence having at least about 75%, 80%, 85%, 90%, 95%, or 99% sequence identity to SEQ ID NO: 6123).

126. The multifunctional molecule of any of embodiments 103-116, wherein the NK cell engager comprises a heavy chain variable region (VH) comprising a heavy chain framework region 1 (VHFWR1) amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, or 6 mutations, e.g., substitutions, additions, or deletions, therefrom), or a VHFWR4 amino acid sequence of SEQ ID NO: 6021 (or a sequence with no more than 1, 2, 3, 4, 5, or 6 mutations, e.g., substitutions, additions, or deletions, therefrom).

127. The multifunctional molecule of embodiment 126, wherein the NK cell engager comprises a heavy chain variable region (VH) comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, or a VHFWR4 amino acid sequence of SEQ ID NO: 6021.

128. The multifunctional molecule of embodiment 127, wherein the NK cell engager comprises a VH comprising the amino acid sequence of SEQ ID NO: 6124 (or an amino acid sequence having at least about 75%, 80%, 85%, 90%, 95%, or 99% sequence identity to SEQ ID NO: 6124).

129. The multifunctional molecule of any of embodiments 103-116, wherein the NK cell engager comprises a heavy chain variable region (VH) comprising a heavy chain framework region 1 (VHFWR1) amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, or 6 mutations, e.g., substitutions, additions, or deletions, therefrom), or a VHFWR4 amino acid sequence of SEQ ID NO: 6025 (or a sequence with no more than 1, 2, 3, 4, 5, or 6 mutations, e.g., substitutions, additions, or deletions, therefrom).

130. The multifunctional molecule of embodiment 129, wherein the NK cell engager comprises a heavy chain variable region (VH) comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, or a VHFWR4 amino acid sequence of SEQ ID NO: 6025.

131. The multifunctional molecule of embodiment 130, wherein the NK cell engager comprises a VH comprising the amino acid sequence of SEQ ID NO: 6125 (or an amino acid sequence having at least about 75%, 80%, 85%, 90%, 95%, or 99% sequence identity to SEQ ID NO: 6125).

132. The multifunctional molecule of any of embodiments 103-116, wherein the NK cell engager comprises a heavy chain variable region (VH) comprising a heavy chain framework region 1 (VHFWR1) amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, or 6 mutations, e.g., substitutions, additions, or deletions, therefrom), or a VHFWR4 amino acid sequence of SEQ ID NO: 6029 (or a sequence with no more than 1, 2, 3, 4, 5, or 6 mutations, e.g., substitutions, additions, or deletions, therefrom).

133. The multifunctional molecule of embodiment 132, wherein the NK cell engager comprises a heavy chain variable region (VH) comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, or a VHFWR4 amino acid sequence of SEQ ID NO: 6029.

134. The multifunctional molecule of embodiment 133, wherein the NK cell engager comprises a VH comprising the amino acid sequence of SEQ ID NO: 6126 (or an amino acid sequence having at least about 75%, 80%, 85%, 90%, 95%, or 99% sequence identity to SEQ ID NO: 6126).

135. The multifunctional molecule of any of embodiments 103-116, wherein the NK cell engager comprises a heavy chain variable region (VH) comprising a heavy chain framework region 1 (VHFWR1) amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, or 6 mutations, e.g., substitutions, additions, or deletions, therefrom), or a VHFWR4 amino acid sequence of SEQ ID NO: 6034 (or a sequence with no more than 1, 2, 3, 4, 5, or 6 mutations, e.g., substitutions, additions, or deletions, therefrom).

136. The multifunctional molecule of embodiment 135, wherein the NK cell engager comprises a heavy chain variable region (VH) comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, or a VHFWR4 amino acid sequence of SEQ ID NO: 6034.

137. The multifunctional molecule of embodiment 136, wherein the NK cell engager comprises a VH comprising the amino acid sequence of SEQ ID NO: 6127 (or an amino acid sequence having at least about 75%, 80%, 85%, 90%, 95%, or 99% sequence identity to SEQ ID NO: 6127).

138. The multifunctional molecule of any of embodiments 103-116, wherein the NK cell engager comprises a heavy chain variable region (VH) comprising a heavy chain framework region 1 (VHFWR1) amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, or 6 mutations, e.g., substitutions, additions, or deletions, therefrom), or a VHFWR4 amino acid sequence of SEQ ID NO: 6038 (or a sequence with no more than 1, 2, 3, 4, 5, or 6 mutations, e.g., substitutions, additions, or deletions, therefrom).

139. The multifunctional molecule of embodiment 138, wherein the NK cell engager comprises a heavy chain variable region (VH) comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, or a VHFWR4 amino acid sequence of SEQ ID NO: 6038.

140. The multifunctional molecule of embodiment 139, wherein the NK cell engager comprises a VH comprising the amino acid sequence of SEQ ID NO: 6128 (or an amino acid sequence having at least about 75%, 80%, 85%, 90%, 95%, or 99% sequence identity to SEQ ID NO: 6128).

141. The multifunctional molecule of any of embodiments 103-116 or 123-140, wherein the NK cell engager comprises a light chain variable region (VL) comprising a light chain framework region 1 (VLFWR1) amino acid sequence of SEQ ID NO: 6077 (or a sequence with no more than 1, 2, 3, 4, 5, or 6 mutations, e.g., substitutions, additions, or deletions, therefrom), a VLFWR2 amino acid sequence of SEQ ID NO: 6078 (or a sequence with no more than 1, 2, 3, 4, 5, or 6 mutations, e.g., substitutions, additions, or deletions, therefrom), a VLFWR3 amino acid sequence of SEQ ID NO: 6079 (or a sequence with no more than 1, 2, 3, 4, 5, or 6 mutations, e.g., substitutions, additions, or deletions, therefrom), or a VLFWR4 amino acid sequence of SEQ ID NO: 6080 (or a sequence with no more than 1, 2, 3, 4, 5, or 6 mutations, e.g., substitutions, additions, or deletions, therefrom).

142. The multifunctional molecule of embodiment 141, wherein the NK cell engager comprises a light chain variable region (VL) comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, or a VLFWR4 amino acid sequence of SEQ ID NO: 6080.

143. The multifunctional molecule of embodiment 142, wherein the NK cell engager comprises a VL comprising the amino acid sequence of SEQ ID NO: 6137 (or an amino acid sequence having at least about 93%, 95%, or 99% sequence identity to SEQ ID NO: 6137).

144. The multifunctional molecule of any of embodiments 103-116 or 123-140, wherein the NK cell engager comprises a light chain variable region (VL) comprising a light chain framework region 1 (VLFWR1) amino acid sequence of SEQ ID NO: 6081 (or a sequence with no more than 1, 2, 3, 4, 5, or 6 mutations, e.g., substitutions, additions, or deletions, therefrom), a VLFWR2 amino acid sequence of SEQ ID NO: 6082 (or a sequence with no more than 1, 2, 3, 4, 5, or 6 mutations, e.g., substitutions, additions, or deletions, therefrom), a VLFWR3 amino acid sequence of SEQ ID NO: 6083 (or a sequence with no more than 1, 2, 3, 4, 5, or 6 mutations, e.g., substitutions, additions, or deletions, therefrom), or a VLFWR4 amino acid sequence of SEQ ID NO: 6084 (or a sequence with no more than 1, 2, 3, 4, 5, or 6 mutations, e.g., substitutions, additions, or deletions, therefrom).

145. The multifunctional molecule of embodiment 144, wherein the NK cell engager comprises a light chain variable region (VL) comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, or a VLFWR4 amino acid sequence of SEQ ID NO: 6084.

146. The multifunctional molecule of embodiment 145, wherein the NK cell engager comprises a VL comprising the amino acid sequence of SEQ ID NO: 6138 (or an amino acid sequence having at least about 93%, 95%, or 99% sequence identity to SEQ ID NO: 6138).

147. The multifunctional molecule of any of embodiments 103-116 or 123-140, wherein the NK cell engager comprises a light chain variable region (VL) comprising a light chain framework region 1 (VLFWR1) amino acid sequence of SEQ ID NO: 6085 (or a sequence with no more than 1, 2, 3, 4, 5, or 6 mutations, e.g., substitutions, additions, or deletions, therefrom), a VLFWR2 amino acid sequence of SEQ ID NO: 6086 (or a sequence with no more than 1, 2, 3, 4, 5, or 6 mutations, e.g., substitutions, additions, or deletions, therefrom), a VLFWR3 amino acid sequence of SEQ ID NO: 6087 (or a sequence with no more than 1, 2, 3, 4, 5, or 6 mutations, e.g., substitutions, additions, or deletions, therefrom), or a VLFWR4 amino acid sequence of SEQ ID NO: 6088 (or a sequence with no more than 1, 2, 3, 4, 5, or 6 mutations, e.g., substitutions, additions, or deletions, therefrom).

148. The multifunctional molecule of embodiment 147, wherein the NK cell engager comprises a light chain variable region (VL) comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, or a VLFWR4 amino acid sequence of SEQ ID NO: 6088.

149. The multifunctional molecule of embodiment 148, wherein the NK cell engager comprises a VL comprising the amino acid sequence of SEQ ID NO: 6139 (or an amino acid sequence having at least about 93%, 95%, or 99% sequence identity to SEQ ID NO: 6139).

150. The multifunctional molecule of any of embodiments 103-116 or 123-140, wherein the NK cell engager comprises a light chain variable region (VL) comprising a light chain framework region 1 (VLFWR1) amino acid sequence of SEQ ID NO: 6089 (or a sequence with no more than 1, 2, 3, 4, 5, or 6 mutations, e.g., substitutions, additions, or deletions, therefrom), a VLFWR2 amino acid sequence of SEQ ID NO: 6090 (or a sequence with no more than 1, 2, 3, 4, 5, or 6 mutations, e.g., substitutions, additions, or deletions, therefrom), a VLFWR3 amino acid sequence of SEQ ID NO: 6091 (or a sequence with no more than 1, 2, 3, 4, 5, or 6 mutations, e.g., substitutions, additions, or deletions, therefrom), or a VLFWR4 amino acid sequence of SEQ ID NO: 6092 (or a sequence with no more than 1, 2, 3, 4, 5, or 6 mutations, e.g., substitutions, additions, or deletions, therefrom).

151. The multifunctional molecule of embodiment 150, wherein the NK cell engager comprises a light chain variable region (VL) comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, or a VLFWR4 amino acid sequence of SEQ ID NO: 6092.

152. The multifunctional molecule of embodiment 151, wherein the NK cell engager comprises a VL comprising the amino acid sequence of SEQ ID NO: 6140 (or an amino acid sequence having at least about 93%, 95%, or 99% sequence identity to SEQ ID NO: 6140).

153. The multifunctional molecule of any of embodiments 103-116 or 123-140, wherein the NK cell engager comprises a light chain variable region (VL) comprising a light chain framework region 1 (VLFWR1) amino acid sequence of SEQ ID NO: 6093 (or a sequence with no more than 1, 2, 3, 4, 5, or 6 mutations, e.g., substitutions, additions, or deletions, therefrom), a VLFWR2 amino acid sequence of SEQ ID NO: 6094 (or a sequence with no more than 1, 2, 3, 4, 5, or 6 mutations, e.g., substitutions, additions, or deletions, therefrom), a VLFWR3 amino acid sequence of SEQ ID NO: 6095 (or a sequence with no more than 1, 2, 3, 4, 5, or 6 mutations, e.g., substitutions, additions, or deletions, therefrom), or a VLFWR4 amino acid sequence of SEQ ID NO: 6096 (or a sequence with no more than 1, 2, 3, 4, 5, or 6 mutations, e.g., substitutions, additions, or deletions, therefrom).

154. The multifunctional molecule of embodiment 153, wherein the NK cell engager comprises a light chain variable region (VL) comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, or a VLFWR4 amino acid sequence of SEQ ID NO: 6096.

155. The multifunctional molecule of embodiment 154, wherein the NK cell engager comprises a VL comprising the amino acid sequence of SEQ ID NO: 6141 (or an amino acid sequence having at least about 93%, 95%, or 99% sequence identity to SEQ ID NO: 6141).

156. The multifunctional molecule of any of embodiments 103-114, wherein the NK cell engager comprises:

(i) a heavy chain variable region (VH) comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions).

157. The multifunctional molecule of embodiment 156, wherein the NK cell engager comprises:

(i) a heavy chain variable region (VH) comprising a heavy chain complementarity determining region 1 (VHCDR1) amino acid sequence of SEQ ID NO: 6007, a VHCDR2 amino acid sequence of SEQ ID NO: 6008, and/or a VHCDR3 amino acid sequence of SEQ ID NO: 6009, and
(ii) a light chain variable region (VL) comprising a light chain complementarity determining region 1 (VLCDR1) amino acid sequence of SEQ ID NO: 6070, a VLCDR2 amino acid sequence of SEQ ID NO: 6071, and/or a VLCDR3 amino acid sequence of SEQ ID NO: 6072.

158. The multifunctional molecule of any of embodiments 103-114, 156, or 157, wherein the NK cell engager comprises:

(1) a heavy chain variable region (VH) comprising a heavy chain framework region 1 (VHFWR1) amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, or 6 mutations, e.g., substitutions, additions, or deletions, therefrom), or a VHFWR4 amino acid sequence of SEQ ID NO: 6013 (or a sequence with no more than 1, 2, 3, 4, 5, or 6 mutations, e.g., substitutions, additions, or deletions, therefrom), and/or
(2) a light chain variable region (VL) comprising a light chain framework region 1 (VLFWR1) amino acid sequence of SEQ ID NO: 6073 (or a sequence with no more than 1, 2, 3, 4, 5, or 6 mutations, e.g., substitutions, additions, or deletions, therefrom), a VLFWR2 amino acid sequence of SEQ ID NO: 6074 (or a sequence with no more than 1, 2, 3, 4, 5, or 6 mutations, e.g., substitutions, additions, or deletions, therefrom), a VLFWR3 amino acid sequence of SEQ ID NO: 6075 (or a sequence with no more than 1, 2, 3, 4, 5, or 6 mutations, e.g., substitutions, additions, or deletions, therefrom), or a VLFWR4 amino acid sequence of SEQ ID NO: 6076 (or a sequence with no more than 1, 2, 3, 4, 5, or 6 mutations, e.g., substitutions, additions, or deletions, therefrom).

159. The multifunctional molecule of embodiment 158, wherein the NK cell engager comprises:

(1) a heavy chain variable region (VH) comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, or a VHFWR4 amino acid sequence of SEQ ID NO: 6013, and
(3) a light chain variable region (VL) comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, or a VLFWR4 amino acid sequence of SEQ ID NO: 6076.

160. The multifunctional molecule of any one of embodiments 103-114 or 156-159, wherein the NK cell engager comprises:

(i) a VH comprising the amino acid sequence of SEQ ID NO: 6122 (or an amino acid sequence having at least about 75%, 80%, 85%, 90%, 95%, or 99% sequence identity to SEQ ID NO: 6122), and/or
(ii) a VL comprising the amino acid sequence of SEQ ID NO: 6136 (or an amino acid sequence having at least about 93%, 95%, or 99% sequence identity to SEQ ID NO: 6136).

161. The multifunctional molecule of any of embodiments 103-114 or 156-160, wherein the NK cell engager comprises a heavy chain comprising the amino acid sequence of SEQ ID NOs: 6151 or 6152 (or an amino acid sequence having at least about 75%, 80%, 85%, 90%, 95%, or 99% sequence identity to SEQ ID NOs: 6151 or 6152).

162. The multifunctional molecule of any of embodiments 103-114 or 156-161, wherein the NK cell engager comprises a light chain comprising the amino acid sequence of SEQ ID NO: 6153 (or an amino acid sequence having at least about 75%, 80%, 85%, 90%, 95%, or 99% sequence identity to SEQ ID NO: 6153).

163. The multifunctional molecule of any of embodiments 103-114 or 156-162, wherein the NK cell engager comprises a heavy chain comprising the amino acid sequence of SEQ ID NOs: 6151 or 6152 (or an amino acid sequence having at least about 75%, 80%, 85%, 90%, 95%, or 99% sequence identity to SEQ ID NOs: 6151 or 6152), and a light chain comprising the amino acid sequence of SEQ ID NO: 6153 (or an amino acid sequence having at least about 75%, 80%, 85%, 90%, 95%, or 99% sequence identity to SEQ ID NO: 6153).

164. The multifunctional molecule of any of embodiments 103-114, 156, or 157, wherein the NK cell engager comprises a heavy chain variable region (VH) comprising a heavy chain framework region 1 (VHFWR1) amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, or 6 mutations, e.g., substitutions, additions, or deletions, therefrom), or a VHFWR4 amino acid sequence of SEQ ID NO: 6042 (or a sequence with no more than 1, 2, 3, 4, 5, or 6 mutations, e.g., substitutions, additions, or deletions, therefrom).

165. The multifunctional molecule of embodiment 164, wherein the NK cell engager comprises a heavy chain variable region (VH) comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, or a VHFWR4 amino acid sequence of SEQ ID NO: 6042.

166. The multifunctional molecule of embodiment 165, wherein the NK cell engager comprises a VH comprising the amino acid sequence of SEQ ID NO: 6129 (or an amino acid sequence having at least about 75%, 80%, 85%, 90%, 95%, or 99% sequence identity to SEQ ID NO: 6129).

167. The multifunctional molecule of any of embodiments 103-114, 156, or 157, wherein the NK cell engager comprises a heavy chain variable region (VH) comprising a heavy chain framework region 1 (VHFWR1) amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, or 6 mutations, e.g., substitutions, additions, or deletions, therefrom), or a VHFWR4 amino acid sequence of SEQ ID NO: 6046 (or a sequence with no more than 1, 2, 3, 4, 5, or 6 mutations, e.g., substitutions, additions, or deletions, therefrom).

168. The multifunctional molecule of embodiment 167, wherein the NK cell engager comprises a heavy chain variable region (VH) comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, or a VHFWR4 amino acid sequence of SEQ ID NO: 6046.

169. The multifunctional molecule of embodiment 168, wherein the NK cell engager comprises a VH comprising the amino acid sequence of SEQ ID NO: 6130 (or an amino acid sequence having at least about 75%, 80%, 85%, 90%, 95%, or 99% sequence identity to SEQ ID NO: 6130).

170. The multifunctional molecule of any of embodiments 103-114, 156, or 157, wherein the NK cell engager comprises a heavy chain variable region (VH) comprising a heavy chain framework region 1 (VHFWR1) amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, or 6 mutations, e.g., substitutions, additions, or deletions, therefrom), or a VHFWR4 amino acid sequence of SEQ ID NO: 6050 (or a sequence with no more than 1, 2, 3, 4, 5, or 6 mutations, e.g., substitutions, additions, or deletions, therefrom).

171. The multifunctional molecule of embodiment 170, wherein the NK cell engager comprises a heavy chain variable region (VH) comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, or a VHFWR4 amino acid sequence of SEQ ID NO: 6050.

172. The multifunctional molecule of embodiment 171, wherein the NK cell engager comprises a VH comprising the amino acid sequence of SEQ ID NO: 6131 (or an amino acid sequence having at least about 75%, 80%, 85%, 90%, 95%, or 99% sequence identity to SEQ ID NO: 6131).

173. The multifunctional molecule of any of embodiments 103-114, 156, or 157, wherein the NK cell engager comprises a heavy chain variable region (VH) comprising a heavy chain framework region 1 (VHFWR1) amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, or 6 mutations, e.g., substitutions, additions, or deletions, therefrom), or a VHFWR4 amino acid sequence of SEQ ID NO: 6054 (or a sequence with no more than 1, 2, 3, 4, 5, or 6 mutations, e.g., substitutions, additions, or deletions, therefrom).

174. The multifunctional molecule of embodiment 173, wherein the NK cell engager comprises a heavy chain variable region (VH) comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, or a VHFWR4 amino acid sequence of SEQ ID NO: 6054.

175. The multifunctional molecule of embodiment 174, wherein the NK cell engager comprises a VH comprising the amino acid sequence of SEQ ID NO: 6132 (or an amino acid sequence having at least about 75%, 80%, 85%, 90%, 95%, or 99% sequence identity to SEQ ID NO: 6132).

176. The multifunctional molecule of any of embodiments 103-114, 156, or 157, wherein the NK cell engager comprises a heavy chain variable region (VH) comprising a heavy chain framework region 1 (VHFWR1) amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, or 6 mutations, e.g., substitutions, additions, or deletions, therefrom), or a VHFWR4 amino acid sequence of SEQ ID NO: 6058 (or a sequence with no more than 1, 2, 3, 4, 5, or 6 mutations, e.g., substitutions, additions, or deletions, therefrom).

177. The multifunctional molecule of embodiment 176, wherein the NK cell engager comprises a heavy chain variable region (VH) comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, or a VHFWR4 amino acid sequence of SEQ ID NO: 6058.

178. The multifunctional molecule of embodiment 177, wherein the NK cell engager comprises a VH comprising the amino acid sequence of SEQ ID NO: 6133 (or an amino acid sequence having at least about 75%, 80%, 85%, 90%, 95%, or 99% sequence identity to SEQ ID NO: 6133).

179. The multifunctional molecule of any of embodiments 103-114, 156, or 157, wherein the NK cell engager comprises a heavy chain variable region (VH) comprising a heavy chain framework region 1 (VHFWR1) amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, or 6 mutations, e.g., substitutions, additions, or deletions, therefrom), or a VHFWR4 amino acid sequence of SEQ ID NO: 6062 (or a sequence with no more than 1, 2, 3, 4, 5, or 6 mutations, e.g., substitutions, additions, or deletions, therefrom).

180. The multifunctional molecule of embodiment 179, wherein the NK cell engager comprises a heavy chain variable region (VH) comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, or a VHFWR4 amino acid sequence of SEQ ID NO: 6062.

181. The multifunctional molecule of embodiment 180, wherein the NK cell engager comprises a VH comprising the amino acid sequence of SEQ ID NO: 6134 (or an amino acid sequence having at least about 75%, 80%, 85%, 90%, 95%, or 99% sequence identity to SEQ ID NO: 6134).

182. The multifunctional molecule of any of embodiments 103-114, 156, 157, or 164-181, wherein the NK cell engager comprises a light chain variable region (VL) comprising a light chain framework region 1 (VLFWR1) amino acid sequence of SEQ ID NO: 6097 (or a sequence with no more than 1, 2, 3, 4, 5, or 6 mutations, e.g., substitutions, additions, or deletions, therefrom), a VLFWR2 amino acid sequence of SEQ ID NO: 6098 (or a sequence with no more than 1, 2, 3, 4, 5, or 6 mutations, e.g., substitutions, additions, or deletions, therefrom), a VLFWR3 amino acid sequence of SEQ ID NO: 6099 (or a sequence with no more than 1, 2, 3, 4, 5, or 6 mutations, e.g., substitutions, additions, or deletions, therefrom), or a VLFWR4 amino acid sequence of SEQ ID NO: 6100 (or a sequence with no more than 1, 2, 3, 4, 5, or 6 mutations, e.g., substitutions, additions, or deletions, therefrom).

183. The multifunctional molecule of embodiment 182, wherein the NK cell engager comprises a light chain variable region (VL) comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, or a VLFWR4 amino acid sequence of SEQ ID NO: 6100.

184. The multifunctional molecule of embodiment 183, wherein the NK cell engager comprises a VL comprising the amino acid sequence of SEQ ID NO: 6142 (or an amino acid sequence having at least about 93%, 95%, or 99% sequence identity to SEQ ID NO: 6142).

185. The multifunctional molecule of any of embodiments 103-114, 156, 157, or 164-181, wherein the NK cell engager comprises a light chain variable region (VL) comprising a light chain framework region 1 (VLFWR1) amino acid sequence of SEQ ID NO: 6101 (or a sequence with no more than 1, 2, 3, 4, 5, or 6 mutations, e.g., substitutions, additions, or deletions, therefrom), a VLFWR2 amino acid sequence of SEQ ID NO: 6102 (or a sequence with no more than 1, 2, 3, 4, 5, or 6 mutations, e.g., substitutions, additions, or deletions, therefrom), a VLFWR3 amino acid sequence of SEQ ID NO: 6103 (or a sequence with no more than 1, 2, 3, 4, 5, or 6 mutations, e.g., substitutions, additions, or deletions, therefrom), or a VLFWR4 amino acid sequence of SEQ ID NO: 6104 (or a sequence with no more than 1, 2, 3, 4, 5, or 6 mutations, e.g., substitutions, additions, or deletions, therefrom).

186. The multifunctional molecule of embodiment 185, wherein the NK cell engager comprises a light chain variable region (VL) comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, or a VLFWR4 amino acid sequence of SEQ ID NO: 6104.

187. The multifunctional molecule of embodiment 186, wherein the NK cell engager comprises a VL comprising the amino acid sequence of SEQ ID NO: 6143 (or an amino acid sequence having at least about 93%, 95%, or 99% sequence identity to SEQ ID NO: 6143).

188. The multifunctional molecule of any of embodiments 103-114, 156, 157, or 164-181, wherein the NK cell engager comprises a light chain variable region (VL) comprising a light chain framework region 1 (VLFWR1) amino acid sequence of SEQ ID NO: 6105 (or a sequence with no more than 1, 2, 3, 4, 5, or 6 mutations, e.g., substitutions, additions, or deletions, therefrom), a VLFWR2 amino acid sequence of SEQ ID NO: 6106 (or a sequence with no more than 1, 2, 3, 4, 5, or 6 mutations, e.g., substitutions, additions, or deletions, therefrom), a VLFWR3 amino acid sequence of SEQ ID NO: 6107 (or a sequence with no more than 1, 2, 3, 4, 5, or 6 mutations, e.g., substitutions, additions, or deletions, therefrom), or a VLFWR4 amino acid sequence of SEQ ID NO: 6108 (or a sequence with no more than 1, 2, 3, 4, 5, or 6 mutations, e.g., substitutions, additions, or deletions, therefrom).

189. The multifunctional molecule of embodiment 188, wherein the NK cell engager comprises a light chain variable region (VL) comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, or a VLFWR4 amino acid sequence of SEQ ID NO: 6108.

190. The multifunctional molecule of embodiment 189, wherein the NK cell engager comprises a VL comprising the amino acid sequence of SEQ ID NO: 6144 (or an amino acid sequence having at least about 93%, 95%, or 99% sequence identity to SEQ ID NO: 6144).

191. The multifunctional molecule of any of embodiments 103-114, 156, 157, or 164-181, wherein the NK cell engager comprises a light chain variable region (VL) comprising a light chain framework region 1 (VLFWR1) amino acid sequence of SEQ ID NO: 6109 (or a sequence with no more than 1, 2, 3, 4, 5, or 6 mutations, e.g., substitutions, additions, or deletions, therefrom), a VLFWR2 amino acid sequence of SEQ ID NO: 6110 (or a sequence with no more than 1, 2, 3, 4, 5, or 6 mutations, e.g., substitutions, additions, or deletions, therefrom), a VLFWR3 amino acid sequence of SEQ ID NO: 6111 (or a sequence with no more than 1, 2, 3, 4, 5, or 6 mutations, e.g., substitutions, additions, or deletions, therefrom), or a VLFWR4 amino acid sequence of SEQ ID NO: 6112 (or a sequence with no more than 1, 2, 3, 4, 5, or 6 mutations, e.g., substitutions, additions, or deletions, therefrom).

192. The multifunctional molecule of embodiment 191, wherein the NK cell engager comprises a light chain variable region (VL) comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, or a VLFWR4 amino acid sequence of SEQ ID NO: 6112.

193. The multifunctional molecule of embodiments 192, wherein the NK cell engager comprises a VL comprising the amino acid sequence of SEQ ID NO: 6145 (or an amino acid sequence having at least about 93%, 95%, or 99% sequence identity to SEQ ID NO: 6145).

194. The multifunctional molecule of any of embodiments 103-114, 156, 157, or 164-181, wherein the NK cell engager comprises a light chain variable region (VL) comprising a light chain framework region 1 (VLFWR1) amino acid sequence of SEQ ID NO: 6113 (or a sequence with no more than 1, 2, 3, 4, 5, or 6 mutations, e.g., substitutions, additions, or deletions, therefrom), a VLFWR2 amino acid sequence of SEQ ID NO: 6114 (or a sequence with no more than 1, 2, 3, 4, 5, or 6 mutations, e.g., substitutions, additions, or deletions, therefrom), a VLFWR3 amino acid sequence of SEQ ID NO: 6115 (or a sequence with no more than 1, 2, 3, 4, 5, or 6 mutations, e.g., substitutions, additions, or deletions, therefrom), or a VLFWR4 amino acid sequence of SEQ ID NO: 6116 (or a sequence with no more than 1, 2, 3, 4, 5, or 6 mutations, e.g., substitutions, additions, or deletions, therefrom).

195. The multifunctional molecule of embodiment 194, wherein the NK cell engager comprises a light chain variable region (VL) comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, or a VLFWR4 amino acid sequence of SEQ ID NO: 6116.

196. The multifunctional molecule of embodiment 195, wherein the NK cell engager comprises a VL comprising the amino acid sequence of SEQ ID NO: 6146 (or an amino acid sequence having at least about 93%, 95%, or 99% sequence identity to SEQ ID NO: 6146).

197. The multifunctional molecule of any of embodiments 103-114, 156, 157, or 164-181, wherein the NK cell engager comprises a light chain variable region (VL) comprising a light chain framework region 1 (VLFWR1) amino acid sequence of SEQ ID NO: 6117 (or a sequence with no more than 1, 2, 3, 4, 5, or 6 mutations, e.g., substitutions, additions, or deletions, therefrom), a VLFWR2 amino acid sequence of SEQ ID NO: 6118 (or a sequence with no more than 1, 2, 3, 4, 5, or 6 mutations, e.g., substitutions, additions, or deletions, therefrom), a VLFWR3 amino acid sequence of SEQ ID NO: 6119 (or a sequence with no more than 1, 2, 3, 4, 5, or 6 mutations, e.g., substitutions, additions, or deletions, therefrom), or a VLFWR4 amino acid sequence of SEQ ID NO: 6120 (or a sequence with no more than 1, 2, 3, 4, 5, or 6 mutations, e.g., substitutions, additions, or deletions, therefrom).

198. The multifunctional molecule of embodiment 197, wherein the NK cell engager comprises a light chain variable region (VL) comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, or a VLFWR4 amino acid sequence of SEQ ID NO: 6120.

199. The multifunctional molecule of embodiment 198, wherein the NK cell engager comprises a VL comprising the amino acid sequence of SEQ ID NO: 6147 (or an amino acid sequence having at least about 93%, 95%, or 99% sequence identity to SEQ ID NO: 6147).

200. The multifunctional molecule of any of embodiments 103-106, wherein the NK cell engager is an antibody molecule, e.g., an antigen binding domain, that binds to NKp46.

201. The multifunctional molecule of embodiment 200, wherein lysis of the lymphoma cell is mediated by NKp46.

202. The multifunctional molecule of either of embodiments 200 or 201, wherein the multifunctional molecule does not activate the NK cell when incubated with the NK cell in the absence of the TCRBV antigen (e.g., a TCRBV antigen corresponding to a biased TCRBV clonotype).

203. The multifunctional molecule of any one of embodiments 200-202, wherein the multifunctional molecule activates the NK cell when the NK cell is a NKp46 expressing NK cell and the TCRBV antigen (e.g., a TCRBV antigen corresponding to a biased TCRBV clonotype) is also present.

204. The multifunctional molecule of any one of embodiments 200-203, wherein the multifunctional molecule does not activate the NK cell when the NK cell is not a NKp46 expressing NK cell and the TCRBV antigen (e.g., a TCRBV antigen corresponding to a biased TCRBV clonotype) is also present.

205. The multifunctional molecule of any one of embodiments 200-204, wherein the NK cell engager comprises a VH comprising the amino acid sequence of SEQ ID NO: 6182 (or an amino acid sequence having at least about 75%, 80%, 85%, 90%, 95%, or 99% sequence identity to SEQ ID NO: 6182).

206. The multifunctional molecule of any one of embodiments 200-205, wherein the NK cell engager comprises a VL comprising the amino acid sequence of SEQ ID NO: 6183 (or an amino acid sequence having at least about 93%, 95%, or 99% sequence identity to SEQ ID NO: 6183).

207. The multifunctional molecule of 200-205, wherein the NK cell engager comprises an scFv comprising the amino acid sequence of SEQ ID NO: 6181 (or an amino acid sequence having at least about 93%, 95%, or 99% sequence identity to SEQ ID NO: 6181).

208. The multifunctional molecule of any of embodiments 103-106, wherein the NK cell engager is an antibody molecule, e.g., an antigen binding domain, that binds to NKG2D.

209. The multifunctional molecule of embodiment 208, wherein lysis of the lymphoma cell is mediated by NKG2D.

210. The multifunctional molecule of either of embodiments 208 or 209, wherein the multifunctional molecule does not activate the NK cell when incubated with the NK cell in the absence of the TCRBV antigen (e.g., a TCRBV antigen corresponding to a biased TCRBV clonotype).

211. The multifunctional molecule of any one of embodiments 208-210, wherein the multifunctional molecule activates the NK cell when the NK cell is a NKG2D expressing NK cell and the TCRBV antigen (e.g., a TCRBV antigen corresponding to a biased TCRBV clonotype) is also present.

212. The multifunctional molecule of any one of embodiments 208-211, wherein the multifunctional molecule does not activate the NK cell when the NK cell is not a NKG2D expressing NK cell and the TCRBV antigen (e.g., a TCRBV antigen corresponding to a biased TCRBV clonotype) is also present.

213. The multifunctional molecule of any one of embodiments 208-212, wherein the NK cell engager comprises a VH comprising the amino acid sequence of SEQ ID NO: 6176 (or an amino acid sequence having at least about 75%, 80%, 85%, 90%, 95%, or 99% sequence identity to SEQ ID NO: 6176).

214. The multifunctional molecule of any one of embodiments 208-213, wherein the NK cell engager comprises a VL comprising the amino acid sequence of SEQ ID NO: 6177 (or an amino acid sequence having at least about 93%, 95%, or 99% sequence identity to SEQ ID NO: 6177).

215. The multifunctional molecule of any of embodiments 208-214, wherein the NK cell engager comprises an scFv comprising the amino acid sequence of SEQ ID NO: 6175 (or an amino acid sequence having at least about 93%, 95%, or 99% sequence identity to SEQ ID NO: 6175).

216. The multifunctional molecule of any one of embodiments 208-212, wherein the NK cell engager comprises a VH comprising the amino acid sequence of SEQ ID NO: 6179 (or an amino acid sequence having at least about 75%, 80%, 85%, 90%, 95%, or 99% sequence identity to SEQ ID NO: 6179).

217. The multifunctional molecule of any one of embodiments 208-212 or 216 wherein the NK cell engager comprises a VL comprising the amino acid sequence of SEQ ID NO: 6180 (or an amino acid sequence having at least about 93%, 95%, or 99% sequence identity to SEQ ID NO: 6180).

218. The multifunctional molecule of any of embodiments 208-212, 216, or 217, wherein the NK cell engager comprises an scFv comprising the amino acid sequence of SEQ ID NO: 6178 (or an amino acid sequence having at least about 93%, 95%, or 99% sequence identity to SEQ ID NO: 6178).

219. The multifunctional molecule of any of embodiments 103-106, wherein the NK cell engager is an antibody molecule, e.g., an antigen binding domain, that binds to CD16.

220. The multifunctional molecule of embodiment 219, wherein lysis of the lymphoma cell is mediated by CD16.

221. The multifunctional molecule of either of embodiments 219 or 220, wherein the multifunctional molecule does not activate the NK cell when incubated with the NK cell in the absence of the TCRBV antigen (e.g., a TCRBV antigen corresponding to a biased TCRBV clonotype).

222. The multifunctional molecule of any one of embodiments 219-221, wherein the multifunctional molecule activates the NK cell when the NK cell is a CD16 expressing NK cell and the TCRBV antigen (e.g., a TCRBV antigen corresponding to a biased TCRBV clonotype) is also present.

223. The multifunctional molecule of any one of embodiments 219-222, wherein the multifunctional molecule does not activate the NK cell when the NK cell is not a CD16 expressing NK cell and the TCRBV antigen (e.g., a TCRBV antigen corresponding to a biased TCRBV clonotype) is also present.

224. The multifunctional molecule of any one of embodiments 219-223, wherein the NK cell engager comprises a VH comprising the amino acid sequence of SEQ ID NO: 6185 (or an amino acid sequence having at least about 75%, 80%, 85%, 90%, 95%, or 99% sequence identity to SEQ ID NO: 6185).

225. The multifunctional molecule of any one of embodiments 219-224, wherein the NK cell engager comprises a VL comprising the amino acid sequence of SEQ ID NO: 6186 (or an amino acid sequence having at least about 93%, 95%, or 99% sequence identity to SEQ ID NO: 6186).

226. The multifunctional molecule of any of embodiments 219-225, wherein the NK cell engager comprises an scFv comprising the amino acid sequence of SEQ ID NO: 6184 (or an amino acid sequence having at least about 93%, 95%, or 99% sequence identity to SEQ ID NO: 6184).

227. The multifunctional molecule of embodiment 103, wherein the NK cell engager is a ligand, optionally, the ligand further comprises an immunoglobulin constant region, e.g., an Fc region.

228. The multifunctional molecule of embodiment 227, wherein the NK cell engager is a ligand of NKp44 or NKp46, e.g., a viral HA.

229. The multifunctional molecule of embodiment 227, wherein the NK cell engager is a ligand of DAP10, e.g., a coreceptor for NKG2D.

230. The multifunctional molecule of embodiment 227, wherein the NK cell engager is a ligand of CD16, e.g., a CD16a/b ligand, e.g., a CD16a/b ligand further comprising an antibody Fc region.

231. The multifunctional molecule of any one of embodiments 98-100, wherein the immune cell engager mediates binding to, or activation of, or both of, one or more of a B cell, a macrophage, and/or a dendritic cell.

232. The multifunctional molecule of embodiment 231, wherein the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., a TLR4, e.g., a constitutively active TLR4 (caTLR4) or a TLR9 agonist); a 41BB; a CD2 agonist; a CD47; or a STING agonist, or a combination thereof.

233. The multifunctional molecule of any one of embodiments 98-100, wherein the immune cell engager is a B cell engager, e.g., a CD40L, an OX40L, or a CD70 ligand, or an antibody molecule that binds to OX40, CD40 or CD70.

234. The multifunctional molecule of any one of embodiments 98-100, wherein the immune cell engager is a macrophage cell engager, e.g., a CD2 agonist; a CD40L; an OX40L; an antibody molecule that binds to OX40, CD40 or CD70; an agonist of a Toll-like receptor (TLR) (e.g., a TLR4, e.g., a constitutively active TLR4 (caTLR4) or a TLR9 agonist); CD47; or a STING agonist.

235. The multifunctional molecule of any one of embodiments 98-100, wherein the immune cell engager is a dendritic cell engager, e.g., a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

236. The multifunctional molecule of embodiment 234 or 235, wherein the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages, e.g., wherein the STING agonist is covalently coupled to the multifunctional molecule.

237. The multifunctional molecule of any one of embodiments 1-97, wherein the multifunctional molecule comprises a cytokine molecule.

238. The multifunctional molecule of embodiment 237, wherein the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines.

239. The multifunctional molecule of embodiment 237 or 238, wherein the cytokine molecule is a monomer or a dimer.

240. The multifunctional molecule of any one of embodiments 237-239, wherein the cytokine molecule further comprises a receptor dimerizing domain, e.g., an IL15Ralpha dimerizing domain.

241. The multifunctional molecule of embodiment 240, wherein the cytokine molecule (e.g., IL-15) and the receptor dimerizing domain (e.g., an IL15Ralpha dimerizing domain) are not covalently linked, e.g., are non-covalently associated.

242. The multifunctional molecule of any of embodiments 1-97, wherein the multifunctional molecule comprises a cytokine inhibitor molecule.

243. The multifunctional molecule of embodiment 242, wherein the cytokine inhibitor molecule is a TGF-beta inhibitor.

244. The multifunctional molecule of either of embodiments 242 or 243, wherein the TGF-beta inhibitor inhibits (e.g., reduces the activity of): (i) TGF-beta 1; (ii) TGF-beta 2; (iii) TGF-beta 3; (iv) (i) and (ii); (v) (i) and (iii); (vi) (ii) and (iii); or (vii) (i), (ii), and (iii).

245. The multifunctional molecule of any of embodiments 242-244, wherein the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof.

246. The multifunctional molecule of embodiment 245, wherein the TGF-beta inhibitor comprises a portion of (i) TGFBR1; (ii) TGFBR2; (iii) TGFBR3; (iv) (i) and (ii); (v) (i) and (iii); (vi) (ii) and (iii); or (vii) (i), (ii), and (iii).

247. The multifunctional molecule of any of embodiments 242-246, wherein the TGF-beta inhibitor comprises an amino acid sequence selected from Table 16, or an amino acid sequence having at least about 93%, 95%, or 99% sequence identity thereto.

248. The multifunctional molecule of any of embodiments 1-97, wherein the multifunctional molecule comprises a death receptor signal engager chosen from a TNF-related apoptosis-inducing ligand (TRAIL) molecule, a death receptor molecule, or an antigen binding domain that specifically binds to a death receptor.

249. The multifunctional molecule of embodiment 248, wherein the death receptor signal engager activates death receptor signaling in the lymphocyte (e.g., T cell) comprising the TCRBV antigen, e.g., and induces apoptosis or cell death in said cell.

250. The multifunctional molecule of either of embodiments 248 or 249, wherein the death receptor signal engager does not activate death receptor signaling on cells other than lymphocytes comprising the TCRBV antigen.

251. The multifunctional molecule of any of embodiments 248-250, wherein the death receptor signal engager comprises a TRAIL molecule, e.g., one or more TRAIL polypeptides or a fragment thereof.

252. The multifunctional molecule of embodiment 251, wherein the TRAIL molecule specifically binds to Death Receptor 4 (DR4) or Death Receptor 5 (DR5).

253. The multifunctional molecule of either of embodiments 251 or 252, wherein the TRAIL molecule comprises a truncated TRAIL polypeptide, e.g., relative to a wild-type TRAIL polypeptide.

254. The multifunctional molecule of embodiment 253, wherein the TRAIL molecule comprises at least residues corresponding to amino acids 95-281 of human TRAIL, e.g., a truncated TRAIL molecule comprising residues corresponding to amino acids 95-281 of human TRAIL.

255. The multifunctional molecule of embodiment 254, wherein the TRAIL molecule comprises a truncated TRAIL polypeptide comprising amino acids 95-281 of human TRAIL, e.g., and not amino acids 1-94 of human TRAIL.

256. The multifunctional molecule of embodiment 253, wherein the TRAIL molecule comprises at least residues corresponding to amino acids 122-281 of human TRAIL, e.g., a truncated TRAIL molecule comprising residues corresponding to amino acids 122-281 of human TRAIL.

257. The multifunctional molecule of embodiment 256, wherein the TRAIL molecule comprises a truncated TRAIL polypeptide comprising amino acids 122-281 of human TRAIL, e.g., and not amino acids 1-121 of human TRAIL.

258. The multifunctional molecule of any of embodiments 251-257, wherein the death receptor signal engager comprises one, two, or three TRAIL molecules.

259. The multifunctional molecule of any of embodiments 248-250, wherein the death receptor signal engager comprises an antigen binding domain that specifically binds to a death receptor, e.g., Death Receptor 4 (DR4) or Death Receptor 5 (DR5).

260. The multifunctional molecule of embodiment 259, wherein the death receptor signal engager comprises one, two, or three antigen binding domains that specifically binds to a death receptor.

261. The multifunctional molecule of either of embodiments 259 or 260, wherein the antigen binding domain that specifically binds to a death receptor binds to DR5.

262. The multifunctional molecule of any of embodiments 259-261, wherein the antigen binding domain that specifically binds to a death receptor comprises tigatuzumab, drozitumab, or conatumumab.

263. The multifunctional molecule of any of embodiments 248-262, wherein the death receptor signal engager comprises an amino acid sequence selected from Table 11, or an amino acid sequence having at least about 75%, 80%, 85%, 90%, 95%, or 99% sequence identity thereto.

264. The multifunctional molecule of any of embodiments 248-263, wherein the death receptor signal engager comprises an amino acid sequence of SEQ ID NO: 6157, or an amino acid sequence having at least about 75%, 80%, 85%, 90%, 95%, or 99% sequence identity thereto.

265. The multifunctional molecule of any of embodiments 248-263, wherein the death receptor signal engager comprises an amino acid sequence of SEQ ID NO: 6158, or an amino acid sequence having at least about 75%, 80%, 85%, 90%, 95%, or 99% sequence identity thereto.

266. The multifunctional molecule of any of embodiments 248-263, wherein the death receptor signal engager comprises an amino acid sequence of SEQ ID NO: 6159, or an amino acid sequence having at least about 75%, 80%, 85%, 90%, 95%, or 99% sequence identity thereto.

267. The multifunctional molecule of any of embodiments 248-263, wherein the death receptor signal engager comprises an amino acid sequence of SEQ ID NO: 6160, or an amino acid sequence having at least about 75%, 80%, 85%, 90%, 95%, or 99% sequence identity thereto.

268. The multifunctional molecule of any of embodiments 248-263, wherein the death receptor signal engager comprises an amino acid sequence of SEQ ID NO: 6161, or an amino acid sequence having at least about 75%, 80%, 85%, 90%, 95%, or 99% sequence identity thereto.

269. The multifunctional molecule of any of embodiments 248-263, wherein the death receptor signal engager comprises an amino acid sequence of SEQ ID NO: 6162, or an amino acid sequence having at least about 75%, 80%, 85%, 90%, 95%, or 99% sequence identity thereto.

270. The multifunctional molecule of any of embodiments 248-263, wherein the death receptor signal engager comprises an amino acid sequence of SEQ ID NO: 6163, or an amino acid sequence having at least about 75%, 80%, 85%, 90%, 95%, or 99% sequence identity thereto.

271. The multifunctional molecule of any of embodiments 248-263, wherein the death receptor signal engager comprises an amino acid sequence of SEQ ID NO: 6164, or an amino acid sequence having at least about 75%, 80%, 85%, 90%, 95%, or 99% sequence identity thereto.

272. The multifunctional molecule of any of embodiments 248-263, wherein the death receptor signal engager comprises an amino acid sequence of SEQ ID NO: 6165, or an amino acid sequence having at least about 75%, 80%, 85%, 90%, 95%, or 99% sequence identity thereto.

273. The multifunctional molecule of embodiment 102, wherein the T cell engager binds to TCRβ.

274. The multifunctional molecule of embodiment 273, wherein the T cell engager comprises an antigen binding domain (e.g., an antibody molecule or fragment thereof) that binds to (e.g., and in some embodiments activates) CD3.

275. The multifunctional molecule of either of embodiments 273 or 274, wherein the T cell engager does not bind to the lymphocyte comprising the TCRBV antigen.

276. The multifunctional molecule of any of embodiments 273-275, wherein the T cell engager does not activate the lymphocyte comprising the TCRBV.

277. The multifunctional molecule of any one of embodiments 1-276, wherein the multifunctional molecule comprises:

(i) an immune cell engager (e.g., a T cell engager, an NK cell engager, a B cell engager, a dendritic cell engager, or a macrophage cell engager) and a cytokine molecule,
(ii) an immune cell engager (e.g., a T cell engager, an NK cell engager, a B cell engager, a dendritic cell engager, or a macrophage cell engager) and a cytokine inhibitor molecule,
(iii) an immune cell engager (e.g., a T cell engager, an NK cell engager, a B cell engager, a dendritic cell engager, or a macrophage cell engager) and a death receptor signal engager,
(iv) a cytokine molecule and a death receptor signal engager,
(v) a cytokine inhibitor molecule and a death receptor signal engager,
(vi) an immune cell engager (e.g., a T cell engager, an NK cell engager, a B cell engager, a dendritic cell engager, or a macrophage cell engager), a cytokine molecule, and a death receptor signal engager, or
(vii) an immune cell engager (e.g., a T cell engager, an NK cell engager, a B cell engager, a dendritic cell engager, or a macrophage cell engager), a cytokine inhibitor molecule, and a death receptor signal engager.

278. The multifunctional molecule of any one of embodiments 1-277, wherein the multifunctional molecule comprises the following configuration:

A, B-[dimerization module]-C, -D, wherein:

(a) the dimerization module comprises an immunoglobulin constant domain, e.g., a heavy chain constant domain (e.g., a homodimeric or heterodimeric heavy chain constant region, e.g., an Fc region), or a constant domain of an immunoglobulin variable region (e.g., a Fab region); and
(b) A, B, C, and D are independently absent; (i) an antigen binding domain that selectively binds to a TCRBV antigen; (ii) an immune cell engager chosen from a T cell engager, an NK cell engager, a B cell engager, a dendritic cell engager, or a macrophage cell engager; (iii) a cytokine molecule or cytokine inhibitor molecule; (iv) a death receptor signal engager; or (v) a stromal modifying moiety, provided that:
at least one, two, or three of A, B, C, and D comprises an antigen binding domain that selectively binds to a TCRBV antigen, and
any of the remaining A, B, C, and D is absent or comprises one of an immune cell engager, a cytokine molecule, a cytokine inhibitor molecule, a death receptor signal engager, or a stromal modifying moiety.

279. The multifunctional molecule of embodiment 278, wherein:

(1) A comprises an antigen binding domain that selectively binds to a TCRBV antigen, and B, C, or D comprises an immune cell engager, e.g., a T cell engager, e.g., an anti-CD3 antibody molecule;
(2) A comprises an antigen binding domain that selectively binds to a TCRBV antigen, and B, C, or D comprises an immune cell engager, e.g., an NK cell engager, e.g., an anti-NKp30, anti-NKp46, anti-NKG2D, or anti-CD16 antibody molecule;
(3) A comprises an antigen binding domain that selectively binds to a TCRBV antigen, and B, C, or D comprises a cytokine molecule;
(4) A comprises an antigen binding domain that selectively binds to a TCRBV antigen, and B, C, or D comprises a cytokine inhibitor molecule;
(5) A comprises an antigen binding domain that selectively binds to a TCRBV antigen, and B, C, or D comprises a death receptor signal engager;
(6) A comprises a first antigen binding domain that selectively binds to a TCRBV antigen, B comprises a second antigen binding domain that selectively binds to a TCRBV antigen, and C or D comprises an immune cell engager, e.g., a T cell engager, e.g., an anti-CD3 antibody molecule;
(7) A comprises a first antigen binding domain that selectively binds to a TCRBV antigen, B comprises a second antigen binding domain that selectively binds to a TCRBV antigen, and C or D comprises an immune cell engager, e.g., an NK cell engager, e.g., an anti-NKp30, anti-NKp46, anti-NKG2D, or anti-CD16 antibody molecule;
(8) A comprises a first antigen binding domain that selectively binds to a TCRBV antigen, B comprises a second antigen binding domain that selectively binds to a TCRBV antigen, and C or D comprises a cytokine molecule;
(9) A comprises a first antigen binding domain that selectively binds to a TCRBV antigen, B comprises a second antigen binding domain that selectively binds to a TCRBV antigen, and C or D comprises a cytokine inhibitor molecule;
(10) A comprises a first antigen binding domain that selectively binds to a TCRBV antigen, B comprises a second antigen binding domain that selectively binds to a TCRBV antigen, and C or D comprises a death receptor signal engager;
(11) A comprises a first antigen binding domain that selectively binds to a TCRBV antigen, C comprises a second antigen binding domain that selectively binds to a TCRBV antigen, and B or D comprises an immune cell engager, e.g., a T cell engager, e.g., an anti-CD3 antibody molecule;
(12) A comprises a first antigen binding domain that selectively binds to a TCRBV antigen, C comprises a second antigen binding domain that selectively binds to a TCRBV antigen, and B or D comprises an immune cell engager, e.g., an NK cell engager, e.g., an anti-NKp30, anti-NKp46, anti-NKG2D, or anti-CD16 antibody molecule;
(13) A comprises a first antigen binding domain that selectively binds to a TCRBV antigen, C comprises a second antigen binding domain that selectively binds to a TCRBV antigen, and B or D comprises a cytokine molecule;
(14) A comprises a first antigen binding domain that selectively binds to a TCRBV antigen, C comprises a second antigen binding domain that selectively binds to a TCRBV antigen, and B or D comprises a cytokine inhibitor molecule;
(15) A comprises a first antigen binding domain that selectively binds to a TCRBV antigen, C comprises a second antigen binding domain that selectively binds to a TCRBV antigen, and B or D comprises a death receptor signal engager;
(16) A comprises a first antigen binding domain that selectively binds to a TCRBV antigen, and B, C, or D comprises (a) an immune cell engager, e.g., an NK cell engager, e.g., an anti-NKp30, anti-NKp46, anti-NKG2D, or anti-CD16 antibody molecule, and (b) a cytokine molecule;
(17) A comprises a first antigen binding domain that selectively binds to a TCRBV antigen, and B, C, or D comprises (a) an immune cell engager, e.g., an NK cell engager, e.g., an anti-NKp30, anti-NKp46, anti-NKG2D, or anti-CD16 antibody molecule, and (b) a cytokine inhibitor molecule;
(18) A comprises a first antigen binding domain that selectively binds to a TCRBV antigen, and B, C, or D comprises (a) an immune cell engager, e.g., an NK cell engager, e.g., an anti-NKp30, anti-NKp46, anti-NKG2D, or anti-CD16 antibody molecule, and (b) a death receptor signal engager;
(19) A comprises a first antigen binding domain that selectively binds to a TCRBV antigen, and B, C, or D comprises (a) an immune cell engager, e.g., a T cell engager, e.g., an anti-CD3 antibody molecule, and (b) a cytokine molecule;
(20) A comprises a first antigen binding domain that selectively binds to a TCRBV antigen, and B, C, or D comprises (a) an immune cell engager, e.g., a T cell engager, e.g., an anti-CD3 antibody molecule, and (b) a cytokine inhibitor molecule;
(21) A comprises a first antigen binding domain that selectively binds to a TCRBV antigen, and B, C, or D comprises (a) an immune cell engager, e.g., a T cell engager, e.g., an anti-CD3 antibody molecule, and (b) a death receptor signal engager;
(22) A comprises a first antigen binding domain that selectively binds to a TCRBV antigen, and B, C, or D comprises (a) a cytokine molecule and (b) a death receptor signal engager;
(23) A comprises a first antigen binding domain that selectively binds to a TCRBV antigen, and B, C, or D comprises (a) a cytokine inhibitor molecule and (b) a death receptor signal engager;
(24) A comprises a first antigen binding domain that selectively binds to a TCRBV antigen, B comprises a second antigen binding domain that selectively binds to a TCRBV antigen, and C or D comprises (a) an immune cell engager, e.g., an NK cell engager, e.g., an anti-NKp30, anti-NKp46, anti-NKG2D, or anti-CD16 antibody molecule, and (b) a cytokine molecule;
(25) A comprises a first antigen binding domain that selectively binds to a TCRBV antigen, B comprises a second antigen binding domain that selectively binds to a TCRBV antigen, and C or D comprises (a) an immune cell engager, e.g., an NK cell engager, e.g., an anti-NKp30, anti-NKp46, anti-NKG2D, or anti-CD16 antibody molecule, and (b) a cytokine inhibitor molecule;
(26) A comprises a first antigen binding domain that selectively binds to a TCRBV antigen, B comprises a second antigen binding domain that selectively binds to a TCRBV antigen, and C or D comprises (a) an immune cell engager, e.g., an NK cell engager, e.g., an anti-NKp30, anti-NKp46, anti-NKG2D, or anti-CD16 antibody molecule, and (b) a death receptor signal engager;
(27) A comprises a first antigen binding domain that selectively binds to a TCRBV antigen, B comprises a second antigen binding domain that selectively binds to a TCRBV antigen, and C or D comprises (a) an immune cell engager, e.g., an NK cell engager, e.g., an a anti-NKp30, anti-NKp46, anti-NKG2D, or anti-CD16 antibody molecule, and (b) a stromal modifying moiety;
(28) A comprises a first antigen binding domain that selectively binds to a TCRBV antigen, B comprises a second antigen binding domain that selectively binds to a TCRBV antigen, and C or D comprises (a) an immune cell engager, e.g., a T cell engager, e.g., an anti-CD3 antibody molecule, and (b) a cytokine molecule;
(29) A comprises a first antigen binding domain that selectively binds to a TCRBV antigen, B comprises a second antigen binding domain that selectively binds to a TCRBV antigen, and C or D comprises (a) an immune cell engager, e.g., a T cell engager, e.g., an anti-CD3 antibody molecule, and (b) a cytokine inhibitor molecule;
(30) A comprises a first antigen binding domain that selectively binds to a TCRBV antigen, B comprises a second antigen binding domain that selectively binds to a TCRBV antigen, and C or D comprises (a) an immune cell engager, e.g., a T cell engager, e.g., an anti-CD3 antibody molecule, and (b) a death receptor signal engager;
(31) A comprises a first antigen binding domain that selectively binds to a TCRBV antigen, B comprises a second antigen binding domain that selectively binds to a TCRBV antigen, and C or D comprises (a) a cytokine molecule and (b) a death receptor signal engager;
(32) A comprises a first antigen binding domain that selectively binds to a TCRBV antigen, B comprises a second antigen binding domain that selectively binds to a TCRBV antigen, and C or D comprises (a) a cytokine inhibitor molecule and (b) a death receptor signal engager;
(33) A comprises a first antigen binding domain that selectively binds to a TCRBV antigen, C comprises a second antigen binding domain that selectively binds to a TCRBV antigen, and B or D comprises (a) an immune cell engager, e.g., an NK cell engager, e.g., an anti-NKp30, anti-NKp46, anti-NKG2D, or anti-CD16 antibody molecule, and (b) a cytokine molecule;
(34) A comprises a first antigen binding domain that selectively binds to a TCRBV antigen, C comprises a second antigen binding domain that selectively binds to a TCRBV antigen, and B or D comprises (a) an immune cell engager, e.g., an NK cell engager, e.g., an anti-NKp30, anti-NKp46, anti-NKG2D, or anti-CD16 antibody molecule, and (b) a cytokine inhibitor molecule;
(35) A comprises a first antigen binding domain that selectively binds to a TCRBV antigen, C comprises a second antigen binding domain that selectively binds to a TCRBV antigen, and B or D comprises (a) an immune cell engager, e.g., an NK cell engager, e.g., an anti-NKp30, anti-NKp46, anti-NKG2D, or anti-CD16 antibody molecule, and (b) a death receptor signal engager;
(36) A comprises a first antigen binding domain that selectively binds to a TCRBV antigen, C comprises a second antigen binding domain that selectively binds to a TCRBV antigen, and B or D comprises (a) an immune cell engager, e.g., a T cell engager, e.g., an anti-CD3 antibody molecule, and (b) a cytokine molecule;
(37) A comprises a first antigen binding domain that selectively binds to a TCRBV antigen, C comprises a second antigen binding domain that selectively binds to a TCRBV antigen, and B or D comprises (a) an immune cell engager, e.g., a T cell engager, e.g., an anti-CD3 antibody molecule, and (b) a cytokine inhibitor molecule;
(38) A comprises a first antigen binding domain that selectively binds to a TCRBV antigen, C comprises a second antigen binding domain that selectively binds to a TCRBV antigen, and B or D comprises (a) an immune cell engager, e.g., a T cell engager, e.g., an anti-CD3 antibody molecule, and (b) a death receptor signal engager;
(39) A comprises a first antigen binding domain that selectively binds to a TCRBV antigen, C comprises a second antigen binding domain that selectively binds to a TCRBV antigen, and B or D comprises (a) a cytokine molecule and (b) a death receptor signal engager;
(40) A comprises a first antigen binding domain that selectively binds to a TCRBV antigen, C comprises a second antigen binding domain that selectively binds to a TCRBV antigen, and B or D comprises (a) a cytokine inhibitor molecule and (b) a death receptor signal engager; or

280. The multifunctional molecule of embodiment 278 or 279, wherein the dimerization module comprises one or more immunoglobulin chain constant regions (e.g., Fc regions) comprising one or more of: a paired cavity-protuberance (“knob-in-a hole”), an electrostatic interaction, or a strand-exchange.

281. The multifunctional molecule of embodiment 280, wherein the one or more immunoglobulin chain constant regions (e.g., Fc regions) comprise an amino acid substitution at a position chosen from one or more of 347, 349, 350, 351, 366, 368, 370, 392, 394, 395, 397, 398, 399, 405, 407, or 409, e.g., of the Fc region of human IgG1, optionally wherein the one or more immunoglobulin chain constant regions (e.g., Fc regions) comprise an amino acid substitution chosen from: T366S, L368A, or Y407V (e.g., corresponding to a cavity or hole), or T366W (e.g., corresponding to a protuberance or knob), or a combination thereof.

282. The multifunctional molecule of any one of embodiments 1-281, further comprising a linker, e.g., a linker between one or more of: the antigen binding domain and the immune cell engager, the antigen binding domain and the cytokine molecule, the antigen binding domain and the stromal modifying moiety, the immune cell engager and the cytokine molecule, the immune cell engager and the stromal modifying moiety, the cytokine molecule and the stromal modifying moiety, the antigen binding domain and the dimerization module, the immune cell engager and the dimerization module, the cytokine molecule and the dimerization module, or the stromal modifying moiety and the dimerization module.

283. The multifunctional molecule of embodiment 282, wherein the linker is chosen from: a cleavable linker, a non-cleavable linker, a peptide linker, a flexible linker, a rigid linker, a helical linker, or a non-helical linker.

284. The multifunctional molecule of embodiment 282 or 283, wherein the linker is a peptide linker.

285. The multifunctional molecule of 284, wherein the peptide linker comprises Gly and Ser.

286. The multifunctional molecule of 285, wherein the peptide linker comprises an amino acid sequence chosen from SEQ ID NOs: 7248-7251 or 7252-7253 and 77-78.

287. A multifunctional molecule, comprising:

(i) a first antigen binding domain that selectively binds to a TCRBV antigen, and
(ii) an NK cell engager, e.g., an anti-NKp30, anti-NKp46, anti-NKG2D, or anti-CD16 antibody molecule.

288. The multifunctional molecule of embodiment 287, wherein the NK cell engager comprises an anti-NKp30 antibody molecule.

289. The multifunctional molecule of embodiment 287, wherein the NK cell engager comprises an anti-NKp46 antibody molecule.

290. A multifunctional molecule, comprising:

(i) a first antigen binding domain that binds to, e.g., selectively binds to, T cell receptor variable beta (TCRBV), e.g., a TCRBV antigen, and
(ii) a death receptor signal engager.

291. A multifunctional molecule, comprising:

(i) a first antigen binding domain that binds to, e.g., selectively binds to, T cell receptor variable beta (TCRBV), e.g., a TCRBV antigen, and
(ii) a cytokine inhibitor molecule, e.g., TGF-beta inhibitor.

292. The multifunctional molecule of any of embodiments 1-291, wherein the multifunctional molecule binds to the TCRBV antigen monovalently.

293. The multifunctional molecule of any one of embodiments 1-291, wherein the multifunctional molecule binds to the TCRBV antigen multivalently, e.g., di-, tri-, tetra-, penta-, hexa-, hepta-, octa-, nona-, or deca-valently.

294. The multifunctional molecule of any of embodiments 2-261, wherein the multifunctional molecule binds to the TCRBV antigen on a lymphocyte expressing the TCRBV antigen.

295. The multifunctional molecule of any preceding embodiment, wherein the multifunctional molecule binds, e.g., via the immune cell engager, to the immune cell monovalently.

296. The multifunctional molecule of any one of embodiments 1-294, wherein the multifunctional molecule binds, e.g., via the immune cell engager, to the immune cell multivalently, e.g., di-, tri-, tetra-, penta-, hexa-, hepta-, octa-, nona-, or deca-valently.

297. The multifunctional molecule of any preceding embodiment, further comprising a heavy chain constant region, e.g., an Fc region, that mediates antibody dependent cellular cytotoxicity (ADCC).

298. The multifunctional molecule of any preceding embodiment, further comprising a heavy chain constant region, e.g., an Fc region, that mediates complement dependent cytotoxicity (e.g., via C1q).

299. A nucleic acid molecule encoding the multifunctional molecule of any one of embodiments 1-298.

300. A vector, e.g., an expression vector, comprising the nucleic acid molecules of embodiment 299.

301. A host cell comprising the nucleic acid molecule of embodiment 299 or the vector of embodiment 300.

302. A method of making, e.g., producing, the multifunctional molecule or antibody molecule of any one of embodiments 1-298, comprising culturing the host cell of embodiment 301, under suitable conditions, e.g., conditions suitable for gene expression and/or homo- or heterodimerization.

303. A pharmaceutical composition comprising the multifunctional molecule of any one of embodiments 1-298 and a pharmaceutically acceptable carrier, excipient, or stabilizer.

304. A method of treating a TCR bias, comprising administering to a subject in need thereof the multifunctional molecule of any one of embodiments 1-298, wherein the multifunctional molecule is administered in an amount effective to treat the TCR bias.

305. A method of treating an autoimmune disease (e.g., an autoimmune disease associated with a TCR bias), comprising administering to a subject in need thereof the multifunctional molecule of any one of embodiments 1-298, wherein the multifunctional molecule is administered in an amount effective to treat the autoimmune disease.

306. The method of either of embodiments 304 or 305, further comprising identifying, evaluating, or selecting a subject in need of treatment, wherein identifying, evaluating, or selecting comprises determining (e.g., directly determining or indirectly determining, e.g., obtaining information regarding) whether a subject has a TCR bias or an autoimmune disease (e.g., an autoimmune disease associated with a TCR bias).

307. The method of embodiment 306, further comprising, responsive to determining that a subject has a TCR bias or an autoimmune disease (e.g., an autoimmune disease associated with a TCR bias):

optionally, selecting the subject for treatment with a multifunctional molecule comprising an antigen binding domain that binds to a TCRBV antigen (e.g., a TCRBV antigen corresponding to the biased TCRBV clonotype), and
administering a multifunctional molecule comprising an antigen binding domain that binds to a TCRBV antigen (e.g., a TCRBV antigen corresponding to the biased TCRBV clonotype).

308. A method of treating a TCR bias, comprising:

responsive to determining that a subject has a TCR bias, administering to a subject in need thereof the multifunctional molecule of any one of claims 1-298, wherein the multifunctional molecule is administered in an amount effective to treat the TCR bias.

309. A method of treating an autoimmune disease (e.g., an autoimmune disease associated with a TCR bias), comprising:

responsive to determining that a subject has an autoimmune disease (e.g., an autoimmune disease associated with a TCR bias), administering to a subject in need thereof the multifunctional molecule of any one of claims 1-298, wherein the multifunctional molecule is administered in an amount effective to treat the autoimmune disease (e.g., an autoimmune disease associated with a TCR bias).

310. The method of any of embodiments 304-309, wherein the subject has a TCR bias (e.g., a biased TCRBV clonotype) and/or an autoimmune disease associated with said bias.

311. A method of identifying a subject in need of treatment for cancer using a multifunctional molecule of any of embodiments 1-298, comprising determining (e.g., directly determining or indirectly determining, e.g., obtaining information regarding) whether a subject has a TCR bias (e.g., a biased TCRBV clonotype) and/or an autoimmune disease associated with said bias, wherein:

responsive to determining that the subject has a TCR bias (e.g., a biased TCRBV clonotype) and/or an autoimmune disease associated with said bias, identifying the subject as a candidate for treatment using a multifunctional molecule comprising an antigen binding domain that binds to the TCRBV antigen, and optionally not as a candidate for treatment using a multifunctional molecule comprising an antigen binding domain that does not bind to the TCRBV antigen (e.g., that binds to a different TCRBV antigen).

312. The method of embodiment 311, further comprising:

responsive to identifying the subject as a candidate for treatment using a multifunctional molecule comprising an antigen binding domain that binds to the TCRBV antigen, treating the subject with (e.g., administering to the subject) a multifunctional molecule comprising an antigen binding domain that binds to the TCRBV antigen.

313. A method of evaluating a subject in need of treatment for a TCR bias (e.g., a biased TCRBV clonotype) and/or an autoimmune disease associated with said bias, comprising determining (e.g., directly determining or indirectly determining, e.g., obtaining information regarding) whether a subject has a TCR bias.

314. The method of embodiment 313, further comprising responsive to the evaluation, treating the subject with (e.g., administering to the subject) a multifunctional molecule comprising an antigen binding domain that binds to the TCRBV antigen.

315. The method of any one of embodiments 304-314, wherein the TCR bias is associated with an autoimmune disease.

316. The method of embodiment 315, wherein the autoimmune disease is selected from Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellar degeneration (PCD), Lambert-Eaton syndrome, psoriasis, scleroderma, CREST (calcinosis, Raynaud phenomenon, esophageal dysmotility, sclerodactyly, and telangiectasia) syndrome, dermatitis herpetiformis, dermatomyositis, bullous pemphigoid, cicatricial pemphigoid/benign mucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis), Takayasu's arteritis, polyarteritis nodosa, microscopic polyangiitis (MPA), leukocytoclastic vasculitis, Cogan's syndrome, uveitis, peripheral uveitis (Pars planitis), scleritis, autoimmune inner ear disease (AIED), Crohn's, ulcerative colitis (UC), Dressler's syndrome, Rheumatic fever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), Sjogren's syndrome, ocular cicatricial pemphigoid, or Hashimoto's thyroiditis.

317. The method of any of embodiments 304-316, further comprising administering a second therapeutic treatment.

318. The method of embodiment 317, wherein the second therapeutic treatment comprises a therapeutic agent (e.g., a chemotherapeutic agent, a biologic agent, hormonal therapy), radiation, or surgery.

319. A method of treating an autoimmune disease (e.g., an autoimmune disease associated with a TCR bias), in a subject in need thereof, comprising administering to said subject an effective amount, e.g., a therapeutically effective amount, of an antibody molecule which binds (e.g., specifically binds) to a T cell receptor beta variable region (TCRβV) (“anti-TCRβV antibody molecule”), thereby treating the disorder.

320. A method of depleting a population of T cells in a subject having an autoimmune disorder (e.g., an autoimmune disease associated with a TCR bias), comprising, contacting the T cell population with an effective amount of an antibody molecule which binds (e.g., specifically binds) to a T cell receptor beta variable region (TCRβV) (“anti-TCRβV antibody molecule”).

321. The method of claim 320, wherein the contacting occurs in vivo or in vitro.

322. The method of any one of claims 319-321, wherein the anti-TCRβV antibody molecule:

(i) is not an antibody molecule disclosed in U.S. Pat. No. 5,861,155;
(ii) binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155;
(iii) binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155;
(iii) binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155 or
(iv) binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

323. The method of any one of claims 319-322, wherein the anti-TCRβV antibody molecule comprises an Fc region, e.g., an Fc region having effector function, e.g., antibody dependent cell-mediated cytotoxicity (ADCC), Antibody-dependent cellular phagocytosis (ADCP) and/or complement dependent cytotoxicity (CDC).

324. The method of any claim 323, wherein the anti-TCRβV antibody molecule comprises an Fc region with enhanced effector function, e.g., as compared to a wildtype Fc region.

325. The method of any one of claims 319-324, wherein the anti-TCRβV antibody molecule comprises a human IgG1 region or a human IgG4 region.

326. The method of any one of claim 319 or 321-325, wherein the autoimmune disease is selected from Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellar degeneration (PCD), Lambert-Eaton syndrome, psoriasis, scleroderma, CREST (calcinosis, Raynaud phenomenon, esophageal dysmotility, sclerodactyly, and telangiectasia) syndrome, dermatitis herpetiformis, dermatomyositis, bullous pemphigoid, cicatricial pemphigoid/benign mucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis), Takayasu's arteritis, polyarteritis nodosa, microscopic polyangiitis (MPA), leukocytoclastic vasculitis, Cogan's syndrome, uveitis, peripheral uveitis (Pars planitis), scleritis, autoimmune inner ear disease (AIED), Crohn's, ulcerative colitis (UC), Dressler's syndrome, Rheumatic fever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), Sjogren's syndrome, ocular cicatricial pemphigoid, or Hashimoto's thyroiditis.

327. The method of any one of claims 319-326, wherein the anti-TCRβV antibody molecule comprises an antigen binding domain comprising one or more (e.g., all three) of a LC CDR1, LC CDR2, and LC CDR3 provided in Tables 1A, 2A, 10A, 11A, 12A or 13A; and/or one or more (e.g., all three) of a HC CDR1, HC CDR2, and HC CDR3 provided in Tables 1A, 2A, 10A, 11A, 12A or 13A, or a sequence with at least 85%, 90%, 95%, 96%, 97%, 98%, or 99% identity thereto.

328. The method of any one of claims 319-327, wherein the anti-TCRβV antibody molecule comprises a variable heavy chain (VH) and/or a variable light chain (VL) provided in Tables 1A, 2A, 10A, 11A, 12A or 13A, or a sequence with at least 85%, 90%, 95%, 96%, 97%, 98%, or 99% identity thereto.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1B shows the alignment of the Antibody A source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 1A shows VH sequences for murine Antibody A (SEQ ID NO: 1) and humanized Antibody A-H (SEQ ID NO: 9). FIG. 1B shows VL sequences for murine Antibody A (SEQ ID NO: 2) and humanized Antibody A-H (SEQ ID NO: 10 and SEQ ID NO: 11).

FIGS. 2A-2B shows the alignment of the Antibody B source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 2A shows the VH sequence for murine Antibody B (SEQ ID NO: 15) and humanized VH sequences B-H.1A to B-H.1C (SEQ ID NOs: 23-25). FIG. 2B shows the VL sequence for murine Antibody B (SEQ ID NO: 16) and humanized VL sequences B-H.1D to B-H.1H (SEQ ID NOs: 26-30).

FIG. 3 depicts the phylogenetic tree of TCRBV gene family and subfamilies with corresponding antibodies mapped. Subfamily identities are as follows: Subfamily A: TCRβ V6; Subfamily B: TCRβ V10; Subfamily C: TCRβ V12; Subfamily D: TCRβ V5; Subfamily E: TCRβ V7; Subfamily F: TCRβ V11; Subfamily G: TCRβ V14; Subfamily H: TCRβ V16; Subfamily I:TCRβ V18; Subfamily J:TCRβ V9; Subfamily K: TCRβ V13; Subfamily L: TCRβ V4; Subfamily M:TCRβ V3; Subfamily N:TCRβ V2; Subfamily O:TCRβ V15; Subfamily P: TCRβ V30; Subfamily Q: TCRβ V19; Subfamily R:TCRβ V27; Subfamily S:TCRβ V28; Subfamily T: TCRβ V24; Subfamily U: TCRβ V20; Subfamily V: TCRβ V25; and Subfamily W:TCRβ V29 subfamily. Subfamily members are described in detail herein in the Section titled “TCR beta V (TCRβV)”.

FIG. 4 is a graph showing binding of NKp30 antibodies to NK92 cells. Data was calculated as the percent-AF747 positive population.

FIG. 5 is a graph showing activation of NK92 cells by NKp30 antibodies. Data were generated using hamster anti-NKp30 mAbs.

FIGS. 6A and 6B are schematics showing the alignment of affinity matured humanized Antibody A-H sequences. FIG. 6A shows the alignment of affinity matured humanized Antibody A-H VL sequences (SEQ ID NOs: 3377-3389, respectively, in order of appearance). FIG. 6B shows the alignment of affinity matured humanized Antibody A-H VH sequences (SEQ ID NOs: 3390-3436, respectively, in order of appearance).

### DETAILED DESCRIPTION OF THE INVENTION

Disclosed herein are multifunctional molecules (also referred to herein as “multispecific molecules”) that include a plurality of (e.g., two or more) functionalities (or binding specificities), comprising (i) an antigen binding domain that binds to, e.g., selectively binds to, T cell receptor variable beta (TCRBV), e.g., a TCRBV antigen, and (ii) one, two, or all of: (a) an immune cell engager chosen from a T cell engager, an NK cell engager (e.g., a molecule that binds to NKp30, NKp46, NKG2D, or CD16), a B cell engager, a dendritic cell engager, or a macrophage cell engager; (b) a cytokine molecule or cytokine inhibitor molecule; and (c) a death receptor signal engager. In some embodiments, the antigen binding domain comprises a sequence or part of a sequence found in Tables 13 or 14. In some embodiments, the immune cell engager comprises an NK cell engager comprising a sequence or part of a sequence found in Tables 7-10. In some embodiments, the antigen binding domain comprises a sequence or part of a sequence found in Tables 13 or 14 and the immune cell engager comprises an NK cell engager comprising a sequence or part of a sequence found in Tables 7-10.

In an embodiment, the multispecific or multifunctional molecule is a bispecific (or bifunctional) molecule, a trispecific (or trifunctional) molecule, or a tetraspecific (or tetrafunctional) molecule.

In some embodiments, the multifunctional molecule comprises an antigen binding domain that binds a TCRBV antigen on the surface of a lymphocyte, e.g., T cell. In some embodiments, the TCRBV antigen corresponds to a biased TCRBV clonotype, e.g., TCRs comprising the TCRBV antigen may be over-represented in the TCR repertoire or lymphocyte (e.g., T cell) pool of a subject (e.g., subjects with autoimmune disease associated with TCR bias), or expressed at a level that is higher than the level in other subjects (e.g., non-autoimmune disease subjects).

Without being bound by theory, the multispecific or multifunctional molecules disclosed herein are expected to localize (e.g., bridge) and/or activate an immune cell (e.g., an immune effector cell chosen from a T cell, an NK cell, a B cell, a dendritic cell or a macrophage), in the presence of a cell (e.g., a lymphocyte, e.g., T cell) expressing the TCRBV antigen (e.g., a TCRBV antigen corresponding to a biased TCRBV clonotype), e.g., on the cell surface. Increasing the proximity and/or activity of the immune cell, in the presence of the cell (e.g., a lymphocyte, e.g., T cell) expressing the TCRBV antigen (e.g., a TCRBV antigen corresponding to a biased TCRBV clonotype) using the multispecific or multifunctional molecules described herein is expected to enhance an immune response against the target cell, thereby providing a more effective therapy (e.g., by decreasing the level of the biased TCR and/or T cell expressing the biased TCR). In another embodiment, targeting a cell (e.g., a lymphocyte, e.g., T cell) expressing the TCRBV antigen (e.g., a TCRBV antigen corresponding to a biased TCRBV clonotype) with a multifunctional molecule also comprising a cell death inducing moiety (e.g., a death receptor signal engager) is thought to promote the death of the target cell (e.g., by decreasing the level of the biased TCR and/or T cell expressing the biased TCR).

Without being bound by theory, by utilizing, in some embodiments, a multispecific or multifunctional molecule specific for a particular TCRBV antigen (e.g., a TCRBV antigen corresponding to a biased TCRBV clonotype), but not with specificity for other or all types of T cell receptors, it is expected that the deleterious effects of increasing the proximity or activity of immune cells toward T cells generally or promoting cell death in T cells generally may be mitigated. In this way, it is thought that use of the multispecific or multifunctional molecules disclosed herein may increase the proximity or activity of immune cells toward cells comprising TCRBV antigen corresponding to a biased TCRBV clonotype without necessarily increasing proximity or activity of immune cells toward T cells generally, or promote cell death in cells comprising TCRBV antigen corresponding to a biased TCRBV clonotype without necessarily increasing cell death in T cells generally.

Accordingly, provided herein are, inter alia, multispecific or multifunctional molecules (e.g., multispecific or multifunctional antibody molecules) that include the aforesaid moieties, nucleic acids encoding the same, methods of producing the aforesaid molecules, and methods of treating a disease or disorder, e.g., an autoimmune disease or a TCR bias, using the aforesaid molecules.

### Definitions

In some embodiments, the multifunctional molecule includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “autoimmune” disease, disorder, or condition refers to a disease where the body's immune system attacks its own cells or tissues. An autoimmune disease can results in the production of autoantibodies that are inappropriately produced and/or excessively produced to a self-antigen or autoantigen. Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases. In some embodiments, the autoimmune disease is mediated by T cells, B cells, innate immune cells (e.g., macrophages, eosinophils, or natural killer cells), or complement-mediated pathways.

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full-length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)2, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full-length nucleotide sequence of a gene, nor does an antigen need to be encoded by a gene at all. In embodiments, an antigen can be synthesized or can be derived from a biological sample, e.g., a tissue sample, a blood sample, a cell, or a fluid with other biological components. As used, herein a “TCRBV antigen” includes any TCR variable beta chain or portion thereof that can provoke an immune response or be targeted by an antigen binding domain. In some embodiments, biased TCR clonotypes can be characterized by one or more TCRBV antigens which most, e.g., all, of the cells comprising the clonotype exhibit, e.g., on their surface.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Antibody Molecules

In one embodiment, the antibody molecule binds to a TCRBV antigen, e.g., a (e.g., a TCRBV antigen corresponding to a biased TCRBV clonotype). In some embodiments, the TCRBV antigen is, e.g., a mammalian, e.g., a human, TCRBV antigen. In some embodiments, the antibody molecule binds to a TCRBV antigen on an lymphocyte, e.g., T cell, e.g., a mammalian, e.g., a human, lymphocyte, e.g., T cell. For example, the antibody molecule binds specifically to a TCRBV antigen expressed, e.g., as part of a TCR comprising the TCRBV, on the surface of an lymphocyte, e.g., T cell.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J. Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., lymphocyte (e.g., T cell) comprising a TCRBV antigen corresponding to a biased TCRBV clonotype) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-la and IL-10; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a TCRBV antigen on lymphocytes, e.g. T cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 1, with additional optional stabilizing Fc cysteine mutations.

TABLE 1
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1 s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017, incorporated herein by reference in its entirety.

In embodiments, the multispecific molecules includes a multispecific antibody molecule, e.g., an antibody molecule comprising two binding specificities, e.g., a bispecific antibody molecule. The multispecific antibody molecule includes:

a lambda light chain polypeptide 1 (LLCP1) specific for a first epitope;

a heavy chain polypeptide 1 (HCP1) specific for the first epitope;

a kappa light chain polypeptide 2 (KLCP2) specific for a second epitope; and

a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:

LLCP1 has a higher affinity for HCP1 than for HCP2; and/or

KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments of the multispecific antibody molecule disclosed herein:

the HCP1 has a greater affinity for HCP2, than for a second molecule of HCP1; and/or

the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, disclosed herein is a method for making, or producing, a multispecific antibody molecule. The method includes:

(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLκ), a lambda light constant chain (VLκ), or both) that preferentially associates with the second heavy chain polypeptide (e.g., the second VH),
under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiments, the method further comprises purifying a cell-expressed antibody molecule, e.g., using a lambda—and/or—kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:

(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLl), a lambda light constant chain (VLl), or both) that preferentially associates with the first heavy chain polypeptide (e.g., the first VH), e.g., wherein the LLCP1 binds to a first epitope; and (iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VL1-CL1 heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### TCR Beta V Antigen Binding Domains

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

This disclosure provides, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7) pp: 493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

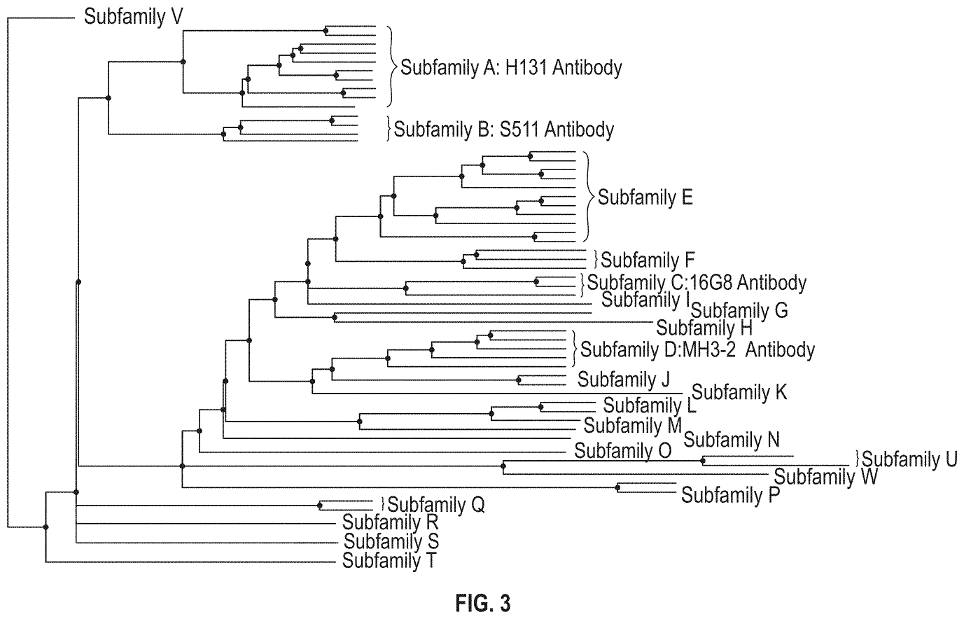

In an aspect, the disclosure provides an anti-TCRβV antibody molecule that binds to human TCRβV, e.g., a TCRβV family, e.g., gene family or a variant thereof. In some embodiments a TCRBV gene family comprises one or more subfamilies, e.g., as described herein, e.g., in FIG. 3, Table 8A or Table 8B. In some embodiments, the TCRβV gene family comprises: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30:

In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01, or a variant thereof.

In some embodiments, the TCRβ V11 subfamily comprises: TCRβ V11-1*01, TCRβ V11-2*01 or TCRβ V11-3*01, or a variant thereof.

In some embodiments, the TCRβ V14 subfamily comprises TCRβ V14*01, or a variant thereof.

In some embodiments, the TCRβ V16 subfamily comprises TCRβ V16*01, or a variant thereof.

In some embodiments, the TCRβ V18 subfamily comprises TCRβ V18*01, or a variant thereof.

In some embodiments, the TCRβ V9 subfamily comprises TCRβ V9*01 or TCRβ V9*02, or a variant thereof.

In some embodiments, the TCRβ V13 subfamily comprises TCRβ V13*01, or a variant thereof.

In some embodiments, the TCRβ V4 subfamily comprises TCRβ V4-2*01, TCRβ V4-3*01, or TCRβ V4-1*01, or a variant thereof.

In some embodiments, the TCRβ V3 subfamily comprises TCRβ V3-1*01, or a variant thereof.

In some embodiments, the TCRβ V2 subfamily comprises TCRβ V2*01, or a variant thereof.

In some embodiments, the TCRβ V15 subfamily comprises TCRβ V15*01, or a variant thereof.

In some embodiments, the TCRβ V30 subfamily comprises TCRβ V30*01, or TCRβ V30*02, or a variant thereof.

In some embodiments, the TCRβ V19 subfamily comprises TCRβ V19*01, or TCRβ V19*02, or a variant thereof.

In some embodiments, the TCRβ V27 subfamily comprises TCRβ V27*01, or a variant thereof.

In some embodiments, the TCRβ V28 subfamily comprises TCRβ V28*01, or a variant thereof.

In some embodiments, the TCRβ V24 subfamily comprises TCRβ V24-1*01, or a variant thereof.

In some embodiments, the TCRβ V20 subfamily comprises TCRβ V20-1*01, or TCRβ V20-1*02, or a variant thereof.

In some embodiments, the TCRβ V25 subfamily comprises TCRβ V25-1*01, or a variant thereof.

In some embodiments, the TCRβ V29 subfamily comprises TCRβ V29-1*01, or a variant thereof

TABLE 8A
List of TCRβV subfamilies and subfamily members

| Reference | | |
|---|---|---|
| in FIG. 3 | Subfamily | Subfamily members |
| A | TCRβ V6 | TCRβ V6-4*01, TCRβ V6-4*02, TCRβ V6-9*01, TCRβ V6- |
| | Also referred to | 8*01, TCRβ V6-5*01, TCRβ V6-6*02, TCRβ V6-6*01, TCRβ |
| | as: TCR VB 13.1 | V6-2*01, TCRβ V6-3*01 or TCRβ V6-1*01. |
| B | TCRβ V10 | TCRβ V10-1*01, TCRβ V10-1*02, TCRβ V10-3*01 or TCRβ |
| | Also referred to | V10-2*01 |
| | as: TCRβ V12 | |
| C | TCRβ V12 | TCRβ V12-4*01, TCRβ V12-3*01, or TCRβ V12-5*01 |
| | Also referred to | |
| | as: TCRβ V8.1 | |
| D | TCRβ V5 | TCRβ V5-5*01, TCRβ V5-6*01, TCRβ V5-4*01, TCRβ V5- |
| | | 8*01, TCRβ V5-1*01 |
| E | TCRβ V7 | TCRβ V7-7*01, TCRβ V7-6*01, TCRβ V7-8*02, TCRβ V7- |
| | | 4*01, TCRβ V7-2*02, TCRβ V7-2*03, TCRβ V7-2*01, TCRβ |
| | | V7-3*01, TCRβ V7-9*03, or TCRβ V7-9*01 |
| F | TCRβ V11 | TCRβ V11-1*01, TCRβ V11-2*01 or TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, or TCRβ V4-1*01 |
| M | TCRβ V3 | TCRβ V3-1*01 |
| N | TCRβ V2 | TCRβ V2*01 |
| O | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| T | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

TABLE 8B
Additional TCRβV subfamilies
Subfamily
TCRβ V1
TCRβ V17
TCRβ V21
TCRβ V23
TCRβ V26

### Anti-TCRβV Antibodies

Disclosed herein, is the discovery of a novel class of antibodies, i.e. anti-TCRβV antibody molecules disclosed herein, which despite having low sequence similarity (e.g., low sequence identity among the different antibody molecules that recognize different TCRβV subfamilies), recognize a structurally conserved region, e.g., domain, on the TCRβV protein and have a similar function (e.g., a similar cytokine profile). Thus, the anti-TCRβV antibody molecules disclosed herein share a structure-function relationship.

In some embodiments, the anti-TCRβV antibody molecules disclosed herein do not recognize, e.g., bind to, an interface of a TCRβV:TCRalpha complex.

In some embodiments, the anti-TCRβV antibody molecules disclosed herein do not recognize, e.g., bind to, a constant region of a TCRβV protein. An exemplary antibody that binds to a constant region of a TCRBV region is JOVI.1 as described in Viney et al., (Hybridoma. 1992 December; 11(6):701-13).

In some embodiments, the anti-TCRβV antibody molecules disclosed herein do not recognize, e.g., bind to, one or more (e.g., all) of a complementarity determining region (e.g., CDR1, CDR2 and/or CDR3) of a TCRβV protein.

In some embodiments, the anti-TCRβV antibody molecules disclosed herein binds (e.g., specifically binds) to a TCRβV region. In some embodiments, binding of anti-TCRβV antibody molecules disclosed herein results in a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In an aspect, the disclosure provides an anti-TCRβV antibody molecule that binds to human TCRβV, e.g., a TCRβV gene family, e.g., one or more of a TCRβV subfamily, e.g., as described herein, e.g., in FIG. 3, Table 8A, or Table 8B. In some embodiments, the anti-TCRβV antibody molecule binds to one or more TCRβV subfamilies chosen from: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, or a variant thereof.

In some embodiments, the anti-TCRβV antibody molecule binds to a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments the TCRβ V6 subfamily comprises TCRβ V6-5*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, the anti-TCRβV antibody molecule binds to a TCRβ V10 subfamily comprising: TCRβ V10-1*01, TCRβ V10-1*02, TCRβ V10-3*01 or TCRβ V10-2*01, or a variant thereof.

In some embodiments, the anti-TCRβV antibody molecule binds to a TCRβ V12 subfamily comprising: TCRβ V12-4*01, TCRβ V12-3*01 or TCRβ V12-5*01, or a variant thereof.

In some embodiments, the anti-TCRβV antibody molecule binds to a TCRβ V5 subfamily comprising: TCRβ V5-5*01, TCRβ V5-6*01, TCRβ V5-4*01, TCRβ V5-8*01, TCRβ V5-1*01, or a variant thereof.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule binds to TCRβ V5-5*01 or TCRβ V5-1*Ol with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

### Anti-TCRβ V6 Antibodies

Accordingly, in one aspect, the disclosure provides an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof.

SEQ ID NO: 43
ATGAGCATCGGCCTCCTGTGCTGTGCAGCCTTGTCTCTCCTGTGGGCAGGT
CCAGTGAATGCTGGTGTCACTCAGACCCCAAAATTCCAGGTCCTGAAGACA
GGACAGAGCATGACACTGCAGTGTGCCCAGGATATGAACCATGAATACATG
TCCTGGTATCGACAAGACCCAGGCATGGGGCTGAGGCTGATTCATTACTCA
GTTGGTGCTGGTATCACTGACCAAGGAGAAGTCCCCAATGGCTACAATGTC
TCCAGATCAACCACAGAGGATTTCCCGCTCAGGCTGCTGTCGGCTGCTCCC
TCCCAGACATCTGTGTACTTCTGTGCCAGCAGTTACTC

In some embodiments, TCRβ V6-5*01 comprises the amino acid sequence of SEQ ID NO: 44, or an amino acid sequence having 85%, 90%, 95%, 99% or more identity thereof.

SEQ ID NO: 44
MSIGLLCCAALSLLWAGPVNAGVTQTPKFQVLKTGQSMTLQCAQDMNHEYM
SWYRQDPGMGLRLIHYSVGAGITDQGEVPNGYNVSRSTTEDFPLRLLSAAP
SQTSVYFCASSY

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.68, e.g., A-H.1, A-H.2 or A-H.68, or an antibody molecule described in Table 1A, or encoded by a nucleotide sequence in Table 1A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 230 or 3289.

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In one embodiment, the heavy chain constant region comprises an amino sequence set forth in Table 3A, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In one embodiment, the light chain constant region comprises an amino sequence set forth in Table 3A, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 1A, or encoded by a nucleotide sequence shown in Table 1A. In one embodiment, one or more of the CDRs (or collectively all of the CDRs) have one, two, three, four, five, six or more changes, e.g., amino acid substitutions or deletions, relative to the amino acid sequence shown in Table 1A, or encoded by a nucleotide sequence shown in Table 1A.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 1A, or encoded by a nucleotide sequence shown in Table 1A. In one embodiment, one or more of the CDRs (or collectively all of the CDRs) have one, two, three, four, five, six or more changes, e.g., amino acid substitutions or deletions, relative to the amino acid sequence shown in Table 1A, or encoded by a nucleotide sequence shown in Table 1A.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 1A, or encoded by a nucleotide sequence shown in Table 1A. In one embodiment, one or more of the CDRs (or collectively all of the CDRs) have one, two, three, four, five, six or more changes, e.g., amino acid substitutions or deletions, relative to the amino acid sequence shown in Table 1A, or encoded by a nucleotide sequence shown in Table 1A.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1A) from a heavy chain variable region of an antibody described herein, e.g., an antibody chosen from any one of A-H.1 to A-H.68, e.g., A-H.1, A-H.2 or A-H.68, or an antibody described in Table 1A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1A.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1A) from a light chain variable region of an antibody described herein, e.g., an antibody chosen from any one of A-H.1 to A-H.68, e.g., A-H.1, A-H.2 or A-H.68, or an antibody described in Table 1A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1A.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 1A) from the heavy and light chain variable regions of an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1A.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 1A) from the heavy and light chain variable regions of an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1A. In one embodiment, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.68, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1A) from a heavy chain variable region of an antibody described herein, e.g., an antibody chosen from any one of A-H.1 to A-H.68, e.g., A-H.1, A-H.2 or A-H.68, or as described in Table 1A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1A.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1A) from a light chain variable region of an antibody described herein, e.g., an antibody chosen from any one of A-H.1 to A-H.68, e.g., A-H.1, A-H.2 or A-H.68, or an antibody described in Table 1A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1A.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 1A) from the heavy and light chain variable regions of an antibody described herein, e.g., an antibody chosen from any one of A-H.1 to A-H.68, e.g., A-H.1, A-H.2 or A-H.68, or an antibody described in Table 1A, or encoded by the nucleotide sequence in Table 1A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 1A.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 1A) from the heavy and light chain variable regions of an antibody described herein, e.g., an antibody chosen from any one of A-H.1 to A-H.68, e.g., A-H.1, A-H.2 or A-H.68, or an antibody described in Table 1A, or encoded by a nucleotide sequence in Table 1A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 1A. In one embodiment, the anti-TCRβ V antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 1A.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 1A is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 1A. In some embodiments, the anti-TCRβ V antibody molecule, e.g., anti-TCRβ V6 (e.g., anti-TCRβ V6-5*01) antibody molecule, can contain any combination of CDRs or hypervariable loops according the “combined” CDRs are described in Table 1A.

In an embodiment, e.g., an embodiment comprising a variable region, a CDR (e.g., a combined CDR, Chothia CDR or Kabat CDR), or other sequence referred to herein, e.g., in Table 1A, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In an embodiment, the anti-TCRβV antibody molecule, e.g., anti-TCRβ V6 (e.g., anti-TCRβ V6-5*01) antibody molecule includes:

(i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In an embodiment, the anti-TCRβV antibody molecule, e.g., anti-TCRβ V6 (e.g., anti-TCRβ V6-5*01) antibody molecule comprises:

(i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises:

(i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In an embodiment, the anti-TCRβV antibody molecule, e.g., anti-TCRβ V6 (e.g., anti-TCRβ V6-5*01) antibody molecule comprises:

(i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises:

(i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In an embodiment, the anti-TCRβV antibody molecule, e.g., anti-TCRβ V6 (e.g., anti-TCRβ V6-5*01) antibody molecule comprises:

(i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises:

(i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In one embodiment, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

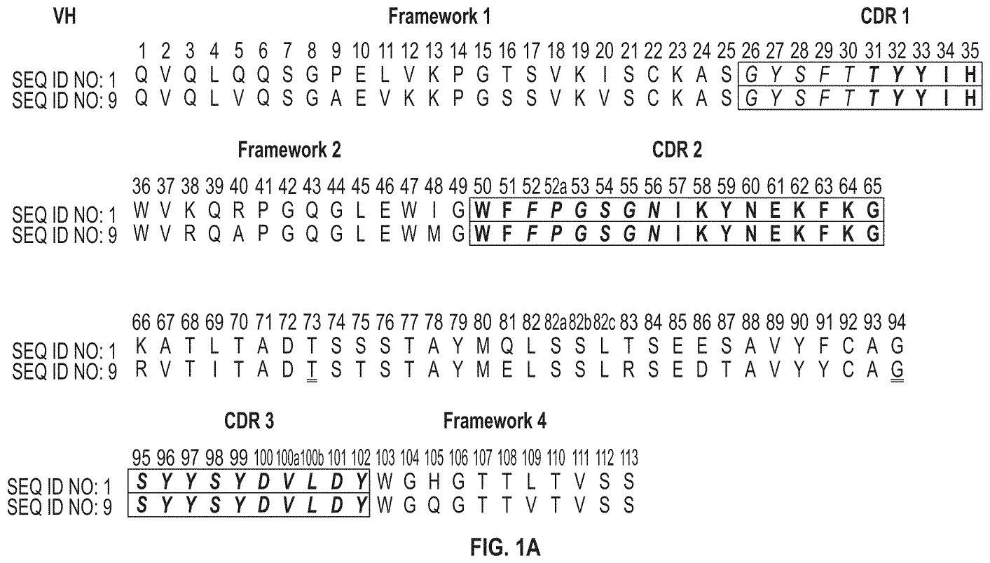

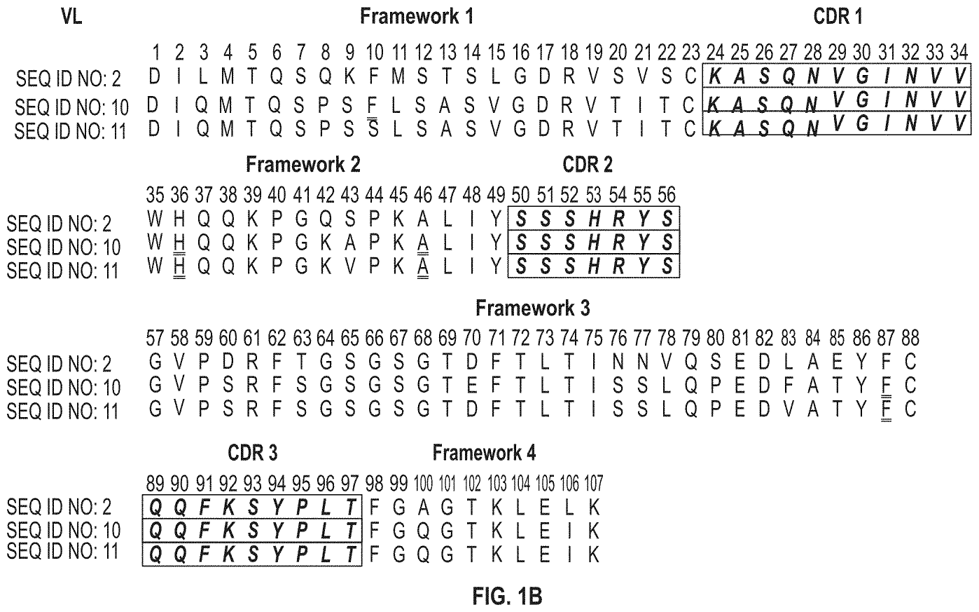

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.68, e.g., A-H.1, A-H.2 or A-H.68, e.g., the amino acid sequence of the FR region in the entire variable region, e.g., shown in FIG. 1A, or in SEQ ID NO: 9.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.68, e.g., A-H.1, A-H.2 or A-H.68, e.g., the amino acid sequence of the FR region in the entire variable region, e.g., shown in FIG. 1B, or in SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, includes one, two, three, or four heavy chain framework regions shown in FIG. 1A, or a sequence substantially identical thereto.

In some embodiments, the anti-TCRβV antibody molecule, e.g., anti-TCRβ V6 (e.g., anti-TCRβ V6-5*01) antibody molecule, includes one, two, three, or four light chain framework regions shown in FIG. 1B, or a sequence substantially identical thereto.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position disclosed herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position disclosed herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering, e.g., a Serine to Phenyalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions disclosed herein according to Kabat numbering, (b) a framework region 2 (FR2) comprising a change, e.g., a substitution (e.g., a conservative substitution) at one or more (e.g., all) position disclosed herein according to Kabat numbering and (c) a framework region 3 (FR3) comprising a change, e.g., a substitution (e.g., a conservative substitution) at one or more (e.g., all) position disclosed herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2, e.g., as shown in FIG. 1A.

In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2, e.g., as shown in FIG. 1A

In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2, e.g., as shown in FIG. 1A.

In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2, e.g., as shown in FIG. 1A.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position disclosed herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9, or as shown in FIGS. 1A and 1B.

In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10, or as shown in FIGS. 1A and 1B.

In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11, or as shown in FIGS. 1A and 1B.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10, or as shown in FIGS. 1A and 1B.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11, or as shown in FIGS. 1A and 1B.

In some embodiments, the heavy or light chain variable domain, or both, of the anti-TCRβ V antibody molecule, e.g., anti-TCRβ V6 (e.g., anti-TCRβ V6-5*01) antibody molecule, includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody chosen from any one of A-H.1 to A-H.68, e.g., A-H.1, A-H.2 or A-H.68, or as described in Table 1A, or encoded by the nucleotide sequence in Table 1A; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1A, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 1A. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1A, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1A.

In some embodiments, the anti-TCRβV antibody molecule, e.g., anti-TCRβ V6 (e.g., anti-TCRβ V6-5*01) antibody molecule, comprises:

a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises:

a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)₂, Fv, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In one embodiment, the constant region is altered, e.g., mutated, to modify the properties of the anti-TCRβ V antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217, or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, or 99% identity thereto.

Additional exemplary humanized anti-TCRβ V6 antibodies are provided in Table 1A. In some embodiments, the anti-TCRβ V6 is antibody A, e.g., humanized antibody A (antibody A-H), as provided in Table 1A. In some embodiments, the anti-TCRβV antibody comprises one or more (e.g., all three) of a LC CDR1, LC CDR2, and LC CDR3 provided in Table 1A; and/or one or more (e.g., all three) of a HC CDR1, HC CDR2, and HC CDR3 provided in Table 1A, or a sequence with at least 85%, 90%, 95%, 96%, 97%, 98%, or 99% identity thereto. In some embodiments, antibody A comprises a variable heavy chain (VH) and/or a variable light chain (VL) provided in Table 1A, or a sequence with at least 85%, 90%, 95%, 96%, 97%, 98%, or 99% identity thereto.

TABLE 1A
Amino acid and nucleotide sequences for murine,
chimeric and humanized antibody molecules which
bind to TCRVB 6, e.g., TCRVB 6-5. The antibody
molecules include murine mAb Antibody A, and
humanized mAb Antibody A-H Clones A-H.1 to A-H.68.
The amino acid the heavy and light chain CDRs,
and the amino acid and nucleotide sequences
of the heavy and light chain variable regions,
and the heavy and light chains are shown.
Antibody A (murine)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIH |
| | | WVKQRPGQGLEWIGWFFPGSGNIKYNEKFKGKAT |
| | | LTADTSSSTAYMQLSSLTSEESAVYFCAGSYYSYD |
| | | VLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: 2 | VL | DILMTQSQKFMSTSLGDRVSVSCKASQNVGINVV |
| | | WHQQKPGQSPKALIYSSSHRYSGVPDRFTGSGSGT |
| | | DFTLTINNVQSEDLAEYFCQQFKSYPLTFGAGTKL |
| | | ELK |

Antibody A humanized (A-H antibody)

| | | |
|---|---|---|
| A-H.1 antibody | | |
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIH |
| | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| SEQ ID NO: 12 | DNA VH | CAGGTGCAGCTGGTTCAGTCTGGCGCCGAAGTG |
| | | AAGAAACCTGGCTCCTCCGTGAAGGTGTCCTGC |
| | | AAGGCTTCCGGCTACTCCTTCACCACCTACTACA |
| | | TCCACTGGGTCCGACAGGCCCCTGGACAAGGAT |
| | | TGGAATGGATGGGCTGGTTCTTCCCCGGCTCCGG |
| | | CAACATCAAGTACAACGAGAAGTTCAAGGGCCG |
| | | CGTGACCATCACCGCCGACACCTCTACCTCTACC |
| | | GCCTACATGGAACTGTCCAGCCTGAGATCTGAG |
| | | GACACCGCCGTGTACTACTGCGCCGGCTCCTACT |
| | | ACTCTTACGACGTGCTGGATTACTGGGGCCAGG |
| | | GCACCACAGTGACAGTGTCCTCT |
| SEQ ID NO: 69 | VH-IgM constant delta | METDTLLLWVLLLWVPGSTGQVQLVQSGAEVKK |
| | CDC | PGSSVKVSCKASGYSFTTYYIHWVRQAPGQGLEW |
| | | MGWFFPGSGNIKYNEKFKGRVTITADTSTSTAYME |
| | | LSSLRSEDTAVYYCAGSYYSYDVLDYWGQGTTVT |
| | | VSSGSASAPTLFPLVSCENSPSDTSSVAVGCLAQDF |
| | | LPDSITFSWKYKNNSDISSTRGFPSVLRGGKYAATS |
| | | QVLLPSKDVMQGTDEHVVCKVQHPNGNKEKNVP |
| | | LPVIAELPPKVSVFVPPRDGFFGNPRKSKLICQATG |
| | | FSPRQIQVSWLREGKQVGSGVTTDQVQAEAKESG |
| | | PTTYKVTSTLTIKESDWLGQSMFTCRVDHRGLTFQ |
| | | QNASSMCVPDQDTAIRVFAIPPSFASIFLTKSTKLTC |
| | | LVTDLTTYDSVTISWTRQNGEAVKTHTNISESHPN |
| | | ATFSAVGEASICEDDWNSGERFTCTVTHTDLASSL |
| | | KQTISRPKGVALHRPDVYLLPPAREQLNLRESATIT |
| | | CLVTGFSPADVFVQWMQRGQPLSPEKYVTSAPMP |
| | | EPQAPGRYFAHSILTVSEEEWNTGETYTCVVAHEA |
| | | LPNRVTERTVDKSTGKPTLYNVSLVMSDTAGTCY |
| SEQ ID NO: 70 | VH-IgGA1 | METDTLLLWVLLLWVPGSTGQVQLVQSGAEVKK |
| | | PGSSVKVSCKASGYSFTTYYIHWVRQAPGQGLEW |
| | | MGWFFPGSGNIKYNEKFKGRVTITADTSTSTAYME |
| | | LSSLRSEDTAVYYCAGSYYSYDVLDYWGQGTTVT |
| | | VSSASPTSPKVFPLSLCSTQPDGNVVIACLVQGFFP |
| | | QEPLSVTWSESGQGVTARNFPPSQDASGDLYTTSS |
| | | QLTLPATQCLAGKSVTCHVKHYTNPSQDVTVPCP |
| | | VPSTPPTPSPSTPPTPSPSCCHPRLSLHRPALEDLLL |
| | | GSEANLTCTLTGLRDASGVTFTWTPSSGKSAVQGP |
| | | PERDLCGCYSVSSVLPGCAEPWNHGKTFTCTAAYP |
| | | ESKTPLTATLSKSGNTFRPEVHLLPPPSEELALNEL |
| | | VTLTCLARGFSPKDVLVRWLQGSQELPREKYLTW |
| | | ASRQEPSQGTTTFAVTSILRVAAEDWKKGDTFSCM |
| | | VGHEALPLAFTQKTIDRLAGKPTHVNVSVVMAEV |
| | | DGTCY |
| SEQ ID NO: 71 | VH-IgGA2 | METDTLLLWVLLLWVPGSTGQVQLVQSGAEVKK |
| | | PGSSVKVSCKASGYSFTTYYIHWVRQAPGQGLEW |
| | | MGWFFPGSGNIKYNEKFKGRVTITADTSTSTAYME |
| | | LSSLRSEDTAVYYCAGSYYSYDVLDYWGQGTTVT |
| | | VSSASPTSPKVFPLSLDSTPQDGNVVVACLVQGFFP |
| | | QEPLSVTWSESGQNVTARNFPPSQDASGDLYTTSS |
| | | QLTLPATQCPDGKSVTCHVKHYTNSSQDVTVPCR |
| | | VPPPPPCCHPRLSLHRPALEDLLLGSEANLTCTLTG |
| | | LRDASGATFTWTPSSGKSAVQGPPERDLCGCYSVS |
| | | SVLPGCAQPWNHGETFTCTAAHPELKTPLTANITK |
| | | SGNTFRPEVHLLPPPSEELALNELVTLTCLARGFSP |
| | | KDVLVRWLQGSQELPREKYLTWASRQEPSQGTTT |
| | | YAVTSILRVAAEDWKKGETFSCMVGHEALPLAFT |
| | | QKTIDRMAGKPTHINVSVVMAEADGTCY |
| SEQ ID NO: | Heavy chain | METDTLLLWVLLLWVPGSTGQVQLVQSGAEVKK |
| 3278 | | PGSSVKVSCKASGYSFTTYYIHWVRQAPGQGLEW |
| | | MGWFFPGSGNIKYNEKFKGRVTITADTSTSTAYME |
| | | LSSLRSEDTAVYYCAGSYYSYDVLDYWGQGTTVT |
| | | VSSASTKGPSVFPLAPSSKSTSGGTAALGCLVKDYF |
| | | PEPVTVSWNSGALTSGVHTFPAVLQSSGLYSLSSV |
| | | VTVPSSSLGTQTYICNVNHKPSNTKVDKRVEPKSC |
| | | DKTHTCPPCPAPELLGGPSVFLFPPKPKDTLMISRT |
| | | PEVTCVVVDVSHEDPEVKFNWYVDGVEVHNAKT |
| | | KPREEQYNSTYRVVSVLTVLHQDWLNGKEYKCK |
| | | VSNKALPAPIEKTISKAKGQPREPQVYTLPPSREEM |
| | | TKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVW |
| | | HQQKPGKAPKALIYSSSHRYSGVPSRFSGSGSGTEF |
| | | TLTISSLQPEDFATYFCQQFKSYPLTFGQGTKLEIK |
| SEQ ID NO: 13 | DNA VL | GACATCCAGATGACCCAGTCTCCATCCTTCCTGT |
| | | CCGCCTCTGTGGGCGACAGAGTGACCATCACAT |
| | | GCAAGGCCTCTCAGAACGTGGGCATCAACGTCG |
| | | TGTGGCACCAGCAGAAGCCTGGCAAGGCTCCTA |
| | | AGGCTCTGATCTACTCCTCCAGCCACCGGTACTC |
| | | TGGCGTGCCCTCTAGATTTTCCGGCTCTGGCTCT |
| | | GGCACCGAGTTTACCCTGACAATCTCCAGCCTGC |
| | | AGCCTGAGGACTTCGCCACCTACTTTTGCCAGCA |
| | | GTTCAAGAGCTACCCTCTGACCTTTGGCCAGGGC |
| | | ACCAAGCTGGAAATCAAG |
| SEQ ID NO: 72 | VL and kappa constant | METDTLLLWVLLLWVPGSTGDIQMTQSPSFLSASV |
| | region/light chain | GDRVTITCKASQNVGINVVWHQQKPGKAPKALIY |
| | | SSSHRYSGVPSRFSGSGSGTEFTLTISSLQPEDFATY |
| | | FCQQFKSYPLTFGQGTKLEIKRTVAAPSVFIFPPSDE |
| | | QLKSGTASVVCLLNNFYPREAKVQWKVDNALQS |
| | | GNSQESVTEQDSKDSTYSLSSTLTLSKADYEKHKV |
| | | YACEVTHQGLSSPVTKSFNRGEC |
| A-H.2 antibody | | |
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIH |
| | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| SEQ ID NO: 12 | DNA VH | CAGGTGCAGCTGGTTCAGTCTGGCGCCGAAGTG |
| | | AAGAAACCTGGCTCCTCCGTGAAGGTGTCCTGC |
| | | AAGGCTTCCGGCTACTCCTTCACCACCTACTACA |
| | | TCCACTGGGTCCGACAGGCCCCTGGACAAGGAT |
| | | TGGAATGGATGGGCTGGTTCTTCCCCGGCTCCGG |
| | | CAACATCAAGTACAACGAGAAGTTCAAGGGCCG |
| | | CGTGACCATCACCGCCGACACCTCTACCTCTACC |
| | | GCCTACATGGAACTGTCCAGCCTGAGATCTGAG |
| | | GACACCGCCGTGTACTACTGCGCCGGCTCCTACT |
| | | ACTCTTACGACGTGCTGGATTACTGGGGCCAGG |
| | | GCACCACAGTGACAGTGTCCTCT |
| SEQ ID NO: | Heavy chain | METDTLLLWVLLLWVPGSTGQVQLVQSGAEVKK |
| 3278 | | PGSSVKVSCKASGYSFTTYYIHWVRQAPGQGLEW |
| | | MGWFFPGSGNIKYNEKFKGRVTITADTSTSTAYME |
| | | LSSLRSEDTAVYYCAGSYYSYDVLDYWGQGTTVT |
| | | VSSASTKGPSVFPLAPSSKSTSGGTAALGCLVKDYF |
| | | PEPVTVSWNSGALTSGVHTFPAVLQSSGLYSLSSV |
| | | VTVPSSSLGTQTYICNVNHKPSNTKVDKRVEPKSC |
| | | DKTHTCPPCPAPELLGGPSVFLFPPKPKDTLMISRT |
| | | PEVTCVVVDVSHEDPEVKFNWYVDGVEVHNAKT |
| | | KPREEQYNSTYRVVSVLTVLHQDWLNGKEYKCK |
| | | VSNKALPAPIEKTISKAKGQPREPQVYTLPPSREEM |
| | | TKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVW |
| | | HQQKPGKVPKALIYSSSHRYSGVPSRFSGSGSGTDF |
| | | TLTISSLQPEDVATYFCQQFKSYPLTFGQGTKLEIK |
| SEQ ID NO: 14 | DNA VL | GACATCCAGATGACCCAGTCTCCATCCTCTCTGT |
| | | CCGCCTCTGTGGGCGACAGAGTGACCATCACAT |
| | | GCAAGGCCTCTCAGAACGTGGGCATCAACGTCG |
| | | TGTGGCACCAGCAGAAACCTGGCAAGGTGCCCA |
| | | AGGCTCTGATCTACTCCTCCAGCCACAGATACTC |
| | | CGGCGTGCCCTCTAGATTCTCCGGCTCTGGCTCT |
| | | GGCACCGACTTTACCCTGACAATCTCCAGCCTGC |
| | | AGCCTGAGGACGTGGCCACCTACTTTTGCCAGC |
| | | AGTTCAAGAGCTACCCTCTGACCTTTGGCCAGGG |
| | | CACCAAGCTGGAAATCAAG |
| SEQ ID NO: | Light chain | METDTLLLWVLLLWVPGSTGDIQMTQSPSSLSASV |
| 3279 | | GDRVTITCKASQNVGINVVWHQQKPGKVPKALIY |
| | | SSSHRYSGVPSRFSGSGSGTDFTLTISSLQPEDVATY |
| | | FCQQFKSYPLTFGQGTKLEIKRTVAAPSVFIFPPSDE |
| | | QLKSGTASVVCLLNNFYPREAKVQWKVDNALQS |
| | | GNQESVTEQDSKDSTYSLSSTLTLSKADYEKHKV |
| | | YACEVTHQGLSSPVTKSFNRGEC |
| A-H.3 antibody | | |
| SEQ ID NO: 80 | VH + VL | QVQLVQSGAEVKKPGSSVKVSCKASGTDFKLTYIH |
| | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: 81 | VL | DIQMTQSPSFLSASVGDRVTITCKASQNVEDRVAW |
| | | YQQKPGKAPKALIYSSSHRYKGVPSRFSGSGSGTE |
| | | FTLTISSLQPEDFATYFCQQFKSYPLTFGQGTKLEIK |
| SEQ ID NO: 82 | VH | QVQLVQSGAEVKKPGSSVKVSCKASGTDFKLTYIH |
| | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSS |
| A-H.4 | | |
| SEQ ID NO: 83 | VH + VL | QVQLVQSGAEVKKPGSSVKVSCKASGTDFDKIYIH |
| | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: 84 | VL | DIQMTQSPSFLSASVGDRVTITCKASQNVEDRVAW |
| | | YQQKPGKAPKALIYSSSHRYKGVPSRFSGSGSGTE |
| | | FTLTISSLQPEDFATYFCQQFKSYPLTFGQGTKLEIK |
| SEQ ID NO: 85 | VH | QVQLVQSGAEVKKPGSSVKVSCKASGTDFDKIYIH |
| | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |
| A-H.5 | | |
| SEQ ID NO: 86 | VH + VL | QVQLVQSGAEVKKPGSSVKVSCKASGHDFRDFYI |
| | | HWVRQAPGQGLEWMGRVYPGSGSYRYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: 87 | VL | DIQMTQSPSFLSASVGDRVTITCKASQNVDDRVAW |
| | | YQQKPGKAPKALIYSSSHRYKGVPSRFSGSGSGTE |
| | | FTLTISSLQPEDFATYFCQQFKSYPLTFGQGTKLEIK |
| SEQ ID NO: 88 | VH | QVQLVQSGAEVKKPGSSVKVSCKASGHDFRDFYI |
| | | HWVRQAPGQGLEWMGRVYPGSGSYRYNEKFKGR |
| | | VTITADTSTSTAYMELSSLRSEDTAVYYCAGSYYS |
| | | YDVLDYWGQGTTVTVSS |
| A-H.6 | | |
| SEQ ID NO: 89 | VH + VL | QVQLVQSGAEVKKPGSSVKVSCKASGHDFKLTYI |
| | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDN |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: 90 | VL | DIQMTQSPSFLSASVGDRVTITCKASQNVDNRVAW |
| | | YQQKPGKAPKALIYSSSHRYKGVPSRFSGSGSGTE |
| | | FTLTISSLQPEDFATYFCQQFKSYPLTFGQGTKLEIK |
| SEQ ID NO: 91 | VH | QVQLVQSGAEVKKPGSSVKVSCKASGHDFKLTYI |
| | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |
| A-H.7 | | |
| SEQ ID NO: 92 | VH + VL | QVQLVQSGAEVKKPGSSVKVSCKASGTDFKLTYIH |
| | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVENK |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: 93 | VL | DIQMTQSPSFLSASVGDRVTITCKASQNVENKVAW |
| | | HQQKPGKAPKALIYSSSHRYKGVPSRFSGSGSGTE |
| | | FTLTISSLQPEDFATYFCQQFKSYPLTFGQGTKLEIK |
| SEQ ID NO: 94 | VH | QVQLVQSGAEVKKPGSSVKVSCKASGTDFKLTYIH |
| | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |
| A-H.8 | | |
| SEQ ID NO: 95 | VH + VL | QVQLVQSGAEVKKPGSSVKVSCKASGTDFDKIYIH |
| | | WVRQAPGQGLEWMGRIFAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: 96 | VL | DIQMTQSPSFLSASVGDRVTITCKASQNVDDRVAW |
| | | YQQKPGKAPKALIYSSSHRYKGVPSRFSGSGSGTE |
| | | FTLTISSLQPEDFATYFCQQFKSYPLTFGQGTKLEIK |
| SEQ ID NO: 97 | VH | QVQLVQSGAEVKKPGSSVKVSCKASGTDFDKIYIH |
| | | WVRQAPGQGLEWMGRIFAGSGNTKYNEKFKGRV |
| | | TITADTSTSTAYMELSSLRSEDTAVYYCAGSYYSY |
| | | DVLDYWGQGTTVTVSS |
| A-H.9 | | |
| SEQ ID NO: 98 | VH + VL | QVQLVQSGAEVKKPGSSVKVSCKASGHDFDKFYI |
| | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASNVG |
| | | NRVAWYQQKPGKAPKALIYSSSHRYSGVPSRFSGS |
| | | GSGTEFTLTISSLQPEDFATYFCQQFKSYPLTFGQG |
| | | TKLEIK |
| SEQ ID NO: 99 | VL | DIQMTQSPSFLSASVGDRVTITCKASQNVGNRVAW |
| | | YQQKPGKAPKALIYSSSHRYSGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFDKFYI |
| 100 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |
| A-H.10 | | |
| SEQ ID NO: | VH + VL | QVQLVQSGAEVKKPGSSVKVSCKASGHDFDKFYI |
| 101 | | HWVRQAPGQGLEWMGRIFAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGD |
| | | RVAWYQQKPGKAPKALIYSSSHRYKGVPSRFSGS |
| | | GSGTEFTLTISSLQPEDFATYFCQQFKSYPLTFGQG |
| | | TKLEIKs |
| SEQ ID NO: | VL | DIQMTQSPSFLSASVGDRVTITCKASQNVGDRVAW |
| 102 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFDKFYI |
| 103 | | HWVRQAPGQGLEWMGRIFAGSGNVKYNEKFKGR |
| | | VTITADTSTSTAYMELSSLRSEDTAVYYCAGSYYS |
| | | YDVLDYWGQGTTVTVSS |
| A-H.11 | | |
| SEQ ID NO: | VH + VL | QVQLVQSGAEVKKPGSSVKVSCKASGTDFKLTYIH |
| 104 | | WVRQAPGQGLEWMGRVSPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 105 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 106 | | WVRQAPGQGLEWMGRVSPGSGNVKYNEKFKGRV |
| | | TITADTSTSTAYMELSSLRSEDTAVYYCAGSYYSY |
| | | DVLDYWGQGTTVTVSS |
| A-H.12 | | |
| SEQ ID NO: | VH + VL | QVQLVQSGAEVKKPGSSVKVSCKASGTDFDKIYIH |
| 107 | | WVRQAPGQGLEWMGRVSAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGN |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVGNRVAW |
| 108 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFDKIYIH |
| 109 | | WVRQAPGQGLEWMGRVSAGSGNTKYNEKFKGR |
| | | VTITADTSTSTAYMELSSLRSEDTAVYYCAGSYYS |
| | | YDVLDYWGQGTTVTVSS |
| A-H.13 | | |
| SEQ ID NO: | VH + VL | QVQLVQSGAEVKKPGSSVKVSCKASGTDFKLTYIH |
| 110 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNRVAW |
| 111 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 112 | | WVRQAPGQGLEWMGRIFPGSGNVKYNEKFKGRV |
| | | TITADTSTSTAYMELSLRSEDTAVYYCAGSYYSY |
| | | DVLDYWGQGTTVTVSS |
| A-H.14 | | |
| SEQ ID NO: | VH + VL | QVQLVQSGAEVKKPGSSVKVSCKASGTDFDKIYIH |
| 113 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 114 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFDKIYIH |
| 115 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSS |
| A-H.15 | | |
| SEQ ID NO: | VH + VL | QVQLVQSGAEVKKPGSSVKVSCKASGTDFRLTYIH |
| 116 | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNK |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNKVA |
| 117 | | WHQQKPGKAPKALIYSSSHRYKGVPSRFSGSGSGT |
| | | EFTLTISSLQPEDFATYFCQQFKSYPLTFGQGTKLEI |
| | | K |
| SEQ ID NO: | VH | QVQLVQSGAEVKKPGSSVKVSCKASGTDFRLTYIH |
| 118 | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSS |
| A-H.16 | | |
| SEQ ID NO: | VH + VL | QVQLVQSGAEVKKPGSSVKVSCKASGGTFRLTYIH |
| 119 | | WVRQAPGQGLEWMGRVYPGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 120 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGGTFRLTYIH |
| 121 | | WVRQAPGQGLEWMGRVYPGSGNTKYNEKFKGR |
| | | VTITADTSTSTAYMELSSLRSEDTAVYYCAGSYYS |
| | | YDVLDYWGQGTTVTVSS |
| A-H.17 | | |
| SEQ ID NO: | VH + VL | QVQLVQSGAEVKKPGSSVKVSCKASGTDFRLTYIH |
| 122 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 123 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFRLTYIH |
| 124 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSS |
| A-H.18 | | |
| SEQ ID NO: | VH + VL | QVQLVQSGAEVKKPGSSVKVSCKASGTDFKLTYIH |
| 125 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVEDRVAW |
| 126 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 127 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |
| A-H.19 | | |
| SEQ ID NO: | VH + VL | QVQLVQSGAEVKKPGSSVKVSCKASGGTFRLTYIH |
| 128 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 129 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGGTFRLTYIH |
| 130 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |
| A-H.20 | | |
| SEQ ID NO: | VH + VL | QVQLVQSGAEVKKPGSSVKVSCKASGGTFDKTYI |
| 131 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 132 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGGTFDKTYI |
| 133 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSS |
| A-H.21 | | |
| SEQ ID NO: | VH + VL | QVQLVQSGAEVKKPGSSVKVSCKASGHDFDKFYI |
| 134 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 135 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFDKFYI |
| 136 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSS |
| A-H.22 | | |
| SEQ ID NO: | VH + VL | QVQLVQSGAEVKKPGSSVKVSCKASGTDFKLTYIH |
| 137 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNK |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNKVA |
| 138 | | WHQQKPGKAPKALIYSSSHRYKGVPSRFSGSGSGT |
| | | EFTLTISSLQPEDFATYFCQQFKSYPLTFGQGTKLEI |
| | | K |
| SEQ ID NO: | VH | QVQLVQSGAEVKKPGSSVKVSCKASGTDFKLTYIH |
| 139 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |
| A-H.23 | | |
| SEQ ID NO: | VH + VL | QVQLVQSGAEVKKPGSSVKVSCKASGHDFRLTYI |
| 140 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVAD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVADRVAW |
| 141 | | YQQKPGKAPKALIYSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFRLTYI |
| 142 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |
| A-H.24 | | |
| SEQ ID NO: | VH + VL | QVQLVQSGAEVKKPGSSVKVSCKASGHDFHLWYI |
| 143 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVD |
| | | NKVAWHQQKPGKAPKALIYSSSHRYKGVPSRFSG |
| | | SGSGTEFTLTISSLQPEDFATYFCQQFKSYPLTFGQ |
| | | GTKLEIK |
| SEQ ID NO: | VL | DIQMTQSPSFLSASVGDRVTITCKASQNVDNKVA |
| 144 | | WHQQKPGKAPKALIYSSSHRYKGVPSRFSGSGSGT |
| | | EFTLTISSLQPEDFATYFCQQFKSYPLTFGQGTKLEI |
| | | K |
| SEQ ID NO: | VH | QVQLVQSGAEVKKPGSSVKVSCKASGHDFHLWYI |
| 145 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |
| A-H.25 | | |
| SEQ ID NO: | VH + VL | QVQLVQSGAEVKKPGSSVKVSCKASGHDFHLWYI |
| 146 | | HWVRQAPGQGLEWMGRVFAGSGNTKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVE |
| | | DKVAWYQQKPGKAPKALIYSSSHRYKGVPSRFSG |
| | | SGSGTEFTLTISSLQPEDFATYFCQQFKSYPLTFGQ |
| | | GTKLEIK |
| SEQ ID NO: | VL | DIQMTQSPSFLSASVGDRVTITCKASQNVEDKVAW |
| 147 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFHLWYI |
| 148 | | HWVRQAPGQGLEWMGRVFAGSGNTKYNEKFKG |
| | | RVTITADTSTSTAYMELSSLRSEDTAVYYCAGSYY |
| | | SYDVLDYWGQGTTVTVSS |
| A-H.26 | | |
| SEQ ID NO: | VH + VL | QVQLVQSGAEVKKPGSSVKVSCKASGTDFKLTYIH |
| 149 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 150 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 151 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSS |
| A-H.27 | | |
| SEQ ID NO: | VH + VL | QVQLVQSGAEVKKPGSSVKVSCKASGTDFKLTYIH |
| 153 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGN |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVGNRVAW |
| 154 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 155 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSS |
| A-H.28 | | |
| SEQ ID NO: | VH + VL | QVQLVQSGAEVKKPGSSVKVSCKASGTDFKLTYIH |
| 156 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 157 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 158 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSS |
| A-H.29 | | |
| SEQ ID NO: | VH + VL | QVQLVQSGAEVKKPGSSVKVSCKASGHDFHLWYI |
| 159 | | HWVRQAPGQGLEWMGRISPGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGD |
| | | RVAWHQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVGDRVAW |
| 160 | | HQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFHLWYI |
| 161 | | HWVRQAPGQGLEWMGRISPGSGNVKYNEKFKGR |
| | | VTITADTSTSTAYMELSSLRSEDTAVYYCAGSYYS |
| | | YDVLDYWGQGTTVTVSS |
| A-H.31 | | |
| SEQ ID NO: | VH + VL | QVQLVQSGAEVKKPGSSVKVSCKASGHDFKLTYI |
| 162 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 163 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFKLTYI |
| 164 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |
| A-H.31 | | |
| SEQ ID NO: | VH + VL | QVQLVQSGAEVKKPGSSVKVSCKASGTDFHLWYI |
| 165 | | HWVRQAPGQGLEWMGRVFAGSGSYRYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 166 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFHLWYI |
| 167 | | HWVRQAPGQGLEWMGRVFAGSGSYRYNEKFKGR |
| | | VTITADTSTSTAYMELSSLRSEDTAVYYCAGSYYS |
| | | YDVLDYWGQGTTVTVSS |
| A-H.32 | | |
| SEQ ID NO: | VH + VL | QVQLVQSGAEVKKPGSSVKVSCKASGTDFDKIYIH |
| 168 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVADR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVADRVAW |
| 169 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFDKIYIH |
| 170 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSS |
| A-H.33 | | |
| SEQ ID NO: | VH + VL | QVQLVQSGAEVKKPGSSVKVSCKASGTDFKLTYIH |
| 171 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVEDRVAW |
| 172 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 173 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSS |
| A-H.34 | | |
| SEQ ID NO: | VH + VL | QVQLVQSGAEVKKPGSSVKVSCKASGTDFRLTYIH |
| 174 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGNRVAW |
| 175 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFRLTYIH |
| 176 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSS |
| A-H.35 | | |
| SEQ ID NO: | VH + VL | QVQLVQSGAEVKKPGSSVKVSCKASGHDFDKTYI |
| 177 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVE |
| | | DRVAWYQQKPGKAPKALIYSSSHRYKGVPSRFSG |
| | | SGSGTEFTLTISSLQPEDFATYFCQQFKSYPLTFGQ |
| | | GTKLEIK |
| SEQ ID NO: | VL | DIQMTQSPSFLSASVGDRVTITCKASQNVEDRVAW |
| 178 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFDKTYI |
| 179 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |
| A-H.36 | | |
| SEQ ID NO: | VH + VL | QVQLVQSGAEVKKPGSSVKVSCKASGHDFKLTYI |
| 180 | | HWVRQAPGQGLEWMGRVSPGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVED |
| | | RVAWHQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVEDRVAW |
| 181 | | HQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFKLTYI |
| 182 | | HWVRQAPGQGLEWMGRVSPGSGNTKYNEKFKGR |
| | | VTITADTSTSTAYMELSSLRSEDTAVYYCAGSYYS |
| | | YDVLDYWGQGTTVTVSS |
| A-H.37 | | |
| SEQ ID NO: | VH + VL | QVQLVQSGAEVKKPGSSVKVSCKASGHDFDKTYI |
| 183 | | HWVRQAPGQGLEWMGRIYPGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVAD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVADRVAW |
| 184 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFDKTYI |
| 185 | | HWVRQAPGQGLEWMGRIYPGSGNVKYNEKFKGR |
| | | VTITADTSTSTAYMELSSLRSEDTAVYYCAGSYYS |
| | | YDVLDYWGQGTTVTVSS |
| A-H.38 | | |
| SEQ ID NO: | VH + VL | QVQLVQSGAEVKKPGSSVKVSCKASGTDFDKTYI |
| 186 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 187 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFDKTYI |
| 188 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |
| A-H.39 | | |
| SEQ ID NO: | VH + VL | QVQLVQSGAEVKKPGSSVKVSCKASGTDFDKIYIH |
| 189 | | WVRQAPGQGLEWMGRISAGSGNIKYNEKFKGRVT |
| | | ITADTSTSTAYMELSSLRSEDTAVYYCAGSYYSYD |
| | | VLDYWGQGTTVTVSSGGGGSGGGGSGGGGSGGG |
| | | GSDIQMTQSPSFLSASVGDRVTITCKASQNVDDRV |
| | | AWYQQKPGKAPKALIYSSSHRYKGVPSRFSGSGSG |
| | | TEFTLTISSLQPEDFATYFCQQFKSYPLTFGQGTKL |
| | | EIK |
| SEQ ID NO: | VL | DIQMTQSPSFLSASVGDRVTITCKASQNVDDRVAW |
| 190 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFDKIYIH |
| 191 | | WVRQAPGQGLEWMGRISAGSGNIKYNEKFKGRVT |
| | | ITADTSTSTAYMELSSLRSEDTAVYYCAGSYYSYD |
| | | VLDYWGQGTTVTVSS |
| A-H.40 | | |
| SEQ ID NO: | VH + VL | QVQLVQSGAEVKKPGSSVKVSCKASGTDFDKIYIH |
| 192 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 193 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFDKIYIH |
| 194 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |
| A-H.41 | | |
| SEQ ID NO: | VH + VL | QVQLVQSGAEVKKPGSSVKVSCKASGGTFKLTYIH |
| 195 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVDDRVAW |
| 196 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGGTFKLTYIH |
| 197 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSS |
| A-H.42 | | |
| SEQ ID NO: | VH + VL | QVQLVQSGAEVKKPGSSVKVSCKASGTDFKLTYIH |
| 198 | | WVRQAPGQGLEWMGRISPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNR |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNRVAW |
| 199 | | HQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGTDFKLTYIH |
| 200 | | WVRQAPGQGLEWMGRISPGSGNVKYNEKFKGRV |
| | | TITADTSTSTAYMELSSLRSEDTAVYYCAGSYYSY |
| | | DVLDYWGQGTTVTVSS |
| A-H.43 | | |
| SEQ ID NO: | VH + VL | QVQLVQSGAEVKKPGSSVKVSCKASGHDFDKFYI |
| 201 | | HWVRQAPGQGLEWMGRVSAGSGNTKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVD |
| | | NRVAWYQQKPGKAPKALIYSSSHRYKGVPSRFSG |
| | | SGSGTEFTLTISSLQPEDFATYFCQQFKSYPLTFGQ |
| | | GTKLEIK |
| SEQ ID NO: | VL | DIQMTQSPSFLSASVGDRVTITCKASQNVDNRVAW |
| 202 | | YQQKPGKAPKALIYSSSHRYKGVPSRFSGSGSGTE |
| | | FTLTISSLQPEDFATYFCQQFKSYPLTFGQGTKLEIK |
| SEQ ID NO: | VH | QVQLVQSGAEVKKPGSSVKVSCKASGHDFDKFYI |
| 203 | | HWVRQAPGQGLEWMGRVSAGSGNTKYNEKFKG |
| | | RVTITADTSTSTAYMELSSLRSEDTAVYYCAGSYY |
| | | SYDVLDYWGQGTTVTVSS |
| A-H.44 | | |
| SEQ ID NO: | VH + VL | QVQLVQSGAEVKKPGSSVKVSCKASGTDFDKFYI |
| 204 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVG |
| | | DRVVWYQQKPGKAPKALIYSSSHRYKGVPSRFSG |
| | | SGSGTEFTLTISSLQPEDFATYFCQQFKSYPLTFGQ |
| | | GTKLEIK |
| SEQ ID NO: | VH | QVQLVQSGAEVKKPGSSVKVSCKASGTDFDKFYI |
| 205 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |
| A-H.45 | | |
| SEQ ID NO: | VH + VL | QVQLVQSGAEVKKPGSSVKVSCKASGYSFTTYYIH |
| 206 | | WVRQAPGQGLEWMGWFSAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVGIN |
| | | VVWHQQKPGKAPKALIYSSSHRYSGVPSRFSGSGS |
| | | GTEFTLTISSLQPEDFATYFCQQFKSYPLTFGQGTK |
| | | LEIK |
| SEQ ID NO: | VH | QVQLVQSGAEVKKPGSSVKVSCKASGYSFTTYYIH |
| 207 | | WVRQAPGQGLEWMGWFSAGSGNTKYNEKFKGR |
| | | VTITADTSTSTAYMELSSLRSEDTAVYYCAVSYYS |
| | | YDVLDYWGQGTTVTVSS |
| A-H.46 | | |
| SEQ ID NO: | VH + VL | QVQLVQSGAEVKKPGSSVKVSCKASGYSFTTYYIH |
| 208 | | WVRQAPGQGLEWMGWFSAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGIN |
| | | VVWHQQKPGKAPKALIYSSSHRYSGVPSRFSGSGS |
| | | GTEFTLTISSLQPEDFATYFCQQFKSYPLTEGQGTK |
| | | LEIK. |
| SEQ ID NO: | VH | QVQLVQSGAEVKKPGSSVKVSCKASGYSFTTYYIH |
| 209 | | WVRQAPGQGLEWMGWFSAGSGNTKYNEKFKGR |
| | | VTITADTSTSTAYMELSSLRSEDTAVYYCAGSYYS |
| | | YDVLDYWGQGTTVTVSS |
| A-H.47 | | |
| SEQ ID NO: | VH + VL | QVQLVQSGAEVKKPGSSVKVSCKASGYSFTTTYYIH |
| 210 | | WVRQAPGQGLEWMGWFFPGSGNTKYNEKEKGRV |
| | | TITADTSTSTAYMELSSLRSEDTAVYYCAGSYYSY |
| | | DVLDYWGQTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKL |
| | | EIK |
| SEQ ID NO: | VH | QVQLVQSGAEVKKPGSSVKVSCKASGYSFTTYYIH |
| 211 | | WVRQAPGQGLEWMGWFFPGSGNTKYNEKFKGRV |
| | | TITADTSTSTAYMELSSLRSEDTAVYYCAGSYYSY |
| | | DVLDYWGQGTTVTVSS |
| A-H.48 | | |
| SEQ ID NO: | VH + VL | QVQLVQSGAEVKKPGSSVKVSCKASGYSFTTYYIH |
| 212 | | WVRQAPGQGLEWMGWFSPGSGNTKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKL |
| | | EIK |
| SEQ ID NO: | VH | QVQLVQSGAEVKKPGSSVKVSCKASGYSFTTYYIH |
| 213 | | WVRQAPGQGLEWMGWFSPGSGNTKYNEKFKGRV |
| | | TITADTSTSTAYMELSSLRSEDTAVYYCAVSYYSY |
| | | DVLDYWGQGTTVTVSS |
| A-H.49 | | |
| SEQ ID NO: | VH + VL | QVQLVQSGAEVKKPGSSVKVSCKASGYSFTTYYIH |
| 214 | | WVRQAPGQGLEWMGWFSPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKL |
| | | EIK |
| SEQ ID NO: | VH | QVQLVQSGAEVKKPGSSVKVSCKASGYSFTTYYIH |
| 215 | | WVRQAPGQGLEWMGWFSPGSGNTKYNEKFKGRV |
| | | TITADTSTSTAYMELSSLRSEDTAVYYCAGSYYSY |
| | | DVLDYWGQGTTVTVSS |
| A-H.50 | | |
| SEQ ID NO: | VH + VL | QVQLVQSGAEVKKPGSSVKVSCKASGYSFTTYYIH |
| 216 | | WVRQAPGQGLEWMGRIFPGSGNIKYNEKFKGRVT |
| | | ITADTSTSTAYMELSSLRSEDTAVYYCAGSYYSYD |
| | | VLDYWGQGTTVTVSSGGGGSGGGGSGGGGSGGG |
| | | GSDIQMTQSPSFLSASVGDRVTITCKASQNVGINVV |
| | | WHQQKPGKAPKALIYSSSHRYSGVPSRFSGSGSGT |
| | | EFTLTISSLQPEDFATYFCQQFKSYPLTFGQGTKLEI |
| | | K |
| SEQ ID NO: | VH | QVQLVQSGAEVKKPGSSVKVSCKASGYSFTTYYIH |
| 217 | | WVRQAPGQGLEWMGRIFPGSGNIKYNEKFKGRVT |
| | | ITADTSTSTAYMELSSLRSEDTAVYYCAGSYYSYD |
| | | VLDYWGQGTTVTVSS |
| A-H.51 | | |
| SEQ ID NO: | VH + VL | QVQLVQSGAEVKKPGSSVKVSCKASGYSFTTYYIH |
| 218 | | WVRQAPGQGLEWMGWFFPGSGNIKYNEKFKGRV |
| | | TITADTSTSTAYMELSSLRSEDTAVYYCAGSIYSAG |
| | | VLDYWGQGTTVTVSSGGGGSGGGGSGGGGSGGG |
| | | GSDIQMTQSPSFLSASVGDRVTTTCKASQNVGINVV |
| | | WHQQKPGKAPKALIYSSSHRYSGVPSRFSGSGSGT |
| | | EFTLTISSLQPEDFATYFCQQFKSYPLTFGQGTKLEI |
| | | K |
| SEQ ID NO: | VH | QVQLVQSGAEVKKPGSSVKVSCKASGYSFTTYYIH |
| 219 | | WVRQAPGQGLEWMGWFFPGSGNIKYNEKFKGRV |
| | | TITADTSTSTAYMELSSLRSEDTAVYYCAGSIYSAG |
| | | VLDYWGQGTTVTVSS |
| A-H.52 | | |
| SEQ ID NO: | VH + VL | QVQLVQSGAEVKKPGSSVKVSCKASGYSFTLGYIH |
| 220 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKL |
| | | EIK |
| SEQ ID NO: | VH | QVQLVQSGAEVKKPGSSVKVSCKASGYSFTLGYIH |
| 221 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| A-H.53 | | |
| SEQ ID NO: | VH + VL | QVQLVQSGAEVKKPGSSVKVSCKASGYSFRLTYIH |
| 222 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKL |
| | | EIk |
| SEQ ID NO: | VH | QVQLVQSGAEVKKPGSSVKVSCKASGYSFRLTYIH |
| 223 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| A-H.54 | | |
| SEQ ID NO: | VH + VL | QVQLVQSGAEVKKPGSSVKVSCKASGYSFHNWYI |
| 224 | | HWVRQAPGQGLEWMGWFFPGSGNIKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGIN |
| | | VVWHQQKPGKAPKALIYSSSHRYSGVPSRFSGSGS |
| | | GTEFTLTISSLQPEDFATYFCQQFKSYPLTFGQGTK |
| | | LEIK |
| SEQ ID NO: | VH | QVQLVQSGAEVKKPGSSVKVSCKASGYSFHNWYI |
| 225 | | HWVRQAPGQGLEWMGWFFPGSGNIKYNEKFKGR |
| | | VTITADTSTSTAYMELSSLRSEDTAVYYCAGSYYS |
| | | YDVLDYWGQGTTVTVSS |
| A-H.55 antibody | | |
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYYIH |
| 1100 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: | VL | QSVLTQPPSVSEAPRQRVTISCKASQNVGINVVWH |
| 1101 | | QQLPGKAPKALIYSSSHRYSGVSDRFSGSGSGTSFS |
| | | LAISGLQSEDEADYFCQQFKSYPLTFGTGTKVTVL |
| A-H.56 | | |
| SEQ ID NO: | VH + VL (ScFv) | QVQLVQSGAEVKKPGSSVKVSCKASGHDFDKFYI |
| 1309 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVG |
| | | NRVAWYQQKPGKAPKALIYSSSHRYKGVPSRFSG |
| | | SGSGTEFTLTISSLQPEDFATYFCQQFKSYPLTFGQ |
| | | GTKLEIK |
| A-H.57 | | |
| SEQ ID NO: | VH + VL (ScFv) | QVQLVQSGAEVKKPGSSVKVSCKASGHDFRLTYI |
| 1326 | | HWVRQAPGQGLEWMGRVSAGSGNTKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVG |
| | | DRVVWHQQKPGKAPKALIYSSSHRYKGVPSRFSG |
| | | SGSGTEFTLTISSLQPEDFATYFCQQFKSYPLTFGQ |
| | | GTKLEIK |
| A-H.58 | | |
| SEQ ID NO: | VH + VL (ScFv) | QVQLVQSGAEVKKPGSSVKVSCKASGHDFRLTYI |
| 1327 | | HWVRQAPGQGLEWMGRVSAGSGNVKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVG |
| | | VNRVVWHQQKPGKAPKALIYSSSHRYKGVPSRFSG |
| | | SGSGTEFTLTISSLQPEDFATYFCQQFKSYPLTFGQ |
| | | VGTKLEIK |
| A-H.59 | | |
| SEQ ID NO: | VH + VL (ScFv) | QVQLVQSGAEVKKPGSSVKVSCKASGHDFRLTYI |
| 1328 | | HWVRQAPGQGLEWMGRIYAGSGNVKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVAD |
| | | RVVWHQQKPGKAPKALIYSSSHRYKGVPSRFSGS |
| | | GSGTEFTLTISSLQPEDFATYFCQQFKSYPLTFGQG |
| | | TKLEIK |
| A-H.60 | | |
| SEQ ID NO: | VH + VL (ScFv) | QVQLVQSGAEVKKPGSSVKVSCKASGHDFKLTYI |
| 1329 | | HWVRQAPGQGLEWMGRVSAGSGNTKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVG |
| | | DRVAWHQQKPGKAPKALIYSSSHRYKGVPSRFSG |
| | | SGSGTEFTLTISSLQPEDFATYFCQQFKSYPLTFGQ |
| | | GTKLEIK |
| A-H.61 | | |
| SEQ ID NO: | VH + VL (ScFv) | QVQLVQSGAEVKKPGSSVKVSCKASGHDFKLTYI |
| 1330 | | HWVRQAPGQGLEWMGRVSAGSGNTKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVD |
| | | NRVAWHQQKPGKAPKALIYSSSHRYKGVPSRFSG |
| | | SGSGTEFTLTISSLQPEDFATYFCQQFKSYPLTFGQ |
| | | GTKLEIK |
| A-H.62 | | |
| SEQ ID NO: | VH + VL (ScFv) | QVQLVQSGAEVKKPGSSVKVSCKASGHDFRLTYI |
| 1331 | | HWVRQAPGQGLEWMGRVSAGSGNVKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVA |
| | | DRVVWHQQKPGKAPKALIYSSSHRYKGVPSRFSG |
| | | SGSGTEFTLTISSLQPEDFATYFCQQFKSYPLTFGQ |
| | | GTKLEIK |
| A-H.63 | | |
| SEQ ID NO: | VH + VL (ScFv) | QVQLVQSGAEVKKPGSSVKVSCKASGHDFRLTYI |
| 1332 | | HWVRQAPGQGLEWMGRVYAGSGNTKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVE |
| | | DRVVWHQQKPGKAPKALIYSSSHRYKGVPSRFSG |
| | | SGSGTEFTLTISSLQPEDFATYFCQQFKSYPLTFGQ |
| | | GTKLEIK |
| A-H.64 | | |
| SEQ ID NO: | VH + VL (ScFv) | QVQLVQSGAEVKKPGSSVKVSCKASGHDFKLTYI |
| 1333 | | HWVRQAPGQGLEWMGRVSAGSGNTKYNEKFKG |
| | | RVTITADTSTSTAYMELSSLRSEDTAVYYCAVSYY |
| | | SYDVLDYWGQGTTVTVSSGGGGSGGGGSGGGGS |
| | | GGGGSDIQMTQSPSFLSASVGDRVTITCKASQNVA |
| | | DRVVWHQQKPGKAPKALIYSSSHRYKGVPSRFSG |
| | | SGSGTEFTLTISSLQPEDFATYFCQQFKSYPLTFGQ |
| | | GTKLEIK |
| A-H.65 | | |
| SEQ ID NO: | VH + VL (ScFv) | QVQLVQSGAEVKKPGSSVKVSCKASGHDFKLTYI |
| 1334 | | HWVRQAPGQGLEWMGRISAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVGD |
| | | RVVWHQQKPGKAPKALIYSSSHRYKGVPSRFSGS |
| | | GSGTEFTLTISSLQPEDFATYFCQQFKSYPLTFGQG |
| | | TKLEIK |
| A-H.66 | | |
| SEQ ID NO: | VH + VL (ScFv) | QVQLVQSGAEVKKPGSSVKVSCKASGHDFKLTYI |
| 1335 | | HWVRQAPGQGLEWMGRIYAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVGD |
| | | RVVWHQQKPGKAPKALIYSSSHRYKGVPSRFSGS |
| | | GSGTEFTLTISSLQPEDFATYFCQQFKSYPLTFGQG |
| | | TKLEIK |
| A-H.67 | | |
| SEQ ID NO: | VH + VL (ScFv) | QVQLVQSGAEVKKPGSSVKVSCKASGTDFKLTYIH |
| 1336 | | WVRQAPGQGLEWMGRIFPGSGNVKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVDNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| A-H.68 | | |
| SEQ ID NO: | VH + VL (ScFv) | QVQLVQSGAEVKKPGSSVKVSCKASGHDFRLTYI |
| 1337 | | HWVRQAPGQGLEWMGRISAGSGNVKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVAD |
| | | RVAWYQQKPGKAPKALIYSSSHRYKGVPSRFSGS |
| | | GSGTEFTLTISSLQPEDFATYFCQQFKSYPLTFGQG |
| | | TKLEIK |
| A-H.69 | | |
| SEQ ID NO: | VH + VL (ScFv) | QVQLVQSGAEVKKPGSSVKVSCKASGTDFKLTYIH |
| 1344 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |

A-H humanized-matured VH

| | | |
|---|---|---|
| SEQ ID NO: | VH-humanized | QVQLVQSGAEVKKPGSSVKVSCKASGTDFKLTYIH |
| 1310 | matured 1 | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |
| SEQ ID NO: | VH-humanized | QVQLVQSGAEVKKPGSSVKVSCKASGTDFKLTYIH |
| 1311 | matured 2 | WVRQAPGQGLEWMGRIFPGSGNVKYNEKFKGRV |
| | | TITADTSTSTAYMELSSLRSEDTAVYYCAVSYYSY |
| | | DVLDYWGQGTTVTVSS |
| SEQ ID NO: | VH-humanized | QVQLVQSGAEVKKPGSSVKVSCKASGHDFRLTYI |
| 1312 | matured 3 | HWVRQAPGQGLEWMGRISAGSGNVKYNEKFKGR |
| | | VTITADTSTSTAYMELSSLRSEDTAVYYCAVSYYS |
| | | YDVLDYWGQGTTVTVSS |

A-H humanized-matured VL

| | | |
|---|---|---|
| SEQ ID NO: | VL-humanized matured | DIQMTQSPSFLSASVGDRVTITCKASQNVDNRVAW |
| 1313 | 1 | YQQKPGKAPKALIYSSSHRYKGVPSRFSGSGSGTE |
| | | FTLTISSLQPEDFATYFCQQFKSYPLTFGQGTKLEIK |
| SEQ ID NO: | VL-humanized matured | DIQMTQSPSFLSASVGDRVTITCKASQNVADRVAW |
| 1314 | 2 | YQQKPGKAPKALIYSSSHRYKGVPSRFSGSGSGTE |
| | | FTLTISSLQPEDFATYFCQQFKSYPLTFGQGTKLEIK |

In some embodiments, the anti-TCRβV antibody molecule, e.g., anti-TCRβ V6 (e.g., anti-TCRβ V6-5*01) antibody molecule comprises a VH and/or a VL of an antibody described in Table 1A, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1A, or a sequence with at least 80%, 85%, 90%, 95%, 96%, 97%, 98%, 99% or more identity thereto.

In some embodiments, an anti-TCRVb antibody disclosed herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody disclosed herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Anti-TCRβ V12 Antibodies

Accordingly, in one aspect, the disclosure provides an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 2A, or encoded by a nucleotide sequence in Table 2A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein, e.g., an antibody as described in Table 2A, or encoded by a nucleotide sequence in Table 2A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two heavy chain variable regions from an antibody described herein, e.g., an antibody as described in Table 2A, or encoded by a nucleotide sequence in Table 2A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two light chain variable regions from an antibody described herein, e.g., an antibody as described in Table 2A, or encoded by a nucleotide sequence in Table 2A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In one embodiment, the heavy chain constant region comprises an amino sequence set forth in Table 3A, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In one embodiment, the light chain constant region comprises an amino sequence set forth in Table 3A, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 2A, or encoded by the nucleotide sequence in Table 2A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 2A, or encoded by a nucleotide sequence shown in Table 2A. In one embodiment, one or more of the CDRs (or collectively all of the CDRs) have one, two, three, four, five, six or more changes, e.g., amino acid substitutions or deletions, relative to the amino acid sequence shown in Table 2A, or encoded by a nucleotide sequence shown in Table 2A.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 2A, or encoded by the nucleotide sequence in Table 2A, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 2A, or encoded by a nucleotide sequence shown in Table 2A. In one embodiment, one or more of the CDRs (or collectively all of the CDRs) have one, two, three, four, five, six or more changes, e.g., amino acid substitutions or deletions, relative to the amino acid sequence shown in Table 2A, or encoded by a nucleotide sequence shown in Table 2A.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 2A, or encoded by a nucleotide sequence shown in Table 2A. In one embodiment, one or more of the CDRs (or collectively all of the CDRs) have one, two, three, four, five, six or more changes, e.g., amino acid substitutions or deletions, relative to the amino acid sequence shown in Table 2A, or encoded by a nucleotide sequence shown in Table 2A.

In some embodiments, the anti-TCRβV antibody molecule, e.g., anti-TCRβ V12 antibody molecule, molecule includes all six CDRs from an antibody described herein, e.g., an antibody as described in Table 2A, or encoded by the nucleotide sequence in Table 2A, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2A) from a heavy chain variable region of an antibody described herein, e.g., an antibody chosen as described in Table 2A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2A.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2A) from a light chain variable region of an antibody described herein, e.g., an antibody as described in Table 2A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2A.

In some embodiments, the anti-TCRβV antibody molecule, e.g., anti-TCRβ V12 antibody molecule includes at least one, two, three, four, five, or six CDRs according to Kabat et al. (e.g., at least one, two, three, four, five, or six CDRs according to the Kabat definition as set out in Table 2A) from the heavy and light chain variable regions of an antibody described herein, e.g., an antibody as described in Table 2A, or encoded by the nucleotide sequence in Table 2A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 2A.

In some embodiments, the anti-TCRβV antibody molecule, e.g., anti-TCRβ V12 antibody molecule includes all six CDRs according to Kabat et al. (e.g., all six CDRs according to the Kabat definition as set out in Table 2A) from the heavy and light chain variable regions of an antibody described herein, e.g., an antibody as described in Table 2A, or encoded by the nucleotide sequence in Table 2A; or encoded by the nucleotide sequence in Table 2A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 2A. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three hypervariable loops that have the same canonical structures as the corresponding hypervariable loop of an antibody described herein, e.g., an antibody described in Table 2A, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2A) from a heavy chain variable region of an antibody described herein, e.g., an antibody chosen as described in Table 2A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2A.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2A) from a light chain variable region of an antibody described herein, e.g., an antibody as described in Table 2A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2A.

In some embodiments, the anti-TCRβV antibody molecule, e.g., anti-TCRβ V12 antibody molecule includes at least one, two, three, four, five, or six CDRs according to Chothia et al. (e.g., at least one, two, three, four, five, or six CDRs according to the Chothia definition as set out in Table 2A) from the heavy and light chain variable regions of an antibody described herein, e.g., an antibody as described in Table 2A, or encoded by the nucleotide sequence in Table 2A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 2A.

In some embodiments, the anti-TCRβV antibody molecule, e.g., anti-TCRβ V12 antibody molecule includes all six CDRs according to Chothia et al. (e.g., all six CDRs according to the Chothia definition as set out in Table 2A) from the heavy and light chain variable regions of an antibody described herein, e.g., an antibody as described in Table 2A, or encoded by the nucleotide sequence in Table 2A; or encoded by the nucleotide sequence in Table 2A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 2A. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2A) from a heavy chain variable region of an antibody described herein, e.g., an antibody chosen as described in Table 2A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 2A.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2A) from a light chain variable region of an antibody described herein, e.g., an antibody as described in Table 2A, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 2A.

In some embodiments, the anti-TCRβV antibody molecule, e.g., anti-TCRβ V12 antibody molecule includes at least one, two, three, four, five, or six CDRs according to a combined CDR. (e.g., at least one, two, three, four, five, or six CDRs according to the combined CDR definition as set out in Table 2A) from the heavy and light chain variable regions of an antibody described herein, e.g., an antibody as described in Table 2A, or encoded by the nucleotide sequence in Table 2A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 2A.

In some embodiments, the anti-TCRβV antibody molecule, e.g., anti-TCRβ V12 antibody molecule includes all six CDRs according to a combined CDR (e.g., all six CDRs according to the combined CDR definition as set out in Table 2A) from the heavy and light chain variable regions of an antibody described herein, e.g., an antibody as described in Table 2A, or encoded by the nucleotide sequence in Table 2A; or encoded by the nucleotide sequence in Table 2A; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 2A. In some embodiments, the anti-TCRβV antibody molecule, e.g., anti-TCRβ V12 antibody molecule may include any CDR described herein.

In some embodiments, a combined CDR as set out in Table 1A is a CDR that comprises a Kabat CDR and a Chothia CDR.

In some embodiments, the anti-TCRβV antibody molecule, e e.g., anti-TCRβ V12 antibody molecule, molecule includes a combination of CDRs or hypervariable loops identified as combined CDRs in Table 1A. In some embodiments, the anti-TCRβV antibody molecule, e.g., anti-TCRβ V12 antibody molecule, can contain any combination of CDRs or hypervariable loops according the “combined” CDRs are described in Table 1A.

In some embodiments, the anti-TCRβV antibody molecule, e.g., anti-TCRβ V12 antibody molecule includes a combination of CDRs or hypervariable loops defined according to the Kabat et al. and Chothia et al., or as described in Table 1A

In some embodiments, the anti-TCRβV antibody molecule, e.g., anti-TCRβ V12 antibody molecule can contain any combination of CDRs or hypervariable loops according to the Kabat and Chothia definitions.

In an embodiment, e.g., an embodiment comprising a variable region, a CDR (e.g., a combined CDR, Chothia CDR or Kabat CDR), or other sequence referred to herein, e.g., in Table 2A, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V12 antibody molecule includes:

(i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24, or SEQ ID NO: 25.

In some embodiments, the anti-TCRβV antibody molecule, e.g., anti-TCRβ V12 antibody molecule comprises:

(i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or
(ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβ V12 antibody molecule comprises:

(i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or
(ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβ V12 antibody molecule comprises:

(i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises:

(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises:

(i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or
(ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβ V12 antibody molecule comprises:

(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In one embodiment, the light or the heavy chain variable framework (e.g., the region encompassing at least FR1, FR2, FR3, and optionally FR4) of the anti-TCRβV antibody molecule, e.g., anti-TCRβ V12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

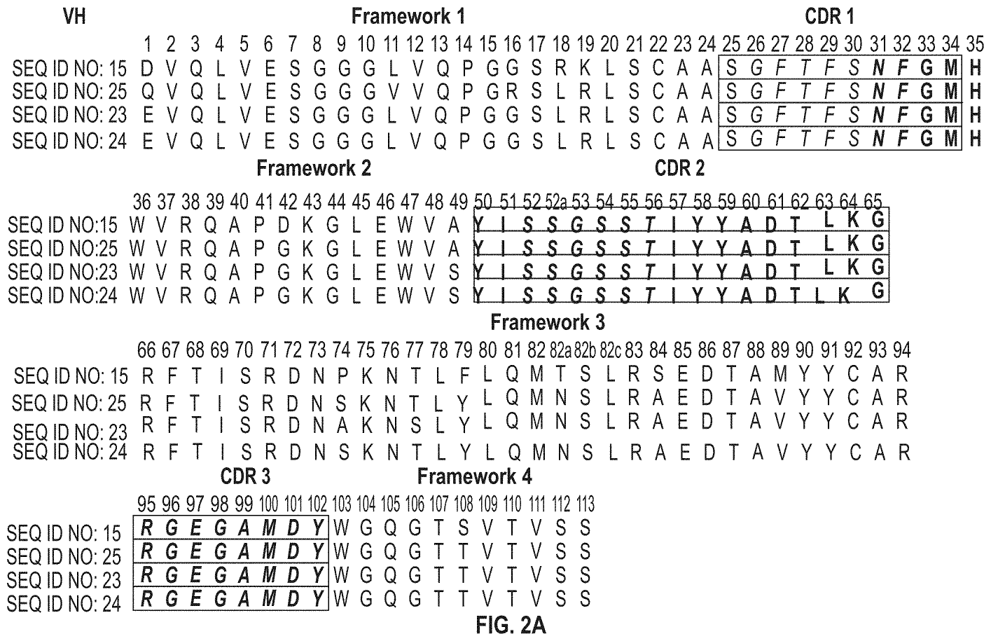

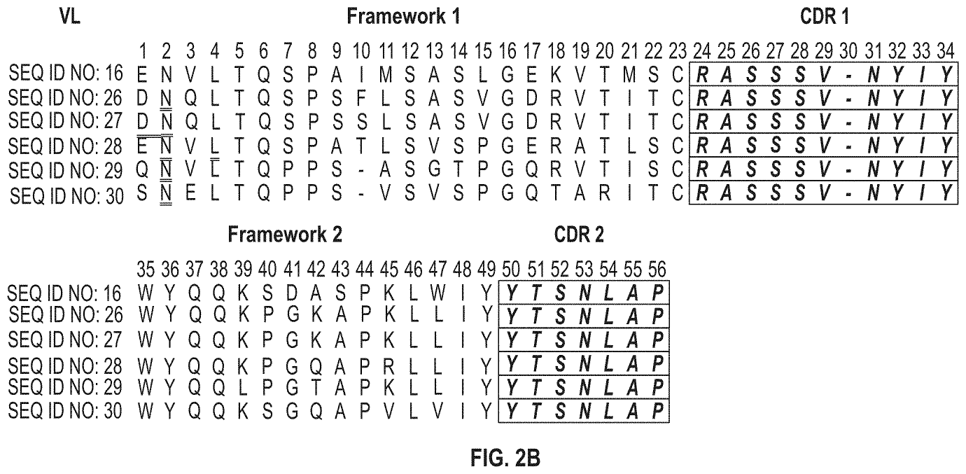

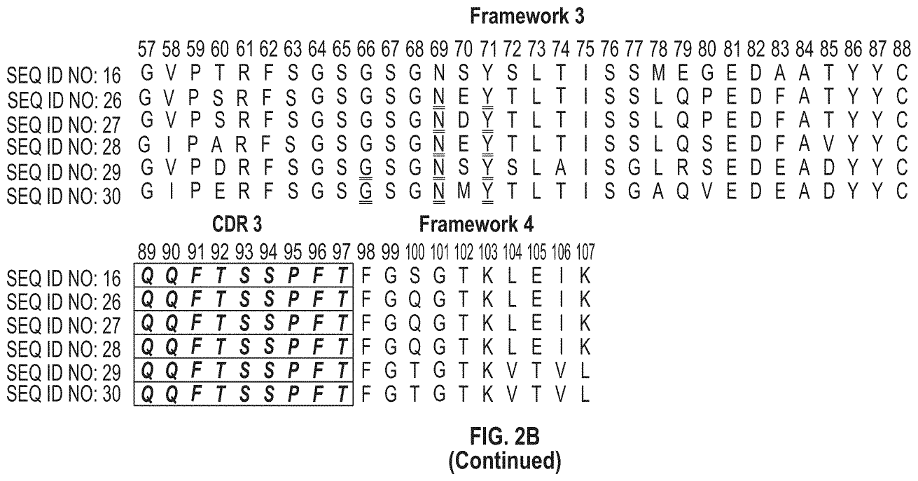

In some embodiments, the anti-TCRβV antibody molecule, e.g., anti-TCRβ V12 antibody molecule, comprises a heavy chain variable domain having at least one, two, three, four, five, six, seven, ten, fifteen, twenty or more changes, e.g., amino acid substitutions or deletions, from an amino acid sequence described in Table 2A. e.g., the amino acid sequence of the FR region in the entire variable region, e.g., shown in FIGS. 2A and 2B, or in SEQ ID NOs: 23-25.

Alternatively, or in combination with the heavy chain substitutions described herein the anti-TCRβV antibody molecule, e.g., anti-TCRβ V12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein. e.g., the amino acid sequence of the FR region in the entire variable region, e.g., shown in FIGS. 2A and 2B, or in SEQ ID NOs: 26-30.

In some embodiments, the anti-TCRβV antibody molecule, e.g., anti-TCRβ V12 antibody molecule includes one, two, three, or four heavy chain framework regions shown in FIG. 2A, or a sequence substantially identical thereto.

In some embodiments, the anti-TCRβV antibody molecule, e.g., anti-TCRβ V12 antibody molecule includes one, two, three, or four light chain framework regions shown in FIG. 2B, or a sequence substantially identical thereto.

In some embodiments, the anti-TCRβV antibody molecule, e.g., anti-TCRβ V12 antibody molecule comprises the light chain framework region 1 e.g., as shown in FIG. 2B.

In some embodiments, the anti-TCRβV antibody molecule, e.g., anti-TCRβ V12 antibody molecule comprises the light chain framework region 2 e.g., as shown in FIG. 2B.

In some embodiments, the anti-TCRβV antibody molecule, e.g., anti-TCRβ V12 antibody molecule comprises the light chain framework region 3, e.g., as shown in FIG. 2B.

In some embodiments, the anti-TCRβV antibody molecule, e.g., anti-TCRβ V12 antibody molecule comprises the light chain framework region 4, e.g., as shown in FIG. 2B.

In some embodiments, the anti-TCRβV antibody molecule, e.g., anti-TCRβ V12 antibody molecule comprises a light chain comprising a framework region, e.g., framework region 1 (FR1), comprising a change, e.g., a substitution (e.g., a conservative substitution) at one or more, e.g., all, position disclosed herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FR1 comprises an Asparagine at position 2, e.g., a substitution at position 2 according to Kabat numbering, e.g., an Isoleucine to Asparagine substitution, Serine to Asparagine substitution or Tyrosine to Asparagine substitution. In some embodiments, FR1 comprises a Leucine at position 4, e.g., a substitution at position 4 according to Kabat numbering, e.g., a Methionine to Leucine substitution.

In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 2 according to Kabat numbering, e.g., an Isoleucine to Asparagine substitution, Serine to Asparagine substitution or Tyrosine to Asparagine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβ V12 antibody molecule comprises a light chain comprising a framework region, e.g., framework region 3 (FR3), comprising a change, e.g., a substitution (e.g., a conservative substitution) at one or more, e.g., all, position disclosed herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments, FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβ V12 antibody molecule comprises a light chain comprising: a framework region 1 (FR1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Tyrosine to Asparagine substitution; and (b) a framework region 3 (FR3) comprising a substitution at position 66 according to Kabat numbering, e.g., a Serine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain variable domain comprising: (a) a framework region 1 (FR1) comprising a change, e.g., a substitution (e.g., a conservative substitution) at one or more (e.g., all) positions disclosed herein according to Kabat numbering, and (b) a framework region 3 (FR3) comprising a change, e.g., a substitution (e.g., a conservative substitution) at one or more (e.g., all) position disclosed herein according to Kabat numbering. In some embodiments, the substitution is relative to a human germline light chain framework region sequence.

In some embodiments, the anti-TCRβV antibody molecule, e.g., anti-TCRβ V12 antibody molecule comprises the heavy chain framework region 1, e.g., as shown in FIG. 2A.

In some embodiments, the anti-TCRβV antibody molecule, e.g., anti-TCRβ V12 antibody molecule comprises the heavy chain framework region 2, e.g., as shown in FIG. 2A.

In some embodiments, the anti-TCRβV antibody molecule, e.g., anti-TCRβ V12 antibody molecule comprises the heavy chain framework region 3, e.g., as shown in FIG. 2A.

In some embodiments, the anti-TCRβV antibody molecule, e.g., anti-TCRβ V12 antibody molecule comprises the heavy chain framework region 4, e.g., as shown in FIG. 2A.

In some embodiments, the anti-TCRβV antibody molecule, e.g., anti-TCRβ V12 antibody molecule comprises the heavy chain framework regions 1-4, e.g., SEQ ID NOS: 20-23, or as shown in FIG. 2A.

In some embodiments, the anti-TCRβV antibody molecule, e.g., anti-TCRβ V12 antibody molecule comprises the light chain framework regions 1-4, e.g., SEQ ID NOs: 26-30, or as shown in FIG. 2B.

In some embodiments, the anti-TCRβV antibody molecule, e.g., anti-TCRβ V12 antibody molecule comprises the heavy chain framework regions 1-4, e.g., SEQ ID NOs: 23-25; and the light chain framework regions 1-4, e.g., SEQ ID NOs: 26-30, or as shown in FIGS. 2A and 2B.

In some embodiments, the heavy or light chain variable domain, or both, of, the anti-TCRβV antibody molecule, e.g., anti-TCRβ V12 antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody as described in Table 2A, or encoded by the nucleotide sequence in Table 2A; or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule comprises at least one, two, three, or four antigen-binding regions, e.g., variable regions, having an amino acid sequence as set forth in Table 2A, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 2A. In another embodiment, the anti-TCRβV antibody molecule, e.g., anti-TCRβ V12 antibody molecule includes a VH and/or VL domain encoded by a nucleic acid having a nucleotide sequence as set forth in Table 2A, or a sequence substantially identical thereto (e.g., a sequence at least about 85%, 90%, 95%, 99% or more identical thereto, or which differs by no more than 3, 6, 15, 30, or 45 nucleotides from the sequences shown in Table 2A.

In some embodiments, the anti-TCRβV antibody molecule, e.g., anti-TCRβ V12 antibody molecule comprises:

a VH domain comprising an amino acid sequence chosen from the amino acid sequence of SEQ ID NO: 23, SEQ ID NO: 24 or SEQ ID NO: 25, an amino acid sequence at least about 85%, 90%, 95%, 99% or more identical to the amino acid sequence SEQ ID NO: 23, SEQ ID NO: 24 or SEQ ID NO: 25, or an amino acid sequence which differs by no more than 1, 2, 5, 10, or 15 amino acid residues from the amino acid sequence of SEQ ID NO: 23, SEQ ID NO: 24, or SEQ ID NO: 25; and/or a VL domain comprising an amino acid sequence chosen from the amino acid sequence of SEQ ID NO: 26, SEQ ID NO: 27, SEQ ID NO: 28, SEQ ID NO: 29, or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29, or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29, or SEQ ID NO: 30.

In some embodiments, the anti-TCRβV antibody molecule, e.g., anti-TCRβ V12 antibody molecule comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises:

a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises:

a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises:

a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule is a full antibody or fragment thereof (e.g., a Fab, F(ab′)2, Fv, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule is a humanized antibody molecule. The heavy and light chains of the anti-TCRβV antibody molecule, e.g., anti-TCRβ V12 antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule is in the form of a multispecific molecule, e.g., a bispecific molecule, e.g., as described herein.

In some embodiments, the anti-TCRβV antibody molecule, e.g., anti-TCRβ V12 antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβ V12 antibody molecule has a light chain constant region chosen from, e.g., the light chain constant regions of kappa or lambda, preferably kappa (e.g., human kappa). In one embodiment, the constant region is altered, e.g., mutated, to modify the properties of the anti-TCRβV antibody molecule, e.g., anti-TCRβ V12 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217, or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβ V12 antibodies of the disclosure are provided in Table 2A. In some embodiments, the anti-TCRβ V12 is antibody B, e.g., humanized antibody B (antibody B-H), as provided in Table 2A. In some embodiments, the anti-TCRβV antibody comprises one or more (e.g., all three) of a LC CDR1, LC CDR2, and LC CDR3 provided in Table 2A; and/or one or more (e.g., all three) of a HC CDR1, HC CDR2, and HC CDR3 provided in Table 2A, or a sequence with at least 95% identity thereto. In some embodiments, antibody B comprises a variable heavy chain (VH) and/or a variable light chain (VL) provided in Table 2A, or a sequence with at least 95% identity thereto.

TABLE 2A
Amino acid and nucleotide sequences for murine and humanized antibody
molecules which bind to TCRVB 12, e.g., TCRVB 12-3 or TCRVB 12-4. The
antibody molecules include murine mAb Antibody B and humanized mAb
Antibody B-H.1to B-H.6. The amino acid the heavy and light chain CDRs,
and the amino acid and nucleotide sequences of the heavy and light
chain variable regions, and the heavy and light chains are shown.
Antibody B (murine)

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFGMH |
| | | WVRQAPDKGLEWVAYISSGSSTIYYADTLKGRFTI |
| | | SRDNPKNTLFLQMTSLRSEDTAMYYCARRGEGAMD |
| | | YWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIYWY |
| | | QQKSDASPKLWIYYTSNLAPGVPTRFSGSGSGNSY |
| | | SLTISSMEGEDAATYYCQQFTSSPFTFGSGTKLEI |
| | | K |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | EVQLVESGGGLVQPGGSLRLSCAASGFTFSNFGMH |
| | | WVRQAPGKGLEWVSYISSGSSTIYYADTLKGRFTI |
| | | SRDNAKNSLYLQMNSLRAEDTAVYYCARRGEGAMD |
| | | YWGQGTTVTVSS |
| SEQ ID NO: 31 | DNA VH | GAGGTGCAGCTGGTTGAATCTGGCGGAGGATTGGT |
| | | TCAGCCTGGCGGCTCTCTGAGACTGTCTTGTGCCG |
| | | CTTCTGGCTTCACCTTCTCCAACTTCGGCATGCAC |
| | | TGGGTCCGACAGGCCCCTGGAAAAGGACTGGAATG |
| | | GGTGTCCTACATCTCCTCCGGCTCCTCCACCATCT |
| | | ACTACGCTGACACCCTGAAGGGCAGATTCACCATC |
| | | TCTCGGGACAACGCCAAGAACTCCCTGTACCTGCA |
| | | GATGAACAGCCTGAGAGCCGAGGACACCGCCGTGT |
| | | ACTACTGTGCTAGAAGAGGCGAGGGCGCCATGGAT |
| | | TATTGGGGCCAGGGAACCACAGTGACCGTGTCTAG |
| | | C |

Antibody B-H.1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 24 | VH | EVQLVESGGGLVQPGGSLRLSCAASGFTFSNFGMH |
| | | WVRQAPGKGLEWVSYISSGSSTIYYADTLKGRFTI |
| | | SRDNSKNTLYLQMNSLRAEDTAVYYCARRGEGAMD |
| | | YWGQGTTVTVSS |
| SEQ ID NO: 32 | DNA VH | GAGGTGCAGCTGGTTGAATCTGGCGGAGGATTGGT |
| | | TCAGCCTGGCGGCTCTCTGAGACTGTCTTGTGCCG |
| | | CTTCTGGCTTCACCTTCTCCAACTTCGGCATGCAC |
| | | TGGGTCCGACAGGCCCCTGGAAAAGGACTGGAATG |
| | | GGTGTCCTACATCTCCTCCGGCTCCTCCACCATCT |
| | | ACTACGCTGACACCCTGAAGGGCAGATTCACCATC |
| | | AGCCGGGACAACTCCAAGAACACCCTGTACCTGCA |
| | | GATGAACTCCCTGAGAGCCGAGGACACCGCCGTGT |
| | | ACTACTGTGCTAGAAGAGGCGAGGGCGCCATGGAT |
| | | TATTGGGGCCAGGGAACCACAGTGACCGTGTCTAG |
| | | C |

Antibody B-H.1C HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | QVQLVESGGGVVQPGRSLRLSCAASGFTFSNFGMH |
| | | WVRQAPGKGLEWVAYISSGSSTIYYADTLKGRFTI |
| | | SRDNSKNTLYLQMNSLRAEDTAVYYCARRGEGAMD |
| | | YWGQGTTVTVSS |
| SEQ ID NO: 33 | DNA VH | CAGGTGCAGCTGGTGGAATCTGGTGGCGGAGTTGT |
| | | GCAGCCTGGCAGATCCCTGAGACTGTCTTGTGCCG |
| | | CCTCTGGCTTCACCTTCTCCAACTTCGGCATGCAC |
| | | TGGGTCCGACAGGCCCCTGGAAAAGGATTGGAGTG |
| | | GGTCGCCTACATCTCCTCCGGCTCCTCCACCATCT |
| | | ACTACGCTGACACCCTGAAGGGCAGATTCACCATC |
| | | AGCCGGGACAACTCCAAGAACACCCTGTACCTGCA |
| | | GATGAACTCCCTGAGAGCCGAGGACACCGCCGTGT |
| | | ACTACTGTGCTAGAAGAGGCGAGGGCGCCATGGAT |
| | | TATTGGGGCCAGGGAACCACAGTGACCGTGTCTAG |
| | | C |

Antibody B-H.1D LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 26 | VL | DNQLTQSPSFLSASVGDRVTITCRASSSVNYIYWY |
| | | QQKPGKAPKLLIYYTSNLAPGVPSRFSGSGSGNEY |
| | | TLTISSLQPEDFATYYCQQFTSSPFTFGQGTKLEI |
| | | K |
| SEQ ID NO: 34 | DNA VL | GATAACCAGCTGACCCAGTCTCCTAGCTTCCTGTC |
| | | TGCCTCTGTGGGCGACAGAGTGACAATTACCTGCC |
| | | GGGCCTCCTCCTCCGTGAACTACATCTACTGGTAT |
| | | CAGCAGAAGCCCGGCAAGGCCCCTAAGCTGCTGAT |
| | | CTACTACACCTCCAATCTGGCCCCTGGCGTGCCCT |
| | | CTAGATTTTCCGGATCTGGCTCCGGCAACGAGTAT |
| | | ACCCTGACAATCTCCAGCCTGCAGCCTGAGGACTT |
| | | CGCCACCTACTACTGCCAGCAGTTCACCTCCTCTC |
| | | CATTCACCTTTGGCCAGGGCACCAAGCTGGAAATC |
| | | AAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 27 | VL | DNQLTQSPSSLSASVGDRVTITCRASSSVNYIYWY |
| | | QQKPGKAPKLLIYYTSNLAPGVPSRFSGSGSGNDY |
| | | TLTISSLQPEDFATYYCQQFTSSPFTFGQGTKLEI |
| | | K |
| SEQ ID NO: 35 | DNA VL | ATAACCAGCTGACCCAGTCTCCTTCCAGCCTGTCT |
| | | GCTTCTGTGGGCGACAGAGTGACAATTACCTGCCG |
| | | GGCCTCCTCCTCCGTGAACTACATCTACTGGTATC |
| | | AGCAGAAGCCCGGCAAGGCCCCTAAGCTGCTGATC |
| | | TACTACACCTCCAATCTGGCCCCTGGCGTGCCCTC |
| | | TAGATTTTCCGGATCTGGCTCCGGCAACGACTATA |
| | | CCCTGACAATCTCCAGCCTGCAGCCTGAGGACTTC |
| | | GCCACCTACTACTGCCAGCAGTTCACCTCCTCTCC |
| | | ATTCACCTTTGGCCAGGGCACCAAGCTGGAAATCA |
| | | AA |

Antibody B-H.1F LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 28 | VL | ENVLTQSPATLSVSPGERATLSCRASSSVNYIYWY |
| | | QQKPGQAPRLLIYYTSNLAPGIPARFSGSGSGNEY |
| | | TLTISSLQSEDFAVYYCQQFTSSPFTFGQGTKLEI |
| | | K |
| SEQ ID NO: 36 | DNA VL | GAGAATGTGCTGACCCAGTCTCCTGCCACACTGTC |
| | | TGTTAGCCCTGGCGAGAGAGCTACCCTGAGCTGCA |
| | | GAGCCTCTTCCTCCGTGAACTACATCTACTGGTAT |
| | | CAGCAGAAGCCCGGCCAGGCTCCTAGACTGCTGAT |
| | | CTACTACACCTCCAATCTGGCCCCTGGCATCCCTG |
| | | CCAGATTTTCCGGATCTGGCTCCGGCAACGAGTAT |
| | | ACCCTGACCATCTCCAGCCTGCAGTCCGAGGACTT |
| | | TGCTGTGTACTATTGCCAGCAGTTCACAAGCAGCC |
| | | CTTTCACCTTTGGCCAGGGCACCAAGCTGGAAATC |
| | | AAA |

Antibody B-H.1G LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 29 | VL | QNVLTQPPSASGTPGQRVTISCRASSSVNYIYWYQ |
| | | QLPGTAPKLLIYYTSNLAPGVPDRFSGSGSGNSYS |
| | | LAISGLRSEDEADYYCQQFTSSPFTFGTGTKVTVL |
| SEQ ID NO: 37 | DNA VL | CAGAATGTGCTGACCCAACCTCCTTCCGCCTCTGG |
| | | CACACCTGGACAGAGAGTGACAATCTCCTGCCGGG |
| | | CCTCCTCCTCCGTGAACTACATCTACTGGTATCAG |
| | | CAGCTGCCCGGCACCGCTCCTAAACTGCTGATCTA |
| | | CTACACCTCCAATCTGGCCCCTGGCGTGCCCGATA |
| | | GATTTTCCGGATCTGGCTCCGGCAACTCCTACAGC |
| | | CTGGCTATCTCTGGCCTGAGATCTGAGGACGAGGC |
| | | CGACTACTACTGCCAGCAGTTCACCTCCTCTCCAT |
| | | TCACCTTTGGCACCGGCACCAAAGTGACAGTTCTT |

Antibody B-H.1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 30 | VL | SNELTQPPSVSVSPGQTARITCRASSSVNYIYWYQ |
| | | QKSGQAPVLVIYYTSNLAPGIPERFSGSGSGNMYT |
| | | LTISGAQVEDEADYYCQQFTSSPFTFGTGTKVTVL |
| SEQ ID NO: 38 | DNA VL | TCTAATGAGCTGACCCAGCCTCCTTCCGTGTCCGT |
| | | GTCTCCTGGACAGACCGCCAGAATTACCTGCCGGG |
| | | CCTCCTCCTCCGTGAACTACATCTACTGGTATCAG |
| | | CAGAAGTCCGGCCAGGCTCCTGTGCTCGTGATCTA |
| | | CTACACCTCCAATCTGGCCCCTGGCATCCCTGAGA |
| | | GATTCTCCGGATCTGGCTCCGGCAACATGTACACC |
| | | CTGACCATCTCTGGCGCCCAGGTGGAAGATGAGGC |
| | | CGACTACTACTGCCAGCAGTTCACCTCCTCTCCAT |
| | | TCACCTTTGGCACCGGCACCAAAGTGACAGTTCTT |

Antibody B-H.1

| | | |
|---|---|---|
| SEQ ID NO: 3280 | Chain1: Fc only | METDTLLLWVLLLWVPGSTGDKTHTCPPCPAPELL |
| | | GGPSVFLFPPKPKDTLMISRTPEVTCVVVDVSHED |
| | | PEVKFNWYVDGVEVHNAKTKPREEQYNSTYRVVSV |
| | | LTVLHQDWLNGKEYKCKVSNKALPAPIEKTISKAK |
| | | GQPREPQVYTLPPCREEMTKNQVSLWCLVKGFYPS |
| | | DIAVEWESNGQPENNYKTTPPVLDSDGSFFLYSKL |
| | | TVDKSRWQQGNVFSCSVMHEALHNRFTQKSLSLSP |
| | | GK |
| SEQ ID NO: 3281 | Chain2. humanized B-H | METDTLLLWVLLLWVPGSTGEVQLVESGGGLVQPG |
| | scFv | GSLRLSCAASGFTFSNFGMHWVRQAPGKGLEWVSY |
| | | ISSGSSTIYYADTLKGRFTISRDNSKNTLYLQMNS |
| | | LRAEDTAVYYCARRGEGAMDYWGQGTTVTVSSGGG |
| | | GSGGGGSGGGGSGGGGSDNQLTQSPSFLSASVGDR |
| | | VTITCRASSSVNYIYWYQQKPGKAPKLLIYYTSNL |
| | | APGVPSRFSGSGSGNEYTLTISSLQPEDFATYYCQ |
| | | QFTSSPFTFGQGTKLEIKGGGGSDKTHTCPPCPAP |
| | | ELLGGPSVFLFPPKPKDTLMISRTPEVTCVVVDVS |
| | | HEDPEVKFNWYVDGVEVHNAKTKPREEQYNSTYRV |
| | | VSVLTVLHQDWLNGKEYKCKVSNKALPAPIEKTIS |
| | | KAKGQPREPQVCTLPPSREEMTKNQVSLSCAVKGF |
| | | YPSDIAVEWESNGQPENNYKTTPPVLDSDGSFFLV |
| | | SKLTVDKSRWQQGNVFSCSVMHEALHNHYTQKSLS |
| | | LSPGKGGGGSGGGGSGLNDIFEAQKIEWHEEVQLV |
| SEQ ID NO: 1343 | scFv | ESGGGLVQPGGSLRLSCAASGFTFSNFGMHWVRQA |
| | | PGKGLEWVSYISSGSSTIYYADTLKGRFTISRDNS |
| | | KNTLYLQMNSLRAEDTAVYYCARRGEGAMDYWGQG |
| | | TTVTVSSGGGGSGGGGSGGGGSGGGGSDNQLTQSP |
| | | SFLSASVGDRVTITCRASSSVNYIYWYQQKPGKAP |
| | | KLLIYYTSNLAPGVPSRFSGSGSGNEYTLTISSLQ |
| | | PEDFATYYCQQFTSSPFTFGQGTKLEIK |

Antibody B-H.2

| | | |
|---|---|---|
| SEQ ID NO: 1338 | scFv | EVQLVESGGGLVQPGGSLRLSCAASGFTFSNFGMH |
| | | WVRQAPGKGLEWVSYISSGSSTIYYADTLKGRFTI |
| | | SRDNSKNTLYLQMNSLRAEDTAVYYCARRGEGAMD |
| | | YWGQGTTVTVSSGGGGSGGGGSGGGGSGGGGSDNQ |
| | | LTQSPSSLSASVGDRVTITCRASSSVNYIYWYQQK |
| | | PGKAPKLLIYYTSNLAPGVPSRFSGSGSGNDYTLT |
| | | ISSLQPEDFATYYCQQFTSSPFTFGQGTKLEIK |

Antibody B-H.3

| | | |
|---|---|---|
| SEQ ID NO: 1339 | scFv | EVQLVESGGGLVQPGGSLRLSCAASGFTFSNFGMH |
| | | WVRQAPGKGLEWVSYISSGSSTIYYADTLKGRFTI |
| | | SRDNSKNTLYLQMNSLRAEDTAVYYCARRGEGAMD |
| | | YWGQGTTVTVSSGGGGSGGGGSGGGGSGGGGSSNE |
| | | LTQPPSVSVSPGQTARITCRASSSVNYIYWYQQKS |
| | | GQAPVLVIYYTSNLAPGIPERFSGSGSGNMYTLTI |
| | | SGAQVEDEADYYCQQFTSSPFTFGTGTKVTVL |

Antibody B-H.4

| | | |
|---|---|---|
| SEQ ID NO: 1340 | scFv | QVQLVESGGGVVQPGRSLRLSCAASGFTFSNFGMH |
| | | WVRQAPGKGLEWVAYISSGSSTIYYADTLKGRFTI |
| | | SRDNSKNTLYLQMNSLRAEDTAVYYCARRGEGAMD |
| | | YWGQGTTVTVSSGGGGSGGGGSGGGGSGGGGSDNQ |
| | | LTQSPSFLSASVGDRVTITCRASSSVNYIYWYQQK |
| | | PGKAPKLLIYYTSNLAPGVPSRFSGSGSGNEYTLT |
| | | ISSLQPEDFATYYCQQFTSSPFTFGQGTKLEIK |

Antibody B-H.5

| | | |
|---|---|---|
| SEQ ID NO: 1341 | scFv | QVQLVESGGGVVQPGRSLRLSCAASGFTFSNFGMH |
| | | WVRQAPGKGLEWVAYISSGSSTIYYADTLKGRFTI |
| | | SRDNSKNTLYLQMNSLRAEDTAVYYCARRGEGAMD |
| | | YWGQGTTVTVSSGGGGSGGGGSGGGGSGGGGSDNQ |
| | | LTQSPSSLSASVGDRVTITCRASSSVNYIYWYQQK |
| | | PGKAPKLLIYYTSNLAPGVPSRFSGSGSGNDYTLT |
| | | ISSLQPEDFATYYCQQFTSSPFTFGQGTKLEIK |

Antibody B-H.6

| | | |
|---|---|---|
| SEQ ID NO: 1342 | scFv | QVQLVESGGGVVQPGRSLRLSCAASGFTFSNFGMH |
| | | WVRQAPGKGLEWVAYISSGSSTIYYADTLKGRFTI |
| | | SRDNSKNTLYLQMNSLRAEDTAVYYCARRGEGAMD |
| | | YWGQGTTVTVSSGGGGSGGGGSGGGGSGGGGSSNE |
| | | LTQPPSVSVSPGQTARITCRASSSVNYIYWYQQKS |
| | | GQAPVLVIYYTSNLAPGIPERFSGSGSGNMYTLTI |
| | | SGAQVEDEADYYCQQFTSSPFTFGTGTKVTVL |

TABLE 3A
Constant region amino acid sequences of human IgG
heavy chains and human kappa light chain

| | | |
|---|---|---|
| Human kappa | LC | RTVAAPSVFIFPPSDEQLKSGTASVVCLLNNFYPREAKVQWKVDNALQSG |
| constant region | | NSQESVTEQDSKDSTYSLSSTLTLSKADYEKHKVYACEVTHQGLSSPVTK |
| SEQ ID NO: 39 | | SFNRGEC |
| IgG4 (S228P) | HC | ASTKGPSVFPLAPCSRSTSESTAALGCLVKDYFPEPVTVSWNSGALTSGV |
| mutant constant | | HTFPAVLQSSGLYSLSSVVTVPSSSLGTKTYTCNVDHKPSNTKVDKRVES |
| region (EU | | KYGPPCPPCPAPEFLGGPSVFLFPPKPKDTLMISRTPEVTCVVVDVSQED |
| Numbering) | | PEVQFNWYVDGVEVHNAKTKPREEQFNSTYRVVSVLTVLHQDWLNGKEYK |
| SEQ ID NO: 40 | | CKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEG |
| | | NVFSCSVMHEALHNHYTQKSLSLSLG |
| IgG1 wild type | HC | ASTKGPSVFPLAPSSKSTSGGTAALGCLVKDYFPEPVTVSWNSGALTSGV |
| SEQ ID NO: 41 | | HTFPAVLQSSGLYSLSSVVTVPSSSLGTQTYICNVNHKPSNTKVDKRVEP |
| | | KSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPGK |
| IgG1 (N297A) | HC | ASTKGPSVFPLAPSSKSTSGGTAALGCLVKDYFPEPVTVSWNSGALTSGV |
| mutant constant | | HTFPAVLQSSGLYSLSSVVTVPSSSLGTQTYICNVNHKPSNTKVDKRVEP |
| region (EU | | KSCDKTHTCPPCPAPELLGGPSVFLFPPKPKDTLMISRTPEVTCVVVDVS |
| Numbering) | | HEDPEVKFNWYVDGVEVHNAKTKPREEQYASTYRVVSVLTVLHQDWLNGK |
| SEQ ID NO: 42 | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPGK |
| IgM constant | HC | GSASAPTLFPLVSCENSPSDTSSVAVGCLAQDFLPDSITFSWKYKNNSDI |
| delta CDC | | SSTRGFPSVLRGGKYAATSQVLLPSKDVMQGTDEHVVCKVQHPNGNKEKN |
| (P311A, P313S) | | VPLPVIAELPPKVSVFVPPRDGFFGNPRKSKLICQATGFSPRQIQVSWLR |
| SEQ ID NO: 73 | | EGKQVGSGVTTDQVQAEAKESGPTTYKVTSTLTIKESDWLGQSMFTCRVD |
| | | HRGLTFQQNASSMCVPDQDTAIRVFAIPPSFASIFLTKSTKLTCLVTDLT |
| | | TYDSVTISWTRQNGEAVKTHTNISESHPNATFSAVGEASICEDDWNSGER |
| | | FTCTVTHTDLASSLKQTISRPKGVALHRPDVYLLPPAREQLNLRESATIT |
| | | CLVTGFSPADVFVQWMQRGQPLSPEKYVTSAPMPEPQAPGRYFAHSILTV |
| | | SEEEWNTGETYTCVVAHEALPNRVTERTVDKSTGKPTLYNVSLVMSDTAG |
| | | TCY |
| IgGA1 | HC | ASPTSPKVFPLSLCSTQPDGNVVIACLVQGFFPQEPLSVTWSESGQGVTA |
| SEQ ID NO: 74 | | RNFPPSQDASGDLYTTSSQLTLPATQCLAGKSVTCHVKHYTNPSQDVTVP |
| | | CPVPSTPPTPSPSTPPTPSPSCCHPRLSLHRPALEDLLLGSEANLTCTLT |
| | | GLRDASGVTFTWTPSSGKSAVQGPPERDLCGCYSVSSVLPGCAEPWNHGK |
| | | TFTCTAAYPESKTPLTATLSKSGNTFRPEVHLLPPPSEELALNELVTLTC |
| | | LARGFSPKDVLVRWLQGSQELPREKYLTWASRQEPSQGTTTFAVTSILRV |
| | | AAEDWKKGDTFSCMVGHEALPLAFTQKTIDRLAGKPTHVNVSVVMAEVDG |
| | | TCY |
| IgGA2 | HC | ASPTSPKVFPLSLDSTPQDGNVVVACLVQGFFPQEPLSVTWSESGQNVTA |
| SEQ ID NO: 75 | | RNFPPSQDASGDLYTTSSQLTLPATQCPDGKSVTCHVKHYTNSSQDVTVP |
| | | CRVPPPPPCCHPRLSLHRPALEDLLLGSEANLTCTLTGLRDASGATFTWT |
| | | PSSGKSAVQGPPERDLCGCYSVSSVLPGCAQPWNHGETFTCTAAHPELKT |
| | | PLTANITKSGNTFRPEVHLLPPPSEELALNELVTLTCLARGFSPKDVLVR |
| | | WLQGSQELPREKYLTWASRQEPSQGTTTYAVTSILRVAAEDWKKGETFSC |
| | | MVGHEALPLAFTQKTIDRMAGKPTHINVSVVMAEADGTCY |
| Human Ig_J | HC | MKNHLLFWGVLAVFIKAVHVKAQEDERIVLVDNKCKCARITSRIIRSSED |
| chain | | PNEDIVERNIRIIVPLNNRENISDPTSPLRTRFVYHLSDLCKKCDPTEVE |
| SEQ ID NO: 76 | | LDNQIVTATQSNICDEDSATETCYTYDRNKCYTAVVPLVYGGETKMVETA |
| | | LTPDACYPD |

### Anti-TCRβ V5 Antibodies

Accordingly, in one aspect, the disclosure provides an anti-TCRβV antibody molecule that binds to human TCRβ V5. In some embodiments, the TCRβ V5 subfamily comprises TCRβ V5-5*01, TCRβ V5-6*01, TCRβ V5-4*01, TCRβ V5-8*01, TCRβ V5-1*01, or a variant thereof.

Exemplary anti-TCRβ V5 antibodies of the disclosure are provided in Table 10A. In some embodiments, the anti-TCRβ V5 is antibody C, e.g., humanized antibody C (antibody C-H), as provided in Table 10A. In some embodiments, the anti-TCRβV antibody comprises one or more (e.g., all three) of a LC CDR1, LC CDR2, and LC CDR3 provided in Table 10A; and/or one or more (e.g., all three) of a HC CDR1, HC CDR2, and HC CDR3 provided in Table 10A, or a sequence with at least 95% identity thereto. In some embodiments, antibody C comprises a variable heavy chain (VH) and/or a variable light chain (VL) provided in Table 10A, or a sequence with at least 95% identity thereto.

TABLE 10A
Amino acid sequences for anti TCRβ V5 antibodies
Amino acid and nucleotide sequences for murine and humanized antibody
molecules which bind to TCRVB 5 (e.g., TCRVBN 5-5 or TCRVB 5-6).
The amino acid the heavy and light chain CRDs, and the amino acid
and nucleotide sequences of the heavy and light chain
varibable regions, and the heavy and light chains are shown.
Murine antibody C

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3 (Combined) | QQYSKLPRT |
| SEQ ID NO: 232 | VH | DIQMTQTTSSLSASLGDRVTISCSASQGISNYLNWYQQ |
| | | KPDGTVKLLIYYTSSLHSGVPSRFSGSGSGTDYSLTIS |
| | | NLEPEDIATYYCQQYSKLPRTFGGGTKVEIK |
| SEQ ID NO: 233 | VL | QVQLKESGPGLVAPSQSLSITCTVSGFSLTAYGVNWVR |
| | | QPPGKGLEWLGMIWGDGNTDYNSALKSRLSISKDNSKS |
| | | QVFLKMNSLQTDDTARYYCARDRVTATLYAMDYWGQGT |
| | | SVTVSS |

Humkanized antibody C
C-H-1 antibody

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSLAKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGSINYLN |
| SEQ ID NO: 1322 | LC CDR2 (Combined) | YTSSLHS |
| SEQ ID NO: 1323 | LC CDR3 (Combined) | QQYSKLPRT |
| SEQ ID NO: 1324 | VL | DIQMTQSPSSLSASVGDRVTITCSASQGISNYLNWYQQ |
| | | TPGKAPKLLIYYTSSLHSGVPSRFSGSGSGTDYTFTIS |
| | | SLQPEDIATYYCQQYSKLPRTFGQGTKLQIT |
| SEQ ID NO: 1325 | VH | QVQLQESGPGLVRPSQTLSLTCTVSGFSLTAYGVNWVR |
| | | QPPGRGLEWLGMIWGDGNTDYNSALKSRVTMLKDTSKN |
| | | QFSLRLSSVTAADTAVYYCARDRVTATLYAMDYW |
| | | GQGSLVTVSS |

Humanized antibody C Variable light chain (VL)

| | | | |
|---|---|---|---|
| SEQ ID NO: 3000 | VL | C-H-VL.1 | DIQMTQSPSFLSASVGDRVTITCSASQGISNYLNWYQQ |
| | | | KPGKAVKLLIYYTSSLHSGVPSRFSGSGSGTEYTLTIS |
| | | | SLQPEDFATYYCQQYSKLPRTFGGGTKVEIK |
| SEQ ID NO: 3001 | VL | C-H-VL.2 | DIQMTQSPSSLSASVGDRVTITCSASQGISNYLNWYQQ |
| | | | KPGKAVKLLIYYTSSLHSGVPSRFSGSGSGTDYTLTIS |
| | | | SLQPEDFATYYCQQYSKLPRTFGGGTKVEIK |
| SEQ ID NO: 3002 | VL | C-H-VL.3 | DIQMTQSPSSLSASVGDRVTITCSASQGISNYLNWYQQ |
| | | | KPGKVVKLLIYYTSSLHSGVPSRFSGSGSGTDYTLTIS |
| | | | SLQPEDVATYYCQQYSKLPRTFGGGTKVEIK |
| SEQ ID NO: 3003 | VL | C-H-VL.4 | DIQMTQSPSSLSASVGDRVTITCSASQGISNYLNWYQQ |
| | | | KPGQAVKLLIYYTSSLHSGVPSRFSGSGSGTDYTLTIS |
| | | | SLQPEDVATYYCQQYSKLPRTFGGGTKVEIK |
| SEQ ID NO: 3004 | VL | C-H-VL.5 | DIQMTQSPSSLSASVGDRVTITCSASQGISNYLNWYQQ |
| | | | KPGKAVKLLIYYTSSLHSGVPSRFSGSGSGTDYTFTIS |
| | | | SLQPEDIATYYCQQYSKLPRTFGGGTKVEIK |
| SEQ ID NO: 3005 | VL | C-H-VL.6 | DIQMTQSPSSLSASVGDRVTITCSASQGISNYLNWYQQ |
| | | | KPGKTVKLLIYYTSSLHSGIPSRFSGSGSGTDYTLTIR |
| | | | SLQPEDFATYYCQQYSKLPRTFGGGTKVEIK |
| SEQ ID NO: 3006 | VL | C-H-VL.7 | AIQMTQSPSSLSASVGDRVTITCSASQGISNYLNWYQQ |
| | | | KPGKAVKLLIYYTSSLHSGVPSRFSGSGSGTDYTLTIS |
| | | | SLQPEDFATYYCQQYSKLPRTFGGGTKVEIK |
| SEQ ID NO: 3007 | VL | C-H-VL.8 | DIQMTQSPSSVSASVGDRVTITCSASQGISNYLNWYQQ |
| | | | KPGKAVKLLIYYTSSLHSGVPSRFSGSGSGTDYTLTIS |
| | | | SLQPEDFATYYCQQYSKLPRTFGGGTKVEIK |
| SEQ ID NO: 3008 | VL | C-H-VL.9 | DIQMTQSPSSLSASVGDRVTITCSASQGISNYLNWYQQ |
| | | | KPGKAVKRLIYYTSSLHSGVPSRFSGSGSGTEYTLTIS |
| | | | NLQPEDFATYYCQQYSKLPRTFGGGTKVEIK |
| SEQ ID NO: 3009 | VL | C-H-VL.10 | AIRMTQSPFSLSASVGDRVTITCSASQGISNYLNWYQQ |
| | | | KPAKAVKLFIYYTSSLHSGVPSRFSGSGSGTDYTLTIS |
| | | | SLQPEDFATYYCQQYSKLPRTFGGGTKVEIK |
| SEQ ID NO: 3010 | VL | C-H-VL.11 | DIQMTQSPSSLSASVGDRVTITCSASQGISNYLNWYQQ |
| | | | KPGKAVKRLIYYTSSLHSGVPSRFSGSGSGTEYTLTIS |
| | | | SLQPEDFATYYCQQYSKLPRTFGGGTKVEIK |
| SEQ ID NO: 3011 | VL | C-H-VL.12 | DIQMTQSPSTLSASVGDRVTITCSASQGISNYLNWYQQ |
| | | | KPGKAVKLLIYYTSSLHSGVPSRFSGSGSGTEYTLTIS |
| | | | SLQPDDFATYYCQQYSKLPRTFGGGTKVEIK |
| SEQ ID NO: 3012 | VL | C-H-VL.13 | DIQMTQSPSSLSASVGDRVTITCSASQGISNYLNWYQQ |
| | | | KPGKAVKSLIYYTSSLHSGVPSRFSGSGSGTDYTLTIS |
| | | | SLQPEDFATYYCQQYSKLPRTFGGGTKVEIK |
| SEQ ID NO: 3013 | VL | C-H-VL.14 | DIQMTQSPSSLSASVGDRVTITCSASQGISNYLNWYQQ |
| | | | KPGKAVKSLIYYTSSLHSGVPSKFSGSGSGTDYTLTIS |
| | | | SLQPEDFATYYCQQYSKLPRTFGGGTKVEIK |
| SEQ ID NO: 3014 | VL | C-H-VL.15 | DIQMTQSPSSLSASVGDRVTITCSASQGISNYLNWYQQ |
| | | | KPEKAVKSLIYYTSSLHSGVPSRFSGSGSGTDYTLTIS |
| | | | SLQPEDFATYYCQQYSKLPRTFGGGTKVEIK |
| SEQ ID NO: 3015 | VL | C-H-VL.16 | DIQMTQSPSAMSASVGDRVTITCSASQGISNYLNWYQQ |
| | | | KPGKVVKRLIYYTSSLHSGVPSRFSGSGSGTEYTLTIS |
| | | | SLQPEDFATYYCQQYSKLPRTFGGGTKVEIK |
| SEQ ID NO: 3016 | VL | C-H-VL.17 | DIVMTQSPDSLAVSLGERATINCSASQGISNYLNWYQQ |
| | | | KPGQPVKLLIYYTSSLHSGVPDRFSGSGSGTDYTLTIS |
| | | | SLQAEDVAVYYCQQYSKLPRTFGGGTKVEIK |
| SEQ ID NO: 3017 | VL | C-H-VL.18 | EIVMTQSPGTLSLSPGERATLSCSASQGISNYLNWYQQ |
| | | | KPGQAVKLLIYYTSSLHSGIPDRFSGSGSGTDYTLTIS |
| | | | RLEPEDFAVYYCQQYSKLPRTFGGGTKVEIK |
| SEQ ID NO: 3018 | VL | C-H-VL.19 | EIVMTQSPPTLSLSPGERVTLSCSASQGISNYLNWYQQ |
| | | | KPGQAVKLLIYYTSSLHSGIPARFSGSGSGTDYTLTIS |
| | | | SLQPEDFAVYYCQQYSKLPRTFGGGTKVEIK |
| SEQ ID NO: 3019 | VL | C-H-VL.20 | EIVMTQSPPTLSLSPGERVTLSCSASQGISNYLNWYQQ |
| | | | KPGQAVKLLIYYTSSLHSSIPARFSGSGSGTDYTLTIS |
| | | | SLQPEDFAVYYCQQYSKLPRTFGGGTKVEIK |
| SEQ ID NO: 3020 | VL | C-H-VL.21 | EIVMTQSPATLSLSPGERATLSCSASQGISNYLNWYQQ |
| | | | KPGQAVKLLIYYTSSLHSGIPARFSGSGSGTDYTLTIS |
| | | | SLEPEDFAVYYCQQYSKLPRTFGGGTKVEIK |
| SEQ ID NO: 3021 | VL | C-H-VL.22 | EIVMTQSPATLSLSPGERATLSCSASQGISNYLNWYQQ |
| | | | KPGQAVKLLIYYTSSLHSGIPARFSGSGSGTDYTLTIS |
| | | | RLEPEDFAVYYCQQYSKLPRTFGGGTKVEIK |
| SEQ ID NO: 3022 | VL | C-H-VL.23 | EIVMTQSPATLSLSPGERATLSCSASQGISNYLNWYQQ |
| | | | KPGQAVKLLIYYTSSLHSGIPDRFSGSGSGTDYTLTIS |
| | | | RLEPEDFAVYYCQQYSKLPRTFGGGTKVEIK |
| SEQ ID NO: 3023 | VL | C-H-VL.24 | EIVMTQSPATLSLSPGERATLSCSASQGISNYLNWYQQ |
| | | | KPGLAVKLLIYYTSSLHSGIPDRFSGSGSGTDYTLTIS |
| | | | RLEPEDFAVYYCQQYSKLPRTFGGGTKVEIK |
| SEQ ID NO: 3024 | VL | C-H-VL.25 | DIQMIQSPSFLSASVGDRVSIICSASQGISNYLNWYLQ |
| | | | KPGKSVKLFIYYTSSLHSGVSSRFSGRGSGTDYTLTII |
| | | | SLKPEDFAAYYCQQYSKLPRTFGGGTKVEIK |
| SEQ ID NO: 3025 | VL | C-H-VL.26 | EIVMTQSPATLSLSPGERATLSCSASQGISNYLNWYQQ |
| | | | KPGQAVKLLIYYTSSLHSGIPARFSGSGSGTDYTLTIS |
| | | | SLQPEDFAVYYCQQYSKLPRTFGGGTKVEIK |
| SEQ ID NO: 3026 | VL | C-H-VL.27 | EIVMTQSPATLSLSPGERATLSCSASQGISNYLNWYQQ |
| | | | KPGQAVKLLIYYTSSLHSGIPARFSGSGPGTDYTLTIS |
| | | | SLEPEDFAVYYCQQYSKLPRTFGGGTKVEIK |
| SEQ ID NO: 3027 | VL | C-H-VL.28 | DIVMTQTPLSLSVTPGQPASISCSASQGISNYLNWYLQ |
| | | | KPGQSVKLLIYYTSSLHSGVPDRFSGSGSGTDYTLKIS |
| | | | RVEAEDVGVYYCQQYSKLPRTFGGGTKVEIK |
| SEQ ID NO: 3028 | VL | C-H-VL.29 | DIVMTQTPLSLSVTPGQPASISCSASQGISNYLNWYLQ |
| | | | KPGQPVKLLIYYTSSLHSGVPDRFSGSGSGTDYTLKIS |
| | | | RVEAEDVGVYYCQQYSKLPRTFGGGTKVEIK |
| SEQ ID NO: 3029 | VL | C-H-VL.30 | DIVMTQSPAFLSVTPGEKVTITCSASQGISNYLNWYQQ |
| | | | KPDQAVKLLIYYTSSLHSGVPSRFSGSGSGTDYTFTIS |
| | | | SLEAEDAATYYCQQYSKLPRTFGGGTKVEIK |
| SEQ ID NO: 3030 | VL | C-H-VL.31 | DIVMTQSPLSLPVTPGEPASISCSASQGISNYLNWYLQ |
| | | | KPGQSVKLLIYYTSSLHSGVPDRFSGSGSGTDYTLKIS |
| | | | RVEAEDVGVYYCQQYSKLPRTFGGGTKVEIK |
| SEQ ID NO: 3031 | VL | C-H-VL.32 | DIVMTQTPLSLPVTPGEPASISCSASQGISNYLNWYLQ |
| | | | KPGQSVKLLIYYTSSLHSGVPDRFSGSGSGTDYTLKIS |
| | | | RVEAEDVGVYYCQQYSKLPRTFGGGTKVEIK |
| SEQ ID NO: 3032 | VL | C-H-VL.33 | EIVMTQSPATLSVSPGERATLSCSASQGISNYLNWYQQ |
| | | | KPGQAVKLLIYYTSSLHSGIPARFSGSGSGTEYTLTIS |
| | | | ILQSEDFAVYYCQQYSKLPRTFGGGTKVEIK |
| SEQ ID NO: 3033 | VL | C-H-VL.34 | EIVMTQSPATLSVSPGERATLSCSASQGISNYLNWYQQ |
| | | | KPGQAVKLLIYYTSSLHSGIPARFSGSGSGTEYTLTIS |
| | | | SLQSEDFAVYYCQQYSKLPRTFGGGTKVEIK |
| SEQ ID NO: 3034 | VL | C-H-VL.35 | DIVMTQSPLSLPVTLGQPASISCSASQGISNYLNWYQQ |
| | | | RPGQSVKRLIYYTSSLHSGVPDRFSGSGSGTDYTLKIS |
| | | | RVEAEDVGVYYCQQYSKLPRTFGGGTKVEIK |
| SEQ ID NO: 3035 | VL | C-H-VL.36 | EITMTQSPAFMSATPGDKVNISCSASQGISNYLNWYQQ |
| | | | KPGEAVKFIIYYTSSLHSGIPPRFSGSGYGTDYTLTTN |
| | | | NIESEDAAYYYCQQYSKLPRTFGGGTKVEIK |
| SEQ ID NO: 3036 | VL | C-H-VL.37 | DIVMTQTPLSSPVTLGQPASISCSASQGISNYLNWYQQ |
| | | | RPGQPVKLLIYYTSSLHSGVPDRFSGSGAGTDYTLKIS |
| | | | RVEAEDVGVYYCQQYSKLPRTFGGGTKVEIK |
| SEQ ID NO: 3037 | VL | C-H-VL.38 | EIVMTQSPDFQSVTPKEKVTITCSASQGISNYLNWYQQ |
| | | | KPDQSVKLLIYYTSSLHSGVPSRFSGSGSGTDYTLTIN |
| | | | SLEAEDAATYYCQQYSKLPRTFGGGTKVEIK |
| SEQ ID NO: 3038 | VL | C-H-VL.39 | EIVMTQTPLSLSITPGEQASISCSASQGISNYLNWYLQ |
| | | | KARPVVKLLIYYTSSLHSGVPDRFSGSGSGTDYTLKIS |
| | | | RVEAEDFGVYYCQQYSKLPRTFGGGTKVEIK |
| SEQ ID NO: 3039 | VL | C-H-VL.40 | EIVMTQTPLSLSITPGEQASMSCSASQGISNYLNWYLQ |
| | | | KARPVVKLLIYYTSSLHSGVPDRFSGSGSGTDYTLKIS |
| | | | RVEAEDFGVYYCQQYSKLPRTFGGGTKVEIK |

Humanized antibody C Variable HEAVY chain (VH)

| | | | |
|---|---|---|---|
| SEQ ID NO: 3040 | VH | C-H-VH.1 | QVTLKESGPVLVKPTETLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWGQGT |
| | | | LVTVSS |
| SEQ ID NO: 3041 | VH | C-H-VH.2 | QVTLKESGPALVKPTETLTLTCTVSGFSLTAYGVNWVR |
| | | | QPPGKALEWLGMIWGDGNTDYNSALKSRLIISKDNSKS |
| | | | QVVLTMTNMDPVDTATYYCARDRVTATLYAMDYWGQGT |
| | | | LVTVSS |
| SEQ ID NO: 3042 | VH | C-H-VH.3 | QVTLKESGPALVKPTQTLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWGQGT |
| | | | LVTVSS |
| SEQ ID NO: 3043 | VH | C-H-VH.4 | QVQLQESGPGLVKPSGTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3044 | VH | C-H-VH.5 | QVTLKESGPTLVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWGQGT |
| | | | LVTVSS |
| SEQ ID NO: 3045 | VH | C-H-VH.6 | QVTLKESGPALVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWGQGT |
| | | | LVTVSS |
| SEQ ID NO: 3046 | VH | C-H-VH.7 | QVQLQESGPGLVKPSQTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3047 | VH | C-H-VH.8 | QVQLQESGPGLVKPSETLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3048 | VH | C-H-VH.9 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3049 | VH | C-H-VH.10 | QVQLQESGPGLVKPSDTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3050 | VH | C-H-VH.11 | QVQLQESGPGLVKPSQTLSLTCTVSGFSLTAYGVNWVR |
| | | | QHPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3051 | VH | C-H-VH.12 | QVQLQESGPGLVKPSQTLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3052 | VH | C-H-VH.13 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQGT |
| | | | LVTVSS |
| SEQ ID NO: 3053 | VH | C-H-VH.14 | QVQLQESGPGLVKPSETLSLTCTVSGFSLTAYGVNWVR |
| | | | QPPGKGLEWLGMIWGDGNTDYNSALKSRLTISKDNSKS |
| | | | HVSLKLSSVTAADTAVYYCARDRVTATLYAMDYWGQGT |
| | | | LVTVSS |
| SEQ ID NO: 3054 | VH | C-H-VH.15 | QVQLQESGPGLVKPSETLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3055 | VH | C-H-VH.16 | QVQLQESGPGLVKPSQTLSLTCAVYGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3056 | VH | C-H-VH.17 | RVQLQESGPGLVKPSETLSLTCTVSGFSLTAYGVNWVR |
| | | | QPPGKGLEWLGMIWGDGNTDYNSALKSRLTISKDNSKS |
| | | | QVPLKLSSVTAADTAVYYCARDRVTATLYAMDYWGQGT |
| | | | LVTVSS |
| SEQ ID NO: 3057 | VH | C-H-VH.18 | QVQLQESGPGLVKPSQTLSLTCTVSGFSLTAYGVNWVR |
| | | | QHPGKGLEWLGMIWGDGNTDYNSALKSLLTISKDNSKS |
| | | | QVSLKLSSVTAADTAVYYCARDRVTATLYAMDYWGQGT |
| | | | LVTVSS |
| SEQ ID NO: 3058 | VH | C-H-VH.19 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTALDTAVYYCARDRVTATLYAMDYWGQGT |
| | | | LVTVSS |
| SEQ ID NO: 3059 | VH | C-H-VH.20 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQGT |
| | | | LVTVSS |
| SEQ ID NO: 3060 | VH | C-H-VH.21 | QVQLQESGSGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3061 | VH | C-H-VH.22 | EVQLVESGGGLVQPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQGT |
| | | | LVTVSS |
| SEQ ID NO: 3062 | VH | C-H-VH.23 | EVQLVESGGGLVQPGPSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQGT |
| | | | LVTVSS |
| SEQ ID NO: 3063 | VH | C-H-VH.24 | QVQLQESGSGLVKPSQTLSLTCAVSGFSLTAYGVNWVR |
| | | | QSPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3064 | VH | C-H-VH.25 | QVQLQESGPGLVKPSETLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3065 | VH | C-H-VH.26 | EVQLVESGGGLVKPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQGT |
| | | | LVTVSS |
| SEQ ID NO: 3066 | VH | C-H-VH.27 | QVQLQESGPGLVKPSETLSLTCAVYGFSLTAYGVNWVR |
| | | | QPPGKGLEWLGMIWGDGNTDYNSALKSRLTISKDNSKS |
| | | | QVYLKLSSVTAADTAVYYCARDRVTATLYAMDYWGQGT |
| | | | LVTVSS |
| SEQ ID NO: 3067 | VH | C-H-VH.28 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTGVYYCARDRVTATLYAMDYWGQGT |
| | | | LVTVSS |
| SEQ ID NO: 3068 | VH | C-H-VH.29 | EVQLVESGGGLVQPGGSLRLSCAVSGFSLTAYGVNWVR |
| | | | QAPGKGLEWLGMIWGDGNTDYNSALKSRLTISKDNSKS |
| | | | SVYLQMNSLKTEDTAVYYCARDRVTATLYAMDYWGQGT |
| | | | LVTVSS |
| SEQ ID NO: 3069 | VH | C-H-VH.30 | EVQLVESGGGLVKPGGSLRLSCAVSGFSLTAYGVNWVR |
| | | | QAPGKGLEWLGMIWGDGNTDYNSALKSRLTISKDNSKS |
| | | | TVYLQMNSLKTEDTAVYYCARDRVTATLYAMDYWGQGT |
| | | | LVTVSS |
| SEQ ID NO: 3070 | VH | C-H-VH.31 | QVQLQQSGPGLVKPSQTLSLTCAVSGFSLTAYGVNWVR |
| | | | QSPSRGLEWLGMIWGDGNTDYNSALKSRLTINKDNSKS |
| | | | QVSLQLNSVTPEDTAVYYCARDRVTATLYAMDYWGQGT |
| | | | LVTVSS |
| SEQ ID NO: 3071 | VH | C-H-VH.32 | QVQLVESGGGLVQPGGSLRLSCSVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQGT |
| | | | LVTVSS |
| SEQ ID NO: 3072 | VH | C-H-VH.33 | QVQLQQWGAGLLKPSETLSLTCAVYGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQGT |
| | | | LVTVSS |
| SEQ ID NO: 3073 | VH | C-H-VH.34 | QVQLVESGGGVVQPGRSLRLSCAVSGFSLTAYGVNWVR |
| | | | QAPGKGLEWLGMIWGDGNTDYNSALKSRLTISKDNSTS |
| | | | TVFLQMNSLRAEDTAVYYCARDRVTATLYAMDYWGQGT |
| | | | LVTVSS |
| SEQ ID NO: 3074 | VH | C-H-VH.35 | EVQLVESGGGLVQPGGSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQGT |
| | | | LVTVSS |
| SEQ ID NO: 3075 | VH | C-H-VH.36 | EVQLVESGGGLVQPGGSLRLSCAVSGFSLTAYGVNWVR |
| | | | QAPGKGLEWLGMIWGDGNTDYNSALKSRLTISKDNAKS |
| | | | SVYLQMNSLRDEDTAVYYCARDRVTATLYAMDYWGQGT |
| | | | LVTVSS |
| SEQ ID NO: 3076 | VH | C-H-VH.37 | EVQLLESGGGLVQPGGSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQGT |
| | | | LVTVSS |
| SEQ ID NO: 3077 | VH | C-H-VH.38 | QVQLVESGGGLVKPGGSLRLSCAVSGFSLTAYGVNWVR |
| | | | QAPGKGLEWLGMIWGDGNTDYNSALKSRLTISKDNAKS |
| | | | SVYLQMNSLRAEDTAVYYCARDRVTATLYAMDYWGQGT |
| | | | LVTVSS |
| SEQ ID NO: 3078 | VH | C-H-VH.39 | EVQLVESGGGLVQPGGSLKLSCAVSGFSLTAYGVNWVR |
| | | | QASGKGLEWLGMIWGDGNTDYNSALKSRLTISKDNSKS |
| | | | TVYLQMNSLKTEDTAVYYCARDRVTATLYAMDYWGQGT |
| | | | LVTVSS |
| SEQ ID NO: 3079 | VH | C-H-VH.40 | QVQLLESGGGLVKPGGSLRLSCAVSGFSLTAYGVNWVR |
| | | | QAPGKGLEWLGMIWGDGNTDYNSALKSRLTISKDNAKS |
| | | | SVYLQMNSLRAEDTAVYYCARDRVTATLYAMDYWGQGT |
| | | | LVTVSS |
| SEQ ID NO: 3080 | VH | C-H-VH.41 | QVQLVESGGGVVQPGRSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQGT |
| | | | LVTVSS |
| SEQ ID NO: 3081 | VH | C-H-VH.42 | QVQLVESGGGVVQPGRSLRLSCAVSGFSLTAYGVNWVR |
| | | | QAPGKGLEWLGMIWGDGNTDYNSALKSRLTISKDNSKS |
| | | | RVYLQMNSLRAEDTAVYYCARDRVTATLYAMDYWGQGT |
| | | | LVTVSS |
| SEQ ID NO: 3082 | VH | C-H-VH.43 | QVQLVESGGGVVQPGRSLRLSCAVSGFSLTAYGVNWVR |
| | | | QAPGKGLEWLGMIWGDGNTDYNSALKSRLAISKDNSKS |
| | | | TVYLQMNSLRAEDTAVYYCARDRVTATLYAMDYWGQGT |
| | | | LVTVSS |
| SEQ ID NO: 3083 | VH | C-H-VH.44 | QVQLVESGGGVVQPGGSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQGT |
| | | | LVTVSS |
| SEQ ID NO: 3084 | VH | C-H-VH.45 | EVQLVESGGGLVQPGGSLRLSCAVSGFSLTAYGVNWVR |
| | | | QAPGKGLEWLGMIWGDGNTDYNSALKSRLTISKDNAKS |
| | | | TVYLQMNSLRAEDTAVYYCARDRVTATLYAMDYWGQGT |
| | | | LVTVSS |
| SEQ ID NO: 3085 | VH | C-H-VH.46 | EVQLVESGGGLVQPGGSLRLSCAVSGFSLTAYGVNWVR |
| | | | QAPGKGLEWLGMIWGDGNTDYNSALKSRLTISKDNAKS |
| | | | SVYLQMNSLRAEDTAVYYCARDRVTATLYAMDYWGQGT |
| | | | LVTVSS |
| SEQ ID NO: 3086 | VH | C-H-VH.47 | EVQLVESGGVVVQPGGSLRLSCAVSGFSLTAYGVNWVR |
| | | | QAPGKGLEWLGMIWGDGNTDYNSALKSRLTISKDNSKS |
| | | | SVYLQMNSLRTEDTALYYCARDRVTATLYAMDYWGQGT |
| | | | LVTVSS |
| SEQ ID NO: 3087 | VH | C-H-VH.48 | EVQLVESGGGLVQPGGSLRLSCAVSGFSLTAYGVNWVR |
| | | | QAPGKGLEWLGMIWGDGNTDYNSALKSRLTISKHNSKS |
| | | | TVYLQMNSLRAEDTAVYYCARDRVTATLYAMDYWGQGT |
| | | | LVTVSS |
| SEQ ID NO: 3088 | VH | C-H-VH.49 | EVQLVESGGGLVKPGGSLRLSCAVSGFSLTAYGVNWVR |
| | | | QAPGKGLEWLGMIWGDGNTDYNSALKSRLTISKDNAKS |
| | | | SVYLQMNSLRAEDTAVYYCARDRVTATLYAMDYWGQGT |
| | | | LVTVSS |
| SEQ ID NO: 3089 | VH | C-H-VH.50 | EVQLVESGGGLIQPGGSLRLSCAVSGFSLTAYGVNWVR |
| | | | QPPGKGLEWLGMIWGDGNTDYNSALKSRLTISKDNSKS |
| | | | TVYLQMNSLRAEDTAVYYCARDRVTATLYAMDYWGQGT |
| | | | LVTVSS |

Exemplary anti-TCRβ V5 antibodies of the disclosure are provided in Table 11A. In some embodiments, the anti-TCRβ V5 is antibody E, e.g., humanized antibody E (antibody E-H), as provided in Table 11A. In some embodiments, the anti-TCRβV antibody comprises one or more (e.g., all three) of a LC CDR1, LC CDR2, and LC CDR3 provided in Table 11A; and/or one or more (e.g., all three) of a HC CDR1, HC CDR2, and HC CDR3 provided in Table 11A, or a sequence with at least 95% identity thereto. In some embodiments, antibody E comprises a variable heavy chain (VH) and/or a variable light chain (VL) provided in Table 11A, or a sequence with at least 95% identity thereto.

In some embodiments, antibody E comprises a heavy chain comprising the amino acid sequence of SEQ ID NO: 3284 and/or a light chain comprising the amino acid sequence of SEQ ID NO: 3285, or sequence with at least 95% identity thereto.

TABLE 11A
Amino acid sequences for anti TCRβ V5 antibodies
Amino acid and nucleotide sequences for murine and humanized
antibody molecules which bind to TCRVB 5 (e.g., TCRVB 5-5 or
TCRVB 5-6). The amino acid the heavy and light chain CDRs, and
the amino acid and nucleotide sequences of the heavy and light
chain variable regions, and the heavy and light chains are shown.
Murine antibody E

| | | |
|---|---|---|
| SEQ ID NO: 1298 | HC CDR1 (Kabat) | SSWMN |
| SEQ ID NO: 1299 | HC CDR2 (Kabat) | RIYPGDGDTKYNGKFKG |
| SEQ ID NO: 1300 | HC CDR3 (Kabat) | RGTGGWYFDV |
| SEQ ID NO: 1302 | HC CDR1 (Chothia) | GYAFSSS |
| SEQ ID NO: 1303 | HC CDR2 (Chothia) | YPGDGD |
| SEQ ID NO: 1301 | HC CDR3 (Chothia) | RGTGGWYFDV |
| SEQ ID NO: 1304 | HC CDR1 (Combined) | GYAFSSSWMN |
| SEQ ID NO: 1299 | HC CDR2 (Combined) | RIYPGDGDTKYNGKFKG |
| SEQ ID NO: 1301 | HC CDR3(Combined) | RGTGGWYFDV |
| SEQ ID NO: 1305 | LC CDR1 (Kabat) | RASESVDSSGNSFMH |
| SEQ ID NO: 1306 | LC CDR2 (Kabat) | RASNLES |
| SEQ ID NO: 1307 | LC CDR3 (Kabat) | QQSFDDPFT |
| SEQ ID NO: 1308 | LC CDR1 (Chothia) | SESVDSSGNSF |
| SEQ ID NO: 1306 | LC CDR2 (Chothia) | RASNLES |
| SEQ ID NO: 1307 | LC CDR3 (Chothia) | QQSFDDPFT |
| SEQ ID NO: 1305 | LC CDR1 (Combined) | RASESVDSSGNSFMH |
| SEQ ID NO: 1306 | LC CDR2 (Combined) | RASNLES |
| SEQ ID NO: 1307 | LC CDR3(Combined) | QQSFDDPFT |
| SEQ ID NO: 3091 | VH | QVQLQQSGPELVKPGASVKISCKASGYAFSSSWMN |
| | | WVKQRPGQGLEWIGRIYPGDGDTKYNGKFKGKATL |
| | | TADKSSSTAYMHLSSLTSVDSAVYFCARRGTGGWY |
| | | FDVWGAGTTVTVSS |
| SEQ ID NO: 3284 | Heavy chain | METDTLLLWVLLLWVPGSTGQVQLQQSGPELVKPG |
| | | ASVKISCKASGYAFSSSWMNWVKQRPGQGLEWIGR |
| | | IYPGDGDTKYNGKFKGKATLTADKSSSTAYMHLSS |
| | | LTSVDSAVYFCARRGTGGWYFDVWGAGTTVTVSSA |
| | | KTTAPSVYPLAPVCGDTTGSSVTLGCLVKGYFPEP |
| | | VTLTWNSGLSLSSGVHTFPAVLQSDLYTLSSSVTV |
| | | SSTWPSQSITCNVAHPASSTKVDKKIEPRGPTIKP |
| | | CPPCKCPAPNLLGGPSVFIFPPKIKDVLMISLSPI |
| | | VTCVVVDVSEDDPDVQISWFVNNVEVHTAQTQTHR |
| | | EDYNSTLRVVSALPIQHQDWMSGKEFKCKVNNKDL |
| | | PAPIERTISKPKGSVRAPQVYVLPPPEEEMTKKQV |
| | | TLTCMVTDFMPEDIYVEETNNGKTELNYKNTEPVL |
| | | DSDGSYFMYSKLRVEKKNWVERNSYSCSVVHEGLH |
| | | NHHTTKSFSRTPGK |
| SEQ ID NO: 3092 | VL | DIVLTQSPASLAVSLGQRATISCRASESVDSSGNS |
| | | FMHWYQQKPGQPPQLLIYRASNLESGIPARFSGSG |
| | | SRTDFTLTINPVEADDVATFYCQQSFDDPFTFGSG |
| | | TKLEIK |
| SEQ ID NO: 3285 | Light chain | MEDTDTLLLWVLLLWVPGSTGDIVLTQSPASLAVS |
| | | LGQRATISCRASESVDSSGNSFMHWYQQKPGQPPQ |
| | | LLIYRASNLESGIPARFSGSGSRTDFTLTINPVEA |
| | | DDVATFYCQQSFDDPFTFGSGTKLEIKRADAAPTV |
| | | SIFPPSSEQLTSGGASVVCLFLNNFYPKDINVKWK |
| | | IDGSERQNGVLNSWTDQDSKDSTYSMSSTLTKTKD |
| | | EYERHNSYTCEATHKTSTSPIVKSFNRNEC |

Humanized antibody E(E-H antibody)
Variable light chain (VL)

| | | | |
|---|---|---|---|
| SEQ ID NO: 3093 | VL | E-H. 1 | DIVLTQSPDSLAVSLGERATINCRASESVDSSGNS |
| | | | FMHWYQQKPGQPPQLLIYRASNLESGVPDRFSGSG |
| | | | SRTDFTLTISSLQAEDVAVYYCQQSFDDPFTFGQG |
| | | | TKLEIK |
| SEQ ID NO: 3094 | VL | E-H.2 | EIVLTQSPATLSLSPGERATLSCRASESVDSSGNS |
| | | | FMHWYQQKPGQAPQLLIYRASNLESGIPARFSGSG |
| | | | SRTDFTLTISSLEPEDFAVYYCQQSFDDPFTFGQG |
| | | | TKLEIK |
| SEQ ID NO: 3095 | VL | E-H.3 | EIVLTQSPATLSLSPGERATLSCRASESVDSSGNS |
| | | | FMHWYQQKPGQAPQLLIYRASNLESGIPARFSGSG |
| | | | SRTDFTLTISRLEPEDFAVYYCQQSFDDPFTFGQG |
| | | | TKLEIK |
| SEQ ID NO: 3096 | VL | E-H.4 | EIVLTQSPATLSLSPGERATLSCRASESVDSSGNS |
| | | | FMHWYQQKPGQAPQLLIYRASNLESGIPARFSGSG |
| | | | SRTDFTLTISSLQPEDFAVYYCQQSFDDPFTFGQG |
| | | | TKLEIK |
| SEQ ID NO: 3097 | VL | E-H.5 | DIQLTQSPSSLSASVGDRVTITCRASESVDSSGNS |
| | | | FMHWYQQKPGQAPQLLIYRASNLESGVPSRFSGSG |
| | | | SRTDFTLTISSLQPEDVATYYCQQSFDDPFTFGQG |
| | | | TKLEIK |
| SEQ ID NO: 3098 | VL | E-H.6 | EIVLTQSPATLSLSPGERATLSCRASESVDSSGNS |
| | | | FMHWYQQKPGQAPQLLIYRASNLESGIPARFSGSG |
| | | | PRTDFTLTISSLEPEDFAVYYCQQSFDDPFTFGQG |
| | | | TKLEIK |
| SEQ ID NO: 3099 | VL | E-H.7 | EIVLTQSPATLSLSPGERATLSCRASESVDSSGNS |
| | | | FMHWYQQKPGQAPQLLIYRASNLESGIPDRFSGSG |
| | | | SRTDFTLTISRLEPEDFAVYYCQQSFDDPFTFGQG |
| | | | TKLEIK |
| SEQ ID NO: 3100 | VL | E-H.8 | DIQLTQSPSSLSASVGDRVTITCRASESVDSSGNS |
| | | | FMHWYQQKPGKVPQLLIYRASNLESGVPSRFSGSG |
| | | | SRTDFTLTISSLQPEDVATYYCQQSFDDPFTFGQG |
| | | | TKLEIK |
| SEQ ID NO: 3101 | VL | E-H.9 | DIQLTQSPSSLSASVGDRVTITCRASESVDSSGNS |
| | | | FMHWYQQKPGKTPQLLIYRASNLESGIPSRFSGSG |
| | | | SRTDFTLTIRSLQPEDFATYYCQQSFDDPFTFGQG |
| | | | TKLEIK |
| SEQ ID NO: 3102 | VL | E-H.10 | EIVLTQSPGTLSLSPGERATLSCRASESVDSSGNS |
| | | | FMHWYQQKPGQAPQLLIYRASNLESGIPDRFSGSG |
| | | | SRTDFTLTISRLEPEDFAVYYCQQSFDDPFTFGQG |
| | | | TKLEIK |
| SEQ ID NO: 3103 | VL | E-H.11 | EIVLTQSPATLSLSPGERATLSCRASESVDSSGNS |
| | | | FMHWYQQKPGLAPQLLIYRASNLESGIPDRFSGSG |
| | | | SRTDFTLTISRLEPEDFAVYYCQQSFDDPFTFGQG |
| | | | TKLEIK |
| SEQ ID NO: 3104 | VL | E-H.12 | DIQLTQSPSSLSASVGDRVTITCRASESVDSSGNS |
| | | | FMHWYQQKPGKAPQLLIYRASNLESGVPSRFSGSG |
| | | | SRTDFTLTISSLQPEDFATYYCQQSFDDPFTFGQG |
| | | | TKLEIK |
| SEQ ID NO: 3105 | VL | E-H.13 | DIQLTQSPSSVSASVGDRVTITCRASESVDSSGNS |
| | | | FMHWYQQKPGKAPQLLIYRASNLESGVPSRFSGSG |
| | | | SRTDFTLTISSLQPEDFATYYCQQSFDDPFTFGQG |
| | | | TKLEIK |
| SEQ ID NO: 3106 | VL | E-H.14 | AIQLTQSPSSLSASVGDRVTITCRASESVDSSGNS |
| | | | FMHWYQQKPGKAPQLLIYRASNLESGVPSRFSGSG |
| | | | SRTDFTLTISSLQPEDFATYYCQQSFDDPFTFGQG |
| | | | TKLEIK |
| SEQ ID NO: 3107 | VL | E-H.15 | DIQLTQSPSFLSASVGDRVTITCRASESVDSSGNS |
| | | | FMHWYQQKPGKAPQLLIYRASNLESGVPSRFSGSG |
| | | | SRTEFTLTISSLQPEDFATYYCQQSFDDPFTFGQG |
| | | | TKLEIK |
| SEQ ID NO: 3108 | VL | E-H.16 | DIQLTQSPSSLSASVGDRVTITCRASESVDSSGNS |
| | | | FMHWYQQKPGKAPQLLIYRASNLESGVPSRFSGSG |
| | | | SRTDFTFTISSLQPEDIATYYCQQSFDDPFTFGQG |
| | | | TKLEIK |
| SEQ ID NO: 3109 | VL | E-H.17 | EIVLTQSPATLSVSPGERATLSCRASESVDSSGNS |
| | | | FMHWYQQKPGQAPQLLIYRASNLESGIPARFSGSG |
| | | | SRTEFTLTISILQSEDFAVYYCQQSFDDPFTFGQG |
| | | | TKLEIK |
| SEQ ID NO: 3110 | VL | E-H.18 | EIVLTQSPATLSVSPGERATLSCRASESVDSSGNS |
| | | | FMHWYQQKPGQAPQLLIYRASNLESGIPARFSGSG |
| | | | SRTEFTLTISSLQSEDFAVYYCQQSFDDPFTFGQG |
| | | | TKLEIK |
| SEQ ID NO: 3111 | VL | E-H.19 | AIRLTQSPFSLSASVGDRVTITCRASESVDSSGNS |
| | | | FMHWYQQKPAKAPQLFIYRASNLESGVPSRFSGSG |
| | | | SRTDFTLTISSLQPEDFATYYCQQSFDDPFTFGQG |
| | | | TKLEIK |
| SEQ ID NO: 3112 | VL | E-H.20 | DIQLTQSPSSLSASVGDRVTITCRASESVDSSGNS |
| | | | FMHWYQQKPGKAPQSLIYRASNLESGVPSRFSGSG |
| | | | SRTDFTLTISSLQPEDFATYYCQQSFDDPFTFGQG |
| | | | TKLEIK |
| SEQ ID NO: 3113 | VL | E-H.21 | DIQLTQSPSSLSASVGDRVTITCRASESVDSSGNS |
| | | | FMHWYQQKPGKAPQRLIYRASNLESGVPSRFSGSG |
| | | | SRTEFTLTISNLQPEDFATYYCQQSFDDPFTFGQG |
| | | | TKLEIK |
| SEQ ID NO: 3114 | VL | E-H.22 | DIQLTQSPSTLSASVGDRVTITCRASESVDSSGNS |
| | | | FMHWYQQKPGKAPQLLIYRASNLESGVPSRFSGSG |
| | | | SRTEFTLTISSLQPDDFATYYCQQSFDDPFTFGQG |
| | | | TKLEIK |
| SEQ ID NO: 3115 | VL | E-H.23 | EIVLTQSPDFQSVTPKEKVTITCRASESVDSSGNS |
| | | | FMHWYQQKPDQSPQLLIYRASNLESGVPSRFSGSG |
| | | | SRTDFTLTINSLEAEDAATYYCQQSFDDPFTFGQG |
| | | | TKLEIK |
| SEQ ID NO: 3116 | VL | E-H.24 | DIQLTQSPSSLSASVGDRVTITCRASESVDSSGNS |
| | | | FMHWYQQKPGKAPQSLIYRASNLESGVPSKFSGSG |
| | | | SRTDFTLTISSLQPEDFATYYCQQSFDDPFTFGQG |
| | | | TKLEIK |
| SEQ ID NO: 3117 | VL | E-H.25 | DIQLTQSPSSLSASVGDRVTITCRASESVDSSGNS |
| | | | FMHWYQQKPGKAPQRLIYRASNLESGVPSRFSGSG |
| | | | SRTEFTLTISSLQPEDFATYYCQQSFDDPFTFGQG |
| | | | TKLEIK |
| SEQ ID NO: 3118 | VL | E-H.26 | DIVLTQTPLSLSVTPGQPASISCRASESVDSSGNS |
| | | | FMHWYLQKPGQPPQLLIYRASNLESGVPDRFSGSG |
| | | | SRTDFTLKISRVEAEDVGVYYCQQSFDDPFTFGQG |
| | | | TKLEIK |
| SEQ ID NO: 3119 | VL | E-H.27 | DIQLTQSPSSLSASVGDRVTITCRASESVDSSGNS |
| | | | FMHWYQQKPEKAPQSLIYRASNLESGVPSRFSGSG |
| | | | SRTDFTLTISSLQPEDFATYYCQQSFDDPFTFGQG |
| | | | TKLEIK |
| SEQ ID NO: 3120 | VL | E-H.28 | EIVLTQSPPTLSLSPGERVTLSCRASESVDSSGNS |
| | | | FMHWYQQKPGQAPQLLIYRASNLESGIPARFSGSG |
| | | | SRTDFTLTISSLQPEDFAVYYCQQSFDDPFTFGQG |
| | | | TKLEIK |
| SEQ ID NO: 3121 | VL | E-H.29 | DIQLTQSPSAMSASVGDRVTITCRASESVDSSGNS |
| | | | FMHWYQQKPGKVPQRLIYRASNLESGVPSRFSGSG |
| | | | SRTEFTLTISSLQPEDFATYYCQQSFDDPFTFGQG |
| | | | TKLEIK |
| SEQ ID NO: 3122 | VL | E-H.30 | DIVLTQSPLSLPVTPGEPASISCRASESVDSSGNS |
| | | | FMHWYLQKPGQSPQLLIYRASNLESGVPDRFSGSG |
| | | | SRTDFTLKISRVEAEDVGVYYCQQSFDDPFTFGQG |
| | | | TKLEIK |
| SEQ ID NO: 3123 | VL | E-H.31 | DIVLTQTPLSLPVTPGEPASISCRASESVDSSGNS |
| | | | FMHWYLQKPGQSPQLLIYRASNLESGVPDRFSGSG |
| | | | SRTDFTLKISRVEAEDVGVYYCQQSFDDPFTFGQG |
| | | | TKLEIK |
| SEQ ID NO: 3124 | VL | E-H.32 | DIVLTQTPLSLSVTPGQPASISCRASESVDSSGNS |
| | | | FMHWYLQKPGQSPQLLIYRASNLESGVPDRFSGSG |
| | | | SRTDFTLKISRVEAEDVGVYYCQQSFDDPFTFGQG |
| | | | TKLEIK |
| SEQ ID NO: 3125 | VL | E-H.33 | EIVLTQSPPTLSLSPGERVTLSCRASESVDSSGNS |
| | | | FMHWYQQKPGQAPQLLIYRASNLESSIPARFSGSG |
| | | | SRTDFTLTISSLQPEDFAVYYCQQSFDDPFTFGQG |
| | | | TKLEIK |
| SEQ ID NO: 3126 | VL | E-H.34 | DIVLTQSPLSLPVTLGQPASISCRASESVDSSGNS |
| | | | FMHWYQQRPGQSPQRLIYRASNLESGVPDRFSGSG |
| | | | SRTDFTLKISRVEAEDVGVYYCQQSFDDPFTFGQG |
| | | | TKLEIK |
| SEQ ID NO: 3127 | VL | E-H.35 | DIVLTQTPLSSPVTLGQPASISCRASESVDSSGNS |
| | | | FMHWYQQRPGQPPQLLIYRASNLESGVPDRFSGSG |
| | | | ARTDFTLKISRVEAEDVGVYYCQQSFDDPFTFGQG |
| | | | TKLEIK |
| SEQ ID NO: 3128 | VL | E-H.36 | DIVLTQSPAFLSVTPGEKVTITCRASESVDSSGNS |
| | | | FMHWYQQKPDQAPQLLIYRASNLESGVPSRFSGSG |
| | | | SRTDFTFTISSLEAEDAATYYCQQSFDDPFTFGQG |
| | | | TKLEIK |
| SEQ ID NO: 3129 | VL | E-H.37 | DIQLIQSPSFLSASVGDRVSIICRASESVDSSGNS |
| | | | FMHWYLQKPGKSPQLFIYRASNLESGVSSRFSGRG |
| | | | SRTDFTLTIISLKPEDFAAYYCQQSFDDPFTFGQG |
| | | | TKLEIK |
| SEQ ID NO: 3130 | VL | E-H.38 | EIVLTQTPLSLSITPGEQASISCRASESVDSSGNS |
| | | | FMHWYLQKARPVPQLLIYRASNLESGVPDRFSGSG |
| | | | SRTDFTLKISRVEAEDFGVYYCQQSFDDPFTFGQG |
| | | | TKLEIK |
| SEQ ID NO: 3131 | VL | E-H.39 | EIVLTQTPLSLSITPGEQASMSCRASESVDSSGNS |
| | | | FMHWYLQKARPVPQLLIYRASNLESGVPDRFSGSG |
| | | | SRTDFTLKISRVEAEDFGVYYCQQSFDDPFTFGQG |
| | | | TKLEIK |
| SEQ ID NO: 3132 | VL | E-H.40 | EITLTQSPAFMSATPGDKVNISCRASESVDSSGNS |
| | | | FMHWYQQKPGEAPQFIIYRASNLESGIPPRFSGSG |
| | | | YRTDFTLTINNIESEDAAYYYCQQSFDDPFTFGQG |
| | | | TKLEIK |

Variable HEAVY chain (VH)

| | | | |
|---|---|---|---|
| SEQ ID NO: 3133 | VH | E-H.1 | QVQLVQSGAEVKKPGASVKVSCKASGYAFSSSWMN |
| | | | WVRQAPGQGLEWIGRIYPGDGDTKYNGKFKGRATL |
| | | | TADKSTSTAYMELSSLRSEDTAVYYCARRGTGGWY |
| | | | FDVWGQGTTVTVSS |
| SEQ ID NO: 3134 | VH | E-H.2 | QVQLVQSGAEVKKPGSSVKVSCKASGYAFSSSWMN |
| | | | WVRQAPGQGLEWIGRIYPGDGDTKYNGKFKGRATL |
| | | | TADKSTSTAYMELSSLRSEDTAVYYCARRGTGGWY |
| | | | FDVWGQGTTVTVSS |
| SEQ ID NO: 3135 | VH | E-H.3 | QVQLVQSGAEVKKPGASVKVSCKASGYAFSSSWMN |
| | | | WVRQAPGKGLEWIGRIYPGDGDTKYNGKFKGRATL |
| | | | TADKSTSTAYMELSSLRSEDTAVYYCARRGTGGWY |
| | | | FDVWGQGTTVTVSS |
| SEQ ID NO: 3136 | VH | E-H.4 | QVQLVQSGAEVKKPGASVKVSCKASGYAFSSSWMN |
| | | | WVRQAPGQELEWIGRIYPGDGDTKYNGKFKGRATL |
| | | | TADKSISTAYMELSSLRSEDTATYYCARRGTGGWY |
| | | | FDVWGQGTTVTVSS |
| SEQ ID NO: 3137 | VH | E-H.5 | EVQLVQSGAEVKKPGATVKISCKASGYAFSSSWMN |
| | | | WVQQAPGKGLEWIGRIYPGDGDTKYNGKFKGRATL |
| | | | TADKSTSTAYMELSSLRSEDTAVYYCARRGTGGWY |
| | | | FDVWGQGTTVTVSS |
| SEQ ID NO: 3138 | VH | E-H.6 | QVQLVQSGAEVKKTGSSVKVSCKASGYAFSSSWMN |
| | | | WVRQAPGQALEWIGRIYPGDGDTKYNGKFKGRATL |
| | | | TADKSMSTAYMELSSLRSEDTAMYYCARRGTGGWY |
| | | | FDVWGQGTTVTVSS |
| SEQ ID NO: 3139 | VH | E-H.7 | QVQLVQSGAEVKKPGASVKVSCKASGYAFSSSWMN |
| | | | WVRQAPGQRLEWIGRIYPGDGDTKYNGKFKGRATL |
| | | | TADKSASTAYMELSSLRSEDMAVYYCARRGTGGWY |
| | | | FDVWGQGTTVTVSS |
| SEQ ID NO: 3140 | VH | E-H. 8 | QVQLVQSGAEVKKPGASVKVSCKASGYAFSSSWMN |
| | | | WVRQAPGQGLEWIGRIYPGDGDTKYNGKFKGRATL |
| | | | TADKSTSTAYMELRSLRSDDMAVYYCARRGTGGWY |
| | | | FDVWGQGTTVTVSS |
| SEQ ID NO: 3141 | VH | E-H.9 | QVQLVQSGAEVKKPGASVKVSCKASGYAFSSSWMN |
| | | | WVRQAPGQRLEWIGRIYPGDGDTKYNGKFKGRATL |
| | | | TADKSASTAYMELSSLRSEDTAVYYCARRGTGGWY |
| | | | FDVWGQGTTVTVSS |
| SEQ ID NO: 3142 | VH | E-H.10 | QVQLVQSGAEVKKPGASVKVSCKASGYAFSSSWMN |
| | | | WVRQAPGQGLEWIGRIYPGDGDTKYNGKFKGRATL |
| | | | TADKSTSTAYMELRSLRSDDTAVYYCARRGTGGWY |
| | | | FDVWGQGTTVTVSS |
| SEQ ID NO: 3143 | VH | E-H.11 | QVQLVQSGAEVKKPGASVKVSCKASGYAFSSSWMN |
| | | | WVRQAPGQGLEWIGRIYPGDGDTKYNGKFKGRATL |
| | | | TADKSISTAYMELSRLRSDDTAVYYCARRGTGGWY |
| | | | FDVWGQGTTVTVSS |
| SEQ ID NO: 3144 | VH | E-H.12 | QVQLVQSGAEVKKPGASVKVSCKASGYAFSSSWMN |
| | | | WVRQAPGQGLEWIGRIYPGDGDTKYNGKFKGRATL |
| | | | TADKSISTAYMELSRLRSDDTVVYYCARRGTGGWY |
| | | | FDVWGQGTTVTVSS |
| SEQ ID NO: 3145 | VH | E-H.13 | QVQLVQSGAEVKKPGASVKVSCKASGYAFSSSWMN |
| | | | WVRQAPGQGLEWIGRIYPGDGDTKYNGKFKGWATL |
| | | | TADKSISTAYMELSRLRSDDTAVYYCARRGTGGWY |
| | | | FDVWGQGTTVTVSS |
| SEQ ID NO: 3146 | VH | E-H.14 | QVQLVQSGAEVKKPGASVKVSCKASGYAFSSSWMN |
| | | | WVRQATGQGLEWIGRIYPGDGDTKYNGKFKGRATL |
| | | | TANKSISTAYMELSSLRSEDTAVYYCARRGTGGWY |
| | | | FDVWGQGTTVTVSS |
| SEQ ID NO: 3147 | VH | E-H.15 | QVLQVQSGSELKKPGASVKVSCKASGYAFSSSWMN |
| | | | WVRQAPGQGLEWIGRIYPGDGDTKYNGKFKGRAVL |
| | | | SADKSVSTAYLQISSLKAEDTAVYYCARRGTGGWY |
| | | | FDVWGQGTTVTVSS |
| SEQ ID NO: 3148 | VH | E-H.16 | QVQLVQSGPEVKKPGTSVKVSCKASGYAFSSSWMN |
| | | | WVRQARGQRLEWIGRIYPGDGDTKYNGKFKGRATL |
| | | | TADKSTSTAYMELSSLRSEDTAVYYCARRGTGGWY |
| | | | FDVWGQGTTVTVSS |
| SEQ ID NO: 3149 | VH | E-H.17 | EVQLVQSGAEVKKPGESLKISCKASGYAFSSSWMN |
| | | | WVRQMPGKGLEWIGRIYPGDGDTKYNGKFKGQATL |
| | | | SADKSISTAYLQWSSLKASDTAMYYCARRGTGGWY |
| | | | FDVWGQGTTVTVSS |
| SEQ ID NO: 3150 | VH | E-H.18 | QVQLVQSGSELKKPGASVKVSCKASGYAFSSSWMN |
| | | | WVRQAPGQGLEWIGRIYPGDGDTKYNGKFKGRAVL |
| | | | SADKSVSMAYLQISSLKAEDTAVYYCARRGTGGWY |
| | | | FDVWGQGTTVTVSS |
| SEQ ID NO: 3151 | VH | E-H.19 | QVQLVQSGHEVKQPGASVKVSCKASGYAFSSSWMN |
| | | | WVPQAPGQGLEWIGRIYPGDGDTKYNGKFKGRAVL |
| | | | SADKSASTAYLQISSLKAEDMAMYYCARRGTGGWY |
| | | | FDVWGQGTTVTVSS |
| SEQ ID NO: 3152 | VH | E-H.20 | EVQLVQSGAEVKKPGESLKISCKASGYAFSSSWMN |
| | | | WVRQMPGKGLEWIGRIYPGDGDTKYNGKFKGQATL |
| | | | SADKPISTAYLQWSSLKASDTAMYYCARRGTGGWY |
| | | | FDVWGQGTTVTVSS |
| SEQ ID NO: 3153 | VH | E-H.21 | EVQLVQSGAEVKKPGESLRISCKASGYAFSSSWMN |
| | | | WVRQMPGKGLEWIGRIYPGDGDTKYNGKFKGQATL |
| | | | SADKSISTAYLQWSSLKASDTAMYYCARRGTGGWY |
| | | | FDVWGQGTTVTVSS |
| SEQ ID NO: 3154 | VH | E-H.22 | EVQLVQSGAEVKKPGESLRISCKASGYAFSSSWMN |
| | | | WVRQMPGKGLEWIGRIYPGDGDTKYNGKFKGHATL |
| | | | SADKSISTAYLQWSSLKASDTAMYYCARRGTGGWY |
| | | | FDVWGQGTTVTVSS |
| SEQ ID NO: 3155 | VH | E-H.23 | QVQLVQSGAEVKKTGSSVKVSCKASGYAFSSSWMN |
| | | | WVRQAPRQALEWIGRIYPGDGDTKYNGKFKGRATL |
| | | | TADKSMSTAYMELSSLRSEDTAMYYCARRGTGGWY |
| | | | FDVWGQGTTVTVSS |
| SEQ ID NO: 3156 | VH | E-H.24 | EVQLVESGGGLVQPGRSLRLSCTASGYAFSSSWMN |
| | | | WVRQAPGKGLEWIGRIYPGDGDTKYNGKFKGRATL |
| | | | SADKSKSIAYLQMNSLKTEDTAVYYCARRGTGGWY |
| | | | FDVWGQGTTVTVSS |
| SEQ ID NO: 3157 | VH | E-H.25 | EVQLVESGGGLVQPGPSLRLSCTASGYAFSSSWMN |
| | | | WVRQAPGKGLEWIGRIYPGDGDTKYNGKFKGRATL |
| | | | SADKSKSIAYLQMNSLKTEDTAVYYCARRGTGGWY |
| | | | FDVWGQGTTVTVSS |
| SEQ ID NO: 3158 | VH | E-E.26 | QVQLQESGPGLVKPSQTLSLTCTASGYAFSSSWMN |
| | | | WVRQPPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3159 | VH | E-E.27 | QVQLQESGPGLVKPSGTLSLTCAASGYAFSSSWMN |
| | | | WVRQPPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3160 | VH | E-H.28 | EVQLVESGGGLVKPGRSLRLSCTASGYAFSSSWMN |
| | | | WVRQAPGKGLEWIGRIYPGDGDTKYNGKFKGRATL |
| | | | SADKSKSIAYLQMNSLKTEDTAVYYCARRGTGGWY |
| | | | FDVWGQGTTVTVSS |
| SEQ ID NO: 3161 | VH | E-H.29 | EVQLVESGGGLVQPGGSLKLSCAASGYAFSSSWMN |
| | | | WVRQASGKGLEWIGRIYPGDGDTKYNGKFKGRATL |
| | | | SADKSKSTAYLQMNSLKTEDTAVYYCARRGTGGWY |
| | | | FDVWGQGTTVTVSS |
| SEQ ID NO: 3162 | VH | E-H.30 | QVQLQESGPGLVKPSQTLSLTCAASGYAFSSSWMN |
| | | | WVRQPPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3163 | VH | E-H.31 | EVQLVESGGGLVKPGGSLRLSCAASGYAFSSSWMN |
| | | | WVRQAPGKGLEWIGRIYPGDGDTKYNGKFKGRATL |
| | | | SADKSKSTAYLQMNSLKTEDTAVYYCARRGTGGWY |
| | | | FDVWGQGTTVTVSS |
| SEQ ID NO: 3164 | VH | E-H.32 | EVQLVESGGALVKPGGSLRLSCAASGYAFSSSWMN |
| | | | WVRQAPGKGLEWIGRIYPGDGDTKYNGKFKGRATL |
| | | | SADKSKSTAYLQMNSLKTEDTAVYYCARRGTGGWY |
| | | | FDVWGQGTTVTVSS |
| SEQ ID NO: 3165 | VH | E-H.33 | QVQLQESGPGLVKPSQTLSLTCAAYGYAFSSSWMN |
| | | | WVRQPPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3166 | VH | E-H.34 | QVQLQESGSGLVKPSQTLSLTCAASGYAFSSSWMN |
| | | | WVRQPPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3167 | VH | E-H.35 | EVQLVESGGGLVQPGGSLRLSCAASGYAFSSSWMN |
| | | | WVRQAPGKGLEWIGRIYPGDGDTKYNGKFKGRATL |
| | | | SADKSKSSAYLQMNSLKTEDTAVYYCARRGTGGWY |
| | | | FDVWGQGTTVTVSS |
| SEQ ID NO: 3168 | VH | E-H.36 | QVQLQESGPGLVKPSDTLSLTCTASGYAFSSSWMN |
| | | | WVRQPPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3169 | VH | E-H.37 | QVQLQESGPGLVKPSQTLSLTCTASGYAFSSSWMN |
| | | | WVRQHPGKGLEWIGRIYPGDGDTKYNGKFKGRATL |
| | | | SADKSKSQASLKLSSVTAADTAVYYCARRGTGGWY |
| | | | FDVWGQGTTVTVSS |
| SEQ ID NO: 3170 | VH | E-H.38 | QVQLQESGPGLVKPSQTLSLTCTASGYAFSSSWMN |
| | | | WVRQHPGKGLEWIGRIYPGDGDTKYNGKFKGLATL |
| | | | SADKSKSQASLKLSSVTAADTAVYYCARRGTGGWY |
| | | | FDVWGWGTTVTVSS |
| SEQ ID NO: 3171 | VH | E-H.39 | QVQLVESGGGVVQPGRSLRLSCAASGYAFSSSWMN |
| | | | WVRQAPGKGLEWIGRIYPGDGDTKYNGKFKGRATL |
| | | | SADKSKSTAYLQMSSLRAEDTAVYYCARRGTGGWY |
| | | | FDVWGQGTTVTVSS |
| SEQ ID NO: 3172 | VH | E-H.40 | QVQLVESGGGLVKPGGSLRLSCAASGYAFSSSWMN |
| | | | WVRQAPGKGLEWIGRIYPGDGDTKYNGKFKGRATL |
| | | | SADKAKSSAYLQMNSLRAEDTAVYYCARRGTGGWY |
| | | | FDVWGQGTTVTVSS |
| SEQ ID NO: 3173 | VH | E-H.41 | QVQLVESGGGLVQPGGSLRLSCSASGYAFSSSWMN |
| | | | WVRQAPGKGLEWIGRIYPGDGDTKYNGKFKGRATL |
| | | | SADKSKSTAYLQMNSLRAEDTAVYYCARRGTGGWY |
| | | | FDVWGQGTTVTVSS |
| SEQ ID NO: 3174 | VH | E-H.42 | QVQLLESGGGLVKPGGSLRLSCAASGYAFSSSWMN |
| | | | WVRQAPGKGLEWIGRIYPGDGDTKYNGKFKGRATL |
| | | | SADKAKSSAYLQMNSLRAEDTAVYYCARRGTGGWY |
| | | | FDVWGQGTTVTVSS |
| SEQ ID NO: 3175 | VH | E-H.43 | EVQLVESGGGLVQPGGSLRLSCSASGYAFSSSWMN |
| | | | WVRQAPGKGLEWIGRIYPGDGDTKYNGKFKGRATL |
| | | | SADKSKSTAYLQMSSLRAEDTAVYYCARRGTGGWY |
| | | | FDVWGQGTTVTVSS |
| SEQ ID NO: 3176 | VH | E-H.44 | QVQLQESGPGLVKPSDTLSLTCAASGYAFSSSWMN |
| | | | WVRQPPGKGLEWIGRIYPGDGDTKYNGKFKGRATL |
| | | | SADKSKSQASLKLSSVTAVDTAVYYCARRGTGGWY |
| | | | FDVWGQGTTVTVSS |
| SEQ ID NO: 3177 | VH | E-H.45 | QVQLQESGPGLVKPSQTLSLTCAASGYAFSSSWMN |
| | | | WVRQPPGKGLEWIGRIYPGDGDTKYNGKFKGRATL |
| | | | SADKSKSQASLKLSSVTAVDTAVYYCARRGTGGWY |
| | | | FDVWGQGTTVTVSS |
| SEQ ID NO: 3178 | VH | E-H.46 | EVQLVESGGGLVQPGGSLRLSCSASGYAFSSSWMN |
| | | | WVRQAPGKGLEWIGRIYPGDGDTKYNGKFKGRATL |
| | | | SADKSKSTAYVQMSSLRAEDTAVYYCARRGTGGWY |
| | | | FDVWGQGTTVTVSS |
| SEQ ID NO: 3179 | VH | E-H.47 | QVQLVDSGGGVVQPGRSLRLSCAASGYAFSSSWMN |
| | | | WVRQAPGKGLEWIGRIYPGDGDTKYNGKFKGRATL |
| | | | SADKSKSTAYLQMNSLRAEDTAVYYCARRGTGGWY |
| | | | FDVWGQGTTVTVSS |
| SEQ ID NO: 3180 | VH | E-H.48 | QVQLVESGGGVVQPGRSLRLSCAASGYAFSSSWMN |
| | | | WVRQAPGKGLEWIGRIYPGDGDTKYNGKFKGRATL |
| | | | SADKSKSTAYLQMNSLRAEGTAVYYCARRGTGGWY |
| | | | FDVWGQGTTVTVSS |
| SEQ ID NO: 3181 | VH | E-H.49 | QVQLVESGGGVVQPGRSLRLSCAASGYAFSSSWMN |
| | | | WVRQAPGKGLEWIGRIYPGDGDTKYNGKFKGRATL |
| | | | SADKSKSTAYLQMNSLRAEDTAVYYCARRGTGGWY |
| | | | FDVWGQGTTVTVSS |
| SEQ ID NO: 3182 | VH | E-H.50 | EVQLVESGGGLVQPGGSLRLSCAASGYAFSSSWMN |
| | | | WVRQAPGKGLEWIGRIYPGDGDTKYNGKFKGRATL |
| | | | SADKSKSTAYLQMNSLRAEDTAVYYCARRGTGGWY |
| | | | FDVWGQGTTVTVSS |

In some embodiments, the anti-TCRβ V5 antibody molecule comprises a VH and/or a VL of an antibody described in Table 10A, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 10A, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 11A, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 11A, or a sequence with at least 80%, 85%, 90%, 95%, 96%, 97%, 98%, 99% or more identity thereto.

### Anti-TCRβ V10 Antibodies

Accordingly, in one aspect, the disclosure provides an anti-TCRβV antibody molecule that binds to a human TCRβ V10 subfamily member. In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

Exemplary anti-TCRβ V10 antibodies of the disclosure are provided in Table 12A. In some embodiments, the anti-TCRβ V10 is antibody D, e.g., humanized antibody D (antibody D-H), as provided in Table 12A. In some embodiments, antibody D comprises one or more (e.g., three) light chain CDRs and/or one or more (e.g., three) heavy chain CDRs provided in Table 12A, or a sequence with at least 95% identity thereto. In some embodiments, antibody D comprises a variable heavy chain (VH) and/or a variable light chain (VL) provided in Table 12A, or a sequence with at least 95% identity thereto.

TABLE 12A
Amino acid sequences for anti TCRf3 V10 antibodies
Amino acid and nucleotide sequences for murine and humanized antibody
molecules which bind to TCRBV 10 (e.g., TCRBV 10-1, TCRBV 10-2 or TCRBV 10-3).
The amino acid the heavy and light chain CDRs, and the amino acid and
nucleotide sequences of the heavy and light chain
variable regions, and the heavy and light chains are shown.
Murine antibody D

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined) | LISSGGSYTYYTDSVKG |
| SEQ ID NO: 1290 | HC CDR3 (Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFRSYG |
| | | MSWVRQTPDKRLEWVALISSGGSYTYYTDSVKG |
| | | RFTISRDNAKNTLYLQMSSLKSEDTAIYYCSRHG |
| | | GNFFDYWGQGTTLTVSS |
| SEQ ID NO: 3184 | VL | QIVLTQSPSIMSASPGEKVTMTCSVSSSVSYMHW |
| | | YQQKSGTSPKRWIYDTSKLASGVPARFSGSGSGT |
| | | SYSLTISSMEAEDAATYYCQQWSSNPQYTFGGG |
| | | TKLEIK |

Humanized antibody D (D-H antibody)
Variable light chain (VL)

| | | | |
|---|---|---|---|
| SEQ ID NO: 3185 | VL | D-VL-H.1 | DIVLTQSPAFLSVTPGEKVTITCSVSSSVSYMHW |
| | | | YQQKPDQAPKLLIYDTSKLASGVPSRFSGSGSGT |
| | | | DYTFTISSLEAEDAATYYCQQWSSNPQYTFGQGT |
| | | | KLEIK |
| SEQ ID NO: 3186 | VL | D-VL-H.2 | AIQLTQSPSSLSASVGDRVTITCSVSSSVSYMHW |
| | | | YQQKPGKAPKLLIYDTSKLASGVPSRFSGSGSGT |
| | | | DYTLTISSLQPEDFATYYCQQWSSNPQYTFGQGT |
| | | | KLEIK |
| SEQ ID NO: 3187 | VL | D-VL-H.3 | DIQLTQSPSFLSASVGDRVTITCSVSSSVSYMHW |
| | | | YQQKPGKAPKLLIYDTSKLASGVPSRFSGSGSGT |
| | | | EYTLTISSLQPEDFATYYCQQWSSNPQYTFGQGT |
| | | | KLEIK |
| SEQ ID NO: 3188 | VL | D-VL-H.4 | DIQLTQSPSSLSASVGDRVTITCSVSSSVSYMHW |
| | | | YQQKPGKAPKLLIYDTSKLASGVPSRFSGSGSGT |
| | | | DYTLTISSLQPEDFATYYCQQWSSNPQYTFGQGT |
| | | | KLEIK |
| SEQ ID NO: 3189 | VL | D-VL-H.5 | DIQLTQSPSSVSASVGDRVTITCSVSSSVSYMHW |
| | | | YQQKPGKAPKLLIYDTSKLASGVPSRFSGSGSGT |
| | | | DYTLTISSLQPEDFATYYCQQWSSNPQYTFGQGT |
| | | | KLEIK |
| SEQ ID NO: 3190 | VL | D-VL-H.6 | DIQLTQSPSSLSASVGDRVTITCSVSSSVSYMHW |
| | | | YQQKPGKVPKLLIYDTSKLASGVPSRFSGSGSGT |
| | | | DYTLTISSLQPEDVATYYCQQWSSNPQYTFGQGT |
| | | | KLEIK |
| SEQ ID NO: 3191 | VL | D-VL-H.7 | DIQLTQSPSSLSASVGDRVTITCSVSSSVSYMHW |
| | | | YQQKPGQAPKLLIYDTSKLASGVPSRFSGSGSGT |
| | | | DYTLTISSLQPEDVATYYCQQWSSNPQYTFGQGT |
| | | | KLEIK |
| SEQ ID NO: 3192 | VL | D-VL-H.8 | EIVLTQSPDFQSVTPKEKVTITCSVSSSVSYMHW |
| | | | YQQKPDQSPKLLIYDTSKLASGVPSRFSGSGSGT |
| | | | DYTLTINSLEAEDAATYYCQQWSSNPQYTFGQG |
| | | | TKLEIK |
| SEQ ID NO: 3193 | VL | D-VL-H.9 | AIRLTQSPFSLSASVGDRVTITCSVSSSVSYMHW |
| | | | YQQKPAKAPKLFIYDTSKLASGVPSRFSGSGSGT |
| | | | DYTLTISSLQPEDFATYYCQQWSSNPQYTFGQGT |
| | | | KLEIK |
| SEQ ID NO: 3194 | VL | D-VL-H.10 | DIQLTQSPSSLSASVGDRVTITCSVSSSVSYMHW |
| | | | YQQKPGKAPKLLIYDTSKLASGVPSRFSGSGSGT |
| | | | DYTFTISSLQPEDIATYYCQQWSSNPQYTFGQGT |
| | | | KLEIK |
| SEQ ID NO: 3195 | VL | D-VL-H.11 | EIVLTQSPATLSLSPGERATLSCSVSSSVSYMHW |
| | | | YQQKPGQAPKLLIYDTSKLASGIPARFSGSGSGT |
| | | | DYTLTISSLEPEDFAVYYCQQWSSNPQYTFGQGT |
| | | | KLEIK |
| SEQ ID NO: 3196 | VL | D-VL-H.12 | DIQLTQSPSTLSASVGDRVTITCSVSSSVSYMHW |
| | | | YQQKPGKAPKLLIYDTSKLASGVPSRFSGSGSGT |
| | | | EYTLTISSLQPDDFATYYCQQWSSNPQYTFGQGT |
| | | | KLEIK |
| SEQ ID NO: 3197 | VL | D-VL-H.13 | DIQLTQSPSSLSASVGDRVTITCSVSSSVSYMHW |
| | | | YQQKPGKTPKLLIYDTSKLASGIPSRFSGSGSGTD |
| | | | YTLTIRSLQPEDFATYYCQQWSSNPQYTFGQGTK |
| | | | LEIK |
| SEQ ID NO: 3198 | VL | D-VL-H.14 | EIVLTQSPPTLSLSPGERVTLSCSVSSSVSYMHWY |
| | | | QQKPGQAPKLLIYDTSKLASGIPARFSGSGSGTD |
| | | | YTLTISSLQPEDFAVYYCQQWSSNPQYTFGQGTK |
| | | | LEIK |
| SEQ ID NO: 3199 | VL | D-VL-H.15 | DIQLTQSPSSLSASVGDRVTITCSVSSSVSYMHW |
| | | | YQQKPGKAPKRLIYDTSKLASGVPSRFSGSGSGT |
| | | | EYTLTISSLQPEDFATYYCQQWSSNPQYTFGQGT |
| | | | KLEIK |
| SEQ ID NO: 3200 | VL | D-VL-H.16 | EIVLTQSPATLSLSPGERATLSCSVSSSVSYMHW |
| | | | YQQKPGQAPKLLIYDTSKLASGIPARFSGSGPGT |
| | | | DYTLTISSLEPEDFAVYYCQQWSSNPQYTFGQGT |
| | | | KLEIK |
| SEQ ID NO: 3201 | VL | D-VL-H.17 | EIVLTQSPATLSLSPGERATLSCSVSSSVSYMHW |
| | | | YQQKPGQAPKLLIYDTSKLASGIPARFSGSGSGT |
| | | | DYTLTISRLEPEDFAVYYCQQWSSNPQYTFGQGT |
| | | | KLEIK |
| SEQ ID NO: 3202 | VL | D-VL-H.18 | EIVLTQSPATLSLSPGERATLSCSVSSSVSYMHW |
| | | | YQQKPGQAPKLLIYDTSKLASGIPARFSGSGSGT |
| | | | DYTLTISSLQPEDFAVYYCQQWSSNPQYTFGQGT |
| | | | KLEIK |
| SEQ ID NO: 3203 | VL | D-VL-H.19 | EIVLTQSPATLSVSPGERATLSCSVSSSVSYMHW |
| | | | YQQKPGQAPKLLIYDTSKLASGIPARFSGSGSGTE |
| | | | YTLTISSLQSEDFAVYYCQQWSSNPQYTFGQGTK |
| | | | LEIK |
| SEQ ID NO: 3204 | VL | D-VL-H.20 | EIVLTQSPATLSVSPGERATLSCSVSSSVSYMHW |
| | | | YQQKPGQAPKLLIYDTSKLASGIPARFSGSGSGTE |
| | | | YTLTISILQSEDFAVYYCQQWSSNPQYTFGQGTK |
| | | | LEIK |
| SEQ ID NO: 3205 | VL | D-VL-H.21 | EIVLTQSPPTLSLSPGERVTLSCSVSSSVSYMHWY |
| | | | QQKPGQAPKLLIYDTSKLASSIPARFSGSGSGTDY |
| | | | TLTISSLQPEDFAVYYCQQWSSNPQYTFGQGTKL |
| | | | EIK |
| SEQ ID NO: 3206 | VL | D-VL-H.22 | DIQLTQSPSSLSASVGDRVTITCSVSSSVSYMHW |
| | | | YQQKPGKAPKSLIYDTSKLASGVPSRFSGSGSGT |
| | | | DYTLTISSLQPEDFATYYCQQWSSNPQYTFGQGT |
| | | | KLEIK |
| SEQ ID NO: 3207 | VL | D-VL-H.23 | DIQLTQSPSSLSASVGDRVTITCSVSSSVSYMHW |
| | | | YQQKPGKAPKRLIYDTSKLASGVPSRFSGSGSGT |
| | | | EYTLTISNLQPEDFATYYCQQWSSNPQYTFGQGT |
| | | | KLEIK |
| SEQ ID NO: 3208 | VL | D-VL-H.24 | DIQLTQSPSAMSASVGDRVTITCSVSSSVSYMHW |
| | | | YQQKPGKVPKRLIYDTSKLASGVPSRFSGSGSGT |
| | | | EYTLTISSLQPEDFATYYCQQWSSNPQYTFGQGT |
| | | | KLEIK |
| SEQ ID NO: 3209 | VL | D-VL-H.25 | EIVLTQSPATLSLSPGERATLSCSVSSSVSYMHW |
| | | | YQQKPGQAPKLLIYDTSKLASGIPDRFSGSGSGT |
| | | | DYTLTISRLEPEDFAVYYCQQWSSNPQYTFGQGT |
| | | | KLEIK |
| SEQ ID NO: 3210 | VL | D-VL-H.26 | EIVLTQSPATLSLSPGERATLSCSVSSSVSYMHW |
| | | | YQQKPGLAPKLLIYDTSKLASGIPDRFSGSGSGTD |
| | | | YTLTISRLEPEDFAVYYCQQWSSNPQYTFGQGTK |
| | | | LEIK |
| SEQ ID NO: 3211 | VL | D-VL-H.27 | EIVLTQSPGTLSLSPGERATLSCSVSSSVSYMHW |
| | | | YQQKPGQAPKLLIYDTSKLASGIPDRFSGSGSGT |
| | | | DYTLTISRLEPEDFAVYYCQQWSSNPQYTFGQGT |
| | | | KLEIK |
| SEQ ID NO: 3212 | VL | D-VL-H.28 | DIQLTQSPSSLSASVGDRVTITCSVSSSVSYMHW |
| | | | YQQKPGKAPKSLIYDTSKLASGVPSKFSGSGSGT |
| | | | DYTLTISSLQPEDFATYYCQQWSSNPQYTFGQGT |
| | | | KLEIK |
| SEQ ID NO: 3213 | VL | D-VL-H.29 | DIQLTQSPSSLSASVGDRVTITCSVSSSVSYMHW |
| | | | YQQKPEKAPKSLIYDTSKLASGVPSRFSGSGSGT |
| | | | DYTLTISSLQPEDFATYYCQQWSSNPQYTFGQGT |
| | | | KLEIK |
| SEQ ID NO: 3214 | VL | D-VL-H.30 | DIVLTQSPDSLAVSLGERATINCSVSSSVSYMHW |
| | | | YQQKPGQPPKLLIYDTSKLASGVPDRFSGSGSGT |
| | | | DYTLTISSLQAEDVAVYYCQQWSSNPQYTFGQG |
| | | | TKLEIK |
| SEQ ID NO: 3215 | VL | D-VL-H.31 | EIVLTQTPLSLSITPGEQASMSCSVSSSVSYMHWY |
| | | | LQKARPVPKLLIYDTSKLASGVPDRFSGSGSGTD |
| | | | YTLKISRVEAEDFGVYYCQQWSSNPQYTFGQGT |
| | | | KLEIK |
| SEQ ID NO: 3216 | VL | D-VL-H.32 | EIVLTQTPLSLSITPGEQASISCSVSSSVSYMHWY |
| | | | LQKARPVPKLLIYDTSKLASGVPDRFSGSGSGTD |
| | | | YTLKISRVEAEDFGVYYCQQWSSNPQYTFGQGT |
| | | | KLEIK |
| SEQ ID NO: 3217 | VL | D-VL-H.33 | DIVLTQSPLSLPVTPGEPASISCSVSSSVSYMHWY |
| | | | LQKPGQSPKLLIYDTSKLASGVPDRFSGSGSGTD |
| | | | YTLKISRVEAEDVGVYYCQQWSSNPQYTFGQGT |
| | | | KLEIK |
| SEQ ID NO: 3218 | VL | D-VL-H.34 | DIVLTQSPLSLPVTLGQPASISCSVSSSVSYMHWY |
| | | | QQRPGQSPKRLIYDTSKLASGVPDRFSGSGSGTD |
| | | | YTLKISRVEAEDVGVYYCQQWSSNPQYTFGQGT |
| | | | KLEIK |
| SEQ ID NO: 3219 | VL | D-VL-H.35 | DIVLTQTPLSLPVTPGEPASISCSVSSSVSYMHWY |
| | | | LQKPGQSPKLLIYDTSKLASGVPDRFSGSGSGTD |
| | | | YTLKISRVEAEDVGVYYCQQWSSNPQYTFGQGT |
| | | | KLEIK |
| SEQ ID NO: 3220 | VL | D-VL-H.36 | DIVLTQTPLSLSVTPGQPASISCSVSSSVSYMHWY |
| | | | LQKPGQSPKLLIYDTSKLASGVPDRFSGSGSGTD |
| | | | YTLKISRVEAEDVGVYYCQQWSSNPQYTFGQGT |
| | | | KLEIK |
| SEQ ID NO: 3221 | VL | D-VL-H.37 | DIVLTQTPLSLSVTPGQPASISCSVSSSVSYMHWY |
| | | | LQKPGQPPKLLIYDTSKLASGVPDRFSGSGSGTD |
| | | | YTLKISRVEAEDVGVYYCQQWSSNPQYTFGQGT |
| | | | KLEIK |
| SEQ ID NO: 3222 | VL | D-VL-H.38 | DIQLIQSPSFLSASVGDRVSIICSVSSSVSYMHWY |
| | | | LQKPGKSPKLFIYDTSKLASGVSSRFSGRGSGTD |
| | | | YTLTIISLKPEDFAAYYCQQWSSNPQYTFGQGTK |
| | | | LEIK |
| SEQ ID NO: 3223 | VL | D-VL-H.39 | DIVLTQTPLSSPVTLGQPASISCSVSSSVSYMHWY |
| | | | QQRPGQPPKLLIYDTSKLASGVPDRFSGSGAGTD |
| | | | YTLKISRVEAEDVGVYYCQQWSSNPQYTFGQGT |
| | | | KLEIK |
| SEQ ID NO: 3224 | VL | D-VL-H.40 | EITLTQSPAFMSATPGDKVNISCSVSSSVSYMHW |
| | | | YQQKPGEAPKFIIYDTSKLASGIPPRFSGSGYGTD |
| | | | YTLTINNIESEDAAYYYCQQWSSNPQYTFGQGT |
| | | | KLEIK |

Variable HEAVY chain (VH)

| | | | |
|---|---|---|---|
| SEQ ID NO: 3225 | VH | D-VH-H.1 | EVQLVESGGGLVKPGGSLRLSCAVSGFTFRSYG |
| | | | MSWVRQAPGKGLEWVALISSGGSYTYYTDSVK |
| | | | GRFTISRDNSKNTLYLQMNSLKTEDTAVYYCSR |
| | | | HGGNFFDYWGQGTTVTVSS |
| SEQ ID NO: 3226 | VH | D-VH-H.2 | EVQLVESGGALVKPGGSLRLSCAVSGFTFRSYG |
| | | | MSWVRQAPGKGLEWVALISSGGSYTYYTDSVK |
| | | | GRFTISRDNSKNTLYLQMNSLKTEDTAVYYCSR |
| | | | HGGNFFDYWGQGTTVTVSS |
| SEQ ID NO: 3227 | VH | D-VH-H.3 | EVQLVESGGGLVQPGGSLRLSCAVSGFTFRSYG |
| | | | MSWVRQAPGKGLEWVALISSGGSYTYYTDSVK |
| | | | GRFTISRDNAKNTLYLQMNSLRAEDTAVYYCSR |
| | | | HGGNFFDYWGQGTTVTVSS |
| SEQ ID NO: 3228 | VH | D-VH-H.4 | EVQLVESGGGLVQPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3229 | VH | D-VH-H.5 | EVQLVESGGGLVQPGGSLRLSCAVSGFTFRSYG |
| | | | MSWVRQAPGKGLEWVALISSGGSYTYYTDSVK |
| | | | GRFTISRDNSKNSLYLQMNSLKTEDTAVYYCSR |
| | | | HGGNFFDYWGQGTTVTVSS |
| SEQ ID NO: 3230 | VH | D-VH-H.6 | EVQLVESGGGLVQPGGSLRLSCAVSGFTFRSYG |
| | | | MSWVRQAPGKGLEWVALISSGGSYTYYTDSVK |
| | | | GRFTISRDNAKNSLYLQMNSLRAEDMAVYYCSR |
| | | | HGGNFFDYWGQGTTVTVSS |
| SEQ ID NO: 3231 | VH | D-VH-H.7 | EVQLVESGGGLVQPGGSLRLSCAVSGFTFRSYG |
| | | | MSWVRQAPGKGLEWVALISSGGSYTYYTDSVK |
| | | | GQFTISRDNAKNTLYLQMNSLRAEDMAVYYCSR |
| | | | HGGNFFDYWGQGTTVTVSS |
| SEQ ID NO: 3232 | VH | D-VH-H.8 | EVQLVESGGGLVKPGRSLRLSCTVSGFTFRSYGM |
| | | | SWVRQAPGKGLEWVALISSGGSYTYYTDSVKGR |
| | | | FTISRDNSKNILYLQMNSLKTEDTAVYYCSRHGG |
| | | | NFFDYWGQGTTVTVSS |
| SEQ ID NO: 3233 | VH | D-VH-H.9 | EVQLVESGGGLVKPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3234 | VH | D-VH-H.10 | EVQLVESGGGLVQPGGSLKLSCAVSGFTFRSYG |
| | | | MSWVRQASGKGLEWVALISSGGSYTYYTDSVK |
| | | | GRFTISRDNSKNTLYLQMNSLKTEDTAVYYCSR |
| | | | HGGNFFDYWGQGTTVTVSS |
| SEQ ID NO: 3235 | VH | D-VH-H.11 | QVQLVESGGGVVQPGGSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3236 | VH | D-VH-H.12 | QVQLVESGGGVVQPGRSLRLSCAVSGFTFRSYG |
| | | | MSWVRQAPGKGLEWVALISSGGSYTYYTDSVK |
| | | | GRFTISRDNSKNTLYLQMSSLRAEDTAVYYCSRH |
| | | | GGNFFDYWGQGTTVTVSS |
| SEQ ID NO: 3237 | VH | D-VH-H.13 | EVQLVESGGGLVQPGGSLRLSCPVSGFTFRSYGM |
| | | | SWVRQAPGKGLEWVALISSGGSYTYYTDSVKGR |
| | | | FTISRDNANNSLYLQMNSLRAEDTAVYYCSRHG |
| | | | GNFFDYWGQGTTVTVSS |
| SEQ ID NO: 3238 | VH | D-VH-H.14 | EVQLVESGGGLVQPGRSLRLSCTVSGFTFRSYGM |
| | | | SWVRQAPGKGLEWVALISSGGSYTYYTDSVKGR |
| | | | FTISRDNSKNILYLQMNSLKTEDTAVYYCSRHGG |
| | | | NFFDYWGQGTTVTVSS |
| SEQ ID NO: 3239 | VH | D-VH-H.15 | EVQLVESGGGLVQPGPSLRLSCTVSGFTFRSYGM |
| | | | SWVRQAPGKGLEWVALISSGGSYTYYTDSVKGR |
| | | | FTISRDNSKNILYLQMNSLKTEDTAVYYCSRHGG |
| | | | NFFDYWGQGTTVTVSS |
| SEQ ID NO: 3240 | VH | D-VH-H.16 | EVQLVESGGGLVQPGGSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3241 | VH | D-VH-H.17 | EVQLVESGGGLVQPGGSLRLSCAVSGFTFRSYG |
| | | | MSWVRQAPGKGLEWVALISSGGSYTYYTDSVK |
| | | | GRFTISRDNAKNSLYLQMNSLRDEDTAVYYCSR |
| | | | HGGNFFDYWGQGTTVTVSS |
| SEQ ID NO: 3242 | VH | D-VH-H.18 | QVQLVESGGGLVKPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3243 | VH | D-VH-H.19 | QVQLVESGGGVVQPGRSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3244 | VH | D-VH-H.20 | EVQLLESGGGLVQPGGSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3245 | VH | D-VH-H.21 | EVQLVESGGGLVQPGGSLRLSCAVSGFTFRSYG |
| | | | MSWVRQAPGKGLEWVALISSGGSYTYYTDSVK |
| | | | GRFTISRHNSKNTLYLQMNSLRAEDTAVYYCSR |
| | | | HGGNFFDYWGQGTTVTVSS |
| SEQ ID NO: 3246 | VH | D-VH-H.22 | EVQLVESGGGLIQPGGSLRLSCAVSGFTFRSYGM |
| | | | SWVRQPPGKGLEWVALISSGGSYTYYTDSVKGR |
| | | | FTISRDNSKNTLYLQMNSLRAEDTAVYYCSRHG |
| | | | GNFFDYWGQGTTVTVSS |
| SEQ ID NO: 3247 | VH | D-VH-H.23 | EVQLVESGGGLIQPGGSLRLSCAVSGFTFRSYGM |
| | | | SWVRQAPGKGLEWVALISSGGSYTYYTDSVKGR |
| | | | FTISRDNSKNTLYLQMNSLRAEDTAVYYCSRHG |
| | | | GNFFDYWGQGTTVTVSS |
| SEQ ID NO: 3248 | VH | D-VH-H.24 | EVQLVESGGGLVQPGRSLRLSCAVSGFTFRSYG |
| | | | MSWVRQAPGKGLEWVALISSGGSYTYYTDSVK |
| | | | GRFTISRDNAKNSLYLQMNSLRAEDTALYYCSR |
| | | | HGGNFFDYWGQGTTVTVSS |
| SEQ ID NO: 3249 | VH | D-VH-H.25 | QVQLVESGGGVVQPGRSLRLSCAVSGFTFRSYG |
| | | | MSWVRQAPGKGLEWVALISSGGSYTYYTDSVK |
| | | | GRFTISRDNSKNRLYLQMNSLRAEDTAVYYCSR |
| | | | HGGNFFDYWGQGTTVTVSS |
| SEQ ID NO: 3250 | VH | D-VH-H.26 | QVQLVESGGGVVQPGRSLRLSCAVSGFTFRSYG |
| | | | MSWVRQAPGKGLEWVALISSGGSYTYYTDSVK |
| | | | GRFTISRDNSKNTLYLQMNSLRAEGTAVYYCSR |
| | | | HGGNFFDYWGQGTTVTVSS |
| SEQ ID NO: 3251 | VH | D-VH-H.27 | QVQLVESGGGVVQPGRSLRLSCAVSGFTFRSYG |
| | | | MSWVRQAPGKGLEWVALISSGGSYTYYTDSVK |
| | | | GRFAISRDNSKNTLYLQMNSLRAEDTAVYYCSR |
| | | | HGGNFFDYWGQGTTVTVSS |
| SEQ ID NO: 3252 | VH | D-VH-H.28 | QVQLVDSGGGVVQPGRSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3253 | VH | D-VH-H.29 | EVQLVESGGGVVRPGGSLRLSCAVSGFTFRSYG |
| | | | MSWVRQAPGKGLEWVALISSGGSYTYYTDSVK |
| | | | GRFTISRDNAKNSLYLQMNSLRAEDTALYHCSR |
| | | | HGGNFFDYWGQGTTVTVSS |
| SEQ ID NO: 3254 | VH | D-VH-H.30 | EVQLVESGGVVVQPGGSLRLSCAVSGFTFRSYG |
| | | | MSWVRQAPGKGLEWVALISSGGSYTYYTDSVK |
| | | | GRFTISRDNSKNSLYLQMNSLRAEDTALYYCSRH |
| | | | GGNFFDYWGQGTTVTVSS |
| SEQ ID NO: 3255 | VH | D-VH-H.31 | EVQLVESGGGVVQPGGSLRLSCAVSGFTFRSYG |
| | | | MSWVRQAPGKGLEWVALISSGGSYTYYTDSVK |
| | | | GRFTISRDNSKNSLYLQMNSLRTEDTALYYCSRH |
| | | | GGNFFDYWGQGTTVTVSS |
| SEQ ID NO: 3256 | VH | D-VH-H.32 | EVQLVESGGVVVQPGGSLRLSCAVSGFTFRSYG |
| | | | MSWVRQAPGKGLEWVALISSGGSYTYYTDSVK |
| | | | GRFTISRDNSKNSLYLQMNSLRTEDTALYYCSRH |
| | | | GGNFFDYWGQGTTVTVSS |
| SEQ ID NO: 3257 | VH | D-VH-H.33 | EVQLVETGGGLIQPGGSLRLSCAVSGFTFRSYGM |
| | | | SWVRQAPGKGLEWVALISSGGSYTYYTDSVKGR |
| | | | FTISRDNSKNTLYLQMNSLRAEDTAVYYCSRHG |
| | | | GNFFDYWGQGTTVTVSS |
| SEQ ID NO: 3258 | VH | D-VH-H.34 | EVQLVESGGGLVQPGGSLRLSCAVSGFTFRSYG |
| | | | MSWVRQATGKGLEWVALISSGGSYTYYTDSVK |
| | | | GRFTISRENAKNSLYLQMNSLRAGDTAVYYCSR |
| | | | HGGNFFDYWGQGTTVTVSS |
| SEQ ID NO: 3259 | VH | D-VH-H.35 | EVQLVESRGVLVQPGGSLRLSCAVSGFTFRSYG |
| | | | MSWVRQAPGKGLEWVALISSGGSYTYYTDSVK |
| | | | GRFTISRDNSKNTLHLQMNSLRAEDTAVYYCSR |
| | | | HGGNFFDYWGQGTTVTVSS |
| SEQ ID NO: 3260 | VH | D-VH-H.36 | EVQLVESGGGLVQPGRSLRLSCAVSGFTFRSYG |
| | | | MSWVRQAPGKGLEWVALISSGGSYTYYTDSVK |
| | | | GRFTISRDNAKNSLYLQMNSLRAEDMALYYCSR |
| | | | HGGNFFDYWGQGTTVTVSS |
| SEQ ID NO: 3261 | VH | D-VH-H.37 | QVQLVESGGGLVQPGGSLRLSCSVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3262 | VH | D-VH-H.38 | EVQLVESGGGLVQPGGSLRLSCSVSGFTFRSYGM |
| | | | SWVRQAPGKGLEWVALISSGGSYTYYTDSVKGR |
| | | | FTISRDNSKNTLYLQMSSLRAEDTAVYYCSRHG |
| | | | GNFFDYWGQGTTVTVSS |
| SEQ ID NO: 3263 | VH | D-VH-H.39 | QVQLVESGGGVVQPGRSLRLSCAVSGFTFRSYG |
| | | | MSWVRQAPGKGLEWVALISSGGSYTYYTDSVK |
| | | | GRFTISRDNSTNTLFLQMNSLRAEDTAVYYCSRH |
| | | | GGNFFDYWGQGTTVTVSS |
| SEQ ID NO: 3264 | VH | D-VH-H.40 | QVQLLESGGGLVKPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3265 | VH | D-VH-H.41 | EVQLVESGEGLVQPGGSLRLSCAVSGFTFRSYG |
| | | | MSWVRQAPGKGLEWVALISSGGSYTYYTDSVK |
| | | | GRFTISRDNSKNTLYLQMGSLRAEDMAVYYCSR |
| | | | HGGNFFDYWGQGTTVTVSS |
| SEQ ID NO: 3266 | VH | D-VH-H.42 | EVQLVESGGGLVQPGGSLRLSCAVSGFTFRSYG |
| | | | MSWVRQAPGKGLEWVALISSGGSYTYYTDSVK |
| | | | GRFTISRDNSKNTLYLQMGSLRAEDMAVYYCSR |
| | | | HGGNFFDYWGQGTTVTVSS |
| SEQ ID NO: 3267 | VH | D-VH-H.43 | EVQLVESGGGLVQPGGSLRLSCSVSGFTFRSYGM |
| | | | SWVRQAPGKGLEWVALISSGGSYTYYTDSVKGR |
| | | | FTISRDNSKNTLYVQMSSLRAEDTAVYYCSRHG |
| | | | GNFFDYWGQGTTVTVSS |
| SEQ ID NO: 3268 | VH | D-VH-H.44 | EVQLVESGGGLVQPGGSLRLSCAVSGFTFRSYG |
| | | | MSWVRQAPGKGLEWVALISSGGSYTYYTDSVK |
| | | | GRFIISRDNSRNSLYLQKNRRRAEDMAVYYCSR |
| | | | HGGNFFDYWGQGTTVTVSS |
| SEQ ID NO: 3269 | VH | D-VH-H.45 | EVQLVESGGGLVQPGGSLRLSCAVSGFTFRSYG |
| | | | MSWVHQAPGKGLEWVALISSGGSYTYYTDSVK |
| | | | GRFIISRDNSRNTLYLQTNSLRAEDTAVYYCSRH |
| | | | GGNFFDYWGQGTTVTVSS |
| SEQ ID NO: 3270 | VH | D-VH-H.46 | EVHLVESGGGLVQPGGALRLSCAVSGFTFRSYG |
| | | | MSWVRQATGKGLEWVALISSGGSYTYYTDSVK |
| | | | GRFTISRENAKNSLYLQMNSLRAGDTAVYYCSR |
| | | | HGGNFFDYWGQGTTVTVSS |
| SEQ ID NO: 3271 | VH | D-VH-H.47 | EVQLVESGGGLVQPRGSLRLSCAVSGFTFRSYG |
| | | | MSWVRQAPGKGLEWVALISSGGSYTYYTDSVK |
| | | | GRFTISRDNSKNTLYLQMNNLRAEGTAVYYCSR |
| | | | HGGNFFDYWGQGTTVTVSS |
| SEQ ID NO: 3272 | VH | D-VH-H.48 | EVQLVESGGGLVQPRGSLRLSCAVSGFTFRSYG |
| | | | MSWVRQAPGKGLEWVALISSGGSYTYYTDSVK |
| | | | GRFTISRDNSKNTLYLQMNNLRAEGTAAYYCSR |
| | | | HGGNFFDYWGQGTTVTVSS |
| SEQ ID NO: 3273 | VH | D-VH-H.49 | QVQLVQSGAEVKKPGASVKVSCKVSGFTFRSYG |
| | | | MSWVRQAPGKGLEWVALISSGGSYTYYTDSVK |
| | | | GRFTITRDNSTNTLYMELSSLRSEDTAVYYCSRH |
| | | | GGNFFDYWGQGTTVTVSS |
| SEQ ID NO: 3274 | VH | D-VH-H.50 | QVQLVQSGSELKKPGASVKVSCKVSGFTFRSYG |
| | | | MSWVRQAPGQGLEWVALISSGGSYTYYTDSVK |
| | | | GRFVISRDNSVNTLYLQISSLKAEDTAVYYCSRH |
| | | | GGNFFDYWGQGTTVTVSS |

In some embodiments, the anti-TCRβ V10 antibody molecule comprises a VH or a VL of an antibody described in Table 12A, or a sequence with at least 80%, 85%, 90%, 95%, 96%, 97%, 98%, 99% or more identity thereto.

In some embodiments, the anti-TCRβ V10 antibody molecule comprises a VH and a VL of an antibody described in Table 12A, or a sequence with at least 80%, 85%, 90%, 95%, 96%, 97%, 98%, 99% or more identity thereto.

### Additional Anti-TCRVβ Antibodies

Additional exemplary anti-TCRβV antibodies of the disclosure are provided in Table 13A. In some embodiments, the anti-TCRβV antibody is a humanized antibody, e.g., as provided in Table 13A. In some embodiments, the anti-TCRβV antibody comprises one or more (e.g., all three) of a LC CDR1, LC CDR2, and LC CDR3 provided in Table 13A; and/or one or more (e.g., all three) of a HC CDR1, HC CDR2, and HC CDR3 provided in Table 13A, or a sequence with at least 95% identity thereto. In some embodiments, the anti-TCRβV antibody comprises a variable heavy chain (VH) and/or a variable light chain (VL) provided in Table 13A, or a sequence with at least 95% identity thereto.

```
TABLE 13A
Amino acid sequences for additional anti-TCRβ V antibodies
Amino acid and nucleotide sequences for murine and humanized antibody molecules
which bind to various TCRVB families are disclosed. The amino acid the heavy
and light chain CDRs, and the amino acid and nucleotide sequences of the heavy and
light chain variable regions, and the heavy and light chains are shown.
  Antibodies disclosed in the table include, MPB2D5, CAS1.1.3, IMMU222, REA1062,
JOVI-3 and IMMU546. MPB2D5 binds human TCRβV 20-1 (TCRβV2 per old nomenclature).
CAS1.1.3 binds human TCRβV 27 (TCRβV14 per old nomenclature).
  IMMU 222 binds human TCRβV 6-5, TCRβV 6-6, or TCRβV 6-9 (TCRβV13.1
per old nomenclature). REA1062 binds human TCRβV 5-1). JOVI-3 binds human TCRβV 28
(TCRβV3.1 per old nomenclature). IMMU546 binds human TCRβV 2.
Antibody G (murine) binds to human TCRVβ 20-1
```

| | | |
|---|---|---|
| SEQ ID NO: 1102 | HC CDR1 (Kabat) | SAYMH |
| SEQ ID NO: 1103 | HC CDR2 (Kabat) | RIDPATGKTKYAPKFQA |
| SEQ ID NO: 1104 | HC CDR3 (Kabat) | SLNWDYGLDY |
| SEQ ID NO: 1105 | HC CDR1 (Chothia) | GFNIKSA |
| SEQ ID NO: 1106 | HC CDR2 (Chothia) | DPATGK |
| SEQ ID NO: 1104 | HC CDR3 (Chothia) | SLNWDYGLDY |
| SEQ ID NO: 7289 | HC CDR1 (Combined) | GFNIKSAYMH |
| SEQ ID NO: 1103 | HC CDR2 (Combined) | RIDPATGKTKYAPKFQA |
| SEQ ID NO: 1104 | HC CDR3 (Combined) | SLNWDYGLDY |
| SEQ ID NO: 1107 | LC CDR1 (Kabat) | RASKSVSILGTHLIH |
| SEQ ID NO: 1108 | LC CDR2 (Kabat) | AASNLES |
| SEQ ID NO: 1109 | LC CDR3 (Kabat) | QQSIEDPWT |
| SEQ ID NO: 1110 | LC CDR1 (Chothia) | SKSVSILGTHL |
| SEQ ID NO: 1108 | LC CDR2 (Chothia) | AASNLES |
| SEQ ID NO: 1109 | LC CDR3 (Chothia) | QQSIEDPWT |
| SEQ ID NO: 1107 | LC CDR1 (Combined) | RASKSVSILGTHLIH |
| SEQ ID NO: 1108 | LC CDR2 (Combined) | AASNLES |
| SEQ ID NO: 1109 | LC CDR3 (Combined) | QQSIEDPWT |
| SEQ ID NO: 1111 | VL | DIVLTQSPASLAVSLGQRATISCRASKSVSILGTHLIH |
| | | WYQQKPGQPPKLLIYAASNLESGVPARFSGSGSETV |
| | | FTLNIHPVEEEDAATYFCQQSIEDPWTFGGGTKLGI |
| | | K |
| SEQ ID NO: 1112 | VH | EVQLQQSVADLVRPGASLKLSCTASGFNIKSAYMH |
| | | WVIQRPDQGPECLGRIDPATGKTKYAPKFQAKATIT |
| | | ADTSSNTAYLQLSSLTSEDTAIYYCTRSLNWDYGLD |
| | | YWGQGTSVTVSS |

Antibody G-H (humanized) VHs binds to human TCRVβ 20-1

| | | |
|---|---|---|
| SEQ ID NO: 1113 | VH-1 | QVQLVQSGAEVKKPGASVKVSCKASGFNIKSAYM |
| | | HWVRQAPGQGLEWMGRIDPATGKTKYAPKFQARV |
| | | TMTADTSTNTAYMELSSLRSEDTAVYYCARSLNW |
| | | DYGLDYWGQGTLVTVSS |
| SEQ ID NO: 1114 | VH-2 | QVQLVQSGAEVKKPGASVKVSCKASGFNIKSAYM |
| | | HWVRQAPGQEPGCMGRIDPATGKTKYAPKFQARV |
| | | TMTADTSINTAYTELSSLRSEDTATYYCARSLNWD |
| | | YGLDYWGQGTLVTVSS |
| SEQ ID NO: 1115 | VH-3 | QVQLVQSGAEVKKPGSSVKVSCKASGFNIKSAYMH |
| | | WVRQAPGQGLEWMGRIDPATGKTKYAPKFQARVT |
| | | ITADTSTNTAYMELSSLRSEDTAVYYCARSLNWDY |
| | | GLDYWGQGTLVTVSS |
| SEQ ID NO: 1116 | VH-4 | QVQLVQSGAEVKKPGASVKVSCKASGFNIKSAYM |
| | | HWVRQAPGQRLEWMGRIDPATGKTKYAPKFQARV |
| | | TITADTSANTAYMELSSLRSEDTAVYYCARSLNWD |
| | | YGLDYWGQGTLVTVSS |

Antibody G-H (humanized) VLs binds to human TCRVβ 20-1

| | | |
|---|---|---|
| SEQ ID NO: 1117 | VL-1 | EIVLTQSPATLSLSPGERATLSCRASKSVSILGTHLIH |
| | | WYQQKPGQAPRLLIYAASNLESGIPARFSGSGSETD |
| | | FTLTISSLEPEDFAVYFCQQSIEDPFGGGTKVEIK |
| SEQ ID NO: 1118 | VL-2 | EIVLTQSPATLSLSPGERATLSCRASKSVSILGTHLIH |
| | | WYQQKPGLAPRLLIYAASNLESGIPDRFSGSGSETD |
| | | FTLTISRLEPEDFAVYFCQQSIEDPFGGGTKVEIK |
| SEQ ID NO: 1119 | VL-3 | EIVLTQSPGTLSLSPGERATLSCRASKSVSILGTHLIH |
| | | WYQQKPGQAPRLLIYAASNLESGIPDRFSGSGSETD |
| | | FTLTISRLEPEDFAVYFCQQSIEDPFGGGTKVEIK |

Antibody H (murine) binds to human TCRVβ 27

| | | |
|---|---|---|
| SEQ ID NO: 1120 | HC CDR1 (Kabat) | DTYMY |
| SEQ ID NO: 1121 | HC CDR2 (Kabat) | RIDPANGNTKYDPKFQD |
| SEQ ID NO: 1122 | HC CDR3 (Kabat) | GSYYYAMDY |
| SEQ ID NO: 1123 | HC CDR1 (Chothia) | GFKTEDT |
| SEQ ID NO: 1124 | HC CDR2 (Chothia) | DPANGN |
| SEQ ID NO: 1122 | HC CDR3 (Chothia) | GSYYYAMDY |
| SEQ ID NO: 1125 | HC CDR1 (Combined) | GFKTEDTYMY |
| SEQ ID NO: 1121 | HC CDR2 (Combined) | RIDPANGNTKYDPKFQD |
| SEQ ID NO: 1122 | HC CDR3 (Combined) | GSYYYAMDY |
| SEQ ID NO: 1126 | LC CDR1 (Kabat) | RASESVDSYGNSFMH |
| SEQ ID NO: 1127 | LC CDR2 (Kabat) | RASNLES |
| SEQ ID NO: 1128 | LC CDR3 (Kabat) | QQSNEDPYT |
| SEQ ID NO: 7290 | LC CDR1 (Chothia) | SESVDSYGNSF |
| SEQ ID NO: 1127 | LC CDR2 (Chothia) | RASNLES |
| SEQ ID NO: 1128 | LC CDR3 (Chothia) | QQSNEDPYT |
| SEQ ID NO: 1126 | LC CDR1 (Combined) | RASESVDSYGNSFMH |
| SEQ ID NO: 1127 | LC CDR2 (Combined) | RASNLES |
| SEQ ID NO: 1128 | LC CDR3 (Combined) | QQSNEDPYT |
| SEQ ID NO: 1129 | VL | DIVLTQSPASLAVSLGQRATISCRASESVDSYGNSF |
| | | MHWYQQKPGQPPKLLIYRASNLESGIPARFSGSGSR |
| | | TDFTLTINPVEADDVATYYCQQSNEDPYTFGGGTK |
| | | LEIK |
| SEQ ID NO: 1130 | VH | EVQLQQSGAELVKPGASVKLSCTASGFKTEDTYMY |
| | | WVKQRPEQGLEWIGRIDPANGNTKYDPKFQDKATI |
| | | TADSSSNTAYLQLSSLPSEDTAVYYCARGSYYYAM |
| | | DYWGQGTSVTVSS |

Antibody H-H (humanized) VHs binds to human TCRVβ 27

| | | |
|---|---|---|
| SEQ ID NO: 1131 | VH-1 | QVQLVQSGAEVKKPGSSVKVSCKASGFKTEDTYM |
| | | YWVRQAPGQGLEWIGRIDPANGNTKYDPKFQDRA |
| | | TITADSSTNTAYMELSSLRSEDTAVYYCARGSYYY |
| | | AMDYWGQGTLVTVSS |
| SEQ ID NO: 1132 | VH-2 | QVQLVQSGAEVKKPGASVKVSCKASGFKTEDTYM |
| | | YWVRQAPGQRLEWIGRIDPANGNTKYDPKFQDRA |
| | | TITADSSANTAYMELSSLRSEDTAVYYCARGSYYY |
| | | AMDYWGQGTLVTVSS |
| SEQ ID NO: 1133 | VH-3 | EVQLVESGGGLVQPGGSLKLSCAASGFKTEDTYMY |
| | | WVRQASGKGLEWIGRIDPANGNTKYDPKFQDRATI |
| | | SADSSKNTAYLQMNSLKTEDTAVYYCARGSYYYA |
| | | MDYWGQGTLVTVSS |
| SEQ ID NO: 1134 | VH-4 | EVQLVQSGAEVKKPGESLRISCKASGFKTEDTYMY |
| | | WVRQMPGKGLEWIGRIDPANGNTKYDPKFQDQATI |
| | | SADSSINTAYLQWSSLKASDTAMYYCARGSYYYA |
| | | MDYWGQGTLVTVSS |
| SEQ ID NO: 1135 | VH-5 | QVQLVQSGSELKKPGASVKVSCKASGFKTEDTYM |
| | | YWVRQAPGQGLEWIGRIDPANGNTKYDPKFQDRA |
| | | VISADSSVNTAYLQISSLKAEDTAVYYCARGSYYY |
| | | AMDYWGQGTLVTVSS |

Antibody H-H (humanized) VLs Binds to human TCRVβ 27

| | | |
|---|---|---|
| SEQ ID NO: 1136 | VL-1 | DIVLTQSPDSLAVSLGERATINCRASESVDSYGNSF |
| | | MHWYQQKPGQPPKLLIYRASNLESGVPDRFSGSGS |
| | | RTDFTLTISSLQAEDVAVYYCQQSNEDPYTFGQGTK |
| | | LEIK |
| SEQ ID NO: 1137 | VL-2 | EIVLTQSPATLSLSPGERATLSCRASESVDSYGNSFM |
| | | HWYQQKPGQAPKLLIYRASNLESGIPARFSGSGSRT |
| | | DFTLTISRLEPEDFAVYYCQQSNEDPYTFGQGTKLEI |
| | | K |
| SEQ ID NO: 1138 | VL-3 | DIQLTQSPSSLSASVGDRVTITCRASESVDSYGNSFM |
| | | HWYQQKPGQAPKLLIYRASNLESGVPSRFSGSGSRT |
| | | DFTLTISSLQPEDVATYYCQQSNEDPYTFGQGTKLEI |
| | | K |
| SEQ ID NO: 1139 | VL-4 | AIQLTQSPSSLSASVGDRVTITCRASESVDSYGNSFM |
| | | HWYQQKPGKAPKLLIYRASNLESGVPSRFSGSGSRT |
| | | DFTLTISSLQPEDFATYYCQQSNEDPYTFGQGTKLEI |
| | | K |
| SEQ ID NO: 1140 | VL-5 | EIVLTQSPDFQSVTPKEKVTITCRASESVDSYGNSFM |
| | | HWYQQKPDQSPKLLIYRASNLESGVPSRFSGSGSRT |
| | | DFTLTINSLEAEDAATYYCQQSNEDPYTFGQGTKLE |
| | | IK |

Antibody I(murine) binds to human TCRVβ 6-5, 6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3 (Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3 (Combined) | LQGSGFPLT |
| SEQ ID NO: 1151 | VL | ENVLTQSPAIMSASPGEKVTMTCSAGSSVSFMHWY |
| | | QQKSSTSPKLWIYDTSKLASGVPGRFSGSGSGNSFS |
| | | LTISSMEAEDVAIYYCLQGSGFPLTFGSGTKLEIK |
| SEQ ID NO: 1152 | VH | DVKLVESGEGLVKPGGSLKLSCAASGFTFRSYAMS |
| | | WVRQTPEKRLEWVAHISNGGDYIYYADTVKGRFTI |
| | | SRDNARNTLYLQMSSLKSEDTAMYYCTRPSYYSDP |
| | | WFFDVWGTGTTVTVSS |

Antibody I-H (humanized) VHs
Binds to human TCRVβ 6-5, 6-6, 6-9

| | | |
|---|---|---|
| SEQ ID NO: 1153 | VH-1 | EVQLVESGGGLVQPGGSLRLSCAASGFTFRSYAMS |
| | | WVRQAPGKGLEWVAHISNGGDYIYYADTVKGRFTI |
| | | SRDNAKNSLYLQMNSLRAEDTAVYYCTRPSYYSDP |
| | | WFFDVWGQGTTVTVSS |
| SEQ ID NO: 1154 | VH-2 | QVQLVESGGGVVQPGRSLRLSCAASGFTFRSYAMS |
| | | WVRQAPGKGLEWVAHISNGGDYIYYADTVKGRFTI |
| | | SRDNSKNTLYLQMSSLRAEDTAVYYCTRPSYYSDP |
| | | WFFDVWGQGTTVTVSS |
| SEQ ID NO: 1155 | VH-3 | EVQLVESGGGLVQPGGSLRLSCAASGFTFRSYAMS |
| | | WVRQAPGKGLEWVAHISNGGDYIYYADTVKGRFTI |
| | | SRDNSKNTLYLQMNSLRAEDTAVYYCTRPSYYSDP |
| | | WFFDVWGQGTTVTVSS |
| SEQ ID NO: 1156 | VH-4 | QVQLVQSGSELKKPGASVKVSCKASGFTFRSYAMS |
| | | WVRQAPGQGLEWVAHISNGGDYIYYADTVKGRFV |
| | | ISRDNSVNTLYLQISSLKAEDTAVYYCTRPSYYSDP |
| | | WFFDVWGQGTTVTVSS |
| SEQ ID NO: 1157 | VH-5 | QVQLVQSGAEVKKPGASVKVSCKASGFTFRSYAMS |
| | | WVRQAPGQRLEWVAHISNGGDYIYYADTVKGRFTI |
| | | TRDNSANTLYMELSSLRSEDTAVYYCTRPSYYSDP |
| | | WFFDVWGQGTTVTVSS |

Antibody I-H (humanized) VLs
Binds to human TCRVβ 6-5, 6-6, 6-9

| | | |
|---|---|---|
| SEQ ID NO: 1158 | VL-1 | ENVLTQSPATLSLSPGERATLSCSAGSSVSFMHWYQ |
| | | QKPGQAPKLLIYDTSKLASGIPARFSGSGSGNDFTLT |
| | | ISSLEPEDFAVYYCLQGSGFPLTFGQGTKLEIK |
| SEQ ID NO: 1159 | VL-2 | ENVLTQSPDFQSVTPKEKVTITCSAGSSVSFMHWYQ |
| | | QKPDQSPKLLIYDTSKLASGVPSRFSGSGSGNDFTLT |
| | | INSLEAEDAATYYCLQGSGFPLTFGQGTKLEIK |
| SEQ ID NO: 1160 | VL-3 | DNQLTQSPSSLSASVGDRVTITCSAGSSVSFMHWYQ |
| | | QKPGKVPKLLIYDTSKLASGVPSRFSGSGSGNDFTL |
| | | TISSLQPEDVATYYCLQGSGFPLTFGQGTKLEIK |
| SEQ ID NO: 1161 | VL-4 | ANQLTQSPSSLSASVGDRVTITCSAGSSVSFMHWYQ |
| | | QKPGKAPKLLIYDTSKLASGVPSRFSGSGSGNDFTL |
| | | TISSLQPEDFATYYCLQGSGFPLTFGQGTKLEIK |
| SEQ ID NO: 1162 | VL-5 | DNVLTQSPDSLAVSLGERATINCSAGSSVSFMHWY |
| | | QQKPGQPPKLLIYDTSKLASGVPDRFSGSGSGNDFT |
| | | LTISSLQAEDVAVYYCLQGSGFPLTFGQGTKLEIK |

Antibody IJ (murine), Binds to human TCRVβ 5-1

| | | |
|---|---|---|
| SEQ ID NO: 1163 | HC CDR1 (Kabat) | DYNIH |
| SEQ ID NO: 1164 | HC CDR2 (Kabat) | YINPYNGRTGYNQKFKA |
| SEQ ID NO: 1165 | HC CDR3 (Kabat) | WDGSSYFDY |
| SEQ ID NO: 1166 | HC CDR1 (Chothia) | GYTFTDYNIH |
| SEQ ID NO: 1167 | HC CDR2 (Chothia) | NPYNGR |
| SEQ ID NO: 1165 | HC CDR3 (Chothia) | WDGSSYFDY |
| SEQ ID NO: 1166 | HC CDR1 (Combined) | GYTFTDYNIH |
| SEQ ID NO: 1164 | HC CDR2 (Combined) | YINPYNGRTGYNQKFKA |
| SEQ ID NO: 1165 | HC CDR3 (Combined) | WDGSSYFDY |
| SEQ ID NO: 1168 | LC CDR1 (Kabat) | SASSSVSYMH |
| SEQ ID NO: 1169 | LC CDR2 (Kabat) | EISKLAS |
| SEQ ID NO: 1170 | LC CDR3 (Kabat) | QQWNYPLLT |
| SEQ ID NO: 1297 | LC CDR1 (Chothia) | SSSVSY |
| SEQ ID NO: 1169 | LC CDR2 (Chothia) | EISKLAS |
| SEQ ID NO: 1170 | LC CDR3 (Chothia) | QQWNYPLLT |
| SEQ ID NO: 1168 | LC CDR1 (Combined) | SASSSVSYMH |
| SEQ ID NO: 1169 | LC CDR2 (Combined) | EISKLAS |
| SEQ ID NO: 1170 | LC CDR3 (Combined) | QQWNYPLLT |
| SEQ ID NO: 1171 | VL | EIVLTQSPAITAASLGQKVTITCSAS SVSYMHWYQ |
| | | QKSGTSPKPWIYEISKLASGVPARFSGSGSGTSYSLT |
| | | ISSMEAEDAAIYYCQQWNYPLLTFGAGTKLELK |
| SEQ ID NO: 1172 | VH | EVQLQQSGPVLVKPGASVRMSCKASGYTFTDYNIH |
| | | WVKQSHGRSLEWVGYINPYNGRTGYNQKFKAKAT |
| | | LTVDKSSSTAYMDLRSLTSEDSAVYYCARWDGSSY |
| | | FDYWGQGTTLTVSS |

Antibody J-H(humanized) VHs
Binds to human TCRVβ 5-1

| | | |
|---|---|---|
| SEQ ID NO: 1173 | VH-1 | QVQLVQSGAEVKKPGSSVKVSCKASGYTFTDYNIH |
| | | WVRQAPGQGLEWVGYINPYNGRTGYNQKFKARAT |
| | | LTVDKSTSTAYMELSSLRSEDTAVYYCARWDGSSY |
| | | FDYWGQGTTVTVSS |
| SEQ ID NO: 1174 | VH-2 | QVQLVQSGAEVKKPGASVKVSCKASGYTFTDYNIH |
| | | WVRQAPGQGLEWVGYINPYNGRTGYNQKFKARAT |
| | | LTVDKSTSTAYMELRSLRSDDMAVYYCARWDGSS |
| | | YFDYWGQGTTVTVSS |
| SEQ ID NO: 1175 | VH-3 | QVQLVQSGAEVKKPGASVKVSCKASGYTFTDYNIH |
| | | WVRQATGQGLEWVGYINPYNGRTGYNQKFKARA |
| | | TLTVNKSISTAYMELSSLRSEDTAVYYCARWDGSS |
| | | YFDYWGQGTTVTVSS |
| SEQ ID NO: 1176 | VH-4 | EVQLVESGGGLVQPGRSLRLSCTASGYTFTDYNIH |
| | | WVRQAPGKGLEWVGYINPYNGRTGYNQKFKARAT |
| | | LSVDKSKSIAYLQMNSLKTEDTAVYYCARWDGSSY |
| | | FDYWGQGTTVTVSS |
| SEQ ID NO: 1177 | VH-5 | QVQLVQSGSELKKPGASVKVSCKASGYTFTDYNIH |
| | | WVRQAPGQGLEWVGYINPYNGRTGYNQKFKARA |
| | | VLSVDKSVSTAYLQISSLKAEDTAVYYCARWDGSS |
| | | YFDYWGQGTTVTVSS |

Antibody J-H (humanized) VLs
Binds to human TCRVβ 5-1

| | | |
|---|---|---|
| SEQ ID NO: 1178 | VL-1 | EIVLTQSPATLSLSPGERATLSCSASSSVSYMHWYQ |
| | | QKPGQAPKLLIYEISKLASGIPARFSGSGSGTDYTLTI |
| | | SSLEPEDFAVYYCQQWNYPLLTFGQGTKLEIK |
| SEQ ID NO: 1179 | VL-2 | EIVLTQSPATLSLSPGERATLSCSASSSVSYMHWYQ |
| | | QKPGQAPKLLIYEISKLASGIPARFSGSGSGTDYTLTI |
| | | SRLEPEDFAVYYCQQWNYPLLTFGQGTKLEIK |
| SEQ ID NO: 1180 | VL-3 | EIVLTQSPDFQSVTPKEKVTITCSASSSVSYMHWYQ |
| | | QKPDQSPKLLIYEISKLASGVPSRFSGSGSGTDYTLTI |
| | | NSLEAEDAATYYCQQWNYPLLTFGQGTKLEIK |
| SEQ ID NO: 1181 | VL-4 | DIQLTQSPSFLSASVGDRVTITCSASSSVSYMHWYQ |
| | | QKPGKAPKLLIYEISKLASGVPSRFSGSGSGTEYTLT |
| | | ISSLQPEDFATYYCQQWNYPLLTFGQGTKLEIK |
| SEQ ID NO: 1182 | VL-5 | AIQLTQSPSSLSASVGDRVTITCSASSSVSYMHWYQ |
| | | QKPGKAPKLLIYEISKLASGVPSRFSGSGSGTDYTLT |
| | | ISSLQPEDFATYYCQQWNYPLLTFGQGTKLEIK |
| SEQ ID NO: 1183 | VL-6 | AIRLTQSPFSLSASVGDRVTITCSASSSVSYMHWYQ |
| | | QKPAKAPKLFIYEISKLASGVPSRFSGSGSGTDYTLT |
| | | ISSLQPEDFATYYCQQWNYPLLTFGQGTKLEIK |
| SEQ ID NO: 1184 | VL-7 | DIVLTQSPDSLAVSLGERATINCSASSSVSYMHWYQ |
| | | QKPGQPPKLLIYEISKLASGVPDRFSGSGSGTDYTLT |
| | | ISSLQAEDVAVYYCQQWNYPLLTFGQGTKLEIK |

Antibody K (murine), binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3 (Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | SANSTVGYIH |
| SEQ ID NO: 1192 | LC CDR2 (Combined) | TTSNLAS |
| SEQ ID NO: 1193 | LC CDR3 (Combined) | HQWSFYPT |
| SEQ ID NO: 1195 | VL | QIVLTQSPAIMSASLGEEIALTCSANSTVGYIHWYQ |
| | | QKSGTSPKLLIYTTSNLASGVPSRFSGSGSGTFYSLTI |
| | | SSVEAEDAADYFCHQWSFYPTFGGGTKLEIK |
| SEQ ID NO: 1196 | VH | QIQLQQSGPEVVKPGASVQISCKASGYTFSGSWMN |
| | | WVKQRPGKGLEWIGRIYPGDGDTDYSGKFKGRAT |
| | | LTADKSSSTAYMRLSSLTSEDSAVYFCARSGYFNY |
| | | VPVFDYWGQGTTLSVSS |

Antibody K-H(humanized) VHs
Binds to human TCRVβ 28

| | | |
|---|---|---|
| SEQ ID NO: 1197 | VH-1 | QIQLVQSGAEVKKPGASVKVSCKASGYTFSGSWM |
| | | NWVRQAPGQGLEWIGRIYPGDGDTDYSGKFKGRA |
| | | TLTADKSTSTAYMELSSLRSEDTAVYYCARSGYFN |
| | | YVPVFDYWGQGTTVTVSS |
| SEQ ID NO: 1198 | VH-2 | QIQLVQSGAEVKKPGSSVKVSCKASGYTFSGSWMN |
| | | WVRQAPGQGLEWIGRIYPGDGDTDYSGKFKGRAT |
| | | LTADKSTSTAYMELSSLRSEDTAVYYCARSGYFNY |
| | | VPVFDYWGQGTTVTVSS |
| SEQ ID NO: 1199 | VH-3 | EIQLVQSGAEVKKPGESLKISCKASGYTFSGSWMN |
| | | WVRQMPGKGLEWIGRIYPGDGDTDYSGKFKGQAT |
| | | LSADKSISTAYLQWSSLKASDTAMYYCARSGYFNY |
| | | VPVFDYWGQGTTVTVSS |
| SEQ ID NO: 1200 | VH-4 | QIQLVQSGSELKKPGASVKVSCKASGYTFSGSWMN |
| | | WVRQAPGQGLEWIGRIYPGDGDTDYSGKFKGRAV |
| | | LSADKSVSTAYLQISSLKAEDTAVYYCARSGYFNY |
| | | VPVFDYWGQGTTVTVSS |
| SEQ ID NO: 1201 | VH-5 | QIQLVQSGSELKKPGASVKVSCKASGYTFSGSWMN |
| | | WVRQAPGQGLEWIGRIYPGDGDTDYSGKFKGRAV |
| | | LSADKSVSMAYLQISSLKAEDTAVYYCARSGYFNY |
| | | VPVFDYWGQGTTVTVSS |
| SEQ ID NO: 1202 | VH-6 | EIQLVESGGGLVQPGRSLRLSCTASGYTFSGSWMN |
| | | WVRQAPGKGLEWIGRIYPGDGDTDYSGKFKGRAT |
| | | LSADKSKSIAYLQMNSLKTEDTAVYYCARSGYFNY |
| | | VPVFDYWGQGTTVTVSS |

Antibody K-H (humanized) VLs
Binds to human TCRVβ 28

| | | |
|---|---|---|
| SEQ ID NO: 1203 | VL-1 | EIVLTQSPATLSLSPGERATLSCSANSTVGYIHWYQ |
| | | QKPGQAPKLLIYTTSNLASGIPARFSGSGSGTDYTLT |
| | | ISSLEPEDFAVYFCHQWSFYPTFGQGTKLEIK |
| SEQ ID NO: 1204 | VL-2 | DIQLTQSPSFLSASVGDRVTITCSANSTVGYIHWYQ |
| | | QKPGKAPKLLIYTTSNLASGVPSRFSGSGSGTEYTLT |
| | | ISSLQPEDFATYFCHQWSFYPTFGQGTKLEIK |
| SEQ ID NO: 1205 | VL-3 | EIVLTQSPATLSLSPGERATLSCSANSTVGYIHWYQ |
| | | QKPGQAPKLLIYTTSNLASGIPARFSGSGPGTDYTLT |
| | | ISSLEPEDFAVYFCHQWSFYPTFGQGTKLEIK |
| SEQ ID NO: 1206 | VL-4 | DIVLTQSPDSLAVSLGERATINCSANSTVGYIHWYQ |
| | | QKPGQPPKLLIYTTSNLASGVPDRFSGSGSGTDYTL |
| | | TISSLQAEDVAVYFCHQWSFYPTFGQGTKLEIK |
| SEQ ID NO: 1207 | VL-5 | EIVLTQSPDFQSVTPKEKVTITCSANSTVGYIHWYQ |
| | | QKPDQSPKLLIYTTSNLASGVPSRFSGSGSGTDYTLT |
| | | INSLEAEDAATYFCHQWSFYPTFGQGTKLEIK |

Antibody L (murine), binds to human TCRVβ 4-1, 4-2, 4-3

| | | |
|---|---|---|
| SEQ ID NO: 1208 | HC CDR1 (Kabat) | DYYMY |
| SEQ ID NO: 1209 | HC CDR2 (Kabat) | TISGGGSYTYSPDSVKG |
| SEQ ID NO: 1210 | HC CDR3 (Kabat) | ERDIYYGNFNAMVY |
| SEQ ID NO: 1211 | HC CDR1 (Chothia) | GFTFSDY |
| SEQ ID NO: 1212 | HC CDR2 (Chothia) | SGGGSY |
| SEQ ID NO: 1210 | HC CDR3 (Chothia) | ERDIYYGNFNAMVY |
| SEQ ID NO: 1213 | HC CDR1 (Combined) | GFTFSDYYMY |
| SEQ ID NO: 1209 | HC CDR2 (Combined) | TISGGGSYTYSPDSVKG |
| SEQ ID NO: 1210 | HC CDR3 (Combined) | ERDIYYGNFNAMVY |
| SEQ ID NO: 1214 | LC CDR1 (Kabat) | RASKSVSTSGYSYMH |
| SEQ ID NO: 1215 | LC CDR2 (Kabat) | LASNLES |
| SEQ ID NO: 1216 | LC CDR3 (Kabat) | QHSRDLPWT |
| SEQ ID NO: 1217 | LC CDR1 (Chothia) | SKSVSTSGYSY |
| SEQ ID NO: 1215 | LC CDR2 (Chothia) | LASNLES |
| SEQ ID NO: 1216 | LC CDR3 (Chothia) | QHSRDLPWT |
| SEQ ID NO: 1214 | LC CDR1 (Combined) | RASKSVSTSGYSYMH |
| SEQ ID NO: 1215 | LC CDR2 (Combined) | LASNLES |
| SEQ ID NO: 1216 | LC CDR3 (Combined) | QHSRDLPWT |
| SEQ ID NO: 1218 | VL | DIVLTQSPVSLTVSLGQRATISCRASKSVSTSGYSYM |
| | | HWYQQKPGQPPKLLIYLASNLESGVPARFSGSGSGT |
| | | DFTLNIHPVEEEDAATYYCQHSRDLPWTFGGGTKL |
| | | EIK |
| SEQ ID NO: 1219 | VH | EVQLVESGGGLVKPGGSLKLSCAASGFTFSDYYMY |
| | | WVRQTPEKRLEWVATISGGGSYTYSPDSVKGRFTIS |
| | | RDNAKNNLYLQMSSLRSEDTAMYFCARERDIYYG |
| | | NFNAMVYWGRGTSVTVSS |

Antibody L-H (humanized) VHs
Binds to human TCRVβ 4-1, 4-2, 4-3

| | | |
|---|---|---|
| SEQ ID NO: 1220 | VH-1 | EVQLLESGGGLVQPGGSLRLSCAASGFTFSDYYMY |
| | | WVRQAPGKGLEWVATISGGGSYTYSPDSVKGRFTI |
| | | SRDNSKNTLYLQMNSLRAEDTAVYYCARERDIYYG |
| | | NFNAMVYWGRGTLVTVSS |
| SEQ ID NO: 1221 | VH-2 | EVQLVESGGGLVQPGGSLRLSCAASGFTFSDYYMY |
| | | WVRQAPGKGLEWVATISGGGSYTYSPDSVKGRFTI |
| | | SRDNAKNSLYLQMNSLRAEDTAVYYCARERDIYY |
| | | GNFNAMVYWGRGTLVTVSS |
| SEQ ID NO: 1222 | VH-3 | QVQLVESGGGVVQPGRSLRLSCAASGFTFSDYYM |
| | | YWVRQAPGKGLEWVATISGGGSYTYSPDSVKGRF |
| | | TISRDNSKNTLYLQMNSLRAEDTAVYYCARERDIY |
| | | YGNFNAMVYWGRGTLVTVSS |
| SEQ ID NO: 1223 | VH-4 | QVQLVESGGGLVKPGGSLRLSCAASGFTFSDYYMY |
| | | WIRQAPGKGLEWVATISGGGSYTYSPDSVKGRFTIS |
| | | RDNAKNSLYLQMNSLRAEDTAVYYCARERDIYYG |
| | | NFNAMVYWGRGTLVTVSS |

Antibody L-H(humanized) VLs
Binds to human TCRVβ 4-1, 4-2, 4-3

| | | |
|---|---|---|
| SEQ ID NO: 1224 | VL-1 | EIVLTQSPGTLSLSPGERATLSCRASKSVSTSGYSYM |
| | | HWYQQKPGQAPRLLIYLASNLESGIPDRFSGSGSGT |
| | | DFTLTISRLEPEDFAVYYCQHSRDLPWTFGGGTKVE |
| | | IK |
| SEQ ID NO: 1225 | VL-2 | EIVLTQSPATLSLSPGERATLSCRASKSVSTSGYSYM |
| | | HWYQQKPGQAPRLLIYLASNLESGIPARFSGSGSGT |
| | | DFTLTISSLEPEDFAVYYCQHSRDLPWTFGGGTKVE |
| | | IK |
| SEQ ID NO: 1226 | VL-3 | DIQLTQSPSTLSASVGDRVTITCRASKSVSTSGYSYM |
| | | HWYQQKPGKAPKLLIYLASNLESGVPSRFSGSGSGT |
| | | EFTLTISSLQPDDFATYYCQHSRDLPWTFGGGTKVE |
| | | IK |
| SEQ ID NO: 1227 | VL-4 | AIQLTQSPSSLSASVGDRVTITCRASKSVSTSGYSYM |
| | | HWYQQKPGKAPKLLIYLASNLESGVPSRFSGSGSGT |
| | | DFTLTISSLQPEDFATYYCQHSRDLPWTFGGGTKVE |
| | | IK |

Antibody M (murine), binds to human TCRVβ 19

| | | |
|---|---|---|
| SEQ ID NO: 1229 | HC CDR1 (Kabat) | GYFWN |
| SEQ ID NO: 1230 | HC CDR2 (Kabat) | YISYDGSNNYNPSLKN |
| SEQ ID NO: 1231 | HC CDR3 (Kabat) | PSPGTGYAVDY |
| SEQ ID NO: 1232 | HC CDR1 (Chothia) | GYSITSGY |
| SEQ ID NO: 1233 | HC CDR2 (Chothia) | SYDGSN |
| SEQ ID NO: 1231 | HC CDR3 (Chothia) | PSPGTGYAVDY |
| SEQ ID NO: 1234 | HC CDR1 (Combined) | GYSITSGYFWN |
| SEQ ID NO: 1230 | HC CDR2 (Combined) | YISYDGSNNYNPSLKN |
| SEQ ID NO: 1231 | HC CDR3 (Combined) | PSPGTGYAVDY |
| SEQ ID NO: 1235 | LC CDR1 (Kabat) | RSSQSLVHSNGNTYLH |
| SEQ ID NO: 1236 | LC CDR2 (Kabat) | KVSNRFS |
| SEQ ID NO: 1237 | LC CDR3 (Kabat) | SQSTHVPFT |
| SEQ ID NO: 1238 | LC CDR1 (Chothia) | SQSLVHSNGNTY |
| SEQ ID NO: 1236 | LC CDR2 (Chothia) | KVSNRFS |
| SEQ ID NO: 1237 | LC CDR3 (Chothia) | SQSTHVPFT |
| SEQ ID NO: 1235 | LC CDR1 (Combined) | RSSQSLVHSNGNTYLH |
| SEQ ID NO: 1236 | LC CDR2 (Combined) | KVSNRFS |
| SEQ ID NO: 1237 | LC CDR3 (Combined) | SQSTHVPFT |
| SEQ ID NO: 1239 | VL | NVVMTQTPLSLPVSLGDQASISCRSSQSLVHSNGNT |
| | | YLHWYLQKPGQSPKFLIYKVSNRFSGVPDRFSGGG |
| | | SGTEFTLKISRVEAEDLGVYFCSQSTHVPFTFGSGTK |
| | | LEIK |
| SEQ ID NO: 1240 | VH | NVQLQESGPGLVKPSQSLSLTCSVAGYSITSGYFWN |
| | | WIRQFPGNKLEWMGYISYDGSNNYNPSLKNRISITR |
| | | DTSKNQFFLKLNSVTTEDTATYYCASPSPGTGYAV |
| | | DYWGQGTSVTVSS |

Antibody M-H (humanized) VHs
Binds to human TCRVβ 19

| | | |
|---|---|---|
| SEQ ID NO: 1241 | VH-1 | QVQLQESGPGLVKPSETLSLTCTVSGYSITSGYFWN |
| | | WIRQPPGKGLEWIGYISYDGSNNYNPSLKNRVTISR |
| | | DTSKNQFSLKLSSVTAADTAVYYCASPSPGTGYAV |
| | | DYWGQGTLVTVSS |
| SEQ ID NO: 1242 | VH-2 | QVQLQESGPGLVKPSETLSLTCTVSGYSITSGYFWN |
| | | WIRQPPGKGLEWIGYISYDGSNNYNPSLKNRVTISR |
| | | DTSKNQFSLKLSSVTAADTAVYYCASPSPGTGYAV |
| | | DYWGQGTLVTVSS |
| SEQ ID NO: 1243 | VH-3 | QVQLVESGGGLVQPGGSLRLSCSVSGYSITSGYFW |
| | | NWVRQAPGKGLEWVGYISYDGSNNYNPSLKNRFTI |
| | | SRDTSKNTFYLQMNSLRAEDTAVYYCASPSPGTGY |
| | | AVDYWGQGTLVTVSS |

Antibody M-H (humanized) VLs
Binds to human TCRVβ 19

| | | |
|---|---|---|
| SEQ ID NO: 1244 | VL-1 | VVMTQSPGTLSLSPGERATLSCRSSQSLVHSNGNTY |
| | | LHWYQQKPGQAPRFLIYKVSNRFSGIPDRFSGSGSG |
| | | TDFTLTISRLEPEDFAVYFCSQSTHVPFTFGQGTKLE |
| | | IK |
| SEQ ID NO: 1245 | VL-2 | EVVMTQSPATLSLSPGERATLSCRSSQSLVHSNGNT |
| | | YLHWYQQKPGQAPRFLIYKVSNRFSGIPARFSGSGS |
| | | GTDFTLTISSLEPEDFAVYFCSQSTHVPFTFGQGTKL |
| | | EIK |
| SEQ ID NO: 1246 | VL-3 | EVVMTQSPATLSVSPGERATLSCRSSQSLVHSNGNT |
| | | YLHWYQQKPGQAPRFLIYKVSNRFSGIPARFSGSGS |
| | | GTEFTLTISSLQSEDFAVYFCQ STHVPFTFGQGTKL |
| | | EIK |
| SEQ ID NO: 1247 | VL-4 | DVQMTQSPSSLSASVGDRVTITCRSSQSLVHSNGNT |
| | | YLHWYQQKPGKAPKFLIYKVSNRFSGVPSRFSS GS |
| | | GTDFTFTISSLQPEDIATYFCSQSTHVPFTFGQGTKL |
| | | EIK |

Antibody N(murine), binds to human TCRVβ 9

| | | |
|---|---|---|
| SEQ ID NO: 1248 | HC CDR1 (Kabat) | DYIVH |
| SEQ ID NO: 1249 | HC CDR2 (Kabat) | WINTYTGTPTYADDFEG |
| SEQ ID NO: 1250 | HC CDR3 (Kabat) | SWRRGIRGIGFDY |
| SEQ ID NO: 1251 | HC CDR1 (Chothia) | GYTFTDY |
| SEQ ID NO: 1252 | HC CDR2 (Chothia) | NTYTGT |
| SEQ ID NO: 1250 | HC CDR3 (Chothia) | SWRRGIRGIGFDY |
| SEQ ID NO: 1253 | HC CDR1 (Combined) | GYTFTDYIVH |
| SEQ ID NO: 1249 | HC CDR2 (Combined) | WINTYTGTPTYADDFEG |
| SEQ ID NO: 1250 | HC CDR3 (Combined) | SWRRGIRGIGFDY |
| SEQ ID NO: 1254 | LC CDR1 (Kabat) | KASKSINKYLA |
| SEQ ID NO: 1255 | LC CDR2 (Kabat) | DGSTLQS |
| SEQ ID NO: 1256 | LC CDR3 (Kabat) | QQHNEYPPT |
| SEQ ID NO: 1257 | LC CDR1 (Chothia) | SKSINKY |
| SEQ ID NO: 1255 | LC CDR2 (Chothia) | DGSTLQS |
| SEQ ID NO: 1256 | LC CDR3 (Chothia) | QQHNEYPPT |
| SEQ ID NO: 1254 | LC CDR1 (Combined) | KASKSINKYLA |
| SEQ ID NO: 1255 | LC CDR2 (Combined) | DGSTLQS |
| SEQ ID NO: 1256 | LC CDR3 (Combined) | QQHNEYPPT |
| SEQ ID NO: 1258 | VL | DVQMTQSPYNLAASPGESVSINCKASKSINKYLAW |
| | | YQQKPGKPNKLLIYDGSTLQSGIPSRFSGSGSGTDFT |
| | | LTIRGLEPEDFGLYYCQQHNEYPPTFGAGTKLELK |
| SEQ ID NO: 1259 | VH | QLQLVQSGPELREPGESVKISCKASGYTFTDYIVHW |
| | | VKQAPGKGLKWMGWINTYTGTPTYADDFEGRFVF |
| | | SLEASASTANLQISNLKNEDTATYFCARSWRRGIRG |
| | | IGFDYWGQGVMVTVSS |

Antibody N-H (humanized) VH's
Binds to human TCRVβ 9

| | | |
|---|---|---|
| SEQ ID NO: 1260 | VH-1 | QLQLVQSGAEVKKPGASVKVSCKASGYTFTDYIVH |
| | | WVRQAPGQGLEWMGWINTYTGTPTYADDFEGWV |
| | | TMTLDASISTAYMELSRLRSDDTAVYYCARSWRRG |
| | | IRGIGFDYWGQGTMVTVSS |
| SEQ ID NO: 1261 | VH-2 | QLQLVQSGAEVKKPGASVKVSCKASGYTFTDYIVH |
| | | WVRQAPGQGLEWMGWINTYTGTPTYADDFEGRVT |
| | | MTLDASTSTAYMELSSLRSEDTAVYYCARSWRRGI |
| | | RGIGFDYWGQGTMVTVSS |
| SEQ ID NO: 1262 | VH-3 | QLQLVQSGAEVKKPGASVKVSCKASGYTFTDYIVH |
| | | WVRQAPGQRLEWMGWINTYTGTPTYADDFEGRVT |
| | | ITLDASASTAYMELSSLRSEDMAVYYCARSWRRGI |
| | | RGIGFDYWGQGTMVTVSS |
| SEQ ID NO: 1263 | VH-4 | QLQLVQSGAEVKKPGASVKVSCKASGYTFTDYIVH |
| | | WVRQATGQGLEWMGWINTYTGTPTYADDFEGRV |
| | | TMTLNASISTAYMELSSLRSEDTAVYYCARSWRRGI |
| | | RGIGFDYWGQGTMVTVSS |

Antibody N-H (humanized) VL's
Binds to human TCRVβ 9

| | | |
|---|---|---|
| SEQ ID NO: 1264 | VL-1 | EVVMTQSPGTLSLSPGERATLSCKASKSINKYLAW |
| | | YQQKPGQAPRLLIYDGSTLQSGIPDRFSGSGSGTDFT |
| | | LTISRLEPEDFAVYYCQQHNEYPPTFGQGTKLEIK |
| SEQ ID NO: 1265 | VL-2 | EVVMTQSPATLSLSPGERATLSCKASKSINKYLAW |
| | | YQQKPGQAPRLLIYDGSTLQSGIPARFSGSGSGTDFT |
| | | LTISSLEPEDFAVYYCQQHNEYPPTFGQGTKLEIK |
| SEQ ID NO: 1266 | VL-3 | DVQMTQSPSSLSASVGDRVTITCKASKSINKYLAW |
| | | YQQKPGKAPKLLIYDGSTLQSGVPSRFSGSGSGTDF |
| | | TLTISSLQPEDFATYYCQQHNEYPPTFGQGTKLEIK |
| SEQ ID NO: 1267 | VL-4 | AVRMTQSPSSFSASTGDRVTITCKASKSINKYLAWY |
| | | QQKPGKAPKLLIYDGSTLQSGVPSRFSGSGSGTDFT |
| | | LTISCLQSEDFATYYCQQHNEYPPTFGQGTKLEIK |

Antibody O (murine) binds to TRVβ 11-2

| | | |
|---|---|---|
| SEQ ID NO: 1268 | HC CDR1 (Kabat) | NYGVH |
| SEQ ID NO: 1269 | HC CDR2 (Kabat) | VIWSDGSTDYDTAFIS |
| SEQ ID NO: 1270 | HC CDR3 (Kabat) | RAVVADFDY |
| SEQ ID NO: 1271 | HC CDR1 (Chothia) | GFSLTN |
| SEQ ID NO: 1272 | HC CDR2 (Chothia) | VIWSDGSTD |
| SEQ ID NO: 1270 | HC CDR3 (Chothia) | RAVVADFDY |
| SEQ ID NO: 1273 | HC CDR1 (combined) | GFSLTNYGVH |
| SEQ ID NO: 1269 | HC CDR2 (combined) | VIWSDGSTDYDTAFIS |
| SEQ ID NO: 1270 | HC CDR3 (combined) | RAVVADFDY |
| SEQ ID NO: 1274 | VH | QVQLKQSGPGLLQPSQSLSITCTVSGFSLTNYGVHW |
| | | VRQSPGKGLEWLGVIWSDGSTDYDTAFISRLSISKD |
| | | NSKSQVFFKLNSLQADDTAIYYCARRAVVADFDY |
| | | WGQGTTLTVSS |
| SEQ ID NO: 1275 | LC CDR1 (Kabat) | KASKEVTIFGSISALH |
| SEQ ID NO: 1276 | LC CDR2 (Kabat) | NGAKLES |
| SEQ ID NO: 1277 | LC CDR3 (Kabat) | LQNKEVPFT |
| SEQ ID NO: 1275 | LC CDR1 (Chothia) | KASKEVTIFGSISALH |
| SEQ ID NO: 1276 | LC CDR2 (Chothia) | NGAKLES |
| SEQ ID NO: 1277 | LC CDR3 (Chothia) | LQNKEVPFT |
| SEQ ID NO: 1275 | LC CDR1 (combined) | KASKEVTIFGSISALH |
| SEQ ID NO: 1276 | LC CDR2 (combined) | NGAKLES |
| SEQ ID NO: 1277 | LC CDR3 (combined) | LQNKEVPFT |
| SEQ ID NO: 1278 | VL | DIVLTQSPASLAVSLGQKATISCKASKEVTIFGSISA |
| | | LHWYQQKPGQPPKLIYNGAKLESGVSARFSDSGSQ |
| | | NRSPFGNQLSFTLTIAPVEADDAATYYCLQNKEVP |
| | | FTFGSGTKLEIK |

Antibody O-H (humanized) VL binds to TRvβ 11-2

| | | |
|---|---|---|
| SEQ ID NO: 1279 | VL-1 | DIVLTQSPDSLAVSLGERATINCKASKEVTIFGSISA |
| | | LHWYQQKPGQPPKLLYNGAKLESGVSARFGVPDR |
| | | FSRSGSGLDFTLTISSLQAEDVAVYYCLQNKEVPFT |
| | | FGQGTKLEIK |
| SEQ ID NO: 1280 | VL-2 | EIVLTQSPDFQSVTPKEKVTITCKASKEVTIFGSISA |
| | | LHWYQQKPDQSPKLLYNGAKLESGVSARFGVPSR |
| | | FSRSGSGLDFTLTINSLEAEDAATYYCLQNKEVPFT |
| | | FGQGTKLEIK |
| SEQ ID NO: 1281 | VL-3 | AIQLTQSPSSLSASVGDRVTITCKASKEVTIFGSISA |
| | | LHWYQQKPGKAPKLLYNGAKLESGVSARFGVPSR |
| | | FSRSGSGLDFTLTISSLQPEDFATYYCLQNKEVPFTF |
| | | GQGTKLEIK |
| SEQ ID NO: 1282 | VL-4 | DIVLTQTPLSLSVTPGQPASISCKASKEVTIFGSISAL |
| | | HWYLQKPGQPPKLLYNGAKLESGVSARFGVPDRF |
| | | SRSGSGLDFTLKISRVEAEDVGVYYCLQNKEVPFT |
| | | FGQGTKLEIK |

Antibody O-H (humanized) VH, binds to TRvβ 11-2

| | | |
|---|---|---|
| SEQ ID NO: 1283 | VH-1 | QVTLKESGPVLVKPTETLTLTCTVSGFSLTNYGVH |
| | | WVRQPPGKALEWLGVIWSDGSTDYDTAFISRLTIS |
| | | KDNSKSQVVLTMTNMDPVDTATYYCARRAVVAD |
| | | FDYWGQGTTVTVSS |
| SEQ ID NO: 1284 | VH-2 | QVQLQESGPGLVKPSGTLSLTCAVSGFSLTNYGVH |
| | | WVRQPPGKGLEWLGVIWSDGSTDYDTAFISRLTIS |
| | | KDNSKSQVSLKLSSVTAADTAVYYCARRAVVADF |
| | | DYWGQGTTVTVSS |
| SEQ ID NO: 1285 | VH-3 | QVQLQQSGPGLVKPSQTLSLTCAVSGFSLTNYGVH |
| | | WVRQSPSRGLEWLGVIWSDGSTDYDTAFISRLTIN |
| | | KDNSKSQVSLQLNSVTPEDTAVYYCARRAVVADF |
| | | DYWGQGTTVTVSS |
| SEQ ID NO: 1286 | VH-4 | EVQLVESGGGLVQPGPSLRLSCTVSGFSLTNYGVH |
| | | WVRQAPGKGLEWLGVIWSDGSTDYDTAFISRLTIS |
| | | KDNSKSIVYLQMNSLKTEDTAVYYCARRAVVADF |
| | | DYWGQGTTVTVSS |
| SEQ ID NO: 1287 | VH-5 | EVQLVQSGAEVKKPGESLRISCKVSGFSLTNYGVH |
| | | WVRQMPGKGLEWLGVIWSDGSTDYDTAFISQLTIS |
| | | KDNSISTVYLQWSSLKASDTAMYYCARRAVVADF |
| | | DYWGQGTTVTVSS |

IMMU546 binds to TRBV 2

### Cytokine Molecules and Cytokine Inhibitor Molecules

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

The present disclosure provides, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-la, IL-10, IL-2, IL-3, IL-4, IL-5, IL-6, IL-7, IL-8, IL-10, IL-12, IL-21, IFN-α, IFN-γ, MIP-la, MIP-10, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-la, MIP-1β and TGF-β. In one embodiment the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In one embodiment, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In one embodiment the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In one embodiment the mutant IL-2 cytokine additionally comprises an amino acid mutation at a position corresponding to position 3 of human IL-2, which eliminates the 0-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

In a specific embodiment the IL-2 cytokine of the multispecific or multifunctional polypeptide comprises the polypeptide sequence of SEQ ID NO: 7227 [APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFAQSIISTLT]. In another specific embodiment the IL-2 cytokine of the multispecific or multifunctional polypeptide comprises the polypeptide sequence of SEQ ID NO: 7228

[APASSSTKKTQLQLEHLLLDLQMILNGINNYKNPKLTRMLTAKFAMPK
KATELKHLQCLEEELKPLEEVLNGAQSKNFHLRPRDLISNINVIVLELK
GSETTFMCEYADETATIVEFLNRWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 7229 [IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSGR FTCWWLTTISTDLTFSVKSSRGSSDPQGVTCGAATLSAERVRGDNKEYEYSVECQEDSA CPAAEESLPIEVMVDAVHKLKYENYTSSFFIRDIIKPDPPKNLQLKPLKNSRQVEVSWEY PDTWSTPHSYFSLTFCVQVQGKSKREKKDRVFTDKTSATVICRKNASISVRAQDRYYSS SWSEWASVPCSGGGGSGGGGSGGGGSRNLPVATPDPGMFPCLHESQNLLRAVSNMLQ KARQTLEFYPCTSEEIDHEDITKDKTSTVEACLPLELTKNESCLNSRETSFITNGSCLASRK TSFMMALCLSSIYEDLKMYQVEFKTMNAKLLMDPKRQIFLDQNMLAVIDELMQALNFN SETVPQKSSLEEPDFYKTKIKLCILLHAFRIRAVTIDRVMSYLNAS]. In one embodiment, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 7230 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENK SKAVEQVKNAFNKLQEKGIYKAMSEFDIFINYIEAYMTMKIRNGGGGSGGGGSGGGGS GGGGSSPGQGTQSENSCTHFPGNLPNMLRDLRDAFSRVKTFFQMKDQLDNLLLKESLLE DFKGYLGCQALSEMIQFYLEEVMPQAENQDPDIKAHVNSLGENLKTLRLRLRRCHRFLP CENKSKAVEQVKNAFNKLQEKGIYKAMSEFDIFINYIEAYMTMKIRN]. In another specific embodiment the IL-10 cytokine is a monomeric IL-10 cytokine. In a more specific embodiment the monomeric IL-10 cytokine comprises the polypeptide sequence of SEQ ID NO: 7231 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENG GGSGGKSKAVEQVKNAFNKLQEKGIYKAMSEFDIFINYIEAYMTMKIRN]. In one embodiment, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In one embodiment the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In one embodiment the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In one embodiment the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 7232 [NWVNVISDLKKIEDLIQSMHIDATLYTESDVHPSCKVTAMKCFLLELQVISLASGDASIH DTVENLIILANNSLSSNGAVTESGCKECEELEEKNIKEFLQSFVHIVQMFINTS]. In one embodiment, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In one embodiment, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In one embodiment the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In one embodiment the cytokine, particularly a single-chain cytokine, of the multispecific or multifunctional polypeptide is IL-7. In a specific embodiment, the IL-7 cytokine can elicit proliferation of T and/or B lymphocytes. In one embodiment, the cytokine, particularly a single-chain cytokine, of the multispecific or multifunctional polypeptide is IL-8. In a specific embodiment, the IL-8 cytokine can elicit chemotaxis in neutrophils. In one embodiment, the cytokine, particularly a single-chain cytokine, of the multispecific or multifunctional polypeptide, is MIP-la. In a specific embodiment, the MIP-la cytokine can elicit chemotaxis in monocytes and T lymphocyte cells. In one embodiment, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In one embodiment, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In one embodiment, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules disclosed herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In one embodiment, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 7017), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7017.

In some embodiments, the cytokine molecule comprises a receptor dimerizing domain, e.g., an IL15Ralpha dimerizing domain. In one embodiment, the IL15Ralpha dimerizing domain comprises the amino acid sequence: MAPRRARGCRTLGLPALLLLLLLRPPATRGITCPPPMSVEHADIWVKSYSLYSRERYICN SGFKRKAGTSSLTECVL (SEQ ID NO: 7018), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7018. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 7019). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 7020), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7020).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGM AVTISVKCEKISTLSCENKIISFKEMNPPDNIKDTKSDIIFFQRSVPGHDNKMQFESSSYEG YFLACEKERDLFKLILKKEDELGDRSIMFTVQNED (SEQ ID NO: 7021), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7021).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKMI HQHLSSRTHGSEDS (SEQ ID NO: 7022), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7022).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIIVIQSQIVSFYFKLFK NFKDDQSIQKSVETIKEDMNVKFFNSNKKKRDDFEKLTNYSVTDLNVQRKAIHELIQVM AELSPAAKTGKRKRSQMLFRG (SEQ ID NO: 7023), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7023).

### TGF-Beta Inhibitors

The present disclosure further provides, inter alia, multispecific (e.g., bi-, tri-, quad-specific) or multifunctional molecules, that include, e.g., are engineered to contain, one or more cytokine inhibitor molecules, e.g., inhibitors of immunomodulatory (e.g., proinflammatory) cytokines and variants, e.g., functional variants, thereof. Accordingly, in some embodiments, the cytokine inhibitor molecule is a TGF-beta inhibitor. In some embodiments, the TGF-beta inhibitor binds to and inhibits TGF-beta, e.g., reduces the activity of TGF-beta. In some embodiments, the TGF-beta inhibitor inhibits (e.g., reduces the activity of) TGF-beta 1. In some embodiments, the TGF-beta inhibitor inhibits (e.g., reduces the activity of) TGF-beta 2. In some embodiments, the TGF-beta inhibitor inhibits (e.g., reduces the activity of) TGF-beta 3. In some embodiments, the TGF-beta inhibitor inhibits (e.g., reduces the activity of) TGF-beta 1 and TGF-beta 3. In some embodiments, the TGF-beta inhibitor inhibits (e.g., reduces the activity of) TGF-beta 1, TGF-beta 2, and TGF-beta 3.

In some embodiments, the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof. In some embodiments, the TGF-beta inhibitor comprises a TGFBR1 polypeptide (e.g., an extracellular domain of TGFBR1 or functional variant thereof). In some embodiments, the TGF-beta inhibitor comprises a TGFBR2 polypeptide (e.g., an extracellular domain of TGFBR2 or functional variant thereof). In some embodiments, the TGF-beta inhibitor comprises a TGFBR3 polypeptide (e.g., an extracellular domain of TGFBR3 or functional variant thereof). In some embodiments, the TGF-beta inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-beta inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-beta inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-beta receptor polypeptides that can be used as TGF-beta inhibitors have been disclosed in U.S. Pat. Nos. 8,993,524, 9,676,863, 8,658,135, US20150056199, US20070184052, and WO2017037634, all of which are herein incorporated by reference in their entirety.

In some embodiments, the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor comprises an extracellular domain of SEQ ID NO: 7257, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7258, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7259, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7266, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7267, or a sequence substantially identical thereto (e.g., a sequence that is at least 80%, 85%, 90%, or 95% identical thereto).

In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of SEQ ID NO: 7260, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7261, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7262, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7263, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7264, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7265, or a sequence substantially identical thereto (e.g., a sequence that is at least 80%, 85%, 90%, or 95% identical thereto).

In some embodiments, the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor comprises an extracellular domain of SEQ ID NO: 7268, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7269, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7270, or a sequence substantially identical thereto (e.g., a sequence that is at least 80%, 85%, 90%, or 95% identical thereto).

In some embodiments, the TGF-beta inhibitor comprises no more than one TGF-beta receptor extracellular domain. In some embodiments, the TGF-beta inhibitor comprises two or more (e.g., two, three, four, five, or more) TGF-beta receptor extracellular domains, linked together, e.g., via a linker.

TABLE 16
Exemplary amino acid sequences of TGF-beta polypeptides or TGF-beta receptor
polypeptides

| SEQ ID NO | Description | Amino acid sequence |
|---|---|---|
| SEQ ID NO: | Immature human | MPPSGLRLLLLLLPLLWLLVLTPGRPAAGLSTCKTIDMELV |
| 7254 | TGF-beta 1 | KRKRIEAIRGQILSKLRLASPPSQGEVPPGPLPEAVLALYNS |
| | (P01137-1) | TRDRVAGESAEPEPEPEADYYAKEVTRVLMVETHNEIYDK |
| | | FKQSTHSIYMFFNTSELREAVPEPVLLSRAELRLLRLKLKVE |
| | | QHVELYQKYSNNSWRYLSNRLLAPSDSPEWLSFDVTGVVR |
| | | QWLSRGGEIEGFRLSAHCSCDSRDNTLQVDINGFTTGRRGD |
| | | LATIHGMNRPFLLLMATPLERAQHLQSSRHRRALDTNYCF |
| | | SSTEKNCCVRQLYIDFRKDLGWKWIHEPKGYHANFCLGPC |
| | | PYIWSLDTQYSKVLALYNQHNPGASAAPCCVPQALEPLPIV |
| | | YYVGRKPKVEQLSNMIVRSCKCS |
| SEQ ID NO: | Human TGF-beta | LSTCKTIDMELVKRKRIEAIRGQILSKLRLASPPSQGEVPPGP |
| 7271 | 1 (P01137-1) | LPEAVLALYNSTRDRVAGESAEPEPEPEADYYAKEVTRVL |
| | | MVETHNEIYDKFKQSTHSIYMFFNTSELREAVPEPVLLSRA |
| | | ELRLLRLKLKVEQHVELYQKYSNNSWRYLSNRLLAPSDSP |
| | | EWLSFDVTGVVRQWLSRGGEIEGFRLSAHCSCDSRDNTLQ |
| | | VDINGFTTGRRGDLATIHGMNRPFLLLMATPLERAQHLQSS |
| | | RHRRALDTNYCFSSTEKNCCVRQLYIDFRKDLGWKWIHEP |
| | | KGYHANFCLGPCPYIWSLDTQYSKVLALYNQHNPGASAAP |
| | | CCVPQALEPLPIVYYVGRKPKVEQLSNMIVRSCKCS |
| SEQ ID NO: | Immature human | MHYCVLSAFLILHLVTVALSLSTCSTLDMDQFMRKRIEAIR |
| 7255 | TGF-beta 2 | GQILSKLKLTSPPEDYPEPEEVPPEVISIYNSTRDLLQEKASR |
| | (P61812-1) | RAAACERERSDEEYYAKEVYKIDMPPFFPSENAIPPTFYRP |
| | | YFRIVRFDVSAMEKNASNLVKAEFRVFRLQNPKARVPEQRI |
| | | ELYQILKSKDLTSPTQRYIDSKVVKTRAEGEWLSFDVTDAV |
| | | HEWLHHKDRNLGFKISLHCPCCTFVPSNNYIIPNKSEELEAR |
| | | FAGIDGTSTYTSGDQKTIKSTRKKNSGKTPHLLLMLLPSYR |
| | | LESQQTNRRKKRALDAAYCFRNVQDNCCLRPLYIDFKRDL |
| | | GWKWIHEPKGYNANFCAGACPYLWSSDTQHSRVLSLYNTI |
| | | NPEASASPCCVSQDLEPLTILYYIGKTPKIEQLSNMIVKSCK |
| | | CS |
| SEQ ID NO: | Human TGF-beta | LSTCSTLDMDQFMRKRIEAIRGQILSKLKLTSPPEDYPEPEE |
| 7272 | 2 (P61812-1) | VPPEVISIYNSTRDLLQEKASRRAAACERERSDEEYYAKEV |
| | | YKIDMPPFFPSENAIPPTFYRPYFRIVRFDVSAMEKNASNLV |
| | | KAEFRVFRLQNPKARVPEQRIELYQILKSKDLTSPTQRYIDS |
| | | KVVKTRAEGEWLSFDVTDAVHEWLHHKDRNLGFKISLHC |
| | | PCCTFVPSNNYIIPNKSEELEARFAGIDGTSTYTSGDQKTIKS |
| | | TRKKNSGKTPHLLLMLLPSYRLESQQTNRRKKRALDAAYC |
| | | FRNVQDNCCLRPLYIDFKRDLGWKWIHEPKGYNANFCAG |
| | | ACPYLWSSDTQHSRVLSLYNTINPEASASPCCVSQDLEPLTI |
| | | LYYIGKTPKIEQLSNMIVKSCKCS |
| SEQ ID NO: | Immature human | MKMHLQRALVVLALLNFATVSLSLSTCTTLDFGHIKKKRV |
| 7256 | TGF-beta 3 | EAIRGQILSKLRLTSPPEPTVMTHVPYQVLALYNSTRELLEE |
| | (P10600-1) | MHGEREEGCTQENTESEYYAKEIHKFDMIQGLAEHNELAV |
| | | CPKGITSKVFRFNVSSVEKNRTNLFRAEFRVLRVPNPSSKR |
| | | NEQRIELFQILRPDEHIAKQRYIGGKNLPTRGTAEWLSFDVT |
| | | DTVREWLLRRESNLGLEISIHCPCHTFQPNGDILENIHEVME |
| | | IKFKGVDNEDDHGRGDLGRLKKQKDHHNPHLILMMIPPHR |
| | | LDNPGQGGQRKKRALDTNYCFRNLEENCCVRPLYIDFRQD |
| | | LGWKWVHEPKGYYANFCSGPCPYLRSADTTHSTVLGLYN |
| | | TLNPEASASPCCVPQDLEPLTILYYVGRTPKVEQLSNMVVK |
| | | SCKCS |
| SEQ ID NO: | Human TGF-beta | LSTCTTLDFGHIKKKRVEAIRGQILSKLRLTSPPEPTVMTHV |
| 7273 | 3 (P10600-1) | PYQVLALYNSTRELLEEMHGEREEGCTQENTESEYYAKEI |
| | | HKFDMIQGLAEHNELAVCPKGITSKVFRFNVSSVEKNRTNL |
| | | FRAEFRVLRVPNPSSKRNEQRIELFQILRPDEHIAKQRYIGG |
| | | KNLPTRGTAEWLSFDVTDTVREWLLRRESNLGLEISIHCPC |
| | | HTFQPNGDILENIHEVMEIKFKGVDNEDDHGRGDLGRLKK |
| | | QKDHHNPHLILMMIPPHRLDNPGQGGQRKKRALDTNYCFR |
| | | NLEENCCVRPLYIDFRQDLGWKWVHEPKGYYANFCSGPCP |
| | | YLRSADTTHSTVLGLYNTLNPEASASPCCVPQDLEPLTILY |
| | | YVGRTPKVEQLSNMVVKSCKCS |
| SEQ ID NO: | Immature human | MEAAVAAPRPRLLLLVLAAAAAAAAALLPGATALQCFCH |
| 7257 | TGFBR1 isoform | LCTKDNFTCVTDGLCFVSVTETTDKVIHNSMCIAEIDLIPRD |
| | 1 (P36897-1) | RPFVCAPSSKTGSVTTTYCCNQDHCNKIELPTTVKSSPGLG |
| | | PVELAAVIAGPVCFVCISLMLMVYICHNRTVIHHRVPNEED |
| | | PSLDRPFISEGTTLKDLIYDMTTSGSGSGLPLLVQRTIARTIV |
| | | LQESIGKGRFGEVWRGKWRGEEVAVKIFSSREERSWFREA |
| | | EIYQTVMLRHENILGFIAADNKDNGTWTQLWLVSDYHEH |
| | | GSLFDYLNRYTVTVEGMIKLALSTASGLAHLHMEIVGTQG |
| | | KPAIAHRDLKSKNILVKKNGTCCIADLGLAVRHDSATDTID |
| | | IAPNHRVGTKRYMAPEVLDDSINMKHFESFKRADIYAMGL |
| | | VFWEIARRCSIGGIHEDYQLPYYDLVPSDPSVEEMRKVVCE |
| | | QKLRPNIPNRWQSCEALRVMAKIMRECWYANGAARLTAL |
| | | RIKKTLSQLSQQEGIKM |
| SEQ ID NO: | Human TGFBR1 | LQCFCHLCTKDNFTCVTDGLCFVSVTETTDKVIHNSMCIAE |
| 7274 | isoform 1 | IDLIPRDRPFVCAPSSKTGSVTTTYCCNQDHCNKIELPTTVK |
| | (P36897-1) | SSPGLGPVELAAVIAGPVCFVCISLMLMVYICHNRTVIHHR |
| | | VPNEEDPSLDRPFISEGTTLKDLIYDMTTSGSGSGLPLLVQR |
| | | TIARTIVLQESIGKGRFGEVWRGKWRGEEVAVKIFSSREER |
| | | SWFREAEIYQTVMLRHENILGFIAADNKDNGTWTQLWLVS |
| | | DYHEHGSLFDYLNRYTVTVEGMIKLALSTASGLAHLHMEI |
| | | VGTQGKPAIAHRDLKSKNILVKKNGTCCIADLGLAVRHDS |
| | | ATDTIDIAPNHRVGTKRYMAPEVLDDSINMKHFESFKRADI |
| | | YAMGLVFWEIARRCSIGGIHEDYQLPYYDLVPSDPSVEEM |
| | | RKVVCEQKLRPNIPNRWQSCEALRVMAKIMRECWYANGA |
| | | ARLTALRIKKTLSQLSQQEGIKM |
| SEQ ID NO: | Immature human | MEAAVAAPRPRLLLLVLAAAAAAAAALLPGATALQCFCH |
| 7258 | TGFBR1 isoform | LCTKDNFTCVTDGLCFVSVTETTDKVIHNSMCIAEIDLIPRD |
| | 2 (P36897-2) | RPFVCAPSSKTGSVTTTYCCNQDHCNKIELPTTGPFSVKSSP |
| | | GLGPVELAAVIAGPVCFVCISLMLMVYICHNRTVIHHRVPN |
| | | EEDPSLDRPFISEGTTLKDLIYDMTTSGSGSGLPLLVQRTIA |
| | | RTIVLQESIGKGRFGEVWRGKWRGEEVAVKIFSSREERSWF |
| | | REAEIYQTVMLRHENILGFIAADNKDNGTWTQLWLVSDYH |
| | | EHGSLFDYLNRYTVTVEGMIKLALSTASGLAHLHMEIVGT |
| | | QGKPAIAHRDLKSKNILVKKNGTCCIADLGLAVRHDSATD |
| | | TIDIAPNHRVGTKRYMAPEVLDDSINMKHFESFKRADIYA |
| | | MGLVFWEIARRCSIGGIHEDYQLPYYDLVPSDPSVEEMRK |
| | | VVCEQKLRPNIPNRWQSCEALRVMAKIMRECWYANGAAR |
| | | LTALRIKKTLSQLSQQEGIKM |
| SEQ ID NO: | Human TGFBR1 | LQCFCHLCTKDNFTCVTDGLCFVSVTETTDKVIHNSMCIAE |
| 7275 | isoform 2 | IDLIPRDRPFVCAPSSKTGSVTTTYCCNQDHCNKIELPTTGP |
| | (P36897-2) | FSVKSSPGLGPVELAAVIAGPVCFVCISLMLMVYICHNRTVI |
| | | HHRVPNEEDPSLDRPFISEGTTLKDLIYDMTTSGSGSGLPLL |
| | | VQRTIARTIVLQESIGKGRFGEVWRGKWRGEEVAVKIFSSR |
| | | EERSWFREAEIYQTVMLRHENILGFIAADNKDNGTWTQLW |
| | | LVSDYHEHGSLFDYLNRYTVTVEGMIKLALSTASGLAHLH |
| | | MEIVGTQGKPAIAHRDLKSKNILVKKNGTCCIADLGLAVR |
| | | HDSATDTIDIAPNHRVGTKRYMAPEVLDDSINMKHFESFKR |
| | | ADIYAMGLVFWEIARRCSIGGIHEDYQLPYYDLVPSDPSVE |
| | | EMRKVVCEQKLRPNIPNRWQSCEALRVMAKIMRECWYAN |
| | | GAARLTALRIKKTLSQLSQQEGIKM |
| SEQ ID NO: | Immature human | MEAAVAAPRPRLLLLVLAAAAAAAAALLPGATALQCFCH |
| 7259 | TGFBR1 isoform | LCTKDNFTCVTDGLCFVSVTETTDKVIHNSMCIAEIDLIPRD |
| | 3 (P36897-3) | RPFVCAPSSKTGSVTTTYCCNQDHCNKIELPTTGLPLLVQR |
| | | TIARTIVLQESIGKGRFGEVWRGKWRGEEVAVKIFSSREER |
| | | SWFREAEIYQTVMLRHENILGFIAADNKDNGTWTQLWLVS |
| | | DYHEHGSLFDYLNRYTVTVEGMIKLALSTASGLAHLHMEI |
| | | VGTQGKPAIAHRDLKSKNILVKKNGTCCIADLGLAVRHDS |
| | | ATDTIDIAPNHRVGTKRYMAPEVLDDSINMKHFESFKRADI |
| | | YAMGLVFWEIARRCSIGGIHEDYQLPYYDLVPSDPSVEEM |
| | | RKVVCEQKLRPNIPNRWQSCEALRVMAKIMRECWYANGA |
| | | ARLTALRIKKTLSQLSQQEGIKM |
| SEQ ID NO: | Human TGFBR1 | LQCFCHLCTKDNFTCVTDGLCFVSVTETTDKVIHNSMCIAE |
| 7276 | isoform 3 | IDLIPRDRPFVCAPSSKTGSVTTTYCCNQDHCNKIELPTTGL |
| | (P36897-3) | PLLVQRTIARTIVLQESIGKGRFGEVWRGKWRGEEVAVKIF |
| | | SSREERSWFREAEIYQTVMLRHENILGFIAADNKDNGTWT |
| | | QLWLVSDYHEHGSLFDYLNRYTVTVEGMIKLALSTASGLA |
| | | HLHMEIVGTQGKPAIAHRDLKSKNILVKKNGTCCIADLGL |
| | | AVRHDSATDTIDIAPNHRVGTKRYMAPEVLDDSINMKHFE |
| | | SFKRADIYAMGLVFWEIARRCSIGGIHEDYQLPYYDLVPSD |
| | | PSVEEMRKVVCEQKLRPNIPNRWQSCEALRVMAKIMREC |
| | | WYANGAARLTALRIKKTLSQLSQQEGIKM |
| SEQ ID NO: | Human TGFBR1 | LQCFCHLCTKDNFTCVTDGLCFVSVTETTDKVIHNSMCIAE |
| 7266 | fragment 1 | IDLIPRDRPFVCAPSSKTGSVTTTYCCNQDHCNKIELPTTVK |
| | | SSPGLGPVEL |
| SEQ ID NO: | Human TGFBR1 | ALQCFCHLCTKDNFTCVTDGLCFVSVTETTDKVIHNSMCIA |
| 7267 | fragment 2 | EIDLIPRDRPFVCAPSSKTGSVTTTYCCNQDHCNKIEL |
| SEQ ID NO: | Immature human | MGRGLLRGLWPLHIVLWTRIASTIPPHVQKSVNNDMIVTD |
| 7260 | TGFBR2 isoform | NNGAVKFPQLCKFCDVRFSTCDNQKSCMSNCSITSICEKPQ |
| | B (short isoform) | EVCVAVWRKNDENITLETVCHDPKLPYHDFILEDAASPKCI |
| | (P37173-1) | MKEKKKPGETFFMCSCSSDECNDNIIFSEEYNTSNPDLLLVI |
| | | FQVTGISLLPPLGVAISVIIIFYCYRVNRQQKLSSTWETGKTR |
| | | KLMEFSEHCAIILEDDRSDISSTCANNINHNTELLPIELDTLV |
| | | GKGRFAEVYKAKLKQNTSEQFETVAVKIFPYEEYASWKTE |
| | | KDIFSDINLKHENILQFLTAEERKTELGKQYWLITAFHAKG |
| | | NLQEYLTRHVISWEDLRKLGSSLARGIAHLHSDHTPCGRPK |
| | | MPIVHRDLKSSNILVKNDLTCCLCDFGLSLRLDPTLSVDDL |
| | | ANSGQVGTARYMAPEVLESRMNLENVESFKQTDVYSMAL |
| | | VLWEMTSRCNAVGEVKDYEPPFGSKVREHPCVESMKDNV |
| | | LRDRGRPEIPSFWLNHQGIQMVCETLTECWDHDPEARLTA |
| | | QCVAERFSELEHLDRLSGRSCSEEKIPEDGSLNTTK |
| SEQ ID NO: | Human TGFBR2 | TIPPHVQKSVNNDMIVTDNNGAVKFPQLCKFCDVRFSTCD |
| 7277 | isoform B (short | NQKSCMSNCSITSICEKPQEVCVAVWRKNDENITLETVCHD |
| | isoform) | PKLPYHDFILEDAASPKCIMKEKKKPGETFFMCSCSSDECN |
| | (P37173-1) | DNIIFSEEYNTSNPDLLLVIFQVTGISLLPPLGVAISVIIIFYCY |
| | | RVNRQQKLSSTWETGKTRKLMEFSEHCAIILEDDRSDISST |
| | | CANNINHNTELLPIELDTLVGKGRFAEVYKAKLKQNTSEQF |
| | | ETVAVKIFPYEEYASWKTEKDIFSDINLKHENILQFLTAEER |
| | | KTELGKQYWLITAFHAKGNLQEYLTRHVISWEDLRKLGSS |
| | | LARGIAHLHSDHTPCGRPKMPIVHRDLKSSNILVKNDLTCC |
| | | LCDFGLSLRLDPTLSVDDLANSGQVGTARYMAPEVLESRM |
| | | NLENVESFKQTDVYSMALVLWEMTSRCNAVGEVKDYEPP |
| | | FGSKVREHPCVESMKDNVLRDRGRPEIPSFWLNHQGIQMV |
| | | CETLTECWDHDPEARLTAQCVAERFSELEHLDRLSGRSCSE |
| | | EKIPEDGSLNTTK |
| SEQ ID NO: | Immature human | MGRGLLRGLWPLHIVLWTRIASTIPPHVQKSDVEMEAQKD |
| 7261 | TGFBR2 isoform | EIICPSCNRTAHPLRHINNDMIVTDNNGAVKFPQLCKFCDV |
| | A (long isoform) | RFSTCDNQKSCMSNCSITSICEKPQEVCVAVWRKNDENITL |
| | (P37173-2) | ETVCHDPKLPYHDFILEDAASPKCIMKEKKKPGETFFMCSC |
| | | SSDECNDNIIFSEEYNTSNPDLLLVIFQVTGISLLPPLGVAISV |
| | | IIIFYCYRVNRQQKLSSTWETGKTRKLMEFSEHCAIILEDDR |
| | | SDISSTCANNINHNTELLPIELDTLVGKGRFAEVYKAKLKQ |
| | | NTSEQFETVAVKIFPYEEYASWKTEKDIFSDINLKHENILQF |
| | | LTAEERKTELGKQYWLITAFHAKGNLQEYLTRHVISWEDL |
| | | RKLGSSLARGIAHLHSDHTPCGRPKMPIVHRDLKSSNILVK |
| | | NDLTCCLCDFGLSLRLDPTLSVDDLANSGQVGTARYMAPE |
| | | VLESRMNLENVESFKQTDVYSMALVLWEMTSRCNAVGEV |
| | | KDYEPPFGSKVREHPCVESMKDNVLRDRGRPEIPSFWLNH |
| | | QGIQMVCETLTECWDHDPEARLTAQCVAERFSELEHLDRL |
| | | SGRSCSEEKIPEDGSLNTTK |
| SEQ ID NO: | Human TGFBR2 | TIPPHVQKSDVEMEAQKDEIICPSCNRTAHPLRHINNDMIVT |
| 7278 | isoform A (long | DNNGAVKFPQLCKFCDVRFSTCDNQKSCMSNCSITSICEKP |
| | isoform) | QEVCVAVWRKNDENITLETVCHDPKLPYHDFILEDAASPK |
| | (P37173-2) | CIMKEKKKPGETFFMCSCSSDECNDNIIFSEEYNTSNPDLLL |
| | | VIFQVTGISLLPPLGVAISVIIIFYCYRVNRQQKLSSTWETGK |
| | | TRKLMEFSEHCAIILEDDRSDISSTCANNINHNTELLPIELDT |
| | | LVGKGRFAEVYKAKLKQNTSEQFETVAVKIFPYEEYASWK |
| | | TEKDIFSDINLKHENILQFLTAEERKTELGKQYWLITAFHAK |
| | | GNLQEYLTRHVISWEDLRKLGSSLARGIAHLHSDHTPCGRP |
| | | KMPIVHRDLKSSNILVKNDLTCCLCDFGLSLRLDPTLSVDD |
| | | LANSGQVGTARYMAPEVLESRMNLENVESFKQTDVYSMA |
| | | LVLWEMTSRCNAVGEVKDYEPPFGSKVREHPCVESMKDN |
| | | VLRDRGRPEIPSFWLNHQGIQMVCETLTECWDHDPEARLT |
| | | AQCVAERFSELEHLDRLSGRSCSEEKIPEDGSLNTTK |
| SEQ ID NO: | Human TGFBR2 | TIPPHVQKSVNNDMIVTDNNGAVKFPQLCKFCDVRFSTCD |
| 7262 | fragment 1 (ECD | NQKSCMSNCSITSICEKPQEVCVAVWRKNDENITLETVCHD |
| | of human | PKLPYHDFILEDAASPKCIMKEKKKPGETFFMCSCSSDECN |
| | TGFBR2 isoform | DNIIFSEEYNTSNPD |
| | B) | |
| SEQ ID NO: | Human TGFBR2 | IPPHVQKSVNNDMIVTDNNGAVKFPQLCKFCDVRFSTCDN |
| 7263 | fragment 2 | QKSCMSNCSITSICEKPQEVCVAVWRKNDENITLETVCHDP |
| | | KLPYHDFILEDAASPKCIMKEKKKPGETFFMCSCSSDECND |
| | | NIIFSEEYNTSNPD |
| SEQ ID NO: | Human TGFBR2 | TIPPHVQKSDVEMEAQKDEIICPSCNRTAHPLRHINNDMIVT |
| 7264 | fragment 3 (ECD | DNNGAVKFPQLCKFCDVRFSTCDNQKSCMSNCSITSICEKP |
| | of human | QEVCVAVWRKNDENITLETVCHDPKLPYHDFILEDAASPK |
| | TGFBR2 isoform | CIMKEKKKPGETFFMCSCSSDECNDNIIFSEEYNTSNPD |
| | A) | |
| SEQ ID NO: | Human TGFBR2 | QLCKFCDVRFSTCDNQKSCMSNCSITSICEKPQEVCVAVW |
| 7265 | fragment 4 | RKNDENITLETVCHDPKLPYHDFILEDAASPKCIMKEKKKP |
| | | GETFFMCSCSSDECNDNIIF |
| SEQ ID NO: | Immature human | MTSHYVIAIFALMSSCLATAGPEPGALCELSPVSASHPVQA |
| 7268 | TGFBR3 isoform | LMESFTVLSGCASRGTTGLPQEVHVLNLRTAGQGPGQLQR |
| | 1 (Q03167-1) | EVTLHLNPISSVHIHHKSVVFLLNSPHPLVWHLKTERLATG |
| | | VSRLFLVSEGSVVQFSSANFSLTAETEERNFPHGNEHLLNW |
| | | ARKEYGAVTSFTELKIARNIYIKVGEDQVFPPKCNIGKNFLS |
| | | LNYLAEYLQPKAAEGCVMSSQPQNEEVHIIELITPNSNPYS |
| | | AFQVDITIDIRPSQEDLEVVKNLILILKCKKSVNWVIKSFDV |
| | | KGSLKIIAPNSIGFGKESERSMTMTKSIRDDIPSTQGNLVKW |
| | | ALDNGYSPITSYTMAPVANRFHLRLENNAEEMGDEEVHTI |
| | | PPELRILLDPGALPALQNPPIRGGEGQNGGLPFPFPDISRRV |
| | | WNEEGEDGLPRPKDPVIPSIQLFPGLREPEEVQGSVDIALSV |
| | | KCDNEKMIVAVEKDSFQASGYSGMDVTLLDPTCKAKMNG |
| | | THFVLESPLNGCGTRPRWSALDGVVYYNSIVIQVPALGDSS |
| | | GWPDGYEDLESGDNGFPGDMDEGDASLFTRPEIVVFNCSL |
| | | QQVRNPSSFQEQPHGNITFNMELYNTDLFLVPSQGVFSVPE |
| | | NGHVYVEVSVTKAEQELGFAIQTCFISPYSNPDRMSHYTIIE |
| | | NICPKDESVKFYSPKRVHFPIPQADMDKKRFSFVFKPVFNT |
| | | SLLFLQCELTLCTKMEKHPQKLPKCVPPDEACTSLDASIIW |
| | | AMMQNKKTFTKPLAVIHHEAESKEKGPSMKEPNPISPPIFH |
| | | GLDTLTVMGIAFAAFVIGALLTGALWYWSHTGETAGRQQ |
| | | VPTSPPASENSSAAHSIGSTQSTPCSSSSTA |
| SEQ ID NO: | Human TGFBR3 | GPEPGALCELSPVSASHPVQALMESFTVLSGCASRGTTGLP |
| 7279 | isoform 1 | QEVHVLNLRTAGQGPGQLQREVTLHLNPISSVHIHHKSVVF |
| | (Q03167-1) | LLNSPHPLVWHLKTERLATGVSRLFLVSEGSVVQFSSANFS |
| | | LTAETEERNFPHGNEHLLNWARKEYGAVTSFTELKIARNIY |
| | | IKVGEDQVFPPKCNIGKNFLSLNYLAEYLQPKAAEGCVMSS |
| | | QPQNEEVHIIELITPNSNPYSAFQVDITIDIRPSQEDLEVVKN |
| | | LILILKCKKSVNWVIKSFDVKGSLKIIAPNSIGFGKESERSMT |
| | | MTKSIRDDIPSTQGNLVKWALDNGYSPITSYTMAPVANRF |
| | | HLRLENNAEEMGDEEVHTIPPELRILLDPGALPALQNPPIRG |
| | | GEGQNGGLPFPFPDISRRVWNEEGEDGLPRPKDPVIPSIQLF |
| | | PGLREPEEVQGSVDIALSVKCDNEKMIVAVEKDSFQASGYS |
| | | GMDVTLLDPTCKAKMNGTHFVLESPLNGCGTRPRWSALD |
| | | GVVYYNSIVIQVPALGDSSGWPDGYEDLESGDNGFPGDMD |
| | | EGDASLFTRPEIVVFNCSLQQVRNPSSFQEQPHGNITFNMEL |
| | | YNTDLFLVPSQGVFSVPENGHVYVEVSVTKAEQELGFAIQT |
| | | CFISPYSNPDRMSHYTIIENICPKDESVKFYSPKRVHFPIPQA |
| | | DMDKKRFSFVFKPVFNTSLLFLQCELTLCTKMEKHPQKLP |
| | | KCVPPDEACTSLDASIIWAMMQNKKTFTKPLAVIHHEAES |
| | | KEKGPSMKEPNPISPPIFHGLDTLTVMGIAFAAFVIGALLTG |
| | | ALWYIYSHTGETAGRQQVPTSPPASENSSAAHSIGSTQSTPC |
| | | SSSSTA |
| SEQ ID NO: | Immature human | MTSHYVIAIFALMSSCLATAGPEPGALCELSPVSASHPVQA |
| 7269 | TGFBR3 isoform | LMESFTVLSGCASRGTTGLPQEVHVLNLRTAGQGPGQLQR |
| | 2 (Q03167-2) | EVTLHLNPISSVHIHHKSVVFLLNSPHPLVWHLKTERLATG |
| | | VSRLFLVSEGSVVQFSSANFSLTAETEERNFPHGNEHLLNW |
| | | ARKEYGAVTSFTELKIARNIYIKVGEDQVFPPKCNIGKNFLS |
| | | LNYLAEYLQPKAAEGCVMSSQPQNEEVHIIELITPNSNPYS |
| | | AFQVDITIDIRPSQEDLEVVKNLILILKCKKSVNWVIKSFDV |
| | | KGSLKIIAPNSIGFGKESERSMTMTKSIRDDIPSTQGNLVKW |
| | | ALDNGYSPITSYTMAPVANRFHLRLENNEEMGDEEVHTIPP |
| | | ELRILLDPGALPALQNPPIRGGEGQNGGLPFPFPDISRRVWN |
| | | EEGEDGLPRPKDPVIPSIQLFPGLREPEEVQGSVDIALSVKC |
| | | DNEKMIVAVEKDSFQASGYSGMDVTLLDPTCKAKMNGTH |
| | | FVLESPLNGCGTRPRWSALDGVVYYNSIVIQVPALGDSSG |
| | | WPDGYEDLESGDNGFPGDMDEGDASLFTRPEIVVFNCSLQ |
| | | QVRNPSSFQEQPHGNITFNMELYNTDLFLVPSQGVFSVPEN |
| | | GHVYVEVSVTKAEQELGFAIQTCFISPYSNPDRMSHYTIIEN |
| | | ICPKDESVKFYSPKRVHFPIPQADMDKKRFSFVFKPVFNTSL |
| | | LFLQCELTLCTKMEKHPQKLPKCVPPDEACTSLDASIIWAM |
| | | MQNKKTFTKPLAVIHHEAESKEKGPSMKEPNPISPPIFHGLD |
| | | TLTVMGIAFAAFVIGALLTGALWYIYSHTGETAGRQQVPTS |
| | | PPASENSSAAHSIGSTQSTPCSSSSTA |
| SEQ ID NO: | Human TGFBR3 | GPEPGALCELSPVSASHPVQALMESFTVLSGCASRGTTGLP |
| 7280 | isoform 2 | QEVHVLNLRTAGQGPGQLQREVTLHLNPISSVHIHHKSVVF |
| | (Q03167-2) | LLNSPHPLVWHLKTERLATGVSRLFLVSEGSVVQFSSANFS |
| | | LTAETEERNFPHGNEHLLNWARKEYGAVTSFTELKIARNIY |
| | | IKVGEDQVFPPKCNIGKNFLSLNYLAEYLQPKAAEGCVMSS |
| | | QPQNEEVHIIELITPNSNPYSAFQVDITIDIRPSQEDLEVVKN |
| | | LILILKCKKSVNWVIKSFDVKGSLKIIAPNSIGFGKESERSMT |
| | | MTKSIRDDIPSTQGNLVKWALDNGYSPITSYTMAPVANRF |
| | | HLRLENNEEMGDEEVHTIPPELRILLDPGALPALQNPPIRGG |
| | | EGQNGGLPFPFPDISRRVWNEEGEDGLPRPKDPVIPSIQLFP |
| | | GLREPEEVQGSVDIALSVKCDNEKMIVAVEKDSFQASGYS |
| | | GMDVTLLDPTCKAKMNGTHFVLESPLNGCGTRPRWSALD |
| | | GVVYYNSIVIQVPALGDSSGWPDGYEDLESGDNGFPGDMD |
| | | EGDASLFTRPEIVVFNCSLQQVRNPSSFQEQPHGNITFNMEL |
| | | YNTDLFLVPSQGVFSVPENGHVYVEVSVTKAEQELGFAIQT |
| | | CFISPYSNPDRMSHYTIIENICPKDESVKFYSPKRVHFPIPQA |
| | | DMDKKRFSFVFKPVFNTSLLFLQCELTLCTKMEKHPQKLP |
| | | KCVPPDEACTSLDASIIWAMMQNKKTFTKPLAVIHHEAES |
| | | KEKGPSMKEPNPISPPIFHGLDTLTVMGIAFAAFVIGALLTG |
| | | ALWYIYSHTGETAGRQQVPTSPPASENSSAAHSIGSTQSTPC |
| | | SSSSTA |
| SEQ ID NO: | Human TGFBR3 | GPEPGALCELSPVSASHPVQALMESFTVLSGCASRGTTGLP |
| 7270 | fragment 1 | QEVHVLNLRTAGQGPGQLQREVTLHLNPISSVHIHHKSVVF |
| | | LLNSPHPLVWHLKTERLATGVSRLFLVSEGSVVQFSSANFS |
| | | LTAETEERNFPHGNEHLLNWARKEYGAVTSFTELKIARNIY |
| | | IKVGEDQVFPPKCNIGKNFLSLNYLAEYLQPKAAEGCVMSS |
| | | QPQNEEVHIIELITPNSNPYSAFQVDITIDIRPSQEDLEVVKN |
| | | LILILKCKKSVNWVIKSFDVKGSLKIIAPNSIGFGKESERSMT |
| | | MTKSIRDDIPSTQGNLVKWALDNGYSPITSYTMAPVANRF |
| | | HLRLENNAEEMGDEEVHTIPPELRILLDPGALPALQNPPIRG |
| | | GEGQNGGLPFPFPDISRRVWNEEGEDGLPRPKDPVIPSIQLF |
| | | PGLREPEEVQGSVDIALSVKCDNEKMIVAVEKDSFQASGYS |
| | | GMDVTLLDPTCKAKMNGTHFVLESPLNGCGTRPRWSALD |
| | | GVVYYNSIVIQVPALGDSSGWPDGYEDLESGDNGFPGDMD |
| | | EGDASLFTRPEIVVFNCSLQQVRNPSSFQEQPHGNITFNMEL |
| | | YNTDLFLVPSQGVFSVPENGHVYVEVSVTKAEQELGFAIQT |
| | | CFISPYSNPDRMSHYTIIENICPKDESVKFYSPKRVHFPIPQA |
| | | DMDKKRFSFVFKPVFNTSLLFLQCELTLCTKMEKHPQKLP |
| | | KCVPPDEACTSLDASIIWAMMQNKKTFTKPLAVIHHEAES |
| | | KEKGPSMKEPNPISPPIFHGLDTLTV |
| SEQ ID NO: | hCH1-hFc_Hole- | ASTKGPSVFPLAPSSKSTSGGTAALGCLVKDYFPEPVTVSW |
| 7281 | 3x4GS-TGFbR2 | NSGALTSGVHTFPAVLQSSGLYSLSSVVTVPSSSLGTQTYIC |
| | | NVNHKPSNTKVDKRVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVCTLPPSREEMTKN |
| | | QVSLSCAVKGFYPSDIAVEWESNGQPENNYKTTPPVLDSD |
| | | GSFFLVSKLTVDKSRWQQGNVFSCSVMHEALHNHYTQKS |
| | | LSLSPGXGGGGSGGGGSGGGGSIPPHVQKSVNNDMIVTDN |
| | | NGAVKFPQLCKFCDVRFSTCDNQKSCMSNCSITSICEKPQE |
| | | VCVAVWRKNDENITLETVCHDPKLPYHDFILEDAASPKCI |
| | | MKEKKKPGETFFMCSCSSDECNDNIIFSEEYNTSNPD, |
| | | wherein X is K or absent |
| SEQ ID NO: | hCH1- | ASTKGPSVFPLAPSSKSTSGGTAALGCLVKDYFPEPVTVSW |
| 7282 | hFc_Knob- | NSGALTSGVHTFPAVLQSSGLYSLSSVVTVPSSSLGTQTYIC |
| | 3x4GS-TGFbR2 | NVNHKPSNTKVDKRVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPCREEMTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGXGGGGSGGGGSGGGGSIPPHVQKSVNNDMIVTDN |
| | | NGAVKFPQLCKFCDVRFSTCDNQKSCMSNCSITSICEKPQE |
| | | VCVAVWRKNDENITLETVCHDPKLPYHDFILEDAASPKCI |
| | | MKEKKKPGETFFMCSCSSDECNDNIIFSEEYNTSNPD, |
| | | wherein X is K or absent |
| SEQ ID NO: | hFc_Hole- | DKTHTCPPCPAPELLGGPSVFLFPPKPKDTLMISRTPEVTCV |
| 7283 | 3x4GS-TGFbR2 | VVDVSHEDPEVKFNWYVDGVEVHNAKTKPREEQYNSTYR |
| | | VVSVLTVLHQDWLNGKEYKCKVSNKALPAPIEKTISKAKG |
| | | QPREPQVCTLPPSREEMTKNQVSLSCAVKGFYPSDIAVEWE |
| | | SNGQPENNYKTTPPVLDSDGSFFLVSKLTVDKSRWQQGNV |
| | | FSCSVMHEALHNHYTQKSLSLSPGXGGGGSGGGGSGGGGS |
| | | IPPHVQKSVNNDMIVTDNNGAVKFPQLCKFCDVRFSTCDN |
| | | QKSCMSNCSITSICEKPQEVCVAVWRKNDENITLETVCHDP |
| | | KLPYHDFILEDAASPKCIMKEKKKPGETFFMCSCSSDECND |
| | | NIIFSEEYNTSNPD, wherein X is K or absent |
| SEQ ID NO: | hFc_Knob- | DKTHTCPPCPAPELLGGPSVFLFPPKPKDTLMISRTPEVTCV |
| 7284 | 3x4GS-TGFbR2 | VVDVSHEDPEVKFNWYVDGVEVHNAKTKPREEQYNSTYR |
| | | VVSVLTVLHQDWLNGKEYKCKVSNKALPAPIEKTISKAKG |
| | | QPREPQVYTLPPCREEMTKNQVSLWCLVKGFYPSDIAVEW |
| | | ESNGQPENNYKTTPPVLDSDGSFFLYSKLTVDKSRWQQGN |
| | | VFSCSVMHEALHNHYTQKSLSLSPGXGGGGSGGGGSGGG |
| | | GSIPPHVQKSVNNDMIVTDNNGAVKFPQLCKFCDVRFSTC |
| | | DNQKSCMSNCSITSICEKPQEVCVAVWRKNDENITLETVCH |
| | | DPKLPYHDFILEDAASPKCIMKEKKKPGETFFMCSCSSDEC |
| | | NDNIIFSEEYNTSNPD, wherein X is K or absent |
| SEQ ID NO: | TGFbR2-3x4GS- | IPPHVQKSVNNDMIVTDNNGAVKFPQLCKFCDVRFSTCDN |
| 7285 | hCH1-hFc_Hole | QKSCMSNCSITSICEKPQEVCVAVWRKNDENITLETVCHDP |
| | | KLPYHDFILEDAASPKCIMKEKKKPGETFFMCSCSSDECND |
| | | NIIFSEEYNTSNPDGGGGSGGGGSGGGGSASTKGPSVFPLA |
| | | PSSKSTSGGTAALGCLVKDYFPEPVTVSWNSGALTSGVHTF |
| | | PAVLQSSGLYSLSSVVTVPSSSLGTQTYICNVNHKPSNTKV |
| | | DKRVEPKSCDKTHTCPPCPAPELLGGPSVFLFPPKPKDTLMI |
| | | SRTPEVTCVVVDVSHEDPEVKFNWYVDGVEVHNAKTKPR |
| | | EEQYNSTYRVVSVLTVLHQDWLNGKEYKCKVSNKALPAPI |
| | | EKTISKAKGQPREPQVCTLPPSREEMTKNQVSLSCAVKGFY |
| | | PSDIAVEWESNGQPENNYKTTPPVLDSDGSFFLVSKLTVDK |
| | | SRWQQGNVFSCSVMHEALHNHYTQKSLSLSPGX, wherein |
| | | X is K or absent |
| SEQ ID NO: | TGFbR2-3x4GS- | IPPHVQKSVNNDMIVTDNNGAVKFPQLCKFCDVRFSTCDN |
| 7286 | hCH1-hFc_Knob | QKSCMSNCSITSICEKPQEVCVAVWRKNDENITLETVCHDP |
| | | KLPYHDFILEDAASPKCIMKEKKKPGETFFMCSCSSDECND |
| | | NIIFSEEYNTSNPDGGGGSGGGGSGGGGSASTKGPSVFPLA |
| | | PSSKSTSGGTAALGCLVKDYFPEPVTVSWNSGALTSGVHTF |
| | | PAVLQSSGLYSLSSVVTVPSSSLGTQTYICNVNHKPSNTKV |
| | | DKRVEPKSCDKTHTCPPCPAPELLGGPSVFLFPPKPKDTLMI |
| | | SRTPEVTCVVVDVSHEDPEVKFNWYVDGVEVHNAKTKPR |
| | | EEQYNSTYRVVSVLTVLHQDWLNGKEYKCKVSNKALPAPI |
| | | EKTISKAKGQPREPQVYTLPPCREEMTKNQVSLWCLVKGF |
| | | YPSDIAVEWESNGQPENNYKTTPPVLDSDGSFFLYSKLTVD |
| | | KSRWQQGNVFSCSVMHEALHNHYTQKSLSLSPGX, wherein |
| | | X is K or absent |
| SEQ ID NO: | TGFbR2-3x4GS- | IPPHVQKSVNNDMIVTDNNGAVKFPQLCKFCDVRFSTCDN |
| 7287 | hCLIg_v1 | QKSCMSNCSITSICEKPQEVCVAVWRKNDENITLETVCHDP |
| | | KLPYHDFILEDAASPKCIMKEKKKPGETFFMCSCSSDECND |
| | | NIIFSEEYNTSNPDGGGGSGGGGSGGGGSGQPKANPTVTLF |
| | | PPSSEELQANKATLVCLISDFYPGAVTVAWKADGSPVKAG |
| | | VETTKPSKQSNNKYAASSYLSLTPEQWKSHRSYSCQVTHE |
| | | GSTVEKTVAPTECS |
| SEQ ID NO: | TGFβR2-3x4GS- | IPPHVQKSVNNDMIVTDNNGAVKFPQLCKFCDVRFSTCDN |
| 7288 | hCLIg_vk | QKSCMSNCSITSICEKPQEVCVAVWRKNDENITLETVCHDP |
| | | KLPYHDFILEDAASPKCIMKEKKKPGETFFMCSCSSDECND |
| | | NIIFSEEYNTSNPDGGGGSGGGGSGGGGSRTVAAPSVFIFPP |
| | | SDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |

### Immune Cell Engagers

The immune cell engagers of the multispecific or multifunctional molecules disclosed herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. For example, the NCRs can initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U (optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis. Without wishing to be bound by theory, it is thought that NK cell engagers that bind, recruit, and/or activate receptors like those disclosed above (e.g., NKp30, NKp36, NKG2D, or CD16) may target immune system activity to a target cell, e.g., a cell comprising a TCRBV antigen (e.g., a TCRBV antigen corresponding to a biased TCRBV clonotype), e.g., promote cell death or lysis of a target cell.

The present disclosure provides, inter alia, multispecific (e.g., bi-, tri-, quad-specific) or multifunctional molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in Tables 7-10. In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in U.S. Pat. Nos. 6,979,546, 9,447,185, PCT Application No. WO2015121383A1, PCT Application No. WO2016110468A1, PCT Application No.

WO2004056392A1, or U.S. Application Publication No. US20070231322A1, the sequences of which are hereby incorporated by reference. In some embodiments, binding of the NK cell engager, e.g., antigen binding domain that binds to NKp30, to the NK cell activates the NK cell. An antigen binding domain that binds to NKp30 (e.g., NKp30 present, e.g., expressed or displayed, on the surface of an NK cell) may be said to target NKp30, the NK cell, or both.

In some embodiments, the antigen binding domain that binds to NKp30 comprises one or more CDRs (e.g., VHCDR1, VHCDR2, VHCDR3, VLCDR1, VLCDR2, and/or VLCDR3) disclosed in Table 7, Table 18, or Table 8, or a sequence having at least 85%, 90%, 95%, or 99% identity thereto. In some embodiments, the antigen binding domain that binds to NKp30 comprises one or more framework regions (e.g., VHFWR1, VHFWR2, VHFWR3, VHFWR4, VLFWR1, VLFWR2, VLFWR3, and/or VLFWR4) disclosed in Table 7, Table 18, or Table 8, or a sequence having at least 85%, 90%, 95%, or 99% identity thereto. In some embodiments, the antigen binding domain that binds to NKp30 comprises a VH and/or a VL disclosed in Table 9, or a sequence having at least 85%, 90%, 95%, or 99% identity thereto. In some embodiments, any of the VH domains disclosed in Table 9 may be paired with any of the VL domains disclosed in Table 9 to form the antigen binding domain that binds to NKp30. In some embodiments, the antigen binding domain that binds to NKp30 comprises an amino acid sequence disclosed in Table 10, or a sequence having at least 85%, 90%, 95%, or 99% identity thereto.

In some embodiments, the antigen binding domain that binds to NKp30 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 7313, 6001, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6001, and 6002, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6008, and 6009, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7385, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7318, and 6009, respectively (or a sequence having at least 85%, 90%, 95%, or 99% identity thereto).

In some embodiments, the VLCDR1, VLCDR2, and VLCDR3 comprise the amino acid sequences of SEQ ID NOs: 7326, 7327, and 7329, respectively (or a sequence having at least 85%, 90%, 95%, or 99% identity thereto). In some embodiments, the VLCDR1, VLCDR2, and VLCDR3 comprise the amino acid sequences of SEQ ID NOs: 6063, 6064, and 7293, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6071, and 6072, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6064, and 7321, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 7313, 6001, 7315, 7326, 7327, and 7329, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6001, 6002, 6063, 6064, and 7293, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6008, 6009, 6070, 6071, and 6072, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7385, 7315, 6070, 6064, and 7321, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7318, 6009, 6070, 6064, and 7321, respectively (or a sequence having at least 85%, 90%, 95%, or 99% identity thereto).

In some embodiments, the VH comprises an amino acid sequence selected from the group consisting of SEQ ID NOs: 7298 or 7300-7304 (or a sequence having at least 85%, 90%, 95%, or 99% identity thereto) and/or the VL comprises an amino acid sequence selected from the group consisting of SEQ ID NOs: 7299 or 7305-7309 (or a sequence having at least 85%, 90%, 95%, or 99% identity thereto). In some embodiments, the VH and VL comprise the amino acid sequences of SEQ ID NOs: 7302 and 7305, respectively (or a sequence having at least 85%, 90%, 95%, or 99% identity thereto). In some embodiments, the VH and VL comprise the amino acid sequences of SEQ ID NOs: 7302 and 7309, respectively (or a sequence having at least 85%, 90%, 95%, or 99% identity thereto).

In some embodiments, the VH comprises an amino acid sequence selected from the group consisting of SEQ ID NOs: 6121 or 6123-6128 (or a sequence having at least 85%, 90%, 95%, or 99% identity thereto) and/or the VL comprises an amino acid sequence selected from the group consisting of SEQ ID NOs: 7294 or 6137-6141 (or a sequence having at least 85%, 90%, 95%, or 99% identity thereto). In some embodiments, the VH comprises an amino acid sequence selected from the group consisting of SEQ ID NOs: 6122 or 6129-6134 (or a sequence having at least 85%, 90%, 95%, or 99% identity thereto) and/or the VL comprises an amino acid sequence selected from the group consisting of SEQ ID NOs: 6136 or 6142-6147 (or a sequence having at least 85%, 90%, 95%, or 99% identity thereto). In some embodiments, the VH and VL comprise the amino acid sequences of SEQ ID NOs: 7295 and 7296, respectively (or a sequence having at least 85%, 90%, 95%, or 99% identity thereto). In some embodiments, the VH and VL comprise the amino acid sequences of SEQ ID NOs: 7297 and 7296, respectively (or a sequence having at least 85%, 90%, 95%, or 99% identity thereto). In some embodiments, the VH and VL comprise the amino acid sequences of SEQ ID NOs: 6122 and 6136, respectively (or a sequence having at least 85%, 90%, 95%, or 99% identity thereto).

In some embodiments, the antigen binding domain that binds to NKp30 comprises the amino acid sequence of SEQ ID NO: 7310 (or a sequence having at least 85%, 90%, 95%, or 99% identity thereto). In some embodiments, the antigen binding domain that binds to NKp30 comprises the amino acid sequence of SEQ ID NO: 7311 (or a sequence having at least 85%, 90%, 95%, or 99% identity thereto). In some embodiments, the antigen binding domain that binds to NKp30 comprises the amino acid sequence of SEQ ID NO: 6187, 6188, 6189 or 6190 (or a sequence having at least 85%, 90%, 95%, or 99% identity thereto). In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002.

In some embodiments, the antigen binding domain that targets NKp30 comprises a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002, and a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009.

In some embodiments, the antigen binding domain that targets NKp30 comprises a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009, and a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006.

In some embodiments, the antigen binding domain that targets NKp30 comprises a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006, and a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006.

In some embodiments, the antigen binding domain that targets NKp30 comprises a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006, and a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013.

In some embodiments, the antigen binding domain that targets NKp30 comprises a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013, and a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013.

In some embodiments, the antigen binding domain that targets NKp30 comprises a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013, and a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, and/or a VHFWR4 amino acid sequence of SEQ ID NO: 6017.

In some embodiments, the antigen binding domain that targets NKp30 comprises a VH comprising a VHFWR1 amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6017.

In some embodiments, the antigen binding domain that targets NKp30 comprises a VL comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6077 (or a sequence with no more than 1, 2, or 3 mutations, e.g., substitutions, additions, or deletions), a VLFWR2 amino acid sequence of SEQ ID NO: 6078 (or a sequence with no more than 1 mutation, e.g., substitution, addition, or deletion), a VLFWR3 amino acid sequence of SEQ ID NO: 6079 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, and/or a VHFWR4 amino acid sequence of SEQ ID NO: 6021.

In some embodiments, the antigen binding domain that targets NKp30 comprises a VH comprising a VHFWR1 amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6021.

In some embodiments, the antigen binding domain that targets NKp30 comprises a VL comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, and/or a VLFWR4 amino acid sequence of SEQ ID NO: 6084.

In some embodiments, the antigen binding domain that targets NKp30 comprises a VL comprising a VLFWR1 amino acid sequence of SEQ ID NO: 6081 (or a sequence with no more than 1, 2, or 3 mutations, e.g., substitutions, additions, or deletions), a VLFWR2 amino acid sequence of SEQ ID NO: 6082 (or a sequence with no more than 1 mutation, e.g., substitution, addition, or deletion), a VLFWR3 amino acid sequence of SEQ ID NO: 6083 (or a sequence with no more than 1 mutation, e.g., substitution, addition, or deletion), and/or a VLFWR4 amino acid sequence of SEQ ID NO: 6084.

In some embodiments, the antigen binding domain that targets NKp30 comprises a VH comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, and/or a VHFWR4 amino acid sequence of SEQ ID NO: 6025.

In some embodiments, the antigen binding domain that targets NKp30 comprises a VH comprising a VHFWR1 amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6025.

In some embodiments, the antigen binding domain that targets NKp30 comprises a VL comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, and/or a VLFWR4 amino acid sequence of SEQ ID NO: 6088.

In some embodiments, the antigen binding domain that targets NKp30 comprises a VL comprising a VLFWR1 amino acid sequence of SEQ ID NO: 6085 (or a sequence with no more than 1, 2, or 3 mutations, e.g., substitutions, additions, or deletions), a VLFWR2 amino acid sequence of SEQ ID NO: 6086 (or a sequence with no more than 1 mutation, e.g., substitution, addition, or deletion), a VLFWR3 amino acid sequence of SEQ ID NO: 6087 (or a sequence with no more than 1 mutation, e.g., substitution, addition, or deletion), and/or a VLFWR4 amino acid sequence of SEQ ID NO: 6088.

In some embodiments, the antigen binding domain that targets NKp30 comprises a VH comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, and/or a VHFWR4 amino acid sequence of SEQ ID NO: 6029.

In some embodiments, the antigen binding domain that targets NKp30 comprises a VH comprising a VHFWR1 amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6029.

In some embodiments, the antigen binding domain that targets NKp30 comprises a VL comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, and/or a VLFWR4 amino acid sequence of SEQ ID NO: 6092.

In some embodiments, the antigen binding domain that targets NKp30 comprises a VL comprising a VLFWR1 amino acid sequence of SEQ ID NO: 6089 (or a sequence with no more than 1, 2, or 3 mutations, e.g., substitutions, additions, or deletions), a VLFWR2 amino acid sequence of SEQ ID NO: 6090 (or a sequence with no more than 1 mutation, e.g., substitution, addition, or deletion), a VLFWR3 amino acid sequence of SEQ ID NO: 6091 (or a sequence with no more than 1 mutation, e.g., substitution, addition, or deletion), and/or a VLFWR4 amino acid sequence of SEQ ID NO: 6092.

In some embodiments, the antigen binding domain that targets NKp30 comprises a VH comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, and/or a VHFWR4 amino acid sequence of SEQ ID NO: 6034.

In some embodiments, the antigen binding domain that targets NKp30 comprises a VH comprising a VHFWR1 amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6034.

In some embodiments, the antigen binding domain that targets NKp30 comprises a VL comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, and/or a VLFWR4 amino acid sequence of SEQ ID NO: 6096.

In some embodiments, the antigen binding domain that targets NKp30 comprises a VL comprising a VLFWR1 amino acid sequence of SEQ ID NO: 6093 (or a sequence with no more than 1, 2, or 3 mutations, e.g., substitutions, additions, or deletions), a VLFWR2 amino acid sequence of SEQ ID NO: 6094 (or a sequence with no more than 1 mutation, e.g., substitution, addition, or deletion), a VLFWR3 amino acid sequence of SEQ ID NO: 6095 (or a sequence with no more than 1 mutation, e.g., substitution, addition, or deletion), and/or a VLFWR4 amino acid sequence of SEQ ID NO: 6096.

In some embodiments, the antigen binding domain that targets NKp30 comprises a VH comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, and/or a VHFWR4 amino acid sequence of SEQ ID NO: 6038.

In some embodiments, the antigen binding domain that targets NKp30 comprises a VH comprising a VHFWR1 amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6038.

In some embodiments, the antigen binding domain that targets NKp30 comprises a VH comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, and/or a VHFWR4 amino acid sequence of SEQ ID NO: 6042.

In some embodiments, the antigen binding domain that targets NKp30 comprises a VH comprising a VHFWR1 amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6042.

In some embodiments, the antigen binding domain that targets NKp30 comprises a VL comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, and/or a VLFWR4 amino acid sequence of SEQ ID NO: 6100.

In some embodiments, the antigen binding domain that targets NKp30 comprises a VL comprising a VLFWR1 amino acid sequence of SEQ ID NO: 6097 (or a sequence with no more than 1, 2, or 3 mutations, e.g., substitutions, additions, or deletions), a VLFWR2 amino acid sequence of SEQ ID NO: 6098 (or a sequence with no more than 1 mutation, e.g., substitution, addition, or deletion), a VLFWR3 amino acid sequence of SEQ ID NO: 6099 (or a sequence with no more than 1 mutation, e.g., substitution, addition, or deletion), and/or a VLFWR4 amino acid sequence of SEQ ID NO: 6100.

In some embodiments, the antigen binding domain that targets NKp30 comprises a VH comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, and/or a VHFWR4 amino acid sequence of SEQ ID NO: 6046.

In some embodiments, the antigen binding domain that targets NKp30 comprises a VH comprising a VHFWR1 amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6046.

In some embodiments, the antigen binding domain that targets NKp30 comprises a VL comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, and/or a VLFWR4 amino acid sequence of SEQ ID NO: 6104.

In some embodiments, the antigen binding domain that targets NKp30 comprises a VL comprising a VLFWR1 amino acid sequence of SEQ ID NO: 6101 (or a sequence with no more than 1, 2, or 3 mutations, e.g., substitutions, additions, or deletions), a VLFWR2 amino acid sequence of SEQ ID NO: 6102 (or a sequence with no more than 1 mutation, e.g., substitution, addition, or deletion), a VLFWR3 amino acid sequence of SEQ ID NO: 6103 (or a sequence with no more than 1 mutation, e.g., substitution, addition, or deletion), and/or a VLFWR4 amino acid sequence of SEQ ID NO: 6104.

In some embodiments, the antigen binding domain that targets NKp30 comprises a VH comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, and/or a VHFWR4 amino acid sequence of SEQ ID NO: 6050.

In some embodiments, the antigen binding domain that targets NKp30 comprises a VH comprising a VHFWR1 amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6050.

In some embodiments, the antigen binding domain that targets NKp30 comprises a VL comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, and/or a VLFWR4 amino acid sequence of SEQ ID NO: 6108.

In some embodiments, the antigen binding domain that targets NKp30 comprises a VL comprising a VLFWR1 amino acid sequence of SEQ ID NO: 6105 (or a sequence with no more than 1, 2, or 3 mutations, e.g., substitutions, additions, or deletions), a VLFWR2 amino acid sequence of SEQ ID NO: 6106 (or a sequence with no more than 1 mutation, e.g., substitution, addition, or deletion), a VLFWR3 amino acid sequence of SEQ ID NO: 6107 (or a sequence with no more than 1 mutation, e.g., substitution, addition, or deletion), and/or a VLFWR4 amino acid sequence of SEQ ID NO: 6108.

In some embodiments, the antigen binding domain that targets NKp30 comprises a VH comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, and/or a VHFWR4 amino acid sequence of SEQ ID NO: 6054.

In some embodiments, the antigen binding domain that targets NKp30 comprises a VH comprising a VHFWR1 amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6054.

In some embodiments, the antigen binding domain that targets NKp30 comprises a VL comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, and/or a VLFWR4 amino acid sequence of SEQ ID NO: 6112.

In some embodiments, the antigen binding domain that targets NKp30 comprises a VL comprising a VLFWR1 amino acid sequence of SEQ ID NO: 6109 (or a sequence with no more than 1, 2, or 3 mutations, e.g., substitutions, additions, or deletions), a VLFWR2 amino acid sequence of SEQ ID NO: 6110 (or a sequence with no more than 1 mutation, e.g., substitution, addition, or deletion), a VLFWR3 amino acid sequence of SEQ ID NO: 6111 (or a sequence with no more than 1 mutation, e.g., substitution, addition, or deletion), and/or a VLFWR4 amino acid sequence of SEQ ID NO: 6112.

In some embodiments, the antigen binding domain that targets NKp30 comprises a VH comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, and/or a VHFWR4 amino acid sequence of SEQ ID NO: 6058.

In some embodiments, the antigen binding domain that targets NKp30 comprises a VH comprising a VHFWR1 amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6058.

In some embodiments, the antigen binding domain that targets NKp30 comprises a VL comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, and/or a VLFWR4 amino acid sequence of SEQ ID NO: 6116.

In some embodiments, the antigen binding domain that targets NKp30 comprises a VL comprising a VLFWR1 amino acid sequence of SEQ ID NO: 6113 (or a sequence with no more than 1, 2, or 3 mutations, e.g., substitutions, additions, or deletions), a VLFWR2 amino acid sequence of SEQ ID NO: 6114 (or a sequence with no more than 1 mutation, e.g., substitution, addition, or deletion), a VLFWR3 amino acid sequence of SEQ ID NO: 6115 (or a sequence with no more than 1 mutation, e.g., substitution, addition, or deletion), and/or a VLFWR4 amino acid sequence of SEQ ID NO: 6116.

In some embodiments, the antigen binding domain that targets NKp30 comprises a VH comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, and/or a VHFWR4 amino acid sequence of SEQ ID NO: 6062.

In some embodiments, the antigen binding domain that targets NKp30 comprises a VH comprising a VHFWR1 amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6062.

In some embodiments, the antigen binding domain that targets NKp30 comprises a VL comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, and/or a VLFWR4 amino acid sequence of SEQ ID NO: 6120.

In some embodiments, the antigen binding domain that targets NKp30 comprises a VL comprising a VLFWR1 amino acid sequence of SEQ ID NO: 6117 (or a sequence with no more than 1, 2, or 3 mutations, e.g., substitutions, additions, or deletions), a VLFWR2 amino acid sequence of SEQ ID NO: 6118 (or a sequence with no more than 1 mutation, e.g., substitution, addition, or deletion), a VLFWR3 amino acid sequence of SEQ ID NO: 6119 (or a sequence with no more than 1 mutation, e.g., substitution, addition, or deletion), and/or a VLFWR4 amino acid sequence of SEQ ID NO: 6120.

In some embodiments, the antigen binding domain that targets NKp30 comprises a VH comprising the amino acid sequence of SEQ ID NO: 6148 (or an amino acid sequence having at least about 77%, 80%, 85%, 90%, 95%, or 99% sequence identity to SEQ ID NO: 6148). In some embodiments, the antigen binding domain that targets NKp30 comprises a VH comprising the amino acid sequence of SEQ ID NO: 6149 (or an amino acid sequence having at least about 77%, 80%, 85%, 90%, 95%, or 99% sequence identity to SEQ ID NO: 6149). In some embodiments, the antigen binding domain that targets NKp30 comprises a VL comprising the amino acid sequence of SEQ ID NO: 6150 (or an amino acid sequence having at least about 93%, 95%, or 99% sequence identity to SEQ ID NO: 6150). In some embodiments, antigen binding domain that targets NKp30 comprises a VH comprising the amino acid sequence of SEQ ID NO: 6148. In some embodiments, antigen binding domain that targets NKp30 comprises a VH comprising the amino acid sequence of SEQ ID NO: 6149. In some embodiments, the antigen binding domain that targets NKp30 comprises a VL comprising the amino acid sequence of SEQ ID NO: 6150.

In some embodiments, the antigen binding domain that targets NKp30 comprises a VH comprising the amino acid sequence of SEQ ID NO: 6148, and a VL comprising the amino acid sequence of SEQ ID NO: 6150. In some embodiments, the antigen binding domain that targets NKp30 comprises a VH comprising the amino acid sequence of SEQ ID NO: 6149, and a VL comprising the amino acid sequence of SEQ ID NO: 6150.

In some embodiments, the antigen binding domain that targets NKp30 comprises a VH comprising the amino acid sequence of SEQ ID NO: 6151 (or an amino acid sequence having at least about 77%, 80%, 85%, 90%, 95%, or 99% sequence identity to SEQ ID NO: 6151). In some embodiments, the antigen binding domain that targets NKp30 comprises a VH comprising the amino acid sequence of SEQ ID NO: 6152 (or an amino acid sequence having at least about 77%, 80%, 85%, 90%, 95%, or 99% sequence identity to SEQ ID NO: 6152). In some embodiments, the antigen binding domain that targets NKp30 comprises a VL comprising the amino acid sequence of SEQ ID NO: 6153 (or an amino acid sequence having at least about 93%, 95%, or 99% sequence identity to SEQ ID NO: 6153). In some embodiments, antigen binding domain that targets NKp30 comprises a VH comprising the amino acid sequence of SEQ ID NO: 6151. In some embodiments, antigen binding domain that targets NKp30 comprises a VH comprising the amino acid sequence of SEQ ID NO: 6152. In some embodiments, the antigen binding domain that targets NKp30 comprises a VL comprising the amino acid sequence of SEQ ID NO: 6153.

In some embodiments, the antigen binding domain that targets NKp30 comprises a VH comprising the amino acid sequence of SEQ ID NO: 6151, and a VL comprising the amino acid sequence of SEQ ID NO: 6153. In some embodiments, the antigen binding domain that targets NKp30 comprises a VH comprising the amino acid sequence of SEQ ID NO: 6152, and a VL comprising the amino acid sequence of SEQ ID NO: 6153.

In some embodiments, the antigen binding domain that targets NKp30 comprises an scFv. In some embodiments, the scFv comprises an amino acid sequence selected from SEQ ID NOs: 6187-6190, or an amino acid sequence having at least about 93%, 95%, or 99% sequence identity thereto.

TABLE 7
Exemplary heavy chain CDRs and FWRs of NKp30-targeting antigen binding domains

| Ab | | VHCDR | | | | VHCDR | |
|---|---|---|---|---|---|---|---|
| ID | VHFWR1 | 1 | VHFWR2 | VHCDR2 | VHFWR3 | 3 | VHFWR4 |
| 9G1- | QIQLQESG | TGGYH | WIRQFP | YIYSSGS | RISITRDTS | GNWHY | WGQGT |
| HC | PGLVKPS | WN | GKKLEW | TSYNPSL | KNQFFLQL | FDF | MVTVSS |
| | QSLSLTCS | (SEQ ID | MG (SEQ | KS (SEQ | NSVTTEDT | (SEQ ID | (SEQ ID |
| | VTGFSIN | NO: | ID NO: | ID NO: | ATYYCAR | NO: | NO: 6006) |
| | (SEQ ID | 6000) | 6004) | 6001) | (SEQ ID | 6002) | |
| | NO: 6003) | | | | NO: 6005) | | |
| 15H6- | QIQLQESG | TGGYH | WIRQFP | YIYSSGT | RISITRDTS | GNWHY | WGQGT |
| HC | PGLVKPS | WN | GKKLEW | TRYNPSL | KNQFFLQL | FDY | LVAVSS |
| | QSLSLTCS | (SEQ ID | MG (SEQ | KS (SEQ | NSVTPEDT | (SEQ ID | (SEQ ID |
| | VTGFSIN | NO: | ID NO: | ID NO: | ATYYCTR | NO: | NO: 6013) |
| | (SEQ ID | 6007) | 6011) | 6008) | (SEQ ID | 6009) | |
| | NO: 6010) | | | | NO: 6012) | | |
| 9G1- | QIQLQESG | TGGYH | WIRQPA | YIYSSGS | RVTMSRD | GNWHY | WGQGT |
| HC_1 | PGLVKPSE | WN | GKGLEW | TSYNPSL | TSKNQFSL | FDF | MVTVSS |
| | TLSLTCTV | (SEQ ID | IG (SEQ | KS (SEQ | KLSSVTAA | (SEQ ID | (SEQ ID |
| | SGFSIN | NO: | ID NO: | ID NO: | DTAVYYC | NO: | NO: 6017) |
| | (SEQ ID | 6000) | 6015) | 6001) | AR | 6002) | |
| | NO: 6014) | | | | (SEQ ID | | |
| | | | | | NO: 6016) | | |
| 9G1- | QIQLQESG | TGGYH | WIRQHP | YIYSSGS | LVTISRDT | GNWHY | WGQGT |
| HC_2 | PGLVKPS | WN | GKGLEW | TSYNPSL | SKNQFSLK | FDF | MVTVSS |
| | QTLSLTCT | (SEQ ID | IG (SEQ | KS (SEQ | LSSVTAAD | (SEQ ID | (SEQ ID |
| | VSGFSIN | NO: | ID NO: | ID NO: | TAVYYCA | NO: | NO: 6021) |
| | (SEQ ID | 6000) | 6019) | 6001) | R | 6002) | |
| | NO: 6018) | | | | (SEQ ID | | |
| | | | | | NO: 6020) | | |
| 9G1- | EIQLLESG | TGGYH | WVRQAP | YIYSSGS | RFTISRDTS | GNWHY | WGQGT |
| HC_3 | GGLVQPG | WN | GKGLEW | TSYNPSL | KNTFYLQ | FDF | MVTVSS |
| | GSLRLSCA | (SEQ ID | VG (SEQ | KS (SEQ | MNSLRAE | (SEQ ID | (SEQ ID |
| | VSGFSIN | NO: | ID NO: | ID NO: | DTAVYYC | NO: | NO: 6025) |
| | (SEQ ID | 6000) | 6023) | 6001) | AR | 6002) | |
| | NO: 6022) | | | | (SEQ ID | | |
| | | | | | NO: 6024) | | |
| 9G1- | QIQLVQSG | TGGYH | WVRQAP | YIYSSGS | RVTITRDT | GNWHY | WGQGT |
| HC_4 | AEVKKPG | WN | GQGLEW | TSYNPSL | STNTFYME | FDF | MVTVSS |
| | SSVKVSC | (SEQ ID | MG (SEQ | KS (SEQ | LSSLRSED | (SEQ ID | (SEQ ID |
| | KVSGFSIN | NO: | ID NO: | ID NO: | TAVYYCA | NO: | NO: 6029) |
| | (SEQ ID | 6000) | 6027) | 6001) | R | 6002) | |
| | NO: 6026) | | | | (SEQ ID | | |
| | | | | | NO: 6028) | | |
| 9G1- | EIQLVESG | TGGYH | WVRQAP | YIYSSGS | RFT1SRDT | GNWHY | WGQGT |
| HC_5 | GGLVQPG | WN | GKGLEW | TSYNPSL | AKNSFYL | FDF | MVTVSS |
| | GSLRLSCA | (SEQ ID | VG (SEQ | KS (SEQ | QMNSLRA | (SEQ ID | (SEQ ID |
| | VSGFSIN | NO: | ID NO: | ID NO: | EDTAVYY | NO: | NO: 6034) |
| | (SEQ ID | 6000) | 6032) | 6001) | CAR (SEQ | 6002) | |
| | NO: 6030) | | | | ID NO: | | |
| | | | | | 6033) | | |
| 9G1- | QIQLVQSG | TGGYH | WVRQAP | YIYSSGS | RVTMTRD | GNWHY | WGQGT |
| HC_6 | AEVKKPG | WN | GQGLEW | TSYNPSL | TSTNTFYM | FDF | MVTVSS |
| | ASVKVSC | (SEQ ID | MG (SEQ | KS (SEQ | ELSSLRSE | (SEQ ID | (SEQ ID |
| | KVSGFSIN | NO: | ID NO: | ID NO: | DTAVYYC | NO: | NO: 6038) |
| | (SEQ ID | 6000) | 6036) | 6001) | AR | 6002) | |
| | NO: 6035) | | | | (SEQ ID | | |
| | | | | | NO: 6037) | | |
| 15H6- | QIQLQESG | TGGYH | WIRQHP | YIYSSGT | LVTISRDT | GNWHY | WGQGT |
| HC_1 | PGLVKPS | WN | GKGLEW | TRYNPSL | SKNQFSLK | FDY | LVTVSS |
| | QTLSLTCT | (SEQ ID | IG (SEQ | KS (SEQ | LSSVTAAD | (SEQ ID | (SEQ ID |
| | VSGFSIN | NO: | ID NO: | ID NO: | TAVYYCA | NO: | NO: 6042) |
| | (SEQ ID | 6007) | 6040) | 6008) | R (SEQ ID | 6009) | |
| | NO: 6039) | | | | NO: 6041) | | |
| 15H6- | QIQLQESG | TGGYH | W1RQPA | YIYSSGT | RVTMSRD | GNWHY | WGQGT |
| HC 2 | PGLVKPSE | WN | GKGLEW | TRYNPSL | TSKNQFSL | FDY | LVTVSS |
| | TLSLTCTV | (SEQ ID | IG (SEQ | KS (SEQ | KLSSVTAA | (SEQ ID | (SEQ ID |
| | SGFSIN | NO: | ID NO: | ID NO: | DTAVYYC | NO: | NO: 6046) |
| | (SEQ ID | 6007) | 6044) | 6008) | AR | 6009) | |
| | NO: 6043) | | | | (SEQ ID | | |
| | | | | | NO: 6045) | | |
| 15H6- | EIQLLESG | TGGYH | WVRQAP | YIYSSGT | RFTISRDTS | GNWHY | WGQGT |
| HC 3 | GGLVQPG | WN | GKGLEW | TRYNPSL | KNTFYLQ | FDY | LVTVSS |
| | GSLRLSCA | (SEQ ID | VG (SEQ | KS (SEQ | MNSLRAE | (SEQ ID | (SEQ ID |
| | VSGFSIN | NO: | ID NO: | ID NO | DTAVYYC | NO: | NO: 6050) |
| | (SEQ ID | 6007) | 6048) | 6008) | AR | 6009) | |
| | NO: 6047) | | | | (SEQ ID | | |
| | | | | | NO: 6049) | | |
| 15H6- | QIQLVESG | TGGYH | WIRQAP | YIYSSGT | RFTISRDT | GNWHY | WGQGT |
| HC_4 | GGLVKPG | WN | GKGLEW | TRYNPSL | AKNSFYL | FDY | LVTVSS |
| | GSLRLSCA | (SEQ ID | VG (SEQ | KS (SEQ | QMNSLRA | (SEQ ID | (SEQ ID |
| | VSGFSIN | NO: | ID NO: | ID NO: | EDTAVYY | NO: | NO: 6054) |
| | (SEQ ID | 6007) | 6052) | 6008) | CAR | 6009) | |
| | NO: 6051) | | | | (SEQ | | |
| | | | | | ID NO: | | |
| | | | | | 6053) | | |
| 15H6- | QIQLVQSG | TGGYH | WVRQAP | YIYSSGT | RVTMTRD | GNWHY | WGQGT |
| HC_5 | AEVKKPG | WN | GQGLEW | TRYNPSL | TSTNTFYM | FDY | LVTVSS |
| | ASVKVSC | (SEQ ID | MG (SEQ | KS (SEQ | ELSSLRSE | (SEQ ID | (SEQ ID |
| | KVSGFSIN | NO: | ID NO: | ID NO: | DTAVYYC | NO: | NO: 6058) |
| | (SEQ ID | 6007) | 6056) | 6008) | AR | 6009) | |
| | NO: 6055) | | | | (SEQ ID | | |
| | | | | | NO: 6057) | | |
| 15H6- | EIQLVQSG | TGGYH | WVQQAP | YIYSSGT | RVT1TRDT | GNWHY | WGQGT |
| HC_6 | AEVKKPG | WN | GKGLEW | TRYNPSL | STNTFYME | FDY | LVTVSS |
| | ATVKJSCK | (SEQ ID | MG (SEQ | KS (SEQ | LSSLRSED | (SEQ ID | (SEQ ID |
| | VSGFSIN | NO: | ID NO: | ID NO: | TAVYYCA | NO: | NO: 6062) |
| | (SEQ ID | 6007) | 6060) | 6008) | R | 6009) | |
| | NO: 6059) | | | | (SEQ ID | | |
| | | | | | NO: 6061) | | |

TABLE 18
Exemplary heavy chain CDRs and FWRs of NKp30-targeting antigen
binding domains (according to the Kabat numbering scheme)

| Ab | | VHCDR | | | | VHCDR | |
|---|---|---|---|---|---|---|---|
| ID | VHFWR1 | 1 | VHFWR2 | VHCDR2 | VHFWR3 | 3 | VHFWR4 |
| 9G1- | QIQLQESG | GYHWN | WIRQFP | YIYSSGS | RISITRDTS | GNWHY | WGQGT |
| HC | PGLVKPS | (SEQ ID | GKKLEW | TSYNPSL | KNQFFLQL | FDF | MVTVSS |
| | QSLSLTCS | NO: | MG (SEQ | KS (SEQ | NSVTTEDT | (SEQ ID | (SEQ ID |
| | VTGFSINT | 7313) | ID NO: | ID NO: | ATYYCAR | NO: | NO: 6006) |
| | G (SEQ ID | | 6004) | 6001) | (SEQ ID | 6002) | |
| | NO: 7317) | | | | NO: 6005) | | |
| 15H6- | QIQLQESG | GYHWN | WIRQFP | YIYSSGT | RISITRDTS | GNWHY | WGQGT |
| HC | PGLVKPS | (SEQ ID | GKKLEW | TRYNPSL | KNQFFLQL | FDY | LVAVSS |
| | QSLSLTCS | NO: | MG (SEQ | KS (SEQ | NSVTPEDT | (SEQ ID | (SEQ ID |
| | VTGFSINT | 7313) | ID NO: | ID NO: | ATYYCTR | NO: | NO: 6013) |
| | G (SEQ ID | | 6011) | 6008) | (SEQ ID | 6009) | |
| | NO: 7317) | | | | NO: 6012) | | |
| 9G1- | QIQLQESG | GYHWN | W1RQPA | YIYSSGS | RVTMSRD | GNWHY | WGQGT |
| HC_1 | PGLVKPSE | (SEQ ID | GKGLEW | TSYNPSL | TSKNQFSL | FDF | MVTVSS |
| | TLSLTCTV | NO: | IG (SEQ | KS (SEQ | KLSSVTAA | (SEQ ID | (SEQ ID |
| | SGFSINTG | 7313) | ID NO: | ID NO: | DTAVYYC | NO: | NO: 6017) |
| | (SEQ ID | | 6015) | 6001) | AR (SEQ ID | 6002) | |
| | NO: 7371) | | | | NO: 6016) | | |
| 9G1- | QIQLQESG | GYHWN | WIRQHP | YIYSSGS | LVTISRDT | GNWHY | WGQGT |
| HC_2 | PGLVKPS | (SEQ ID | GKGLEW | TSYNPSL | SKNQFSLK | FDF | MVTVSS |
| | QTLSLTCT | NO: | IG (SEQ | KS (SEQ | LSSVTAAD | (SEQ ID | (SEQ ID |
| | VSGFSINT | 7313) | ID NO: | ID NO | TAVYYCA | NO: | NO: 6021) |
| | G (SEQ ID | | 6019) | 6001) | R (SEQ ID | 6002) | |
| | NO: 7372) | | | | NO: 6020) | | |
| 9G1- | EIQLLESG | GYHWN | WVRQAP | YIYSSGS | RFTISRDTS | GNWHY | WGQGT |
| HC_3 | GGLVQPG | (SEQ ID | GKGLEW | TSYNPSL | KNTFYLQ | FDF | MVTVSS |
| | GSLRLSCA | NO: | VG (SEQ | KS (SEQ | MNSLRAE | (SEQ ID | (SEQ ID |
| | VSGFSINT | 7313) | ID NO: | ID NO: | DTAVYYC | NO: | NO: 6025) |
| | G (SEQ ID | | 6023) | 6001) | AR (SEQ ID | 6002) | |
| | NO: 7373) | | | | NO: 6024) | | |
| 9G1- | QIQLVQSG | GYHWN | WVRQAP | YIYSSGS | RVTITRDT | GNWHY | WGQGT |
| HC_4 | AEVKKPG | (SEQ ID | GQGLEW | TSYNPSL | STNTFYME | FDF | MVTVSS |
| | SSVKVSC | NO: | MG (SEQ | KS (SEQ | LSSLRSED | (SEQ ID | (SEQ ID |
| | KVSGFSIN | 7313) | ID NO: | ID NO: | TAVYYCA | NO: | NO: 6029) |
| | TG (SEQ ID | | 6027) | 6001) | R(SEQ ID | 6002) | |
| | NO: 7374) | | | | NO: 6028) | | |
| 9G1- | E1QLVESG | GYHWN | WVRQAP | YIYSSGS | RFTISRDT | GNWHY | WGQGT |
| HC_5 | GGLVQPG | (SEQ ID | GKGLEW | TSYNPSL | AKNSFYL | FDF | MVTVSS |
| | GSLRLSCA | NO: | VG (SEQ | KS (SEQ | QMNSLRA | (SEQ ID | (SEQ ID |
| | VSGFSINT | 7313) | ID NOL | ID NO: | EDTAVYY | NO: | NO: 6034) |
| | G (SEQ ID | | 6032) | 6001) | CAR (SEQ | 6002) | |
| | NO: 7375) | | | | ID NO: | | |
| | | | | | 6033) | | |
| 9G1- | QIQLVQSG | GYHWN | WVRQAP | YIYSSGS | RVTMTRD | GNWHY | WGQGT |
| HC_6 | AEVKKPG | (SEQ ID | GQGLEW | TSYNPSL | TSTNTFYM | FDF | MVTVSS |
| | ASVKVSC | NO: | MG (SEQ | KS (SEQ | ELSSLRSE | (SEQ ID | (SEQ ID |
| | KVSGFSIN | 7313) | ID NO: | ID NO: | DTAVYYC | NO: | NO: 6038) |
| | TG (SEQ ID | | 6036) | 6001) | AR (SEQ ID | 6002) | |
| | NO: 7376) | | | | NO: 6037) | | |
| 15H6- | QIQLQESG | GYHWN | WIRQHP | YIYSSGT | LVTISRDT | GNWHY | WGQGT |
| HC_1 | PGLVKPS | (SEQ ID | GKGLEW | TRYNPSL | SKNQFSLK | FDY | LVTVSS |
| | QTLSLTCT | NO: | IG (SEQ | KS (SEQ | LSSVTAAD | (SEQ ID | (SEQ ID |
| | VSGFSINT | 7313) | ID NO: | ID NO: | TAVYYCA | NO: | NO: 6042) |
| | G (SEQ ID | | 6040) | 6008) | R (SEQ ID | 6009) | |
| | NO: 7372) | | | | NO: 6041) | | |
| 15H6- | QIQLQESG | GYHWN | WIRQPA | YIYSSGT | RVTMSRD | GNWHY | WGQGT |
| HC_2 | PGLVKPSE | (SEQ ID | GKGLEW | TRYNPSL | TSKNQFSL | FDY | LVTVSS |
| | TLSLTCTV | NO: | IG (SEQ | KS (SEQ | KLSSVTAA | (SEQ ID | (SEQ ID |
| | SGFSINTG | 7313) | ID NO: | ID NO: | DTAVYYC | NO: | NO: 6046) |
| | (SEQ ID | | 6044) | 6008) | AR | 6009) | |
| | NO: 7371) | | | | (SEQ ID | | |
| | | | | | NO: 6045) | | |
| 15H6- | E1QLLESG | GYHWN | WVRQAP | YIYSSGT | RFTISRDTS | GNWHY | WGQGT |
| HC_3 | GGLVQPG | (SEQ ID | GKGLEW | TRYNPSL | KNTFYLQ | FDY | LVTVSS |
| | GSLRLSCA | NO: | VG (SEQ | KS (SEQ | MNSLRAE | (SEQ ID | (SEQ ID |
| | VSGFSINT | 7313) | ID NO: | ID NO: | DTAVYYC | NO: | NO: 6050) |
| | G (SEQ ID | | 6048) | 6008) | AR | 6009) | |
| | NO: 7373) | | | | (SEQ ID | | |
| | | | | | NO: 6049) | | |
| 15H6- | QIQLVESG | GYHWN | WIRQAP | YIYSSGT | RFTISRDT | GNWHY | WGQGT |
| HC_4 | GGLVKPG | (SEQ ID | GKGLEW | TRYNPSL | AKNSFYL | FDY | LVTVSS |
| | GSLRLSCA | NO: | VG (SEQ | KS (SEQ | QMNSLRA | (SEQ ID | (SEQ ID |
| | VSGFSINT | 7313) | ID NO: | ID NO | EDTAVYY | NO: | NO: 6054) |
| | G (SEQ ID | | 6052) | 6008) | CAR | 6009) | |
| | NO: 7377) | | | | (SEQ | | |
| | | | | | ID NO: | | |
| | | | | | 6053) | | |
| 15H6- | QIQLVQSG | GYHWN | WVRQAP | YIYSSGT | RVTMTRD | GNWHY | WGQGT |
| HC_5 | AEVKKPG | (SEQ ID | GQGLEW | TRYNPSL | TSTNTFYM | FDY | LVTVSS |
| | ASVKVSC | NO: | MG (SEQ | KS (SEQ | ELSSLRSE | (SEQ ID | (SEQ ID |
| | KVSGFSIN | 7313) | ID NO: | ID NO: | DTAVYYC | NO: | NO: 6058) |
| | TG (SEQ ID | | 6056) | 6008) | AR (SEQ ID | 6009) | |
| | NO: 7376) | | | | NO: 6057) | | |
| 15H6- | EIQLVQSG | GYHWN | WVQQAP | YIYSSGT | RVTITRDT | GNWHY | WGQGT |
| HC_6 | AEVKKPG | (SEQ ID | GKGLEW | TRYNPSL | STNTFYME | FDY | LVTVSS |
| | ATVKISCK | NO: | MG (SEQ | KS (SEQ | LSSLRSED | (SEQ ID | (SEQ ID |
| | VSGFSINT | 7313) | ID NO: | ID NO: | TAVYYCA | NO: | NO: 6062) |
| | G (SEQ ID | | 6060) | 6008) | R | 6009) | |
| | NO: 7378) | | | | (SEQ ID | | |
| | | | | | NO: 6061) | | |
| 9D9- | QIQLQESG | GYHWN | WIRQFP | YIYSSGT | RISITRDTS | GDWHY | WGQGT |
| HC | PGLVKPS | (SEQ ID | GKKVE | TKYNPSL | KNQFFLQL | FDY | MVAVSS |
| | QSLSLSCS | NO: | WMG | KS (SEQ | NSVTTEDT | (SEQ ID | (SEQ ID |
| | VTGFSINT | 7313) | (SEQ ID | ID NO: | ATYYCAR | NO: | NO: 7316) |
| | G (SEQ ID | | NO: 7314) | 7385) | (SEQ ID | 7315) | |
| | NO: 7312) | | | | NO: 6005) | | |
| 3A12- | QIQLQESG | GYHWN | WIRQFP | YIYSSGS | RFSITRDTS | GNWHY | WGQGT |
| HC | PGLVKPS | (SEQ ID | GKKLEW | TRYNPSL | KNQFFLQL | FDY | LVAVSS |
| | QSLSLTCS | NO: | MG (SEQ | KS (SEQ | NSVTTEDT | (SEQ ID | (SEQ ID |
| | VTGFSINT | 7313) | ID NO: | ID NO: | ATYYCTR | NO: | NO: 6013) |
| | G (SEQ ID | | 6004) | 7318) | (SEQ ID | 6009) | |
| | NO: 7317) | | | | NO: 7319) | | |
| 12D10- | QIQLQESG | GYHWN | WIRQFP | YIYSSGT | RISITRDTS | GNWHY | WGQGT |
| HC | PGLVKPS | (SEQ ID | GKKLEW | TRYNPSL | KNQFFLQL | FDY | LVAVSS |
| | QSLSLTCS | NO: | MG (SEQ | KS (SEQ | NSVTPEDT | (SEQ ID | (SEQ ID |
| | VTGFSINT | 7313) | ID NO: | ID NO: | ATYYCTR | NO: | NO: 6013) |
| | G (SEQ ID | | 6004) | 6008) | (SEQ ID | 6009) | |
| | NO: 7317) | | | | NO: 6012) | | |
| 15E1- | QIQLQESG | GYHWN | WIRQFP | YIYSSGS | RFSITRDTS | GDWHY | WGPGT |
| HC | PGLVKPS | (SEQ ID | GKKLEW | TSYNPSL | KNQFFLQL | FDY | MVTVSS |
| | QSLSLSCS | NO: | MG (SEQ | KS (SEQ | NSVTTEDT | (SEQ ID | (SEQ ID |
| | VTGFSITT | 7313) | ID NO: | ID NO: | ATYYCAR | NO: | NO: 7324) |
| | T (SEQ ID | | 6004) | 6001) | (SEQ ID | 7315) | |
| | NO: 7322) | | | | NO: 7323) | | |
| 15E1_ | QIQLQESG | GYHWN | WIRQHP | YIYSSGS | LVTISRDT | GDWHY | WGQGT |
| Humanized | PGLVKPS | (SEQ ID | GKGLEW | TSYNPSL | SKNQFSLK | FDY | MVTVSS |
| variant_ | QTLSLTCT | NO: | IG | KS (SEQ | LSSVTAAD | (SEQ ID | (SEQ ID |
| VH1 | VSGFSITT | 7313) | (SEQ | ID NO: | TAVYYCA | NO: | NO: 6006) |
| | T (SEQ ID | | ID NO: | 6001) | R | 7315) | |
| | NO: 7330) | | 6019) | | (SEQ ID | | |
| | | | | | NO: 6020) | | |
| 15E1_ | QIQLVESG | GYHWN | WIRQAP | YIYSSGS | RFTISRDT | GDWHY | WGQGT |
| Humanized | GGLVKPG | (SEQ ID | GKGLEW | TSYNPSL | AKNSFYL | FDY | MVTVSS |
| variant_ | GSLRLSCA | NO: | VG | KS (SEQ | QMNSLRA | (SEQ ID | (SEQ ID |
| VH2 | VSGFSITT | 7313) | (SEQ | ID NO | EDTAVYY | NO: | NO: 6006) |
| | T (SEQ ID | | ID NO: | 6001) | CAR (SEQ | 7315) | |
| | NO: 7331) | | 6052) | | ID NO: | | |
| | | | | | 6033) | | |
| 15E1_ | EIQLLESG | GYHWN | WVRQAP | YIYSSGS | RFTISRDTS | GDWHY | WGQGT |
| Humanized | GGLVQPG | (SEQ ID | GKGLEW | TSYNPSL | KNTFYLQ | FDY | MVTVSS |
| variant_ | GSLRLSCA | NO: | VG | KS (SEQ | MNSLRAE | (SEQ ID | (SEQ ID |
| VH3 | VSGFSITT | 7313) | (SEQ | ID NO: | DTAVYYC | NO: | NO: 6006) |
| | T (SEQ ID | | ID NO: | 6001) | AR | 7315) | |
| | NO: 7332) | | 6023) | | (SEQ ID | | |
| | | | | | NO: 6024) | | |
| 15E1_ | EIQLVESG | GYHWN | WVRQAP | YIYSSGS | RFTISRDT | GDWHY | WGQGT |
| Humanized | GGLVQPG | (SEQ ID | GKGLEW | TSYNPSL | AKNSFYL | FDY | MVTVSS |
| variant_ | GSLRLSCA | NO: | VG | KS (SEQ | QMNSLRA | (SEQ ID | (SEQ ID |
| VH4 | VSGFSITT | 7313) | (SEQ | ID NO: | EDTAVYY | NO: | NO: 6006) |
| | T (SEQ ID | | ID NO: | 6001) | CAR | 7315) | |
| | NO: 7333) | | 6023) | | (SEQ | | |
| | | | | | ID NO: | | |
| | | | | | 6033) | | |
| 15E1_ | QIQLVQSG | GYHWN | WVRQAP | YIYSSGS | RVTMTRD | GDWHY | WGQGT |
| Humanized | AEVKKPG | (SEQ ID | GQGLEW | TSYNPSL | TSTNTFYM | FDY | MVTVSS |
| variant_ | ASVKVSC | NO: | MG | KS | ELSSLRSE | (SEQ ID | (SEQ ID |
| VH5 | KVSGFSIT | 7313) | (SEQ | (SEQ | DTAVYYC | NO: | NO: 6006) |
| | TT (SEQ ID | | ID NO: | ID NO: | AR | 7315) | |
| | NO: 7334) | | 6027) | 6001) | (SEQ ID | | |
| | | | | | NO: 6037) | | |

TABLE 8
Exemplary light chain CDRs and FWRs of NKp30-targeting antigen binding domains

| Ab | | | | | | | |
|---|---|---|---|---|---|---|---|
| ID | VLFWR1 | VLCDR1 | VLFWR2 | VLCDR2 | VLFWR3 | VLCDR3 | VLFWR4 |
| 9G1- | SYTLTQPP | SGERLS | WYQQKP | ENDKRP | GIPDQFSG | QSWDS | FGSGTQ |
| LC | LLSVALG | DKYVH | GRAPVM | S (SEQ ID | SNSGNIAT | TNSAV | LTVL |
| | HKATITC | (SEQ ID | VIY (SEQ | NO: 6064) | LTISKAQA | (SEQ ID | (SEQ ID |
| | (SEQ ID | NO: | ID NO: | | GYEADYY | NO: | NO: 6069) |
| | NO: 6066) | 6063) | 6067) | | C | 7293) | |
| | | | | | (SEQ ID | | |
| | | | | | NO: 7292) | | |
| 15H6- | SYTLTQPP | SGENLS | WYQQKP | ENEKRPS | GIPDQFSG | HYWESI | FGSGTH |
| LC | SLSVAPG | DKYVH | GRAPVM | (SEQ ID | SNSGNIAT | NSW | LTVL |
| | QKATITC | (SEQ ID | VIY | NO: 6071) | LTISKAQP | (SEQ ID | (SEQ ID |
| | (SEQ ID | NO: | (SEQ | | GSEADYY | NO: | NO: 6076) |
| | NO: 6073) | 6070) | ID NO: | | C | 6072) | |
| | | | 6074) | | (SEQ ID | | |
| | | | | | NO: 6075) | | |
| 9G1- | QSVTTQPP | SGERLS | WYQQLP | ENDKRP | GVPDRFSG | QSWDS | FGGGTQ |
| LC_1 | SVSGAPG | DKYVH | GTAPKM | S (SEQ ID | SNSGNSAS | TNSAV | LTVL |
| | QRVTISC | (SEQ ID | LIY | NO: 6064) | LAITGLQA | (SEQ ID | (SEQ ID |
| | (SEQ ID | NO: | (SEQ | | EDEADYY | NO: | NO: 6080) |
| | NO: 6077) | 6063) | ID NO: | | C | 7293) | |
| | | | 6078) | | (SEQ ID | | |
| | | | | | NO: 6079) | | |
| 9G1- | QSVTTQPP | SGERLS | WYQQLP | ENDKRP | GVPDRFSG | QSWDS | FGGGTQ |
| LC_2 | SASGTPG | DKYVH | GTAPKM | S (SEQ ID | SNSGNSAS | TNSAV | LTVL |
| | QRVTISC | (SEQ ID | LIY | NO: 6064) | LAISGLQS | (SEQ ID | (SEQ ID |
| | (SEQ ID | NO: | (SEQ | | EDEADYY | NO: | NO: 6084) |
| | NO: 6081) | 6063) | ID NO: | | C | 7293) | |
| | | | 6082) | | (SEQ ID | | |
| | | | | | NO: 6083) | | |
| 9G1- | QSVTTQPP | SGERLS | WYQQLP | ENDKRP | GVPDRFSG | QSWDS | FGGGTQ |
| LC_3 | SASGTPG | DKYVH | GTAPKM | S (SEQ ID | SNSGNSAS | TNSAV | LTVL |
| | QRVTISC | (SEQ ID | LIY | NO: 6064) | LAISGLRS | (SEQ ID | (SEQ ID |
| | (SEQ ID | NO: | (SEQ | | EDEADYY | NO: | NO: 6088) |
| | NO: 6085) | 6063) | ID NO: | | C | 7293) | |
| | | | 6086) | | (SEQ ID | | |
| | | | | | NO: 6087) | | |
| 9G1- | SSETTQPH | SGERLS | WYQQKP | ENDKRP | GIPERFSGS | QSWDS | FGGGTQ |
| LC_4 | SVSVATA | DKYVH | GQDPVM | S (SEQ ID | NPGNTATL | TNSAV | LTVL |
| | QMARITC | (SEQ ID | VIY | NO: 6064) | TISRIEAGD | (SEQ ID | (SEQ ID |
| | (SEQ ID | NO: | (SEQ | | EADYYC | NO: | NO: 6092) |
| | NO: 6089) | 6063) | ID NO: | | (SEQ ID | 7293) | |
| | | | 6090) | | NO: 6091) | | |
| 9G1- | DIQMTQSP | SGERLS | WYQQKP | ENDKRP | GVPSRFSG | QSWDS | FGQGTK |
| LC 5 | STLSASVG | DKYVH | GKAPKM | S (SEQ ID | SNSGNEAT | TNSAV | VEIK |
| | DRVTITC | (SEQ ID | LIY | NO: 6064) | LTISSLQPD | (SEQ ID | (SEQ ID |
| | (SEQ ID | NO: | (SEQ | | DFATYYC | NO: | NO: 6096) |
| | NO: 6093) | 6063) | ID NO: | | (SEQ ID | 7293) | |
| | | | 6094) | | NO: 6095) | | |
| 15H6- | QYVLTQP | SGENLS | WYQQLP | ENEKRPS | GVPDRFSG | HYWESI | FGEGTE |
| LC_1 | PSASGTPG | DKYVH | GTAPKM | (SEQ ID | SNSGNSAS | NSW | LTVL |
| | QRVTISC | (SEQ ID | LIY | NO: 6071) | LAISGLQS | (SEQ ID | (SEQ ID |
| | (SEQ ID | NO: | (SEQ | | EDEADYY | NO: | NO: 6100) |
| | NO: 6097) | 6070) | ID NO: | | C | 6072) | |
| | | | 6098) | | (SEQ ID | | |
| | | | | | NO: 6099) | | |
| 15H6- | QYVLTQP | SGENLS | WYQQLP | ENEKRPS | GVPDRFSG | HYWESI | FGEGTE |
| LC_2 | PSASGTPG | DKYVH | GTAPKM | (SEQ ID | SNSGNSAS | NSW | LTVL |
| | QRVTISC | (SEQ ID | LIY | NO: 6071) | LAISGLRS | (SEQ ID | (SEQ ID |
| | (SEQ ID | NO: | (SEQ | | EDEADYY | NO: | NO: 6104) |
| | NO: 6101) | 6070) | ID NO: | | C | 6072) | |
| | | | 6102) | | (SEQ ID | | |
| | | | | | NO: 6103) | | |
| 15H6- | SYELTQPP | SGENLS | WYQQKP | ENEKRPS | GIPERFSGS | HYWESI | FGEGTE |
| LC_3 | SVSVSPGQ | DKYVH | GQSPVM | (SEQ ID | NSGNTATL | NSW | LTVL |
| | TASITC | (SEQ ID | VIY | NO: 6071) | TISGTQAM | (SEQ ID | (SEQ ID |
| | (SEQ ID | NO: | (SEQ | | DEADYYC | NO: | NO: 6108) |
| | NO: 6105) | 6070) | ID NO: | | (SEQ ID | 6072) | |
| | | | 6106) | | NO: 6107) | | |
| 15H6- | DYVLTQS | SGENLS | WYLQKP | ENEKRPS | GVPDRFSG | HYWESI | FGQGTK |
| LC_4 | PLSLPVTP | DKYVH | GQSPQM | (SEQ ID | SNSGNDA | NSW | VEIK |
| | GEPASISC | (SEQ ID | LIY | NO: 6071) | TLKISRVE | (SEQ ID | (SEQ ID |
| | (SEQ ID | NO: | (SEQ | | AEDVGVY | NO: | NO: 6112) |
| | NO: 6109) | 6070) | ID NO: | | YC (SEQ ID | 6072) | |
| | | | 6110) | | NO: 6111) | | |
| 15H6- | AYQLTQS | SGENLS | WYQQKP | ENEKRPS | GVPSRFSG | HYWESI | FGQGTK |
| LC_5 | PSSLSASV | DKYVH | GKAPKM | (SEQ ID | SNSGNDA | NSW | VEIK |
| | GDRVTITC | (SEQ ID | LIY | NO: 6071) | TLTISSLQP | (SEQ ID | (SEQ ID |
| | (SEQ ID | NO: | (SEQ | | EDFATYY | NO: | NO: 6116) |
| | NO: 6113) | 6070) | ID NO: | | C (SEQ ID | 6072) | |
| | | | 6114) | | NO: 6115) | | |
| 15H6- | EYVLTQSP | SGENLS | WYQQKP | ENEKRPS | GIPARFSG | HYWESI | FGQGTK |
| LC_6 | ATLSVSPG | DKYVH | GQAPRM | (SEQ ID | SNSGNEAT | NSW | VEIK |
| | ERATLSC | (SEQ ID | LIY | NO: 6071) | LTISSLQSE | (SEQ ID | (SEQ ID |
| | (SEQ ID | NO: | (SEQ | | DFAVYYC | NO: | NO: 6120) |
| | NO: 6117) | 6070) | ID NO: | | (SEQ ID | 6072) | |
| | | | 6118) | | NO: 6119) | | |
| 9D9- | SYTLTQPP | SGENLS | WYQQKP | ENDKRP | GIPDQFSG | HCWDS | FGSGTH |
| LC | LVSVALG | DKYVH | GRAPVM | S (SEQ ID | SNSGNIAT | TNSAV | LTVL |
| | QKATIIC | (SEQ ID | VIY | NO: 6064) | LTISKAQA | (SEQ ID | (SEQ ID |
| | (SEQ ID | NO: | (SEQ | | GYEADYY | NO: | NO: 6076) |
| | NO: 7320) | 6070) | ID NO: | | C (SEQ ID | 7321) | |
| | | | 6067) | | NO: 7292) | | |
| 3A12- | SYTLTQPP | SGENLS | WYQQKP | ENDKRP | GIPDQFSG | HCWDS | FGSGTH |
| LC | LVSVALG | DKYVH | GRAPVM | S (SEQ ID | SNSGNIAT | TNSAV | LTVL |
| | QKATIIC | (SEQ ID | VIY | NO: 6064) | LTISKAQA | (SEQ ID | (SEQ ID |
| | (SEQ ID | NO: | (SEQ | | GYEADYY | NO: | NO: 6076) |
| | NO: 7320) | 6070) | ID NO: | | C (SEQ ID | 7321) | |
| | | | 6067) | | NO: 7292) | | |
| 12D10- | SYTLTQPP | SGENLS | WYQQKP | ENEKRPS | GIPDQFSG | HYWESI | FGSGTH |
| LC | SLSVAPG | DKYVH | GRAPVM | (SEQ ID | SNSGNIAT | NSW | LTVL |
| | QKATIIC | (SEQ ID | VIY | NO: 6071) | LTISKAQP | (SEQ ID | (SEQ ID |
| | (SEQ ID | NO: | (SEQ | | GSEADYY | NO: | NO: 6076) |
| | NO: 6073) | 6070) | ID NO: | | C (SEQ ID | 6072) | |
| | | | 6074) | | NO: 6075) | | |
| 15E1- | SFTLTQPP | SGEKLS | WYQQKP | ENDRRPS | GIPDQFSG | QFWDS | FGGGTQ |
| LC | LVSVAVG | DKYVH | GRAPVM | (SEQ ID | SNSGNIAS | TNSAV | LTVL |
| | QVATITC | (SEQ ID | VIY | NO: 7327) | LTISKAQA | (SEQ ID | (SEQ ID |
| | (SEQ ID | NO: | (SEQ | | GDEADYF | NO: | NO: 6080) |
| | NO: 7325) | 7326) | ID NO: | | C (SEQ ID | 7329) | |
| | | | 6067) | | NO: 7328) | | |
| 15E1_ | SSETTQPP | SGEKLS | WYQQKP | ENDRRPS | GIPERFSGS | QFWDS | FGGGTQ |
| Humanized | SVSVSPGQ | DKYVH | GQSPVM | (SEQ ID | NSGNTATL | TNSAV | LTVL |
| variant_ | TASITC | (SEQ ID | VIY | NO: 7327) | TISGTQAM | (SEQ ID | (SEQ ID |
| VL1 | (SEQ ID | NO: | (SEQ | | DEADYFC | NO: | NO: 6080) |
| | NO: 7335) | 7326) | ID NO: | | (SEQ ID | 7329) | |
| | | | 6106) | | NO: 7336) | | |
| 15E1_ | SSETTQPH | SGEKLS | WYQQKP | ENDRRPS | GIPERFSGS | QFWDS | FGGGTQ |
| Humanized | SVSVATA | DKYVH | GQDPVM | (SEQ ID | NPGNTATL | TNSAV | LTVL |
| variant_ | QMAR1TC | (SEQ ID | VIY | NO: 7327) | TISRIEAGD | (SEQ ID | (SEQ ID |
| VL2 | (SEQ ID | NO: | (SEQ | | EADYFC | NO: | NO: 6080) |
| | NO: 6089) | 7326) | ID NO: | | (SEQ ID | 7329) | |
| | | | 6090) | | NO: 7337) | | |
| 15E1_ | QSVTTQPP | SGEKLS | WYQQLP | ENDRRPS | GVPDRFSG | QFWDS | FGGGTQ |
| Humanized | SASGTPG | DKYVH | GTAPKM | (SEQ ID | SNSGNSAS | TNSAV | LTVL |
| variant_ | QRVTISC | (SEQ ID | LIY | NO: 7327) | LAISGLRS | (SEQ ID | (SEQ ID |
| VL3 | (SEQ ID | NO: | (SEQ | | EDEADYF | NO: | NO: 6080) |
| | NO: 6081) | 7326) | ID NO: | | C | 7329) | |
| | | | 6078) | | (SEQ ID | | |
| | | | | | NO: 7338) | | |
| 15E1_ | QSVTTQPP | SGEKLS | WYQQLP | ENDRRPS | GVPDRFSG | QFWDS | FGGGTQ |
| Humanized | SVSGAPG | DKYVH | GTAPKM | (SEQ ID | SNSGNSAS | TNSAV | LTVL |
| variant_ | QRVTISC | (SEQ ID | LIY | NO: 7327) | LAITGLQA | (SEQ ID | (SEQ ID |
| VL4 | (SEQ ID | NO: | (SEQ | | EDEADYF | NO: | NO: 6080) |
| | NO: 6077) | 7326) | ID NO: | | C | 7329) | |
| | | | 6078) | | (SEQ ID | | |
| | | | | | NO: 7339) | | |
| 15E1_ | DSVTTQSP | SGEKLS | WYQQRP | ENDRRPS | GVPDRFSG | QFWDS | FGGGTK |
| Humanized | LSLPVTLG | DKYVH | GQSPRM | (SEQ ID | SNSGNDA | TNSAV | VEIK |
| variant_ | QPASISC | (SEQ ID | LIY | NO: 7327) | TLKISRVE | (SEQ ID | (SEQ ID |
| VL5 | (SEQ ID | NO: | (SEQ | | AEDVGVY | NO: | NO: 233) |
| | NO: 7340) | 7326) | ID NO: | | FC (SEQ ID | 7329) | |
| | | | 7341) | | NO: 7342) | | |

TABLE 9
Exemplary variable regions of NKp30-
targeting antigen binding domains

| SEQ | | Descrip- | |
|---|---|---|---|
| ID NO | Ab ID | tion | Sequence |
| SEQ | 9G1-HC | 9G1 | QIQLQESGPGLVKPSQ |
| ID NO: | | heavy | SLSLTCSVTGFSINTG |
| 6121 | | chain | GYHWNWIRQFPGKKLE |
| | | variable | WMGYIYSSGSTSYNPS |
| | | region | LKSRISITRDTSKNQF |
| | | | FLQLNSVTTEDTATYY |
| | | | CARGNWHYFDFWGQGT |
| | | | MVTVSS |
| SEQ | 15H6-HC | 15H6 | QIQLQESGPGLVKPSQ |
| ID NO: | | heavy | SLSLTCSVTGFSINTG |
| 6122 | | chain | GYHWNWIRQFPGKKLE |
| | | variable | WMGYIYSSGTTRYNPS |
| | | region | LKSRISITRDTSKNQF |
| | | | FLQLNSVTPEDTATYY |
| | | | CTRGNWHYFDYWGQGT |
| | | | LVAVSS |
| SEQ | 9G1-HC1 | 9G1 | QIQLQESGPGLVKPSE |
| ID NO: | | heavy | TLSLTCTVSGFSINTG |
| 6123 | | chain | GYHWNWIRQPAGKGLE |
| | | variable | WIGYIYSSGSTSYNPS |
| | | region | LKSRVTMSRDTSKNQF |
| | | humanized | SLKLSSVTAADTAVYY |
| | | variant 1 | CARGNWHYFDFWGQGT |
| | | | MVTVSS |
| SEQ | 9G1-HC2 | 9G1 | QIQLQESGPGLVKPSQ |
| ID NO: | | heavy | TLSLTCTVSGFSINTG |
| 6124 | | chain | GYHWNWIRQHPGKGLE |
| | | variable | WIGYIYSSGSTSYNPS |
| | | region | LKSLVTISRDTSKNQF |
| | | humanized | SLKLSSVTAADTAVYY |
| | | variant 2 | CARGNWHYFDFWGQGT |
| | | | MVTVSS |
| SEQ | 9G1-HC3 | 9G1 | EIQLLESGGGLVQPGG |
| ID NO: | | heavy | SLRLSCAVSGFSINTG |
| 6125 | | chain | GYHWNWVRQAPGKGLE |
| | | variable | WVGYIYSSGSTSYNPS |
| | | region | LKSRFTISRDTSKNTF |
| | | humanized | YLQMNSLRAEDTAVYY |
| | | variant 3 | CARGNWHYFDFWGQGT |
| | | | MVTVSS |
| SEQ | 9G1-HC4 | 9G1 | QIQLVQSGAEVKKPGS |
| ID NO: | | heavy | SVKVSCKVSGFSINTG |
| 6126 | | chain | GYHWNWVRQAPGQGLE |
| | | variable | WMGYIYSSGSTSYNPS |
| | | region | LKSRVTITRDTSTNTF |
| | | humanized | YMELSSLRSEDTAVYY |
| | | variant 4 | CARGNWHYFDFWGQGT |
| | | | MVTVSS |
| SEQ | 9G1-HC5 | 9G1 | EIQLVESGGGLVQPGG |
| ID NO: | | heavy | SLRLSCAVSGFSINTG |
| 6127 | | chain | GYHWNWVRQAPGKGLE |
| | | variable | WVGYIYSSGSTSYNPS |
| | | region | LKSRFTISRDTAKNSF |
| | | humanized | YLQMNSLRAEDTAVYY |
| | | variant 5 | CARGNWHYFDFWGQGT |
| | | | MVTVSS |
| SEQ | 9G1-HC6 | 9G | QIQLVQSGAEVKKPGA |
| ID NO: | | 1 heavy | SVKVSCKVSGFSINTG |
| 6128 | | chain | GYHWNWVRQAPGQGLE |
| | | variable | WMGYIYSSGSTSYNPS |
| | | region | LKSRVTMTRDTSTNTF |
| | | humanized | YMELSSLRSEDTAVYY |
| | | variant 6 | CARGNWHYFDFWGQGT |
| | | | MVTVSS |
| SEQ | 15H6- | 15H6 | QIQLQESGPGLVKPSQ |
| ID NO: | HC_1 | heavy | TLSLTCTVSGFSINTG |
| 6129 | | chain | GYHWNWIRQHPGKGLE |
| | | variable | WIGYIYSSGTTRYNPS |
| | | region | LKSLVTISRDTSKNQF |
| | | humanized | SLKLSSVTAADTAVYY |
| | | variant 1 | CARGNWHYFDYWGQGT |
| | | | LVTVSS |
| SEQ | 15H6- | 15H6 | QIQLQESGPGLVKPSE |
| ID NO: | HC_2 | heavy | TLSLTCTVSGFSINTG |
| 6130 | | chain | GYHWNWIRQPAGKGLE |
| | | variable | WIGYIYSSGTTRYNPS |
| | | region | LKSRVTMSRDTSKNQF |
| | | humanized | SLKLSSVTAADTAVYY |
| | | variant 2 | CARGNWHYFDYWGQGT |
| | | | LVTVSS |
| SEQ | 15H6- | 15H6 | EIQLLESGGGLVQPGG |
| ID NO: | HC_3 | heavy | SLRLSCAVSGFSINTG |
| 6131 | | chain | GYHWNWVRQAPGKGLE |
| | | variable | WVGYIYSSGTTRYNPS |
| | | region | LKSRFTISRDTSKNTF |
| | | humanized | YLQMNSLRAEDTAVYY |
| | | variant 3 | CARGNWHYFDYWGQGT |
| | | | LVTVSS |
| SEQ | I5H6- | 15H6 | QIQLVESGGGLVKPGG |
| ID NO: | HC 4 | heavy | SLRLSCAVSGFSINTG |
| 6132 | | chain | GYHWNWIRQAPGKGLE |
| | | variable | WVGYIYSSGTTRYNPS |
| | | region | LKSRFTISRDTAKNSF |
| | | humanized | YLQMNSLRAEDTAVYY |
| | | variant 4 | CARGNWHYFDYWGQGT |
| | | | LVTVSS |
| SEQ | 15H6- | 15H6 | QIQLVQSGAEVKKPGA |
| ID NO: | HC_5 | heavy | SVKVSCKVSGFSINTG |
| 6133 | | chain | GYHWNWVRQAPGQGLE |
| | | variable | WMGYIYSSGTTRYNPS |
| | | region | LKSRVTMTRDTSTNTF |
| | | humanized | YMELSSLRSEDTAVYY |
| | | variant 5 | CARGNWHYFDYWGQGT |
| | | | LVTVSS |
| SEQ | 15H6- | 15H6 | EIQLVQSGAEVKKPGA |
| ID NO: | HC_6 | heavy | TVKISCKVSGFSINTG |
| 6134 | | chain | GYHWNWVQQAPGKGLE |
| | | variable | WMGYIYSSGTTRYNPS |
| | | region | LKSRVTITRDTSTNTF |
| | | humanized | YMELSSLRSEDTAVYY |
| | | variant 6 | CARGNWHYFDYWGQGT |
| | | | LVTVSS |
| SEQ | 9G1-LC | 9G1 | SYTLTQPPLLSVALGH |
| ID NO: | | light | KATITCSGERLSDKYV |
| 7294 | | chain | HWYQQKPGRAPVMVIY |
| | | variable | ENDKRPSGIPDQFSGS |
| | | region | NSGNIATLTISKAQAG |
| | | | YEADYYCQSWDSTNSA |
| | | | VFGSGTQLTVL |
| SEQ | 15H6-LC | 15H6 | SYTLTQPPSLSVAPGQ |
| ID NO: | | light | KATIICSGENLSDKYV |
| 6136 | | chain | HWYQQKPGRAPVMVIY |
| | | variable | ENEKRPSGIPDQFSGS |
| | | region | NSGNIATLTISKAQPG |
| | | | SEADYYCHYWESINSV |
| | | | VFGSGTHLTVL |
| SEQ | 9G1-LC_1 | 9G1 | QSVTTQPPSVSGAPG |
| ID NO: | | light | QRVTISCSGERLSDK |
| 6137 | | chain | YVHWYQQLPGTAPKM |
| | | variable | LIYENDKRPSGVPDR |
| | | region | FSGSNSGNSASLAIT |
| | | humanized | GLQAEDEADYYCQSW |
| | | variant 1 | DSTNSAVFGGGTQLT |
| | | | VL |
| SEQ | 9G1-LC_2 | 9G1 | QSVTTQPPSASGTPG |
| ID NO: | | light | QRVTISCSGERLSDK |
| 6138 | | chain | YVHWYQQLPGTAPKM |
| | | variable | LIYENDKRPSGVPDR |
| | | region | FSGSNSGNSASLAIS |
| | | humanized | GLQSEDEADYYCQSW |
| | | variant 2 | DSTNSAVFGGGTQLT |
| | | | VL |
| SEQ | 9G1-LC_3 | 9G1 | QSVTTQPPSASGTPG |
| ID NO: | | light | QRVTISCSGERLSDK |
| 6139 | | chain | YVHWYQQLPGTAPKM |
| | | variable | LIYENDKRPSGVPDR |
| | | region | FSGSNSGNSASLAIS |
| | | humanized | GLRSEDEADYYCQSW |
| | | variant 3 | DSTNSAVFGGGTQLT |
| | | | VL |
| SEQ | 9G1-LC_4 | 9G1 | SSETTQPHSVSVATA |
| ID NO: | | light | QMARITCSGERLSDK |
| 6140 | | chain | YVHWYQQKPGQDPVM |
| | | variable | VIYENDKRPSGIPER |
| | | region | FSGSNPGNTATLTIS |
| | | humanized | RIEAGDEADYYCQSW |
| | | variant 4 | DSTNSAVFGGGTQLT |
| | | | VL |
| SEQ | 9G1-LC_5 | 9G1 | DIQMTQSPSTLSASV |
| ID NO: | | light | GDRVTITCSGERLSD |
| 6141 | | chain | KYVHWYQQKPGKAPK |
| | | variable | MLIYENDKRPSGVPS |
| | | region | RFSGSNSGNEATLTI |
| | | humanized | SSLQPDDFATYYCQS |
| | | variant 5 | WDSTNSAVFGQGTKV |
| | | | EIK |
| SEQ | 15H6- | 15H6 | QYVLTQPPSASGTPG |
| ID NO: | LC_1 | light | QRVTISCSGENLSDK |
| 6142 | | chain | YVHWYQQLPGTAPKM |
| | | variable | LIYENEKRPSGVPDR |
| | | region | FSGSNSGNSASLAIS |
| | | humanized | GLQSEDEADYYCHYW |
| | | variant 1 | ESINSVVFGEGTELT |
| | | | VL |
| SEQ | 15H6- | 15H6 | QYVLTQPPSASGTPG |
| ID NO: | LC_2 | light | QRVTISCSGENLSDK |
| 6143 | | chain | YVHWYQQLPGTAPKM |
| | | variable | LIYENEKRPSGVPDR |
| | | region | FSGSNSGNSASLAIS |
| | | humanized | GLRSEDEADYYCHYW |
| | | variant 2 | ESINSVVFGEGTELT |
| | | | VL |
| SEQ | I5H6- | 15H6 | SYELTQPPSVSVSPG |
| ID NO: | LC_3 | light | QTASITCSGENLSDK |
| 6144 | | chain | YVHWYQQKPGQSPVM |
| | | variable | VIYENEKRPSGIPER |
| | | region | FSGSNSGNTATLTIS |
| | | humanized | GTQAMDEADYYCHYW |
| | | variant 3 | ESINSVVFGEGTELT |
| | | | VL |
| SEQ | 15H6- | 15H6 | DYVLTQSPLSLPVTP |
| ID NO: | LC_4 | light | GEPASISCSGENLSD |
| 6145 | | chain | KYVHWYLQKPGQSPQ |
| | | variable | MLIYENEKRPSGVPD |
| | | region | RFSGSNSGNDATLKI |
| | | humanized | SRVEAEDVGVYYCHY |
| | | variant 4 | WESINSVVFGQGTKV |
| | | | EIK |
| SEQ | 15H6- | 15H6 | AYQLTQSPSSLSASV |
| ID NO: | LC_5 | light | GDRVTITCSGENLSD |
| 6146 | | chain | KYVHWYQQKPGKAPK |
| | | variable | MLIYENEKRPSGVPS |
| | | region | RFSGSNSGNDATLTI |
| | | humanized | SSLQPEDFATYYCHY |
| | | variant 5 | WESINSVVFGQGTKV |
| | | | EIK |
| SEQ | 15H6- | 15H6 | EYVLTQSPATLSVSP |
| ID NO: | LC_6 | light | GERATLSCSGENLSD |
| 6147 | | chain | KYVHWYQQKPGQAPR |
| | | variable | MLIYENEKRPSGIPA |
| | | region | RFSGSNSGNEATLTI |
| | | humanized | SSLQSEDFAVYYCHY |
| | | variant 6 | WESINSVVFGQGTKV |
| | | | EIK |
| SEQ | 9D9-HC | 9D9 | QIQLQESGPGLVKPS |
| ID NO: | | heavy | QSLSLSCSVTGFSIN |
| 7295 | | chain | TGGYHWNWIRQFPGK |
| | | variable | KVEWMGYIYSSGTTK |
| | | region | YNPSLKSRISITRDT |
| | | | SKNQFFLQLNSVTTE |
| | | | DTATYYCARGDWHYF |
| | | | DYWGQGTMVAVSS |
| SEQ | 9D9-LC | 9D9 | SYTLTQPPLVSVALG |
| ID NO: | | light | QKATIICSGENLSDK |
| 7296 | | chain | YVHWYQQKPGRAPVM |
| | | variable | VIYENDKRPSGIPDQ |
| | | region | FSGSNSGNIATLTIS |
| | | | KAQAGYEADYYCHCW |
| | | | DSTNSAVFGSGTHLT |
| | | | VL |
| SEQ | 3A12-HC | 3A12 | QIQLQESGPGLVKPS |
| ID NO: | | heavy | QSLSLTCSVTGFSIN |
| 7297 | | chain | TGGYHWNWIRQFPGK |
| | | variable | KLEWMGYIYSSGSTR |
| | | region | YNPSLKSRFSITRDT |
| | | | SKNQFFLQLNSVTTE |
| | | | DTATYYCTRGNWHYF |
| | | | DYWGQGTLVAVSS |
| SEQ | 3A12-LC | 3A12 | SYTLTQPPLVSVALG |
| ID NO: | | light | QKATIICSGENLSDK |
| 7296 | | chain | YVHWYQQKPGRAPVM |
| | | variable | VIYENDKRPSGIPDQ |
| | | region | FSGSNSGNIATLTIS |
| | | | KAQAGYEADYYCHCW |
| | | | DSTNSAVFGSGTHLT |
| | | | VL |
| SEQ | 12D10-HC | 12D10 | QIQLQESGPGLVKPS |
| ID NO: | | heavy | QSLSLTCSVTGFSIN |
| 6122 | | chain | TGGYHWNWIRQFPGK |
| | | variable | KLEWMGYIYSSGTTR |
| | | region | YNPSLKSRISITRDT |
| | | | SKNQFFLQLNSVTPE |
| | | | DTATYYCTRGNWHYF |
| | | | DYWGQGTLVAVSS |
| SEQ | 12D10-LC | 12D10 | SYTLTQPPSLSVAPG |
| ID NO: | | light | QKATIICSGENLSDK |
| 6136 | | chain | YVHWYQQKPGRAPVM |
| | | variable | VIYENEKRPSGIPDQ |
| | | region | FSGSNSGNIATLTIS |
| | | | KAQPGSEADYYCHYW |
| | | | ESINSVVFGSGTHLT |
| | | | VL |
| SEQ | 15E1-HC | 15E1 | QIQLQESGPGLVKPS |
| ID NO: | | heavy | QSLSLSCSVTGFSIT |
| 7298 | | chain | TTGYHWNWIRQFPGK |
| | | variable | KLEWMGYIYSSGSTS |
| | | region | YNPSLKSRFSITRDT |
| | | | SKNQFFLQLNSVTTE |
| | | | DTATYYCARGDWHYF |
| | | | DYWGPGTMVTVSS |
| SEQ | 15E1-LC | 15E1 | SFTLTQPPLVSVAVG |
| ID NO: | | light | QVATITCSGEKLSDK |
| 7299 | | chain | YVHWYQQKPGRAPVM |
| | | variable | VIYENDRRPSGIPDQ |
| | | region | FSGSNSGNIASLTIS |
| | | | KAQAGDEADYFCQFW |
| | | | DSTNSAVFGGGTQLT |
| | | | VL |
| SEQ | 15E1_Hum | 15E1 | QIQLQESGPGLVKPS |
| ID NO: | anized | heavy | QTLSLTCTVSGFSIT |
| 7300 | variant VH | chain | TTGYHWNWIRQHPGK |
| | 1 | variable | GLEWIGYIYSSGSTS |
| | | region | YNPSLKSLVTISRDT |
| | | humanized | SKNQFSLKLSSVTAA |
| | | variant 1 | DTAVYYCARGDWHYF |
| | | | DYWGQGTMVTVSS |
| SEQ | 15E1_ | 15E1 | QIQLVESGGGLVKPG |
| ID NO: | Humanized | heavy | GSLRLSCAVSGFSIT |
| 7301 | variant | chain | TTGYHWNWIRQAPGK |
| | VH 2 | variable | GLEWVGYIYSSGSTS |
| | | region | YNPSLKSRFTISRDT |
| | | humanized | AKNSFYLQMNSLRAE |
| | | variant 2 | DTAVYYCARGDWHYF |
| | | | DYWGQGTMVTVSS |
| SEQ | 15E1_Hum | 15E1 | EIQLLESGGGLVQPG |
| ID NO: | anized | heavy | GSLRLSCAVSGFSIT |
| 7302 | variant VH | chain | TTGYHWNWVRQAPGK |
| | 3 | variable | GLEWVGYIYSSGSTS |
| | (BJM0407 | region | YNPSLKSRFTISRDT |
| | VH and | humanized | SKNTFYLQMNSLRAE |
| | BJM0411 | variant 3 | DTAVYYCARGDWHYF |
| | VH) | | DYWGQGTMVTVSS |
| SEQ | 15E1_Hum | 15E1 | EIQLVESGGGLVQPG |
| ID NO: | anized | heavy | GSLRLSCAVSGFSIT |
| 7303 | variant VH | chain | TTGYHWNWVRQAPGK |
| | 4 | variable | GLEWVGYIYSSGSTS |
| | | region | YNPSLKSRFTISRDT |
| | | humanized | AKNSFYLQMNSLRAE |
| | | variant 4 | DTAVYYCARGDWHYF |
| | | | DYWGQGTMVTVSS |
| SEQ | 15E1_ | 15E1 | QIQLVQSGAEVKKPG |
| ID NO: | Humanized | heavy | ASVKVSCKVSGFSIT |
| 7304 | variant_ | chain | TTGYHWNWVRQAPGQ |
| | VH | variable | GLEWMGYIYSSGSTS |
| | 5 | region | YNPSLKSRVTM |
| | | humanized | TRDTSTNTFYMELSS |
| | | variant 5 | LRSEDTAVYYCARGD |
| | | | WHYFDYWGQGTMVTV |
| | | | SS |
| SEQ | 15E1_ | 15E1 | SSETTQPPSVSVSPG |
| ID NO: | Humanized | light | QTASITCSGEKLSDK |
| 7305 | variant_VL | chain | YVHWYQQKPGQSPVM |
| | 1 | variable | VIYENDRRPSGIPER |
| | (BJM0407 | region | FSGSNSGNTATLTIS |
| | VL) | humanized | GTQAMDEADYFCQFW |
| | | variant 1 | DSTNSAVFGGGTQLT |
| | | | VL |
| SEQ | 15E1_ | 15E1 | SSETTQPHSVSVATA |
| ID NO: | Humanized | light | QMARITCSGEKLSDK |
| 7306 | variant_VL | chain | YVHWYQQKPGQDPVM |
| | 2 | variable | VIYENDRRPSGIPER |
| | | region | FSGSNPGNTATLTIS |
| | | humanized | RIEAGDEADYFCQFW |
| | | variant 2 | DSTNSAVFGGGTQLT |
| | | | VL |
| SEQ | 15E1_ | 15E1 | QSVTTQPPSASGTPG |
| ID NO: | Humanized | light | QRVTISCSGEKLSDK |
| 7307 | variant_VL | chain | YVHWYQQLPGTAPKM |
| | 3 | variable | LIYENDRRPSGVPDR |
| | | region | FSGSNSGNSASLAIS |
| | | humanized | GLRSEDEADYFCQFW |
| | | variant 3 | DSTNSAVFGGGTQLT |
| | | | VL |
| SEQ | 15E1_ | 15E1 | QSVTTQPPSVSGAPG |
| ID NO: | Humanized | light | QRVTISCSGEKLSDK |
| 7308 | variant_VL | chain | YVHWYQQLPGTAPKM |
| | 4 | variable | LIYENDRRPSGVPDR |
| | | region | FSGSNSGNSASLAIT |
| | | humanized | GLQAEDEADYFCQFW |
| | | variant 4 | DSTNSAVFGGGTQLT |
| | | | VL |
| SEQ | 15E1_ | 15E1 | DSVTTQSPLSLPVTL |
| ID NO: | Humanized | light | GQPASISCSGEKLSD |
| 7309 | variant_VL | chain | KYVHWYQQRPGQSPR |
| | 5 | variable | ML1YENDRRPSGVPD |
| | (BJM0411 | region | RFSGSNSGNDATLKI |
| | VL) | humanized | SRVEAEDVGVYFCQF |
| | | variant 5 | WDSTNSAVFGGGTKV |
| | | | EIK |

TABLE 10
Exemplary NKp30-targeting
antigen binding domains/
antibody molecules

| SEQ | Ab | Descrip- | |
|---|---|---|---|
| ID NO | ID | tion | Sequence |
| SEQ | Ch(anti- | 9G1 | QIQLQESGPGLVKPS |
| ID NO: | NKp30 | Heavy | QSLSLTCSVTGFSIN |
| 6148 | 9G1)HC | chain | TGGYHWNWIRQFPGK |
| | N297A | | KLEWMGYIYSSGSTS |
| | | | YNPSLKSRISITRDT |
| | | | SKNQFFLQLNSVTTE |
| | | | DTATYYCARGNWHYF |
| | | | DFWGQGTMVTVSSAS |
| | | | TKGPSVFPLAPSSKS |
| | | | TSGGTAALGCLVKDY |
| | | | FPEPVTVSWNSGALT |
| | | | SGVHTFPAVLQSSGL |
| | | | YSLSSWTVPSSSLGT |
| | | | QTYICNVNHKPSNTK |
| | | | VDKRVEPKSCDKTHT |
| | | | CPPCPAPELLGGPSV |
| | | | FLFPPKPKDTLMISR |
| | | | TPEVTCVVVDVSHED |
| | | | PEVKFNWYVDGVEVH |
| | | | NAKTKPREEQYASTY |
| | | | RVVSVLTVLHQDWLN |
| | | | GKEYKCKVSNKALPA |
| | | | PIEKTISKAKGQPRE |
| | | | PQVCTLPPSREEMTK |
| | | | NQVSLSCAVKGFYPS |
| | | | DIAVEWESNGQPENN |
| | | | YKTTPPVLDSDGSFF |
| | | | LVSKLTVDKSRWQQG |
| | | | NVFSCSVMHEALHNH |
| | | | YTQKSLSLSPGK |
| SEQ | Ch(anti- | 9G1 | QIQLQESGPGLVKPS |
| ID NO: | NKp30 | heavy | QSLSLTCSVTGFSIN |
| 6149 | 9G1)HC | chain | TGGYHWNWIRQFPGK |
| | | | KLEWMGYIYSSGSTS |
| | | | YNPSLKSRISITRDT |
| | | | SKNQFFLQLNSVTTE |
| | | | DTATYYCARGNWHYF |
| | | | DFWGQGTMVTVSSAS |
| | | | TKGPSVFPLAPSSKS |
| | | | TSGGTAALGCLVKDY |
| | | | FPEPVTVSWNSGALT |
| | | | SGVHTFPAVLQSSGL |
| | | | YSLSSVVTVPSSSLG |
| | | | TQTYICNVNHKPSNT |
| | | | KVDKRVEPK |
| | | | SCDKTHTCPPCPAPE |
| | | | LLGGPSVFLFPPKPK |
| | | | DTLMISRTPEVTCVV |
| | | | VDVSHEDPEVKFNWY |
| | | | VDGVEVHNAKTKPRE |
| | | | EQYNSTYRVVSVLTV |
| | | | LHQDWLNGKEYKCKV |
| | | | SNKALPAPIEKTISK |
| | | | AKGQPREPQVCTLPP |
| | | | SREEMTKNQVSLSCA |
| | | | VKGFYPSDIAVEWES |
| | | | NGQPENNYKTTPPVL |
| | | | DSDGSFFLVSKLTVD |
| | | | KSRWQQGNVFSCSVM |
| | | | HEALHNHYTQKSLSL |
| | | | SPGK |
| SEQ | Ch(anti- | 9G1 | SYTLTQPPLLSVALGH |
| ID NO: | NKp30 | Light | KATITCSGERLSDKYV |
| 6150 | 9G1 )LC | chain | HWYQQKPGRAPVMVIY |
| | | | ENDKRPSGIPDQFSGS |
| | | | NSGNIATLTISKAQAG |
| | | | YEADYYCQSWDSTNSA |
| | | | VFGSGTQLTVLGQPKA |
| | | | NPTVTLFPPSSEELQA |
| | | | NKATLVCLISDFYPGA |
| | | | VTVAWKADGSPVKAGV |
| | | | ETTKPSKQSNNKYAAS |
| | | | SYLSLTPEQWKSHRSY |
| | | | SCQVTHEGSTVEKTVA |
| | | | PTECS |
| SEQ | Ch(anti- | 15H6 | QIQLQESGPGLVKPSQ |
| ID NO: | NKp30 | heavy | SLSLTCSVTGFSINTG |
| 6151 | 15H6)HC | chain | GYHWNWIRQFPGKKLE |
| | N297A | | WMGYIYSSGTTRYNPS |
| | | | LKSRISITRDTSKNQF |
| | | | FLQLNSVTPEDTATYY |
| | | | CTRGNWHYFDYWGQGT |
| | | | LVAVSSASTKGPSVFP |
| | | | LAPSSKSTSGGTAALG |
| | | | CLVKDYFPEPVTVSWN |
| | | | SGALTSGVHTFPAVLQ |
| | | | SSGLYSLSSVVTVPSS |
| | | | SLGTQTYICNVNHKPS |
| | | | NTKVDKRVEPKSCDKT |
| | | | HTCPPCPAPELLGGPS |
| | | | VFLFPPKPKDTLMISR |
| | | | TPEVTCVVVDVSHEDP |
| | | | EVKFNWYVDGVEVHNA |
| | | | KTKPREEQYASTYRVV |
| | | | SVLTVLHQDWLNGKEY |
| | | | KCKVSNKALPAPIEKT |
| | | | ISKAKGQPREPQVCTL |
| | | | PPSREEMTKNQVSLSC |
| | | | AVKGFYPSDIAVEWES |
| | | | NGQPENNYKTTPPVLD |
| | | | SDGSFFLVSKLTVDKS |
| | | | RWQQGNVFSCSVMHEA |
| | | | LHNHYTQKSLSLSPGK |
| SEQ | Ch(anti- | 15H6 | QIQLQESGPGLVKPSQ |
| ID NO: | NKp30 | heavy | SLSLTCSVTGFSINTG |
| 6152 | 15H6)HC | chain | GYHWNWIRQFPGKKLE |
| | (hole) | | WMGYIYSSGTTRYNPS |
| | | | LKSRISITRDTSKNQF |
| | | | FLQLNSVTPEDTATYY |
| | | | CTRGNWHYFDYWGQGT |
| | | | LVAVSSASTKGPSVFP |
| | | | LAPSSKSTSGGTAALG |
| | | | CLVKDYFPEPVTVSWN |
| | | | SGALTSGVHTFPAVLQ |
| | | | SSGLYSLSSWTVPSSS |
| | | | LGTQTYICNVNHKPSN |
| | | | TKVDKRVEPKSCDKTH |
| | | | TCPPCPAPELLGGPSV |
| | | | FLFPPKPKDTLMISRT |
| | | | PEVTCVVVDVSHEDPE |
| | | | VKFNWYVDGVEVHNAK |
| | | | TKPREEQYNSTYRVVS |
| | | | VLTVLHQDWLNGKEYK |
| | | | CKVSNKALPAPIEKTI |
| | | | SKAKGQPREPQVCTLP |
| | | | PSREEMTKNQVSLSCA |
| | | | VKGFYPSDIAVEWESN |
| | | | GQPENNYKTTPPVLDS |
| | | | DGSFFLVSKLTVDKSR |
| | | | WQQGNVFSCSVMHEAL |
| | | | HNHYTQKSLSLSPGK |
| SEQ | Ch(anti- | 15H6 | SYTLTQPPSLSVAPG |
| ID NO: | NKp30 | Light | QKATIICSGENLSDK |
| 6153 | 15H6)LC | chain | YVHWYQQKPGRAPVM |
| | | | VIYENEKRPSGIPDQ |
| | | | FSGSNSGNIATLTIS |
| | | | KAQPGSEADYYCHYW |
| | | | ESINSVVFGSGTHLT |
| | | | VLGQPKANPTVTLFP |
| | | | PSSEELQANKATLVC |
| | | | LISDFYPGAVTVAWK |
| | | | ADGSPVKAGVETTKP |
| | | | SKQSNNKYAASSYLS |
| | | | LTPEQWKSHRSYSCQ |
| | | | VTHEGSTVEKTVAPT |
| | | | ECS |
| SEQ | BJM0859 | | EIQLLESGGGLVQPG |
| ID NO: | lambda | | GSLRLSCAVSGFSIT |
| 7310 | scFv | | TTGYHWNWVRQAPGK |
| | | | GLEWVGYIYSSGSTS |
| | | | YNPSLKSRFTISRDT |
| | | | SKNTFYLQMNSLRAE |
| | | | DTAVYYCARGDWHYF |
| | | | DYWGQGTMVTVSSGG |
| | | | GGSGGGGSGGGGSGG |
| | | | GGSSSETTQPPSVSV |
| | | | SPGQTASITCSGEKL |
| | | | SDKYVHWYQQKPGQS |
| | | | PVMVIYENDRRPSGI |
| | | | PERFSGSNSGNTATL |
| | | | TISGTQAMDEADYFC |
| | | | QFWDSTNSAVFGGGT |
| | | | QLTVL |
| SEQ | BJM0860 | | EIQLLESGGGLVQPG |
| ID NO: | kappa | | GSLRLSCAVSGFSIT |
| 7311 | scFv | | TTGYHWNWVRQAPGK |
| | | | GLEWVGYIYSSGSTS |
| | | | YNPSLKSRFT1SRDT |
| | | | SKNTFYLQMNSLRAE |
| | | | DTAVYYCARGDWHYF |
| | | | DYWGQGTMVTVSSGG |
| | | | GGSGGGGSGGGGSGG |
| | | | GGSDSVTTQSPLSLP |
| | | | VTLGQPASISCSGEK |
| | | | LSDKYVHWYQQRPGQ |
| | | | SPRML1YENDRRPSG |
| | | | VPDRFSGSNSGNDAT |
| | | | LKISRVEAEDVGVYF |
| | | | CQFWDSTNSAVFGGG |
| | | | TKVEIK |

In some embodiments, the NK cell engager is an antigen binding domain that binds to NKp46 (e.g., NKp46 present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to NKp46, to the NK cell activates the NK cell. An antigen binding domain that binds to NKp46 (e.g., NKp46 present, e.g., expressed or displayed, on the surface of an NK cell) may be said to target NKp46, the NK cell, or both.

In some embodiments, the NK cell engager is an antigen binding domain that binds to NKG2D (e.g., NKG2D present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to NKG2D, to the NK cell activates the NK cell. An antigen binding domain that binds to NKG2D (e.g., NKG2D present, e.g., expressed or displayed, on the surface of an NK cell) may be said to target NKG2D, the NK cell, or both.

In some embodiments, the NK cell engager is an antigen binding domain that binds to CD16 (e.g., CD16 present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to CD16, to the NK cell activates the NK cell. An antigen binding domain that binds to CD16 (e.g., CD16 present, e.g., expressed or displayed, on the surface of an NK cell) may be said to target CD16, the NK cell, or both.

TABLE 15
Exemplary variable regions of NKp46,
NKG2D, or CD16-targeting antigen
binding domains

| SEQ ID | | Descrip- | |
|---|---|---|---|
| NO | Ab ID | tion | Sequence |
| SEQ ID | NKG2D_ | scFv that | QVHLQESGPGLVKPS |
| NO: 6175 | 1scFv | binds | ETLSLTCTVSDDSIS |
| | | NKG2D | SYYWSWIRQPPGKGL |
| | | | EWIGHISYSGSANYN |
| | | | PSLKSRVTISVDTSK |
| | | | NQFSLKLSSVTAADT |
| | | | AVYYCANWDDAFNIW |
| | | | GQGTMVTVSSGGGGS |
| | | | GGGGSGGGGSGGGGS |
| | | | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QYGSSPWTFGQGTKV |
| | | | EIK |
| SEQ ID | NKG2D | VH that | QVHLQESGPGLVKPS |
| NO: 6176 | 1VH | binds | ETLSLTCTVSDDSIS |
| | | NKG2D | SYYWSWIRQPPGKGL |
| | | | EWIGHISYSGSANYN |
| | | | PSLKSRVTISVDTSK |
| | | | NQFSLKLSSVTAADT |
| | | | AVYYCANWDDAFNIW |
| | | | GQGTMVTVSS |
| SEQ ID | NKG2D | VL that | EIVLTQSPGTLSLSP |
| NO: 6177 | 1VL | binds | GERATLSCRASQSVS |
| | | NKG2D | SSYLAWYQQKPGQAP |
| | | | RLLIYGASSRATGIP |
| | | | DRFSGSGSGTDFTLT |
| | | | ISRLEPEDFAVYYCQ |
| | | | QYGSSPWTFGQGTKV |
| | | | EIK |
| SEQ ID | NKG2D_ | scFv that | EVQLVQSGAEVKEPG |
| NO: 6178 | 2scFv | binds | ESLKISCKNSGYSFT |
| | | NKG2D | NYWVGWVRQMPGKGL |
| | | | EWMGIIYPGDSDTRY |
| | | | SPSFQGQVTISADKS |
| | | | INTAYLQWSSLKASD |
| | | | TAMYYCGRLTMFRGI |
| | | | IIGYFDYWGQGTLVT |
| | | | VSSGGGGSGGGGSGG |
| | | | GGSGGGGSEIVLTQS |
| | | | PATLSLSPGERATLS |
| | | | CRASQSVSSYLAWYQ |
| | | | QKPGQAPRLLIYDAS |
| | | | NRATGIPARFSGSGS |
| | | | GTDFTLTISSLEPED |
| | | | FAVYYCQQRSNWPWT |
| | | | FGQGTKVEIK |
| SEQ ID | NKG2D | VH that | EVQLVQSGAEVKEPG |
| NO: 6179 | 2VH | binds | ESLKISCKNSGYSFT |
| | | NKG2D | NYWVGWVRQMPGKGL |
| | | | EWMGIIYPGDSDTRY |
| | | | SPSFQGQVTISADKS |
| | | | INTAYLQWSSLKASD |
| | | | TAMYYCGRLTMFRGI |
| | | | IIGYFDYWGQGTLVT |
| | | | VSS |
| SEQ ID | NKG2D | VL that | EIVLTQSPATLSLSP |
| NO: 6180 | 2VL | binds | GERATLSCRASQSVS |
| | | NKG2D | SYLAWYQQKPGQAPR |
| | | | LLIYDASNRATGIPA |
| | | | RFSGSGSGTDFTLTI |
| | | | SSLEPEDFAVYYCQQ |
| | | | RSNWPWTFGQGTKVE |
| | | | IK |
| SEQ ID | NKp46scF | scFv | QVQLQQSGPELVKPG |
| NO: 6181 | V | that | ASVKMSCKASGYTFT |
| | | binds | DYVINWGKQRSGQGL |
| | | NKp46 | EWIGEIYPGSGTNYY |
| | | | NEKFKAKATLTADKS |
| | | | SNIAYMQLSSLTSED |
| | | | SAVYFCARRGRYGLY |
| | | | AMDYWGQGTSVTVSS |
| | | | GGGGSGGGGSGGGGS |
| | | | GGGGSDIQMTQTTSS |
| | | | LSASLGDRVTISCRA |
| | | | SQDISNYLNWYQQKP |
| | | | DGTVKLLIYYTSRLH |
| | | | SGVPSRFSGSGSGTD |
| | | | YSLTINNLEQEDIAT |
| | | | YFCQQGNTRPWTFGG |
| | | | GTKLEIK |
| SEQ ID | NKp46V | VH that | QVQLQQSGPELVKPG |
| NO: 6182 | H | binds | ASVKMSCKASGYTFT |
| | | NKp46 | DYVINWGKQRSGQGL |
| | | | EWIGEIYPGSGTNYY |
| | | | NEKFKAKATLTADKS |
| | | | SNIAYMQLSSLTSED |
| | | | SAVYFCARRGRYGLY |
| | | | AMDYWGQGTSVTVSS |
| SEQ ID | NKp46VL | VL that | DIQMTQTTSSLSASL |
| NO: 6183 | | binds | GDRVTISCRASQDIS |
| | | NKp46 | NYLNWYQQKPDGTVK |
| | | | LLIYYTSRLHSGVPS |
| | | | RFSGSGSGTDYSLTI |
| | | | NNLEQEDIATYFCQQ |
| | | | GNTRPWTFGGGTKLE |
| | | | IK |
| SEQ ID | CD16scFv | scFv | EVQLVESGG |
| NO: 6184 | | that | GVVRPGGSLR |
| | | binds | LSCAASGFTF |
| | | CD16 | DDYGMSWVRQ |
| | | | APGKGLEWVS |
| | | | GINWNGGSTG |
| | | | YADSVKGRFT |
| | | | ISRDNAKNSL |
| | | | YLQMNSLRAE |
| | | | DTAVYYCARG |
| | | | RSLLFDYWGQ |
| | | | GTLVTVSRGG |
| | | | GGSGGGGSGG |
| | | | GGSSELTQDP |
| | | | AVSVALGQTV |
| | | | RITCQGDSLR |
| | | | SYYASWYQQK |
| | | | PGQAPVLVIY |
| | | | GKNNRPSGIP |
| | | | DRFSGSSSGN |
| | | | TASLT1TGAQ |
| | | | AEDEADYYCN |
| | | | SRDSSGNHVV |
| | | | FGGGTKLTVL |
| SEQ ID | CD16VH | VH that | EVQLVESGG |
| NO: 6185 | | binds | GVVRPGGSLR |
| | | CD16 | LSCAASGFTF |
| | | | DDYGMSWVRQ |
| | | | APGKGLEWVS |
| | | | GINWNGGSTG |
| | | | YADSVKGRFT |
| | | | ISRDNAKNSL |
| | | | YLQMNSLRAE |
| | | | DTAVYYCARG |
| | | | RSLLFDYWGQ |
| | | | GTLVTVSR |
| SEQ ID | CD16VL | VL that | SSELTQDP |
| NO: 6186 | | binds | AVSVALGQTV |
| | | CD16 | RITCQGDSLR |
| | | | SYYASWYQQK |
| | | | PGQAPVLVIY |
| | | | GKNNRPSGIP |
| | | | DRFSGSSSGNT |
| | | | ASLTITGAQ |
| | | | AEDEADYYCN |
| | | | SRDSSGNHVV |
| | | | FGGGTKLTVL |

In one embodiment, the NK cell engager is a ligand of NKp30, e.g., is a B7-6, e.g., comprises the amino acid sequence of: DLKVEMMAGGTQITPLNDNVTIFCNIFYSQPLNITSMGITWFWKSLTFDKEVKVFEFFGD HQEAFRPGAIVSPWRLKSGDASLRLPGIQLEEAGEYRCEVVVTPLKAQGTVQLEVVASP ASRLLLDQVGMKENEDKYMCES SGFYPEAINITWEKQTQKFPHPIEISEDVITGPTIKNM DGTFNVTSCLKLNSSQEDPGTVYQCVVRHASLHTPLRSNFTLTAARHSLSETEKTDNFS (SEQ ID NO: 7233), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7233.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:

(i) MICA comprises the amino acid sequence: EPHSLRYNLTVLSWDGSVQSGFLTEVHLDGQPFLRCDRQKCRAKPQGQWAEDVLGNK TWDRETRDLTGNGKDLRMTLAHIKDQKEGLHSLQEIRVCEIHEDNSTRSSQHFYYDGEL FLSQNLETKEWTPQSSRAQTLAMNVRNFLKEDAMKTKTHYHAMHADCLQELRRYLK SGVVLRRTVPPMVNVTRSEASEGNITVTCRASGFYPWNITLSWRQDGVSLSHDTQQWG DVLPDGNGTYQTWVATRICQGEEQRFTCYMEHSGNHSTHPVPSGKVLVLQSHW (SEQ ID NO: 7234), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7234;
(ii) MICB comprises the amino acid sequence: AEPHSLRYNLMVLSQDESVQSGFLAEGHLDGQPFLRYDRQKRRAKPQGQWAEDVLGA KTWDTETEDLTENGQDLRRTLTHIKDQKGGLHSLQEIRVCEIHEDSSTRGSRHFYYDGEL FLSQNLETQESTVPQS SRAQTLAMNVTNFWKEDAMKTKTHYRAMQADCLQKLQRYLK SGVAIRRTVPPMVNVTCSEVSEGNITVTCRASSFYPRNITLTWRQDGVSLSHNTQQWGD VLPDGNGTYQTWVATRIRQGEEQRFTCYMEHSGNHGTHPVPSGKVLVLQSQRTD (SEQ ID NO: 7235), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7235; or
(iii) ULBP1 comprises the amino acid sequence: GWVDTHCLCYDFIITPKSRPEPQWCEVQGLVDERPFLHYDCVNHKAKAFASLGKKVNV TKTWEEQTETLRDVVDFLKGQLLDIQVENLIPIEPLTLQARMSCEHEAHGHGRGSWQFL FNGQKFLLFDSNNRKWTALHPGAKKMTEKWEKNRDVTMFFQKISLGDCKMWLEEFL MYWEQMLDPTKPPSLAPG (SEQ ID NO: 7236), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7236.

In other embodiments, the NK cell engager is a ligand of DNAM1 chosen from NECTIN2 or NECL5, e.g., wherein:

(i) NECTIN2 comprises the amino acid sequence: QDVRVQVLPEVRGQLGGTVELPCHLLPPVPGLYISLVTWQRPDAPANHQNVAAFHPKM GPSFPSPKPGSERLSFVSAKQSTGQDTEAELQDATLALHGLTVEDEGNYTCEFATFPKGS VRGMTWLRVIAKPKNQAEAQKVTFSQDPTTVALCISKEGRPPARISWLSSLDWEAKETQ VSGTLAGTVTVTSRFTLVPSGRADGVTVTCKVEHESFEEPALIPVTLSVRYPPEVSISGYD DNWYLGRTDATLSCDVRSNPEPTGYDWSTTSGTFPTSAVAQGSQLVIHAVDSLFNTTFV CTVTNAVGMGRAEQVIFVRETPNTAGAGATGG (SEQ ID NO: 7237), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7237; or
(ii) NECL5 comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7238.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16,the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSS ELKVSLTNVSISDEGRYFCQLYTDPPQESYTTITVLVPPRNLMIDIQKDTAVEGEEIEVNC TAMASKPATTIRWFKGNTELKGKSEVEEWSDMYTVTSQLMLKVHKEDDGVPVICQVE HPAVTGNLQTQRYLEVQYKPQVHIQMTYPLQGLTREGDALELTCEAIGKPQPVMVTWV RVDDEMPQHAVLSGPNLFINNLNKTDNGTYRCEASNIVGKAHSDYMLYVYDPPTTIPPP TTTTTTTTTTTTTILTIITDSRAGEEGSIRAVDH (SEQ ID NO: 7239), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7239.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQ LRIHRDGIYMVHIQVTLAICSSTTASRHHPTTLAVGICSPASRSISLLRLSFHQGCTIASQR LTPLARGDTLCTNLTGTLLPSRNTDETFFGVQWVRP (SEQ ID NO: 7240), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7240.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGI WTWVGTNKSLTEEAENWGDGEPNNKKNKEDCVEIYIKRNKDAGKWNDDACHKLKAA LCYTASCQPWSCSGHGECVEIINNYTCNCDVGYYGPQCQFVIQCEPLEAPELGTMDCTH PLGNFSFSSQCAFSCSEGTNLTGIEETTCGPFGNWSSPEPTCQVIQCEPLSAPDLGIMNCSH PLASFSFTSACTFICSEGTELIGKKKTICESSGIWSNPSPICQKLDKSFSMIKEGDYN (SEQ ID NO: 7241), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7241.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7239.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQ RAHQAAEGQLQACQADRQKTKETLQSEEQQRRALEQKLSNMENRLKPFFTCGSADTCC PSGWIMHQKSCFYISLTSKNWQESQKQCETLSSKLATFSEIYPQSHSYYFLNSLLPNGGS GNSYWTGLSSNKDWKLTDDTQRTRTYAQSSKCNKVHKTWSWWTLESESCRSSLPYICE MTAFRFPD (SEQ ID NO: 7242), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7242.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYF SKEEGDWNSSKYNCSTQHADLTIIDNIEEMNFLRR YKCSSDHWIGLKMAKNRTGQWVDGATFTKSFGMRGSEGCAYLSDDGAATARCYTER KWICRKRIH (SEQ ID NO: 7243), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7243.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGR VRLDPQSGALYISKVQKEDNSTYIMIRVLKKTGNEQEWKIKLQVLDPVPKPVIKIEKIEDM DDNCYLKLSCVIPGESVNYTWYGDKRPFPKELQNSVLETTLMPHNYSRCYTCQVSNSVS SKNGTVCLSPPCTLARS (SEQ ID NO: 7244), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7244.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

Death Receptor Signal Engagers

Death receptors, e.g., death receptors 4 and 5 (DR4 and DR5, also known as TRAIL-R1 and TRAIL-R2 respectively), are trimeric type I transmembrane proteins widely expressed in normal human tissues. Activation of death receptors causes intracellular signaling that induces cell death. TNF-related apoptosis-inducing ligand (TRAIL) (also known as Apo2L) is a trimeric protein that binds to Death receptors, activating their cell death-inducing signaling (Amarante-Mendes and Griffith. Pharmacol Ther. 2015 November; 155:117-31).

The present disclosure provides, inter alia, multispecific (e.g., bi-, tri-, quad-specific) or multifunctional molecules, that are engineered to contain one or more death receptor signal engagers that mediate binding to death receptors and/or activation of death receptor signaling on a target cell (e.g., the T cell comprising a TCRBV antigen (e.g., a TCRBV antigen corresponding to a biased TCRBV clonotype). Accordingly, in some embodiments, the death receptor signal engager comprises one or more TRAIL polypeptides or a fragment thereof (TRAIL molecule), one or more death receptors or a fragment thereof (death receptor molecule), or one or more antigen binding domains that specifically binds to a death receptor (e.g., and activates death receptor signaling). Without wishing to be bound by theory, it is thought that a death receptor signal engager that can activate death receptor signaling on a target cell can induce the death of the target cell, e.g., a target disease cell (e.g., a T cell comprising a TCRBV antigen (e.g., a TCRBV antigen corresponding to a biased TCRBV clonotype).

Death receptor signal engagers may comprise TRAIL molecules and/or death receptor molecules from or derived from versions of TRAIL and death receptors known to those skilled in the art. In some embodiments, the death receptor signal engager comprises a human TRAIL molecule or death receptor molecule. In some embodiments, the death receptor signal engager comprises a mouse TRAIL molecule or death receptor molecule. In some embodiments, the death receptor signal engager comprises a mammalian TRAIL molecule or death receptor molecule. In some embodiments, the death receptor signal engager comprises a truncated TRAIL molecule or death receptor molecule (e.g., relative to a wild-type TRAIL molecule or death receptor molecule).

In some embodiments, the death receptor signal engager comprises a truncated TRAIL molecule comprising at least residues corresponding to amino acids 95-281 of human TRAIL, e.g., a truncated TRAIL molecule comprising residues corresponding to amino acids 95-281 of human TRAIL. In some embodiments, the death receptor signal engager comprises a truncated TRAIL molecule comprising residues of 95-281 of human TRAIL.

In some embodiments, the death receptor signal engager comprises a truncated TRAIL molecule comprising at least residues corresponding to amino acids 122-281 of human TRAIL, e.g., a truncated TRAIL molecule comprising residues corresponding to amino acids 122-281 of human TRAIL. In some embodiments, the death receptor signal engager comprises a truncated TRAIL molecule comprising residues of 122-281 of human TRAIL.

In some embodiments, the death receptor signal engager comprises one, two, or three TRAIL molecules (e.g., the death receptor signal engager is a monomeric, dimeric, or trimeric TRAIL molecule, respectively). In some embodiments, the death receptor signal engager comprises one, two, or three death receptor molecules (e.g., the death receptor signal engager is a monomeric, dimeric, or trimeric death receptor molecule, respectively). In some embodiments, the death receptor signal engager comprises one, two, or three antigen binding domains that specifically bind to a death receptor (e.g., to one or more death receptors, e.g., the same or different death receptors)

In some embodiments, the death receptor signal engager comprises an amino acid sequence selected from Table 11 (or an amino acid sequence having at least about 77%, 80%, 85%, 90%, 95%, or 99% sequence identity to a sequence selected from Table 11).

In some embodiments, the death receptor signal engager comprises an amino acid sequence of SEQ ID NO: 6157 (or an amino acid sequence having at least about 77%, 80%, 85%, 90%, 95%, or 99% sequence identity to SEQ ID NO: 6157).

In some embodiments, the death receptor signal engager comprises an amino acid sequence of SEQ ID NO: 6158 (or an amino acid sequence having at least about 77%, 80%, 85%, 90%, 95%, or 99% sequence identity to SEQ ID NO: 6158).

In some embodiments, the death receptor signal engager comprises an amino acid sequence of SEQ ID NO: 6159 (or an amino acid sequence having at least about 77%, 80%, 85%, 90%, 95%, or 99% sequence identity to SEQ ID NO: 6159).

In some embodiments, the death receptor signal engager comprises an amino acid sequence of SEQ ID NO: 6160 (or an amino acid sequence having at least about 77%, 80%, 85%, 90%, 95%, or 99% sequence identity to SEQ ID NO: 6160).

In some embodiments, the death receptor signal engager comprises an amino acid sequence of SEQ ID NO: 6161 (or an amino acid sequence having at least about 77%, 80%, 85%, 90%, 95%, or 99% sequence identity to SEQ ID NO: 6161).

In some embodiments, the death receptor signal engager comprises an amino acid sequence of SEQ ID NO: 6162 (or an amino acid sequence having at least about 77%, 80%, 85%, 90%, 95%, or 99% sequence identity to SEQ ID NO: 6162).

In some embodiments, the death receptor signal engager comprises an amino acid sequence of SEQ ID NO: 6163 (or an amino acid sequence having at least about 77%, 80%, 85%, 90%, 95%, or 99% sequence identity to SEQ ID NO: 6163).

In some embodiments, the death receptor signal engager comprises an amino acid sequence of SEQ ID NO: 6164 (or an amino acid sequence having at least about 77%, 80%, 85%, 90%, 95%, or 99% sequence identity to SEQ ID NO: 6164).

In some embodiments, the death receptor signal engager comprises an amino acid sequence of SEQ ID NO: 6165 (or an amino acid sequence having at least about 77%, 80%, 85%, 90%, 95%, or 99% sequence identity to SEQ ID NO: 6165).

In some embodiments, the death receptor signal engager is comprised on the same polypeptide chain as another component of a multifunctional molecule of the present disclosure, e.g., the death receptor signal engager is comprised on the same polypeptide chain as a heavy and/or light chain of a first antigen binding domain that preferentially binds to a TCRBV antigen (e.g., a TCRBV antigen corresponding to a biased TCRBV clonotype) on a lymphocyte cell (e.g., T cell), an immune cell engager, a cytokine molecule, or a cytokine inhibitor molecule, e.g., as a fusion protein. In some embodiments, the multifunctional molecule comprises a fusion protein comprising a death receptor signal engager and light chain of a first antigen binding domain that preferentially binds to a TCRBV antigen (e.g., a TCRBV antigen corresponding to a biased TCRBV clonotype). In some embodiments, the multifunctional molecule comprises a fusion protein comprising a death receptor signal engager and a light chain of a first antigen binding domain that selectively targets lymphocytes expressing a TCRBV antigen (e.g., a TCRBV antigen corresponding to a biased TCRBV clonotype).

TABLE 11
Exemplary death receptor signal engagers

| SEQ | | Descrip- | |
|---|---|---|---|
| ID NO | ID Ref. | tion | Sequence |
| SEQ | monomeric_ | Monomeric | METDTLLLWVLLLWV |
| ID NO: | hTRAIL_ | human | PGSTGDYKDDDDKGG |
| 6157 | aa122_ | TRAIL | GGSGTGGAAAHITGT |
| | 281- | comprising | RGRSNTLSSPNSKNE |
| | hFc_ | residues | KALGRKINSWESSRS |
| | Knob_ | 122-281 | GHSFLSNLHLRNGEL |
| | Cys- | | VIHEKGFYYIYSQTY |
| | Blank | | FRFQEEIKENTKNDK |
| | | | QMVQYIYKYTSYPDP |
| | | | ILLMKSARNSCWSKD |
| | | | AEYGLYSIYQGGIFE |
| | | | LKENDRIFVSVTNEH |
| | | | LIDMDHEASFFGAFA |
| | | | VSGSGNGTSNGTSGS |
| | | | SGGDKTHTCPPCPAP |
| | | | ELLGGPSVFLFPPKP |
| | | | KDTLMISRTPEVTCV |
| | | | VVDVSHEDPEVKFNW |
| | | | YVDGVEVHNAKTKPR |
| | | | EEQYNSTYRVVSVLT |
| | | | VLHQDWLNGKEYKCK |
| | | | VSNKALPAPIEKTIS |
| | | | KAKGQPREPQVYTLP |
| | | | PCREEMTKNQVSLWC |
| | | | LVKGFYPSDIAVEWE |
| | | | SNGQPENNYKTTPPV |
| | | | LDSDGSFFLYSKLTV |
| | | | DKSRWQQGNVFSCSV |
| | | | MHEALHNHYTQKSLS |
| | | | LSPGK |
| SEQ | dimeric_ | Dimeric | METDTLLLWVLLLWV |
| ID NO: | hTRAIL_ | human | PGSTGDYKDDDDKGG |
| 6158 | aa122_ | TRAIL | GGSGTGGAAAHITGT |
| | 281- | comprising | RGRSNTLSSPNSKNE |
| | hFc_ | residues | KALGRKINSWESSRS |
| | Knob_ | 122-281 | GHSFLSNLHLRNGEL |
| | Cys- | | VIHEKGFYYIYSQTY |
| | Blank | | FRFQEEIKENTKNDK |
| | | | QMVQYIYKYTSYPDP |
| | | | ILLMKSARNSCWSKD |
| | | | AEYGLYSIYQGGIFE |
| | | | LKENDRIFVSVTNEH |
| | | | LIDMDHEASFFGAFA |
| | | | VSGAAAHITGTRGRS |
| | | | NTLSSPNSKNEKALG |
| | | | RKlNSWESSRSGHSF |
| | | | LSNLHLRNGELVIHE |
| | | | KGFYYIYSQTYFRFQ |
| | | | EEIKENTKNDKQMVQ |
| | | | YIYKYTSYPDPILLM |
| | | | KSARNSCWSKDAEYG |
| | | | LYSIYQGGIFELKEN |
| | | | DRIFVSVTNEHLIDM |
| | | | DHEASFFGAFAVSGS |
| | | | GNGTSNGTSGSSGGD |
| | | | KTHTCPPCPAPELLG |
| | | | GPSVFLFPPKPKDTL |
| | | | MISRTPEVTCVVVDV |
| | | | SHEDPEVKFNWYVDG |
| | | | VEVHNAKTKPREEQY |
| | | | NSTYRVVSVLTVLHQ |
| | | | DWLNGKEYKCKVSNR |
| | | | ALPAPIEKTISKAKG |
| | | | QPREPQVYTLPPCRE |
| | | | EMTKNQVSLWCLVKG |
| | | | FYPSDIAVEWESNGQ |
| | | | PENNYKTTPPVLDSD |
| | | | GSFFLYSKLTVDKSR |
| | | | WQQGNVFSCSVMHEA |
| | | | LHNHYTQKSLSLSPG |
| | | | K |
| SEQ | Trimerich_ | Trimeric | METDTLLLWVLLLWV |
| ID NO: | TRAIL_ | human | PGSTGDYKDDDDKGG |
| 6159 | Aa122_ | TRAIL | GGSGTGGAAAHITGT |
| | 281- | comprising | RGRSNTLSSPNSKNE |
| | hFc_ | residues | KALGRKINSWESSRS |
| | Knob_ | 122-281 | GHSFLSNLHLRNGEL |
| | Cys- | | VIHEKGFYYIYSQTY |
| | Blank | | FRFQEEIKENTKNDK |
| | | | QMVQYIYKYTSYPDP |
| | | | ILLMKSARNSCWSKD |
| | | | AEYGLYSIYQGGIFE |
| | | | LKENDRIFVSVTNEH |
| | | | LIDMDHEASFFGAFA |
| | | | VSGAAAHITGTRGRS |
| | | | NTLSSPNSKNEKALG |
| | | | RKINSWESSRSGHSF |
| | | | LSNLHLRNGELVIHE |
| | | | KGFYYIYSQTYFRFQ |
| | | | EEIKENTKNDKQMVQ |
| | | | YIYKYTSYPDPILLM |
| | | | KSARNSCWSKDAEYG |
| | | | LYSIYQGGIFELKEN |
| | | | DRIFVSVTNEHLIDM |
| | | | DHEASFFGAFAVSGA |
| | | | AAHITGTRGRSNTLS |
| | | | SPNSKNEKALGRKIN |
| | | | SWESSRSGHSFLSNL |
| | | | HLRNGELVIHEKGFY |
| | | | YIYSQTYFRFQEEIK |
| | | | ENTKNDKQMVQYIYK |
| | | | YTSYPDPILLMKSAR |
| | | | NSCWSKDAEYGLYSI |
| | | | YQGGIFELKENDRIF |
| | | | VSVTNEHLIDMDHEA |
| | | | SFFGAFAVSGSGNGT |
| | | | SNGTSGSSGGDKTHT |
| | | | CPPCPAPELLGGPSV |
| | | | FLFPPKPKDTLMISR |
| | | | TPEVTCVVVDVSHED |
| | | | PEVKFNWYVDGVEVH |
| | | | NAKTKPREEQYNSTY |
| | | | RVVSVLTVLHQDWLN |
| | | | GKEYKCKVSNKALPA |
| | | | PIEKTISKAKGQPRE |
| | | | PQVYTLPPCREEMTK |
| | | | NQVSLWCLVKGFYPS |
| | | | DIAVEWESNGQPENN |
| | | | YKTTPPVLDSDGSFF |
| | | | LYSKLTVDKSRWQQG |
| | | | NVFSCSVMHEALHNH |
| | | | YTQKSLSLSPGK |
| SEQ | Monomeric_ | Monomeric | METDTLLLWVLLLWV |
| ID NO: | hTRAIL_ | human | PGSTGTSEETISTVQ |
| 6160 | 95-281- | TRAIL | EKQQNISPLVRERGP |
| | hFc_ | comprising | QRVAAHITGTRGRSN |
| | Knob_ | residues | TLSSPNSKNEKALGR |
| | Cys- | 95-281 | KINSWESSRSGHSFL |
| | Blank | | SNLHLRNGELVIHEK |
| | | | GFYYIYSQTYFRFQE |
| | | | EIKENTKNDKQMVQY |
| | | | IYKYTSYPDPILLMK |
| | | | SARNSCWSKDAEYGL |
| | | | YSIYQGGIFELKEND |
| | | | RIFVSVTNEHLIDMD |
| | | | HEASFFGAFLVGGGG |
| | | | GSGGGGSDKTHTCPP |
| | | | CPAPELLGGPSVFLF |
| | | | PPKPKDTLMISRTPE |
| | | | VTCVVVDVSHEDPEV |
| | | | KFNWYVDGVEVHNAK |
| | | | TKPREEQYNSTYRVV |
| | | | SVLTVLHQDWLNGKE |
| | | | YKCKVSNKALPAPIE |
| | | | KTISKAKGQPREPQV |
| | | | YTLPPCREEMTKNQV |
| | | | SLWCLVKGFYPSDIA |
| | | | VEWESNGQPENNYKT |
| | | | TPPVLDSDGSFFLYS |
| | | | KLTVDKSRWQQGNVF |
| | | | SCSVMHEALHNHYTQ |
| | | | KSLSLSPGK |
| SEQ | Dimeric_ | Dimeric | METDTLLLWVLLLWV |
| ID NO: | hTRAIL_ | human | PGSTGTSEETISTVQ |
| 6161 | 95-281- | TRAIL | EKQQNISPLVRERGP |
| | hFc_ | comprising | QRVAAHITGTRGRSN |
| | Knob_ | residues | TLSSPNSKNEKALGR |
| | Cys- | 95-281 | KINSWESSRSGHSFL |
| | Blank | | SNLHLRNGELVIHEK |
| | | | GFYYIYSQTYFRFQE |
| | | | EIKENTKNDKQMVQY |
| | | | IYKYTSYPDPILLMK |
| | | | SARNSCWSKDAEYGL |
| | | | YSIYQGGIFELKEND |
| | | | RIFVSVTNEHLIDMD |
| | | | HEASFFGAFLVGGGG |
| | | | GSGGGGSGTSEETIS |
| | | | TVQEKQQNISPLVRE |
| | | | RGPQRVAAHITGTRG |
| | | | RSNTLSSPNSKNEKA |
| | | | LGRKINSWESSRSGH |
| | | | SFLSNLHLRNGELVI |
| | | | HEKGFYYIYSQTYFR |
| | | | FQEEIKENTKNDKQM |
| | | | VQYIYKYTSYPDPIL |
| | | | LMKSARNSCWSKDAE |
| | | | YGLYSIYQGGIFELK |
| | | | ENDRIFVSVTNEHLI |
| | | | DMDHEASFFGAFLVG |
| | | | GGGGSGGGGSDKTHT |
| | | | CPPCPAPELLGGPSV |
| | | | FLFPPKPKDTLMISR |
| | | | TPEVTCVVVDVSHED |
| | | | PEVKFNWYVDGVEVH |
| | | | NAKTKPREEQYNSTY |
| | | | RVVSVLTVLHQDWLN |
| | | | GKEYKCKVSNKALPA |
| | | | PIEKTISKAKGQPRE |
| | | | PQVYTLPPCREEMTK |
| | | | NQVSLWCLVKGFYPS |
| | | | DIAVEWESNGQPENN |
| | | | YKTTPPVLDSDGSFF |
| | | | LYSKLTVDKSRWQQG |
| | | | NVFSCSVMHEALHNH |
| | | | YTQKSLSLSPGK |
| SEQ | Trimeric_ | Trimeric | METDTLLLWVLLL |
| ID NO: | hTRAIL_ | human | WVPGSTGTSEETI |
| 6162 | 95-281- | TRAIL | STVQEKQQNISPL |
| | hFc_ | comprising | VRERGPQRVAAHI |
| | Knob_ | residues | TGTRGRSNTLSSP |
| | Cys- | 95-281 | NSKNEKALGRKIN |
| | Blank | | SWESSRSGHSFLS |
| | | | NLHLRNGELVIHE |
| | | | KGFYYIYSQTYFR |
| | | | FQEEIKENTKNDK |
| | | | QMVQYIYKYTSYP |
| | | | DPILLMKSARNSC |
| | | | WSKDAEYGLYSIY |
| | | | QGGIFELKENDRI |
| | | | FVSVTNEHLIDMD |
| | | | HEASFFGAFLVGG |
| | | | GGGSGGGGSGTSE |
| | | | ETISTVQEKQQNI |
| | | | SPLVRERGPQRVA |
| | | | AHITGTRGRSNTL |
| | | | SSPNSKNEKALGR |
| | | | KINSWESSRSGHS |
| | | | FLSNLHLRNGELV |
| | | | IHEKGFYYIYSQT |
| | | | YFRFQEEIKENTK |
| | | | NDKQMVQYIYKYT |
| | | | SYPDPILLMKSAR |
| | | | NSCWSKDAEYGLY |
| | | | SIYQGGIFELKEN |
| | | | DRIFVSVTNEHLI |
| | | | DMDHEASFFGAFL |
| | | | VGGGGGSGGGGSG |
| | | | TSEETISTVQEKQ |
| | | | QNISPLVRERGPQ |
| | | | RVAAHITGTRGRS |
| | | | NTLSSPNSKNEKA |
| | | | LGRKINSWESSRS |
| | | | GHSFLSNLHLRNG |
| | | | ELVIHEKGFYYIY |
| | | | SQTYFRFQEEIKE |
| | | | NTKNDKQMVQYIY |
| | | | KYTSYPDPILLMK |
| | | | SARNSCWSKDAEY |
| | | | GLYSIYQGGIFEL |
| | | | KENDRIFVSVTNE |
| | | | HLIDMDHEASFFG |
| | | | AFLVGGGGGSGGG |
| | | | GSDKTHTCPPCPA |
| | | | PELLGGPSVFLFP |
| | | | PKPKDTLMISRTP |
| | | | EVTCVVVDVSHED |
| | | | PEVKFNWYVDGVE |
| | | | VHNAKTKPREEQY |
| | | | NSTYRVVSVLTVL |
| | | | HQDWLNGKEYKCK |
| | | | VSNKALPAPIEKT |
| | | | ISKAKGQPREPQV |
| | | | YTLPPCREEMTKN |
| | | | QVSLWCLVKGFYP |
| | | | SDIAVEWESNGQP |
| | | | ENNYKTTPPVLDS |
| | | | DGSFFLYSKLTVD |
| | | | KSRWQQGNVFSCS |
| | | | VMHEALHNHYTQK |
| | | | SLSLSPGK |
| SEQ | a_hDR5_ | Antigen | METDTLLLWVLLL |
| ID NO: | Tigatuzumab_ | binding | WVPGSTGEVQLVE |
| 6163 | scFv_VH_ | domain | SGGGLVQPGGSLR |
| | VL- | specific | LSCAASGFTFSSY |
| | hFc_Knob_ | to | VMSWVRQAPGKGL |
| | Cys-Blank | DR5, | EWVATISSGGSYT |
| | | a.k.a. | YYPDSVKGRFTIS |
| | | tigatuzumab | RDNAKNTLYLQMN |
| | | | SLRAEDTAVYYCA |
| | | | RRGDSMITTDYWG |
| | | | QGTLVTVSSGGGG |
| | | | SGGGGSGGGGSGG |
| | | | GGSDIQMTQSPSS |
| | | | LSASVGDRVTITC |
| | | | KASQDVGTAVAWY |
| | | | QQKPGKAPKLLIY |
| | | | WASTRHTGVPSRF |
| | | | SGSGSGTDFTLTI |
| | | | SSLQPEDFATYYC |
| | | | QQYSSYRTFGQGT |
| | | | KVEIKGGGGSGGG |
| | | | GSDKTHTCPPCPA |
| | | | PELLGGPSVFLFP |
| | | | PKPKDTLMISRTP |
| | | | EVTCVVVDVSHED |
| | | | PEVKFNWYVDGVE |
| | | | VHNAKTKPREEQY |
| | | | NSTYRVVSVLTVL |
| | | | HQDWLNGKEYKCK |
| | | | VSNKALPAPIEKT |
| | | | ISKAKGQPREPQV |
| | | | YTLPPCREEMTKN |
| | | | QVSLWCLVKGFYP |
| | | | SDIAVEWESNGQP |
| | | | ENNYKTTPPVLDS |
| | | | DGSFFLYSKLTVD |
| | | | KSRWQQGNVFSCS |
| | | | VMHEALHNHYTQK |
| | | | SLSLSPGK |
| SEQ | a_hDR5_ | Antigen | METDTLLLWVLLL |
| ID NO: | Drozitumab_ | binding | WVPGSTGEVQLVQ |
| 6164 | scFv_VH_VL | domain | SGGGVERPGGSLR |
| | hFc_Knob_ | specific | LSCAASGFTFDDY |
| | Cys | to | AMSWVRQAPGKGL |
| | | DR5, | EWVSGINWQGGST |
| | | a.k.a. | GYADSVKGRVTIS |
| | | drozitumab | RDNAKNSLYLQMN |
| | | | SLRAEDTAVYYCA |
| | | | KILGAGRGWYFDY |
| | | | WGKGTTVTVSSGG |
| | | | GGSGGGGSGGGGS |
| | | | GGGGSSELTQDPA |
| | | | VSVALGQTVRITC |
| | | | SGDSLRSYYASWY |
| | | | QQKPGQAPVLVIY |
| | | | GANNRPSGIPDRF |
| | | | SGSSSGNTASLTI |
| | | | TGAQAEDEADYYC |
| | | | NSADSSGNHVVFG |
| | | | GGTKLTVLGGGGS |
| | | | GGGGSDKTHTCPP |
| | | | CPAPELLGGPSVF |
| | | | LFPPKPKDTLMIS |
| | | | RTPEVTCVVVDVS |
| | | | HEDPEVKFNWYVD |
| | | | GVEVHNAKTKPRE |
| | | | EQYNSTYRVVSVL |
| | | | TVLHQDWLNGKEY |
| | | | KCKVSNKALPAPI |
| | | | EKTISKAKGQPRE |
| | | | PQVYTLPPCREEM |
| | | | TKNQVSLWCLVKG |
| | | | FYPSDIAVEWESN |
| | | | GQPENNYKTTPPV |
| | | | LDSDGSFFLYSKL |
| | | | TVDKSRWQQGNVF |
| | | | SCSVMHEALHNHY |
| | | | TQKSLSLSPGK |
| SEQ | A_hDR5_ | Antigen | METDTLLLWVLLL |
| ID NO: | Conatumumab_ | binding | WVPGSTGQVQLQE |
| 6165 | scFv_VH | domain | SGPGLVKPSQTLS |
| | VL- | specific | LTCTVSGGSISSG |
| | hFc_ | to | DYFWSWIRQLPGK |
| | Knob | DR5, | GLEWIGHIHNSGT |
| | Cys | a.k.a. | TYYNPSLKSRVTI |
| | | conatumumab | SVDTSKKQFSLRL |
| | | | SSVTAADTAVYYC |
| | | | ARDRGGDYYYGMD |
| | | | VWGQGTTVTVSSG |
| | | | GGGSGGGGSGGGG |
| | | | SGGGGSEIVLTQS |
| | | | PGTLSLSPGERAT |
| | | | LSCRASQGISRSY |
| | | | LAWYQQKPGQAPS |
| | | | LLIYGASSRATGI |
| | | | PDRFSGSGSGTDF |
| | | | TLTISRLEPEDFA |
| | | | VYYCQQFGSSPWT |
| | | | FGQGTKVEIKRGG |
| | | | GGSGGGGSDKTHT |
| | | | CPPCPAPELLGGP |
| | | | SVFLFPPKPKDTL |
| | | | MISRTPEVTCVVV |
| | | | DVSHEDPEVKFNW |
| | | | YVDGVEVHNAKTK |
| | | | PREEQYNSTYRVV |
| | | | SVLTVLHQDWLNG |
| | | | KEYKCKVSNKALP |
| | | | APIEKTISKAKGQ |
| | | | PREPQVYTLPPCR |
| | | | EEMTKNQVSLWCL |
| | | | VKGFYPSDIAVEW |
| | | | ESNGQPENNYKTT |
| | | | PPVLDSDGSFFLY |
| | | | SKLTVDKSRWQQG |
| | | | NVFSCSVMHEALH |
| | | | NHYTQKSLSLSPGK |

### T Cell Engagers

The present disclosure provides, inter alia, multispecific (e.g., bi-, tri-, quad-specific) or multifunctional molecules, that are engineered to contain one or more T cell engager that mediate binding to and/or activation of a T cell. Accordingly, in some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIMI, SLAM, CD2, or CD226. In other embodiments, the T cell engager is selected from an antigen binding domain or ligand that binds to and does not activate one or more of CD3, TCRα, TCRβ, TCRγ, TCRζ, ICOS, CD28, CD27, HVEM, LIGHT, CD40, 4-1BB, OX40, DR3, GITR, CD30, TIMI, SLAM, CD2, or CD226.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, multispecific (e.g., bi-, tri-, quad-specific) or multifunctional molecules, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence: QVSHRYPRIQSIKVQFTEYKKEKGFILTSQKEDEIMKVQNNSVIINCDGFYLISLKGYFSQ EVNISLHYQKDEEPLFQLKKVRSVNSLMVASLTYKDKVYLNVTTDNTSLDDFHVNGGE LILIHQNPGEFCVL (SEQ ID NO: 7245), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7245.

In another embodiment, the CD40L comprises the amino acid sequence: MQKGDQNPQIAAHVISEASSKTTSVLQWAEKGYYTMSNNLVTLENGKQLTVKRQGLY YIYAQVTFCSNREASSQAPFIASLCLKSPGRFERILLRAANTHSSAKPCGQQSIHLGGVFE LQPGASVFVNVTDPSQVSHGTGFTSFGLLKL (SEQ ID NO: 7246), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7246.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In one embodiment, the immune cell engager includes 41BB ligand, e.g., comprising the amino acid sequence: ACPWAVSGARASPGSAASPRLREGPELSPDDPAGLLDLRQGMFAQLVAQNVLLIDGPLS WYSDPGLAGVSLTGGLSYKEDTKELVVAKAGVYYVFFQLELRRVVAGEGSGSVSLALH LQPLRSAAGAAALALTVDLPPASSEARNSAFGFQGRLLHLSAGQRLGVHLHTEARARH AWQLTQGATVLGLFRVTPEIPAGLPSPRSE (SEQ ID NO: 7247), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7247.

### Toll-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63.)

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

TLR-9

TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

TLR Agonists

A TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Linkers

The multispecific or multifunctional molecule disclosed herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In embodiments, the linker is chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser. In some embodiments, the peptide linker is selected from GGGGS (SEQ ID NO: 7248); GGGGSGGGGS (SEQ ID NO: 7249); GGGGSGGGGSGGGGS (SEQ ID NO: 7250); and DVPSGPGGGGGSGGGGS (SEQ ID NO: 7251). In some embodiments, the peptide linker is a A(EAAAK)nA (SEQ ID NO: 7291) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 7252); AEAAAKEAAAKEAAAKAAA (SEQ ID NO: 7253); AEAAAKEAAAKEAAAKEAAAKAAA (SEQ ID NO: 77); and AEAAAKEAAAKEAAAKEAAAKEAAAKAAA (SEQ ID NO: 78).

### Nucleic Acids

Nucleic acids encoding the aforementioned multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding a multispecific or multifunctional molecule described herein. In one embodiment, the vectors comprise nucleotides encoding a multispecific or multifunctional molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

Methods described herein include treating an immune condition or disorder, e.g., an autoimmune disease, in a subject by using a multispecific molecule described herein, e.g., using a pharmaceutical composition described herein. Also provided are methods for reducing or ameliorating a symptom of an autoimmune disorder, e.g., autoimmune disease, in a subject, as well as methods for correcting or decreasing a TCR bias (e.g., re-establishing a balanced TCR repertoire or a TCR repertoire more similar to a person without an autoimmune disease).

In one embodiment, the immune condition or disorder is chosen from Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellar degeneration (PCD), Lambert-Eaton syndrome, psoriasis, scleroderma, CREST (calcinosis, Raynaud phenomenon, esophageal dysmotility, sclerodactyly, and telangiectasia) syndrome, dermatitis herpetiformis, dermatomyositis, bullous pemphigoid, cicatricial pemphigoid/benign mucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis), Takayasu's arteritis, polyarteritis nodosa, microscopic polyangiitis (MPA), leukocytoclastic vasculitis, Cogan's syndrome, uveitis, peripheral uveitis (Pars planitis), scleritis, autoimmune inner ear disease (AIED), Crohn's, ulcerative colitis (UC), Dressler's syndrome, Rheumatic fever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), Sjogren's syndrome, ocular cicatricial pemphigoid, or Hashimoto's thyroiditis.

In some embodiments, the immune condition or disorder, e.g., autoimmune disease, is diabetes (e.g., type 1 diabetes).

In embodiments, the multispecific molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific molecules) to be administered can be determined by a physician with consideration of individual differences in severity/character of immune disorder, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the multispecific molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation. In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multispecific or multifunctional molecules disclosed herein can be used in combination with a second therapeutic agent or procedure.

In embodiments, the multispecific or multifunctional molecule and the second therapeutic agent or procedure are administered/performed after a subject has been diagnosed with an autoimmune disorder. In embodiments, the multispecific or multifunctional molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or T cells comprising a TCRBV antigen corresponding to a biased TCRBV clonotype cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multispecific or multifunctional molecule is administered in combination with a therapy, e.g., an autoimmune disease therapy known in the art. The administration of the multispecific or multifunctional molecule and the therapy can be sequential (with or without overlap) or simultaneous. Administration of the multispecific or multifunctional molecule can be continuous or intermittent during the course of therapy.

### Immune Checkpoint Inhibitors

In other embodiments, methods described herein comprise use of an immune checkpoint inhibitor in combination with the multispecific or multifunctional molecule. The methods can be used in a therapeutic protocol in vivo.

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GALS, VISTA, BTLA, TIGIT, LAIR1, and A2aR. See, e.g., Pardoll. Nat. Rev. Cancer 12.4 (2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.570. The YW243.55.570 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech /Roche), which is a human Fc-optimized IgG1 monoclonal antibody against PD-L1. See, e.g., U.S. Pat. No. 7,943,743 and U.S Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### INCORPORATION BY REFERENCE

All publications and patents mentioned herein are hereby incorporated by reference in their entirety as if each individual publication or patent was specifically and individually indicated to be incorporated by reference.

### EXAMPLES

### Example 1: Immunization of Armenian Hamster to Generate Anti-NKp30 Antibodies

Briefly, armenian hamster were immunized with the extracellular domain of human NKp30 protein in complete Freund's adjuvant and boosted twice on day 14 and day 28 with NKp30 in incomplete Freund's adjuvant (IFA). On day 56 one more boost in IFA was given and the animals harvested three days later. Spleens were collected and fused with P3X63Ag8.653 murine myeloma cell line. 0.9×10\hat{ }5 cells/well in 125 ul were seated in 96 well plate and feed with 125 μl of I-20+2ME HAT (IMDM (4 g/L, glucose) supplemented with 20% fetal bovine serum, 4 mM L-glutamine, 1 mM sodium pyruvate, 50 U penicillin, 50 μg streptomycin and 50 μM 2-ME in the absence or presence of HAT or HT for selection, and Hybridoma Cloning Factor (1% final) on days 7, 11 and thereafter as needed. At approximately 2 weeks after fusion (cells are about 50% confluent) supernatant was collected and assayed for binding.

### Example 2: Hybridoma Screen for NKp30 mAbs

Expi293 cells were transfected with BG160 (hNKp30 cell antigen) 18 hours prior to screening. The day of screening, transfected cells were diluted to 0.05×10\hat{ }6/mL and anti-Armenian hamster Fc Alexa Fluor 488 added to a final concentration of 0.4 ug/mL. 50 (2,500 cells) of this mixture was added to each well of a 384 well plate. The same density of untransfected 293 cells with secondary were used as a negative control. 5 uL of hybridoma supernatant was added to the cell mixture and the plate incubated for 1 hour at 37° C. The plates were then imaged on Mirrorball. Positive clones were identified and subcloned by serial dilution to obtain clonal selected hybridoma, After reconfirmation using the same protocols the hybridoma cells were harvested and the corresponding heavy and light chain sequences recovered. The DNA was subcloned into pcDNA3.4 for subsequent expression of the corresponding antibodies and further validation.

### Example 3: Binding of NKp30 Antibodies to NK92 Cells

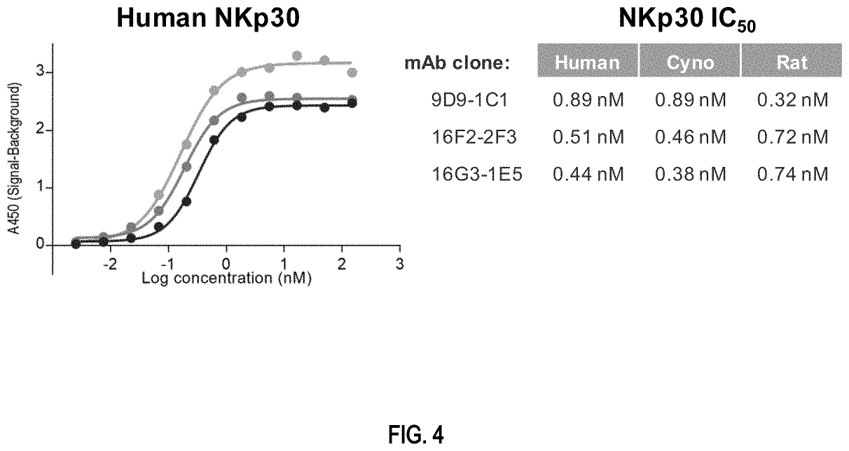

NK-92 cells were washed with PBS containing 0.5% BSA and 0.1% sodium azide (staining buffer) and added to 96-well V-bottom plates with 200,000 cells/Well. Hamster NKp30 antibodies were added to the cells in 2.0 fold serial dilutions and incubated for 1 hour at room temperature. The plates were washed twice with staining buffer. The secondary antibody against hamster Fc conjugated to AF647 (Jackson, 127-605-160) was added at 1:100 dilution (1.4 mg/ml stock) and incubated with the cells for 30 minutes at 4° C.′ followed by washing with staining buffer. Cells were subsequently were fixed for 10 minutes with 4% paraformaldehyde at room temperature. The plates were read on CytoFLEX LS (Beckman Coulter). Data was calculated as the percent-AF747 positive population (FIG. 4).

### Example 4: Bioassay to Measure Activity of NKp30 Antibodies Using NK92 Cell Line

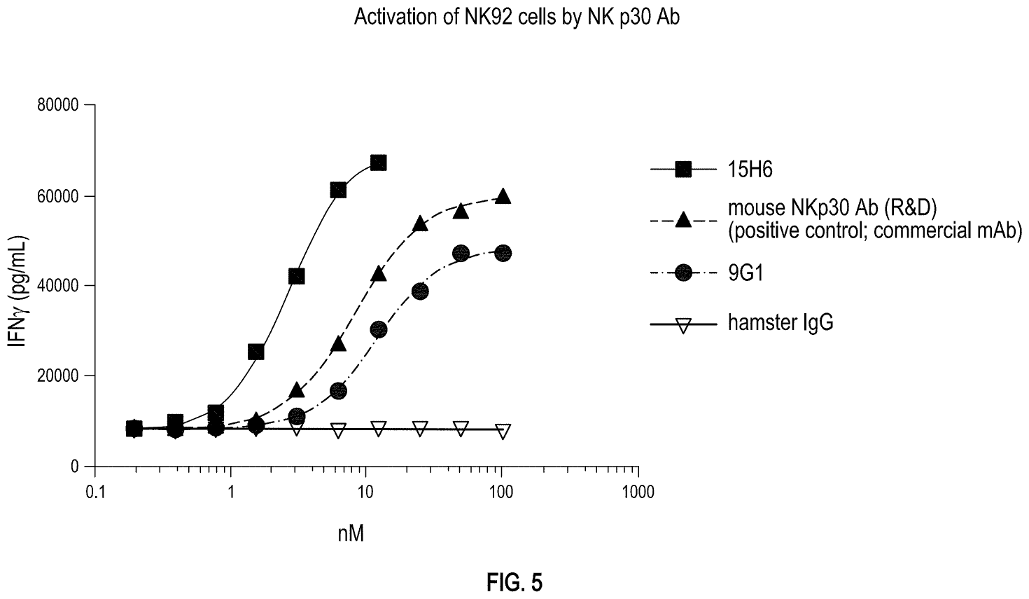

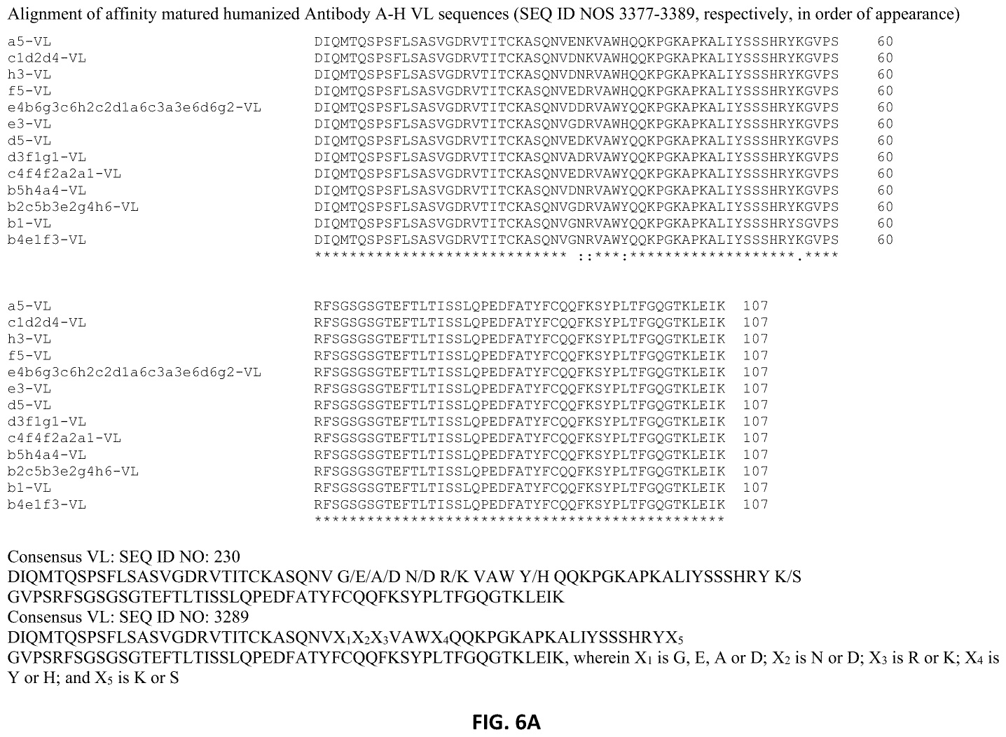

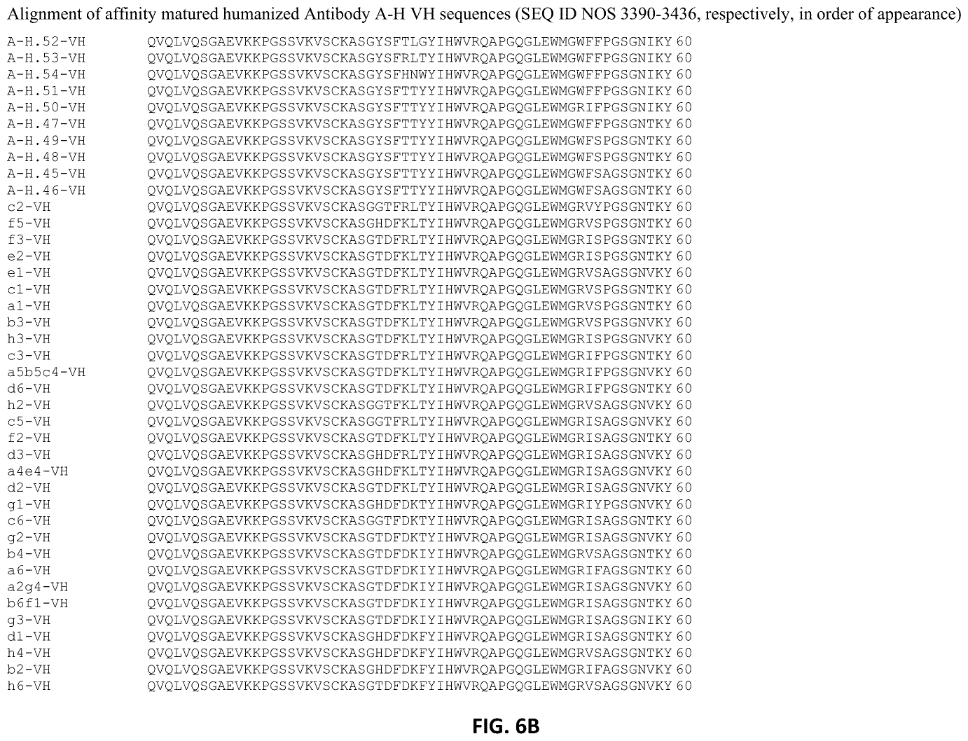

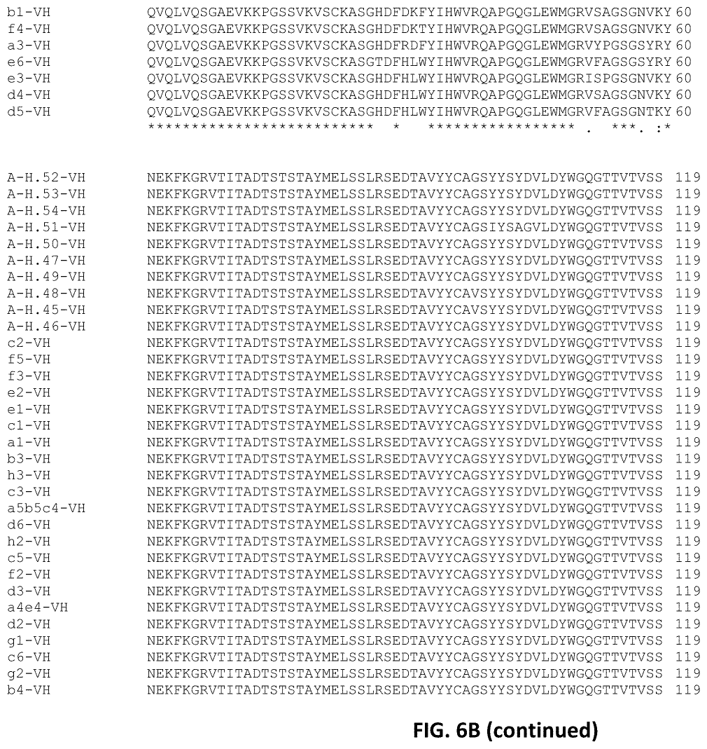

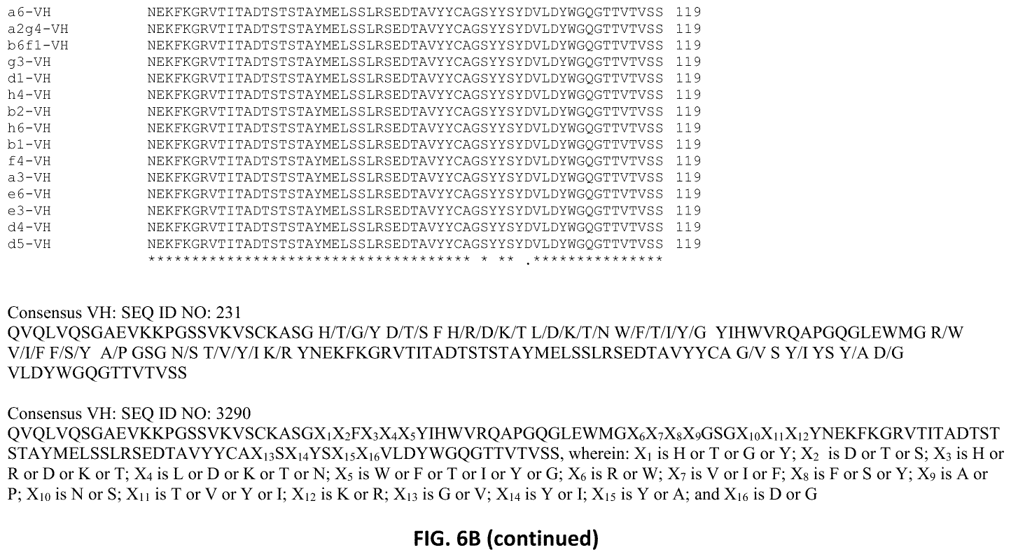

NKp30 antibodies were three-fold serially diluted in PBS and incubated at 2-8° C. overnight in flat bottom 96 well plates. Plates were washed twice in PBS and 40,000 NK-92 cells were added in growth medium containing IL-2. Plates were incubated at 37° C., 5% CO2, humidified incubator for 16-24 hours before supernatants were collected. IFNγ levels in supernatants was measured following MSD assay instructions (FIG. 5). Supernatant collected from cells incubated with hamster isotype IgG was used as negative control and supernatants from cells incubated with NKp30 monoclonal antibody (R&D, clone 210847) was utilized as a positive control. Data were generated using hamster anti-NKp30 mABs.

### Example 5: Generation and Characterization of Humanized Anti-NKp30 Antibodies

A series of hamster anti-NKp30 antibodies were selected. These antibodies were shown to bind to human NKp30 and cynomolgus NKp30 and induce IFNγ production from NK-90 cells (data not shown). The VH and VL sequences of exemplary hamster anti-NKp30 antibodies 15E1, 9G1, 15H6, 9D9, 3A12, and 12D10 are disclosed in Table 9. The VH and VL sequences of exemplary humanized anti-NKp30 antibodies based on 15E1, 9G1, and 15H6 are also disclosed in Table 9. The Kabat CDRs of these antibodies are disclosed in Table 18 and Table 8.

Two humanized constructs based on 15E1 were selected. The first construct BJM0407 is a Fab comprising a heavy chain variable region comprising the amino acid sequence of SEQ ID NO: 7302 and a lambda light chain variable region comprising the amino acid sequence of SEQ ID NO: 7305. Its corresponding scFv construct BJM0859 comprises the amino acid sequence of SEQ ID NO: 7310. The second construct BJM0411 is a Fab comprising a heavy chain variable region comprising the amino acid sequence of SEQ ID NO: 7302 and a kappa light chain variable region comprising the amino acid sequence of SEQ ID NO: 7309. Its corresponding scFv construct BJM0860 comprises the amino acid sequence of SEQ ID NO: 7311. BJM0407 and BJM0411 showed comparable biophysical characteristics, e.g., binding affinity to NKp30 and thermal stability. The scFv constructs BJM0859 and BJM0860 also showed comparable biophysical properties.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A multifunctional molecule, comprising:
(i) a first antigen binding domain that binds to, e.g., selectively binds to, T cell receptor variable beta (TCRBV), e.g., a TCRBV antigen,
and
(ii) one, two, or all of:
(a) an immune cell engager chosen from an NK cell engager, a T cell engager, a B cell engager, a dendritic cell engager, or a macrophage cell engager;
(b) a cytokine molecule or cytokine inhibitor molecule; and
(c) a death receptor signal engager.

2. A multifunctional molecule, comprising:
(i) a first antigen binding domain that binds to, e.g., selectively binds to, T cell receptor variable beta (TCRBV), e.g., a TCRBV antigen, and
(ii) an NK cell engager, e.g., an anti-NKp30, anti-NKp46, anti-NKG2D, or anti-CD16 antibody molecule.

3. The multifunctional molecule of claim 2, wherein the NK cell engager comprises an anti-NKp30 antibody molecule.

4. The multifunctional molecule of claim 2, wherein the NK cell engager comprises an anti-NKp46 antibody molecule.

5. A multifunctional molecule, comprising:
(i) a first antigen binding domain that binds to, e.g., selectively binds to, T cell receptor variable beta (TCRBV), e.g., a TCRBV antigen, and
(ii) a death receptor signal engager.

6. A multifunctional molecule, comprising:
(i) a first antigen binding domain that binds to, e.g., selectively binds to, T cell receptor variable beta (TCRBV), e.g., a TCRBV antigen, and
(ii) a cytokine inhibitor molecule.

7. A nucleic acid molecule encoding the multifunctional molecule of any one of claims 1-6.

8. A vector, e.g., an expression vector, comprising the nucleic acid molecules of claim 7.

9. A host cell comprising the nucleic acid molecule of claim 7 or the vector of claim 8.

10. A method of making, e.g., producing, the multifunctional molecule or antibody molecule of any one of claims 1-6, comprising culturing the host cell of claim 9, under suitable conditions, e.g., conditions suitable for gene expression and/or homo- or heterodimerization.

11. A pharmaceutical composition comprising the multifunctional molecule of any one of claims 1-6 and a pharmaceutically acceptable carrier, excipient, or stabilizer.

12. A method of treating a TCR bias, comprising administering to a subject in need thereof the multifunctional molecule of any one of claims 1-6, wherein the multifunctional molecule is administered in an amount effective to treat the TCR bias.

13. A method of treating an autoimmune disease (e.g., an autoimmune disease associated with a TCR bias), comprising administering to a subject in need thereof the multifunctional molecule of any one of claims 1-6, wherein the multifunctional molecule is administered in an amount effective to treat the autoimmune disease.

14. A method of treating a TCR bias, comprising:
responsive to determining that a subject has a TCR bias, administering to a subject in need thereof the multifunctional molecule of any one of claims 1-6, wherein the multifunctional molecule is administered in an amount effective to treat the TCR bias.

15. A method of treating an autoimmune disease (e.g., an autoimmune disease associated with a TCR bias), comprising:
responsive to determining that a subject has an autoimmune disease (e.g., an autoimmune disease associated with a TCR bias), administering to a subject in need thereof the multifunctional molecule of any one of claims 1-6, wherein the multifunctional molecule is administered in an amount effective to treat the autoimmune disease (e.g., an autoimmune disease associated with a TCR bias).

16. A method of identifying a subject in need of treatment for cancer using a multifunctional molecule of any of claims 1-6, comprising determining (e.g., directly determining or indirectly determining, e.g., obtaining information regarding) whether a subject has a TCR bias (e.g., a biased TCRBV clonotype) and/or an autoimmune disease associated with said bias, wherein:
responsive to determining that the subject has a TCR bias (e.g., a biased TCRBV clonotype) and/or an autoimmune disease associated with said bias, identifying the subject as a candidate for treatment using a multifunctional molecule comprising an antigen binding domain that binds to the TCRBV antigen, and optionally not as a candidate for treatment using a multifunctional molecule comprising an antigen binding domain that does not bind to the TCRBV antigen (e.g., that binds to a different TCRBV antigen).

17. A method of evaluating a subject in need of treatment for a TCR bias (e.g., a biased TCRBV clonotype) and/or an autoimmune disease associated with said bias, comprising determining (e.g., directly determining or indirectly determining, e.g., obtaining information regarding) whether a subject has a TCR bias.

18. A method of treating an autoimmune disease (e.g., an autoimmune disease associated with a TCR bias), in a subject in need thereof, comprising administering to said subject an effective amount, e.g., a therapeutically effective amount, of an antibody molecule which binds (e.g., specifically binds) to a T cell receptor beta variable region (TCRβV) (“anti-TCRβV antibody molecule”), thereby treating the disorder.

19. A method of depleting a population of T cells in a subject having an autoimmune disorder (e.g., an autoimmune disease associated with a TCR bias), comprising, contacting the T cell population with an effective amount of an antibody molecule which binds (e.g., specifically binds) to a T cell receptor beta variable region (TCRβV) (“anti-TCRβV antibody molecule”).

20. The method of claim 19, wherein the contacting occurs in vivo or in vitro.

21. The method of any one of claims 18-20, wherein the anti-TCRβV antibody molecule:
(i) is not an antibody molecule disclosed in U.S. Pat. No. 5,861,155;
(ii) binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155;
(iii) binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155;
(iii) binds to TCRβ V5-5*01 or TCRβ V5-1*Ol with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155 or
(iv) binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

22. The method of any one of claims 18-21, wherein the anti-TCRβV antibody molecule comprises an Fc region, e.g., an Fc region having effector function, e.g., antibody dependent cell-mediated cytotoxicity (ADCC), Antibody-dependent cellular phagocytosis (ADCP) and/or complement dependent cytotoxicity (CDC).

23. The method of any claim 22, wherein the anti-TCRβV antibody molecule comprises an Fc region with enhanced effector function, e.g., as compared to a wildtype Fc region.

24. The method of any one of claims 18-23, wherein the anti-TCRβV antibody molecule comprises a human IgG1 region or a human IgG4 region.
